(19) United States
(12) Patent Application Publication
(10) Pub. No.: US 2022/0390432 A1
(43) Pub. Date: Dec. 8, 2022

(54) METHODS OF CHARACTERIZING AND UTILIZING AGENT-CONDENSATE INTERACTIONS

(71) Applicant: Whitehead Institute for Biomedical Research, Cambridge, MA (US)

(72) Inventors: Richard A. Young, Boston, MA (US); Ann Boija, Somerville, MA (US); Isaac Klein, Milton, MA (US)

(21) Appl. No.: 17/611,560

(22) PCT Filed: May 15, 2020
(86) PCT No.: PCT/US2020/033295

§ 371 Date: Nov. 15, 2021

Related U.S. Application Data
(60) Provisional application No. 62/927,073, filed on Oct. 28, 2019.
(60) Provisional application No. 62/848,539, filed on May 15, 2019.

(51) Int. Cl. G01N 33/50; C12Q 1/6841

(52) U.S. Cl. CPC G01N 33/5011; C12Q 1/6841; C12Q 2600/158; G01N 2500/02

(57) ABSTRACT

Described herein are methods of characterizing agent incorporation into condensates, methods of reducing transcription of oncogenes associated with condensates, and methods of using peptides to inhibit nuclear receptor and cofactor binding in condensates.

### RELATED APPLICATIONS

This application claims the benefit of U.S. Provisional Application Ser. No. 62/848,539, filed May 15, 2019, and U.S. Provisional Application Ser. No. 62/927,073, filed Oct. 28, 2019, the contents of which is hereby incorporated by reference in its entirety.

### GOVERNMENT SUPPORT

This invention was made with government support under GM123511, CA213333, and CA155258 awarded by The National Institutes of Health, as well as PHY1743900 awarded by The National Science Foundation. The government has certain rights in the invention.

### BACKGROUND OF THE INVENTION

The transcription factors and cofactors that occupy super-enhancers form liquid-like condensates that compartmentalize and concentrate the transcription apparatus at key cell identity genes. Tumor cells acquire large super-enhancers at driver oncogenes, thus contributing to the transcriptional dysregulation that is a hallmark of cancer.

### SUMMARY OF THE INVENTION

Herein it is shown that transcriptional condensates are implicated in driving oncogenesis and provide a framework for new entry points in cancer therapeutics. The finding that multiple proteins key to the transcriptional machinery reside within these structures may make previously undruggable targets (due to their disorder nature) attractive as drug targets. Unexpectedly, it is shown herein that some agents (e.g., small molecules) enter transcriptional condensates independent of the presence of the agent target. The methods disclosed herein measuring the degree to which agents can enter condensates and the specificity the agents have for different types of condensates (e.g. transcriptional condensates, heterochromatin or repressive condensates, splicing speckle condensate, nucleolus, chromatin condensate, polycomb condensate, DNA damage repair condensate) provide valuable information regarding drug exposure and off target effects. Methods disclosed herein can determine how much of a drug is partitioned in a condensate and how much is partitioned outside a condensate to aid in determining efficacy of a candidate in a cell or organism. Also provided are methods of modulating incorporation of agents into condensates via modulating the number of aromatic sidechains on agents or condensate components. In addition, ascertaining how drugs work in condensates may enable adoption of known drugs for new purposes.

Some aspects of the invention are related to a method of characterizing an agent, comprising contacting the agent with a composition comprising a condensate having at least one component, and measuring incorporation of the agent into the condensate. In some embodiments, incorporation of the agent into the condensate is detected without using a detectable tag on the agent. In some embodiments, incorporation of the agent into the condensate is detected using Raman spectroscopy, spectrophotometry and quantitative phase microscopy, or spin down assay. In some embodiments, the agent comprises a detectable tag. In some embodiments, the component or condensate comprises a detectable tag. In some embodiments, the detectable tag is a fluorescent tag.

In some embodiments, the method comprises contacting the agent having a detectable tag with the composition comprising the condensate, measuring incorporation of the agent having a detectable tag into the condensate, contacting the composition comprising the condensate and the agent having a detectable tag with a control agent not having a detectable tag, and again measuring incorporation of the agent having a detectable tag into the condensate.

In some embodiments, the method comprises contacting the agent with a plurality of condensates having one or more different components. In some embodiments, the method comprises contacting the agent with a plurality of compositions each having a condensate having at least one different component. In some embodiments, the method comprises contacting a plurality of agents with a plurality of compositions each having condensates comprising the same components.

In some embodiments, the least one component is a transcriptional condensate component, a heterochromatin condensate component, a component of a condensate physically associated with mRNA initiation, a component of a condensate physically associated with mRNA elongation, a component of a chromatin condensate, a component of a polycomb condensate, or a component of a DNA damage repair condensate. In some embodiments, the least one component is mediator, a mediator component, MED1, BRD4, POLII (i.e., POL2), SRSF2, FIB1, NPM1, HP1α, histone, histone tail portion, a polycomb repressive complex 1 (PRC1) component (e.g., CBX2), or 53BP1. In some embodiments, the at least one component is a component or functional portion thereof of a super-enhancer condensate, splicing speckle condensate, heterochromatin condensate, nucleolus, chromatin condensate, polycomb condensate, or DNA damage repair condensate. In some embodiments, the component comprises an intrinsically disordered region (IDR).

In some embodiments, the component comprises a different detectable tag than the agent. In some embodiments, the incorporation of the agent is measured relative to a control. In some embodiments, the incorporation of a plurality of agents is measured and compared to each other.

In some embodiments, the agent is capable of binding to a target. In some embodiments, the condensate does not comprise the target. In some embodiments, the target is present primarily outside the condensate. In some embodiments, the target is present primarily in the condensate. In some embodiments, the target is a therapeutic target. In some embodiments, the target is an enzyme, a receptor, a ligand, an oncogene, an oncogene product, or a transcription factor. In some embodiments, the target is genomic DNA. In some embodiments, the composition comprises the target.

In some embodiments, the relative amount of agent incorporated in the condensate, or not incorporated in the condensate, is measured. In some embodiments, the condensate is physically associated with DNA.

In some embodiments, the condensate is in a cell. In some embodiments, the cell is a diseased cell. In some embodiments, the condensate is in vitro. In some embodiments, the agent is a small molecule, a polypeptide, or a nucleic acid. In some embodiments, the agent is a known chemotherapeutic agent. In some embodiments, the agent is a candidate chemotherapeutic agent. In some embodiments, the agent is or comprises cisplatin or a derivative thereof. In some embodiments, the agent is or comprises JQ1 ((S)-tert-butyl 2-(4-(4-chlorophenyl)-2,3,9-trimethyl-6H-thieno[3,2-ƒ][1,2,4]triazolo[4,3-a][1,4]diazepin-6-yl)acetate) or a derivative thereof. In some embodiments, the agent is or comprises tamoxifen or a derivative thereof.

Some aspects of the invention are directed to a method of characterizing a first agent, comprising contacting the first agent with a composition comprising a condensate having at least one component, wherein the condensate contains at least a second agent, and measuring the ability of the first agent to cause eviction of the second agent from the condensate. In some embodiments, the second agent comprises a detectable tag. In some embodiments, the detectable tag is a fluorescent tag. In some embodiments, the condensate component is a target for the second agent.

Some aspects of the invention are directed to a composition comprising a condensate and an agent having a therapeutic target, wherein the condensate does not comprise the therapeutic target. In some embodiments, the therapeutic target is genomic DNA.

As shown in the examples below, a dye that does not preferentially partition into a condensate can be modified to preferentially partition into a condensate by coupling with an agent or moiety. Some aspects of the invention are directed to a method of modulating the partitioning of a first agent into a condensate comprising coupling the first agent to a second agent, thereby modulating the partitioning of the first agent into the condensate. In some embodiments, the condensate is selected from a super-enhancer condensate, splicing speckle condensate, heterochromatin condensate, or nucleolus. In some embodiments, the partitioning of the first agent into the condensate is increased. In some embodiments, the partitioning of the first agent into the condensate is decreased. In some embodiments, the therapeutic efficacy of the coupled first agent is increased as compared to uncoupled first agent. In some embodiments, side effects of the coupled first agent are decreased as compared to uncoupled first agent.

As also shown in the examples below, increasing the aromatic side chain content of an agent increases partitioning of the agent in MED1 in vitro condensates (i.e., droplets). Some aspects of the invention are directed to a method of modulating partitioning of an agent in a condensate by modifying the agent to increase or decrease the number of aromatic side chains. In some embodiments, the partitioning of the modified agent into the condensate is increased as compared to an unmodified agent. In some embodiments, the portioning of the modified agent is decreased as compared to an unmodified agent.

Some aspects of the present disclosure are directed to a method of screening for a candidate agent with modulated condensate partitioning comprising modifying an agent with a condensate partition coefficient and measuring the condensate partition coefficient of the modified agent, wherein if the modified agent has a different partition coefficient than the agent, then the modified agent is identified as a candidate agent with modulated condensate partitioning. In some embodiments, the condensate partition coefficient of the modified agent is measured in an in vitro condensate. In some embodiments, the condensate partition coefficient of the modified agent is measured in a condensate in a cell. In some embodiments, the candidate agent is identified as an improved candidate agent if the candidate agent has increased partitioning into a condensate having a therapeutic target for the candidate agent. In some embodiments, the candidate agent is identified as an improved candidate agent if the candidate agent has decreased partitioning into a condensate not having a therapeutic target for the candidate agent. In some embodiments, the candidate agent with modulated condensate partitioning is a chemotherapeutic agent. In some embodiments, the modification comprises an increase or decrease in the number of aromatic side chains of the agent.

Some aspects of the invention are directed to a method of reducing transcription of an oncogene, comprising modulating the composition of, dissolving, or disassociating a transcriptional condensate associated with the oncogene by contacting the transcriptional condensate with an agent.

In some embodiments, the agent dissolves the transcriptional condensate, causes the transcriptional condensate to uncouple from genomic DNA comprising the oncogene, or evicts one or more components of the transcriptional condensate. In some embodiments, the agent is an inhibitor, intercalator, or cyclin dependent kinase inhibitor. In some embodiments, the agent binds to a component of the transcriptional condensate. In some embodiments, the agent preferentially concentrates in the transcriptional condensate. In some embodiments, the condensate is located in a cell. In some embodiments, the cell is a cancer cell.

In some embodiments, the agent is administered to a subject having cancer. In some embodiments, the cancer is colon cancer, lymphoma, multiple myeloma, prostate cancer, or breast cancer.

Some aspects of the present invention are related to a method of treating a subject in need of treatment for cancer characterized by transcription of an oncogene, the method comprising administering to the subject an agent that modulates the composition of, dissolves, or disassociates a transcriptional condensate associated with the oncogene. In some embodiments, the agent is an inhibitor, intercalator, or cyclin dependent kinase inhibitor. In some embodiments, the agent binds to a component of the transcriptional condensate. In some embodiments, the agent preferentially concentrates in the transcriptional condensate. In some embodiments, the cancer is colon cancer, lymphoma, multiple myeloma, prostate cancer, or breast cancer.

In some embodiments, the subject is human. In some embodiments, the agent is administered orally, subcutaneously, topically, or intravenously to the subject. In some embodiments, the agent is a small molecule, a polypeptide, or a nucleic acid.

Some aspects of the disclosure are directed to a method of inhibiting transcription associated with a transcriptional condensate, comprising inhibiting the binding of a nuclear receptor associated with the transcriptional condensate to a cofactor having an LXXLL domain, wherein the binding is inhibited by contacting the condensate with a peptide that binds to the LXXLL domain.

In some embodiments, the nuclear receptor is a nuclear hormone receptor, an Estrogen Receptor, or a Retinoic Acid Receptor-Alpha. In some embodiments, the cofactor is MED1. In some embodiments, the transcription of an oncogene is inhibited. In some embodiments, the transcriptional condensate is located in a cell. In some embodiments, the cell is a cancer cell. In some embodiments, the peptide is administered to a subject. In some embodiments, the subject has cancer.

Some aspects of the invention are directed to a method of inhibiting transcription associated with a transcriptional condensate, comprising inhibiting the binding of a nuclear receptor having an LXXLL binding domain and associated with the transcriptional condensate to a cofactor having an LXXLL domain, wherein the binding is inhibited by contacting the condensate with a peptide that binds to the LXXLL domain.

In some embodiments, the nuclear receptor is a nuclear hormone receptor, an Estrogen Receptor, or a Retinoic Acid Receptor-Alpha. In some embodiments, the cofactor is MED1. In some embodiments, the transcription of an oncogene is inhibited. In some embodiments, the transcriptional condensate is located in a cell. In some embodiments, the cell is a cancer cell. In some embodiments, the peptide is administered to a subject. In some embodiments, the subject has cancer.

Some aspects of the invention are directed to a composition comprising a cell having a first condensate comprising a first detectable label and a second condensate having a different second detectable label, wherein the first and second condensate are different condensate types selected from a super-enhancer condensate, splicing speckle condensate, heterochromatin condensate, nucleolus, chromatin condensate, polycomb condensate, or DNA damage repair condensate. In some embodiments, the composition further comprises an agent contacted with the cell. In some embodiments, the agent is a known therapeutic agent. In some embodiments, the agent is a candidate therapeutic agent. In some embodiments, the second detectable label is detectably distinguishable from the first detectable label.

Some aspects of the invention are directed to a composition comprising a first in vitro condensate, a second in vitro condensate and an agent contacted with the first and second in vitro condensate. In some embodiments, at least one of the first in vitro condensate, second in vitro condensate, and the agent comprises a detectable label. In some embodiments, the composition further comprises a third in vitro condensate, and optionally a fourth in vitro condensate, each contacted with the agent. In some embodiments, at least one of the in vitro condensates comprises a component, or functional fragment thereof, of a transcriptional condensate, super-enhancer condensate, splicing speckle condensate, heterochromatin condensate, nucleolus, chromatin condensate, polycomb condensate, or DNA damage repair condensate. Some embodiments are directed towards an article comprising a first in vitro condensate contacted with an agent, a second in vitro condensate contacted with the same agent, and a multi-well plate separating the first and second in vitro condensates into separate wells. In some embodiments, the article further comprises at least a third in vitro condensate contacted with the agent. In some embodiments, the article further comprises at least a fourth in vitro condensate contacted with the agent. The first, second, third and fourth in vitro condensates can each comprise a component or functional fragment thereof of a different condensate (e.g., a super-enhancer condensate, splicing speckle condensate, heterochromatin condensate, nucleolus, chromatin condensate, polycomb condensate, or DNA damage repair condensate). The first, second, third and fourth in vitro condensates can each comprise a different detectable label.

Some aspects of the invention are directed to a method of assessing whether differential expression of one or more condensate components by a cell resistant to an agent causes or contributes to the resistance, comprising providing an agent-resistant cell, contacting the agent-resistant cell with the agent, and assessing localization, concentration, and/or therapeutic activity of the agent as compared to a control.

Some aspects of the invention are directed to a method of assessing whether differential expression of one or more condensate components by a cell resistant to an agent causes or contributes to the resistance, comprising providing a condensate isolated from an agent-resistance cell, contacting the condensate with the agent, and assessing localization, concentration, and/or therapeutic activity of the agent as compared to a control.

Some aspects of the invention are directed to a method of assessing whether differential expression of one or more condensate components by a cell resistant to an agent causes or contributes to the resistance, comprising providing an in vitro condensate (e.g., droplet) comprising a differential amount of a condensate component, or fragment thereof, that is differentially expressed in an agent-resistant cell, contacting the condensate with the agent, and assessing localization, concentration, and/or therapeutic activity of the agent as compared to a control.

Some aspects of the invention are directed to a method of assessing whether differential expression of one or more condensate components by a cell resistant to an agent causes or contributes to the resistance, comprising providing an in vitro condensate (e.g., droplet) comprising a mutant condensate component, or fragment thereof, corresponding to a mutant condensate component in an agent-resistant cell, contacting the condensate with the agent, and assessing localization, concentration, and/or therapeutic activity of the agent as compared to a control.

Some aspects of the invention are directed to a method of characterizing an agent-resistance condensate comprising contacting the condensate with one or more second agents and assessing at least one of agent localization, concentration, or therapeutic activity and/or condensate morphology, stability, or dissolution. In some embodiments, the second agent is contacted with a cell comprising the agent-resistant condensate. In some embodiments, the condensate has been isolated from a cell. In some embodiments, the condensate is an in vitro condensate (e.g., droplet). In some embodiments, the condensate comprises a mutant form of a condensate component or fragment thereof associated with resistance to the agent.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic showing that alterations in the transcription apparatus are a hallmark of cancer. Adapted from Bradner, Hnisz and Young, Cell 2017.

FIG. 2 shows ChIP-seq data identifies super-enhancers. Super-enhancers are large clusters of enhancers that regulate genes with prominent roles in cell identity that are occupied by an exceptionally high density of proteins enriched in intrinsic disorder domains and high levels of eRNA. Adapted from Hnisz et al., Cell (2013).

FIG. 3 shows tumor cells acquire exceptionally large super-enhancers at driver oncogenes that can be nucleated by small changes in DNA, and are especially sensitive to transcriptional drugs. Illustrations adapted from Mansour et al. Science (2014) and Loven et al. Cell (2013).

FIG. 4 shows transcription factors and Mediator coactivator contribute to formation of condensates at super-enhancers. See, Sabari, Dall'Agnese et al., Science 2018; Cho, Spille et al., Science 2018; and Boija, Klein et al., Cell 2018.

FIG. 5 shows biomolecular condensates can be produced through phase separation. Adapted from Brangwynne C P. JCB 2013.

FIG. 6 shows transcriptional condensates are involved in oncogene expression and are exploitable therapeutic targets. TxEx-transcription enzymes; TF-transcription factor; CoA-co-activator; SE-driven oncogene-super-enhancer driven oncogene.

FIGS. 7A-7C show transcriptional condensates containing driver transcription factor (TF) and MED1 subunit of Mediator occur at the MYC oncogene in human tumor tissue. FIG. 7A shows a breast cancer carcinoma and H&E staining of ER+ breast cancer. FIG. 7B shows immunofluorescent microscopy of ER+ breast cancer tissue using an antibody against MED1 (MED1 IF) or an antibody against estrogen receptor (ER IF) followed by Myc RNA FISH. Top right panel (Merge Zoom) show that MED1 and Myc transcription are co-localized in condensates. Bottom right panel shows estrogen receptor and Myc transcription are co-localized in condensates. FIG. 7C shows immunofluorescent microscopy on ER+ breast cancer tissue using an antibody against MED1 (MED1 IF) or an antibody against estrogen receptor (ER IF) followed by Myc DNA FISH. Top right panel (Merge Zoom) show that MED1 and the Myc gene are co-localized in condensates. Bottom right panel shows estrogen receptor and the Myc gene are co-localized in condensates.

FIG. 8 shows Mediator condensates are present on MYC in a variety of cancer cell types.

FIG. 9 shows ER bound DNA facilitates MED1 condensate formation. MED1 and ER formed droplets in an in vitro droplet assay in the presence of DNA having an ER binding site but not in the presence of control DNA or no DNA. All tests performed in the presence of estrogen.

FIGS. 10A-10D show ligand dependent condensate formation links phase separation to oncogene expression. FIG. 10A shows MED1 co-localizes with Myc DNA in condensates in the presence of estrogen, but not in the absence of estrogen or in the presence of estrogen and tamoxifen. FIG. 10B shows MYC expression is increased in the presence of estrogen and decreased to constitutive levels in the present of estrogen and tamoxifen. FIG. 10C shows ER is incorporated into condensates in the presence of estrogen but not in the presence of estrogen and tamoxifen. ER droplets are depicted in the top row, MED1 droplets in the middle row and a merge of ER and MED1 droplets in the lower row. FIG. 10D shows that the enrichment ratio of ER in MED1 condensates is significantly increased in the presence of estrogen.

FIG. 11 shows transcriptional condensates are multicomponent structures. Using IF and Myc FISH, co-localization in condensates of BRD4, p300, CDK7, CDK6, Proteosome, and Topo-isomerase with Myc transcription was demonstrated. p300 and CDK7 were detected in ovarian cancer cells. All other components detected in breast cancer cell line MCF7.

FIG. 12 shows transcriptional condensates are multicomponent structures.

FIG. 13 shows tools for assaying small molecule effects on transcriptional condensates. HCT116 colon cancer cell lines endogenously tagged with MED1-GFP, BRD4-GFP, POL2-GFP, or HP1a-GFP (Mock), all forming condensates in the nucleus.

FIGS. 14A-14D show JQ1 dissolves genomic transcriptional condensates. FIG. 14A shows JQ1 reduces the number of, or eliminates, MED1, BRD4 and POL2 condensates. FIG. 14B shows the results of a Fluorescence Recovery After Photobleaching (FRAP) assay with fluorescently labeled BRD4. The presence of JQ1 significantly increased the rate of BRD4 turnover in the light irradiated condensate, resulting in significantly quicker (10 s versus 120 s) recovery via replacement of photobleached BRD4 with fluorescing BRD4. FIG. 14C shows higher levels of BRD4 at super-enhancers (SE) than at typical enhancers (TE). FIG. 14D, left panel, shows that gene expression by super-enhancers is more sensitive to JQ1 inhibition than typical enhancers. FIG. 14D, right panel, shows JQ1 reduces BRD4 genomic occupancy in super-enhancers to a greater extent than typical enhancers.

FIG. 15 shows antimetabolites have no effect on transcriptional condensates. Specifically, neither 5 uM 5-FU nor 5 uM 5-Aza had a detectable effect on MED1, BRD4 or POL2 condensates.

FIG. 16 shows the effect of various inhibitors on MED1, BRD4, and POL2 condensates in HCT116 colon cancer cell lines endogenously tagged with MED1-GFP, BRD4-GFP, and POL2-GFP.

FIG. 17 shows the effect of various intercalators on MED1, BRD4, and POL2 condensates in HCT116 colon cancer cell lines endogenously tagged with MED1-GFP, BRD4-GFP, and POL2-GFP.

FIG. 18 shows the effect of various CDK inhibitors on MED1, BRD4, and POL2 containing condensates in HCT116 colon cancer cell lines endogenously tagged with MED1-GFP, BRD4-GFP, and POL2-GFP.

FIG. 19 provides models of drug effects on transcriptional condensates. Bortezomib, Mitoxantrone, Daunorubicin, THZ1, and Dinaciclib cause global dissolution of condensates. See FIGS. 16-18. Long exposure (e.g., 24 hours) to JQ1, as well as exposure to A485 and Palbociclib, caused genomic release and consolidation of condensates. See, FIGS. 14, 16, and 18. Short exposure (e.g., 5 minutes) to JQ1, as well as exposure to U0216, caused eviction of some condensate components (i.e., selective eviction). See, FIGS. 14 and 16.

FIGS. 20A-20B show small molecules access condensates in vitro. FIG. 20A, left panel, shows Estrogen receptor (ER) (green) and MED1 (red) co-localize in in vitro droplets in the presence of estrogen while estrogen receptor did not incorporate into condensates in the presence of estrogen and tamoxifen. FIG. 20A, right column-top, shows cells with LAC arrays with attached ER have reduced ER (green) and MED1 (red) containing condensates in the presence of Tamoxifen. Right column, bottom, show the relative fluorescent intensity of ER and MED1 in the presence and absence of tamoxifen. FIG. 20B, top, show the structures of fluorescently labeled tamoxifen (FLTX1) and Cy5 dye, having similar molecular weights. FIG. 20B, bottom, shows FLTX1 is incorporated into MED1 containing condensates while the similarly sized Cy5 dye is not incorporated.

FIG. 21 shows tamoxifen “chases” fluorescent tamoxifen from MED1 droplets. Top row shows that MED1 droplets are not affected by addition of FLTX1, or FLTX1 and tamoxifen. Bottom row shows that FLTX1 is incorporated into MED1 droplets but is diluted out by the addition of a 10-fold excess of tamoxifen, confirming FLTX1 and tamoxifen have similar condensate incorporation properties.

FIG. 22 shows fluorescent tamoxifen enriches specifically in MED1 condensates. Left bottom panel shows FLTX1 incorporates into MED1 droplets. MED1 is a component of transcriptional condensates. Right bottom panel shows FLTX1 does not incorporate into heterochromatin protein 1 (HP1α) droplets. HP1a is a component of heterochromatic condensates. Significantly, FLTX1 was incorporated into MED1 droplets in the absence of its target, estrogen receptor.

FIG. 23 shows condensate dissolving drugs enrich in MED1 condensates. Mitoxantrone, curcumin, and daunorubicin each have fluorescent activity and cause condensate dissolution. FIG. 23, bottom panel, shows these drugs immediately incorporate into MED1 droplets.

FIG. 24 illustrates ER/MED1 droplets contacted with a fluorescent peptide (left side). Upon estrogen exposure, the Estrogen Receptor undergoes a conformational change that allows it to interact with MED1 LXXLL domain (right side).

FIG. 25 shows that upon addition of the LXXLL peptide (QNPILTSLLQITG; SEQ ID NO: 1) to ER/MED1 droplets, the peptide is incorporated into MED1 droplets resulting in a decrease in partitioning of ER into the MED1 droplet.

FIG. 26 shows incorporation of peptides into MED1/ER droplets in the presence of estrogen. Poly-proline (Poly P) and RNA polymerase II CTD repeat YSPTSPS peptide (CTD) did not have an effect on ER/MED1 droplet formation, while poly-glutamate (Poly-E) (acid) and poly-lysine (Poly-K) peptides (basic) abolished MED1/ER droplet formation.

FIG. 27 shows incorporation of a cell penetrating LXXLL peptide having an HIV-TAT tag into U2OS cells demonstrating that peptides can be visualized in live cells.

FIGS. 28A-28E show nuclear condensates in human tissue and in vitro. FIG. 28A shows a model illustrating potential behaviors of small molecules in nuclear condensates. FIGS. 28B-28C shows immunofluorescence of scaffold proteins of various nuclear condensates in tissue biopsies from benign and malignant human breast (FIG. 28B), and benign and malignant colon tissue (FIG. 28C), in nuclei stained with Hoechst, imaged at 100× on a fluorescent confocal microscope. FIG. 28D shows a schematic of an in vitro droplet formation assay to measure small molecule partitioning into nuclear condensates. FIG. 28E shows an in vitro droplet assay showing the behavior of fluorescein dye in the presence of six protein condensates formed in 125 mM NaCl and 10% PEG, with 10 μM protein and 5 μM fluorescein, imaged at 150× on a confocal fluorescent microscope. Quantification of enrichment of the drug is shown to the right, error bars represent SEM.

FIGS. 29A-29E show the partitioning behavior of small molecule drugs in nuclear condensates in a droplet assay. Six nuclear condensates formed in 125 mM NaCl and 10% PEG, with 10 μM protein treated with either (FIG. 29A) 5 μM Cisplatin-TMR, (FIG. 29B) 50 μM Mitoxantrone, (FIG. 29C) 100 μM FLTX1, (FIG. 29D) 5 μM THZ1-TMR, or (FIG. 29E) 1 μM JQ1-ROX imaged at 150× on a confocal fluorescent microscope. Quantification of enrichment of the drug within droplets is shown to the right of each panel, error bars represent SEM.

FIGS. 30A-3F show a small molecule concentration within condensates influences drug activity. FIG. 30A shows an in vitro droplet assay of MED1 and HP1α condensates formed in 125 mM NaCl and 10% PEG, 5 nM of 450 bp DNA, 10 μM MED1, and 5 μM cisplatin-TR, imaged at 150× on a confocal fluorescent microscope. FIG. 30B shows bio analyzer tracings of DNA contained within either MED1 or HP1α droplets exposed to the indicated concentration of cisplatin. FIG. 30C shows (Top) a schematic of an assay to determine the location of platinated DNA relative to various nuclear condensates. (Bottom) Co-immunofluorescence of platinated DNA and the indicated protein in HCT116 cells treated with 50 μM cisplatin for 6 hours. Imaged at 100× on a confocal fluorescent microscope. Quantification of overlap shown to the right. FIG. 30D shows (Top) a schematic of a live cell condensate dissolution assay. (Bottom) HCT116 cells bearing endogenously mEGFP-tagged MED1, HP1α, or FIB1 treated with 50 μM cisplatin for 12 hours. Quantification of MED1, HP1α, or FIB1 condensate score is shown to the right. FIG. 30E shows a MED1 ChIP-seq in HCT116 cells treated with vehicle or 50 μM cisplatin for 6 hours. (Left) Plotted are mean read density of MED1 at super-enhancers and typical-enhancers (error bars show min and max) and (Right) gene tracks of MED1 ChIP at the MYC super-enhancer and AQPEP typical-enhancer. FIG. 30F shows a metaplot of cisplatin-DNA-Seq in cisplatin treated Hela cells comparing super-enhancers and typical enhancers.

FIGS. 31A-31F show tamoxifen action and resistance in MED1 condensates. FIG. 31A shows a schematic showing tamoxifen resistance by ER mutation and MED1 overexpression in breast cancer. FIG. 31B shows in vitro droplets assay of the indicated form of GFP-tagged ER in the presence of estrogen, +/−100 μM tamoxifen. Droplets are formed in 125 mM NaCl and 10% PEG with 10 μM each protein and 100 μM estrogen. FIG. 31C shows (Left) Immunofluorescence of MED1 in tamoxifen sensitive (MCF7) and resistant (TAMR7) ER+ breast cancer cell lines imaged at 100× on a confocal fluorescent microscope. (Top right) Quantification of MED1 condensate size in breast cancer cells. (Bottom right) Relative quantities of MED1 in the indicated breast cancer cell line by western blot, error bars show SEM. FIG. 31D shows in vitro droplets assays of ER in the presence of 100 μM estrogen, +/−100 μM tamoxifen with either 5 μM (Low) or 20 μM (High) MED1. Droplets are formed with 5 μM ER in 125 mM NaCl and 10% PEG, imaged at 150× on a confocal fluorescent microscope, error bars are SEM. FIG. 31E shows an in vitro droplets assay with either 5 μM (Low) or 20 μM (High) MED1 with 100 μM FLTX1 in 125 mM NaCl and 10% PEG, error bars are SD. FIG. 31F shows models for tamoxifen resistance due to altered drug affinity (via ER mutation) or concentration (via MED1 overexpression).

FIGS. 32A-332C show nuclear condensates in cell lines and human tumor tissue. FIG. 32A shows mouse embryonic stem cells expressing either endogenously mEGFP-tagged proteins (MED1, BRD4, SRSF2), mCherrytagged proteins (HP1α) or transfected with constructs expressing GFP-tagged proteins (NPM1, FIB1) were imaged by confocal fluorescent microscopy. FIG. 32B shows clinical data from biopsied breast and colon cancer specimen. FIG. 32C shows H&E staining of ER positive breast carcinoma and colon adenocarcinoma.

FIGS. 33A-33C show the volume and number of nuclear condensates in normal and tumor tissue. FIG. 33A shows the volume of nuclear condensates in normal and malignant breast tissue (upper) and in normal and malignant colon tissue (lower). Values indicate percent nuclear volume and standard deviation. There were no significant differences between the individual nuclear condensates in normal and malignant states. FIG. 33B shows a table showing average volume of nuclear condensates in normal and malignant tissue. FIG. 33C shows a table showing average number of nuclear condensates in normal and malignant tissue.

FIGS. 34A-34B show nuclear condensate forming proteins. FIG. 34A shows a schematic representation of constructs used for purifying nuclear condensate proteins. The IDR (intrinsically disordered region) alone was used for MED1 and BRD4 proteins and the full length was used for HP1α, SRSF2, NPM1, and FIB1 proteins. FIG. 34B shows (Upper) the number of hydrophobic amino acids Phenylalanine (F), Tryptophan (W), and Tyrosine (Y) in the IDR and full-length protein. MED1 IDR has the highest number of hydrophobic residues. (Lower) Table of Positive Charged Interaction Elements (CIE+) and Negative Charged Interaction Elements (CIE-) of the IDR or full-length nuclear condensate protein. These results indicate that MED1 protein might participate in interactions govern by the pi-system.

FIGS. 35A-35B show in vitro droplets of condensate forming proteins. FIG. 35A shows confocal microscopy of in vitro droplet formation assays of the indicated GFP-tagged protein in 125 mM NaCl and 10% PEG. MED1 and BRD4 proteins are the IDR portion only. FIG. 35B shows confocal microscopy images of MED1, BRD4, SRSF2, HP1α, FIB1, and NPM1 nuclear condensates at the indicated concentration of salt (125 mM, 350 mM, 650 mM, 1000 mM NaCl), experiments were performed with 10 μM protein in 10% PEG.

FIG. 36 shows a schematic representation of enrichment ratio calculations. Droplets are defined in the protein channel and maximum intensity of drug is measured in that area to obtain drugᵢₙ (left panel), background is measured in the drug channel in areas defined by the protein channel in an in vitro droplet reaction containing protein but no drug (middle panel), and drug_{diffuse} intensity is measured in a droplet reaction without the protein (right panel).

FIGS. 37A-37D show small molecule partitioning in nuclear condensates. FIG. 37A shows confocal microscopy of in vitro droplet formation assays of the indicated small molecule alone (4.4 kDa dextran, fluorescein, and hoechst) without any protein added to the reaction. All small molecules alone show a diffuse fluorescent signal indicating that the molecule alone does not form droplets. FIGS. 37B-37C show confocal microscopy images showing the behavior of hoechst (FIG. 37B) and 4.4 kDa dextran (FIG. 37C) relative to six nuclear condensates formed in vitro, in 125 mM NaCl and 10% PEG. Quantification shown to the right, error bars represent SEM. Both hoechst and dextran diffuse freely through the condensates tested without being excluded or concentrated. Schematic of the assay shown at top. FIG. 37D shows confocal microscopy images of fluorescently-labeled 4.4 kDa, 10 kDa, 40 kDa, and 70 kDa dextran in MED1 condensates. Experiments were performed with 10 μM protein and 0.1 mg/ml TRITC-labeled dextran, in 125 mM salt and 16% ficoll. Dextran of smaller sizes (4.4 kDa and 10 kDa) are able to freely diffuse through the condensates while larger sizes of dextran (40 kDa and 70 kDa) are partially excluded from MED1 condensates. This indicates that the effective pore sizes of the condensates studied is at least 10 kDa.

FIGS. 38A-38D show properties of small molecule drugs, not their fluorescent moiety, govern partitioning into condensates. FIG. 38A shows confocal microscopy of in vitro droplet formation assays of the indicated small molecule drug alone (cisplatin, FLTX1, THZ1, mitoxantrone, and JQ1) without any protein added to the reaction. All small molecule drugs alone show a diffuse fluorescent signal indicating that the molecules alone do not form droplets. FIG. 38B shows ROX and Texas Red enrichment in MED1 droplets formed in 125 mM NaCl and 10% PEG measured by confocal microscopy. Neither of the two dyes used to visualized drugs were enriched in MED1 condensates. FIG. 38C shows a schematic of an in vitro droplet drug chase out experiment. Labeled cisplatin is added to MED1 droplets to form MED1 droplets concentrated with cisplatin-TR. Unlabeled transplatin or unlabeled cisplatin is added to the droplet mixture and the amount of labeled cisplatin-TR remaining in the droplet is measured after chase out. Transplatin, a clinically ineffective trans-isomer of cisplatin, is not able to chase out cisplatin-TR, while high concentrations of unlabeled cisplatin is able to chase out cisplatin-TR. FIG. 38D shows a schematic of an in vitro droplet drug chase out experiment. Graph showing FLTX1 enrichment in MED1 droplets upon tamoxifen addition measured by confocal microscopy. Tamoxifen was able to chase-out FLTX1 from MED1 droplets. All error bars shown represent SEM.

FIGS. 39A-39C show small molecule drugs can be concentrated into MED1 condensates by 100-folds. FIG. 39A shows quantitative phase microscopy of MED1 droplets formed in 125 mM NaCl and 10% PEG. Colorbar indicates optical phase delay, φ, in degrees. From phase images, the average MED1 concentration in individual condensates was calculated. FIG. 39B shows a graph showing MED1 concentration in in vitro droplets upon the addition of no drug, 5 μM cisplatin or 50 μM mitoxantrone. Datapoints are population averages (n=272, 115 and 85 individual condensates for each condition). Error bars denote standard deviation. FIG. 39C shows varying concentration of cisplatin or Mitoxantrone was added to MED1 droplets and the concentration of drug remaining in solution was measured by uv spectroscopy. Combining the spectroscopy measurements with an estimate of the total volume of the MED1 condensate phase obtained from the measurements in (FIG. 39B), the partition ratio of cisplatin was estimated to be up to 600-fold and the partitioning ratio of Mitoxantrone to be approximately 100-fold.

FIGS. 40A-40B show the association of drug targets with transcriptional condensates. FIG. 40A shows immunofluorescence of MED1, HP1α, CDK7, ER, and BRD4 together with MYC RNA FISH. Consistent with the finding that MED1, a marker of transcriptional condensates, is present in puncta at the MYC oncogene, CDK7, ER, and BRD4 are also found in puncta at MYC. These results mirror those obtained by ChIP-Seq at this locus. In contrast, signal for HP1α, a marker of heterochromatin condensates, is not found at MYC. Average and random image analysis shown to the right. FIG. 40B shows (Top) a schematic of in vitro droplet assay showing mixing of nuclear condensate protein (MED1 or HP1α) with various drug target proteins (CDK7, ER, or BRD4), with partitioning into the nuclear condensate measured by confocal microscopy. (Middle) In vitro droplet assays with MED1, ER, HP1α and BRD4 at 10 μM, CDK7 at 200 nM. Droplets are formed in 125 mM NaCl, 10% PEG and droplet formation buffer. All drug targets tested were concentrated in MED1 condensates. ER was found to be concentrated both in MED1 and HP1α condensates, consistent with previous reports and its ability to associate with both co-activators and co-repressors. (Bottom) Quantification of target protein enrichment in the indicated condensates, error bars represent SEM.

FIG. 41 shows partitioning behavior of various small molecule drugs in whole Mediator complex. Confocal microscopy images of drugs (THZ1, mitoxantrone, cisplatin, FLTX1, fluorescein, and 4.4 kDa dextran) in whole mediator complex condensates. Mediator was imaged in brightfield while the small molecule was imaged by the channel in which it fluoresces. Experiments were performed in 10% PEG and 125 mM NaCl. The partitioning behavior of various small molecule drugs into whole Mediator complex recapitulate the partitioning behavior of drugs into MED1 condensates. Quantification of enrichment shown to the right, error bars represent SEM.

FIG. 42 shows partitioning behavior of various small molecule drugs into MED1 condensates formed in ficoll. Confocal microscopy images of small molecule drugs (THZ1, mitoxantrone, cisplatin, FLTX1, fluorescein, and JQ1) concentration behavior in MED1 condensates in the presence of 125 mM NaCl and 20% ficoll. The partitioning behavior of small molecules are similar regardless of crowder used to form MED1 droplets. Quantification of enrichment shown to the right, error bars represent SEM.

FIGS. 43A-43B show cisplatin molecules are highly mobile in MED1 droplets. FIG. 43A shows confocal microscopy images showing fluorescence recovery after photobleaching (FRAP) of TR-cisplatin and MED1 in condensates formed in the presence of 125 mM NaCl and 10% PEG with 5 μM TR-cisplatin and 10 μM protein. FIG. 43B shows quantification of FRAP (error bars represent SEM).

FIGS. 44A-44D show specific chemical moieties govern concentration in MED1 condensates. FIG. 44A is a depiction of small molecule boron-dipyrromethene (BODIPY) library. FIG. 44B shows the fluorescence intensity of probe library in MED1 droplets measured by confocal microscopy. Experiments were performed in 125 mM NaCl and 10% PEG, with 10 μM MED1 and 1 μM small molecule. The fluorescent of the BODIPY molecule alone is highlighted in red. FIG. 44C shows the fluorescent intensity of a random selection of 18 probes from the library without MED1 protein demonstrating they have similar fluorescent intensity. FIG. 44D shows Top 5 (left) and bottom 5 (right), R2 and R1 sidechains, ranked by fluorescent intensity.

FIGS. 45A-45E show aromatic residues of MED1 contribute to small molecule partitioning into MED1 condensates but are dispensable for condensate formation. FIG. 45A shows confocal microscopy images of MED1, BRD4, SRSF2, HP1α, FIB1, and NPM1 nuclear condensates formed in 125 mM NaCl and 10% PEG together with 5 μM of the small molecule probe that ranked the highest in fluorescent intensity within MED1 condensates. The probe was specifically concentrated into MED1 condensates, indicating that chemical features of the probe selectively interact with those of MED1 condensates. The top-ranking probes that concentrated in MED1 condensates showed a preference for BODIPY molecules that are modified with an aromatic ring. This suggests that the pi-system might be contributing to the interaction between small molecules and MED1. FIG. 45B shows a schematic of the MED1 IDR mutant proteins. The pi-system governs the interactions of supramolecular assemblies, where pi-pi or pi-polar interactions play prominent roles. To test if these interactions govern small molecule partitioning into MED1 condensates, and encouraged by the observation that the MED1 IDR is enriched for both aromatic and basic amino acids residues relative to other proteins studied here, an aromatic MED1 IDR mutant was generated (all 30 aromatic residues changed to alanine) and a basic MED1 IDR mutant (all 114 basic residues changed to alanine). In FIG. 45C, the ability of MED1 mutants was tested to form droplets by confocal microscopy using MED1 wildtype, MED1 basic mutant (all basic amino acids replaced with alanine), and MED1 aromatic mutant (all aromatic amino acids replaced with alanine) in the presence of 125 mM NaCl and 10% PEG. The MED1 basic mutant showed an impaired ability to form droplets in vitro, indicating that the basic residues of MED1 are required for the homotypic interactions that govern droplet formation. The MED1 aromatic mutant formed droplets similar to those of MED1 wildtype protein. FIG. 45D shows the role of MED1 aromatic residues in incorporation of aromatic small molecule probes. Confocal microscopy images and their quantification for the top hit BODIPY probe together with MED1 or MED1 aromatic mutant, which show that the partitioning behavior of the aromatic probe into MED1 aromatic mutant droplets is substantially reduced. Experiments were performed in 10% PEG and 125 mM NaCl with 10 μM protein and 5 μM small molecule. FIG. 45E shows confocal microscopy images and their quantification for cisplatin together with MED1 or MED1 aromatic mutant, which show that the partitioning behavior of cisplatin into MED1 aromatic mutant droplets is substantially reduced. Experiments were performed in 10% PEG and 125 mM NaCl with 10 μM protein and 5 μM cisplatin-TR. Taken together, these results suggest that the pi-system contributes to small molecule partitioning into MED1 condensates. All error bars represent SEM.

FIG. 46 shows DNA can be compartmentalized and concentrated in nuclear condensates. (Top) Schematic of droplet assay showing protein, DNA, and cisplatin mixed in droplet forming conditions, then spun down to separate the droplet phase from the dilute phase. The amount of DNA in the two phases is subsequently measured using a Bioanalyzer. DNA is enriched in MED1 and HP1α droplet phase (left) compared to MED1 and HP1α dilute phase (right).

FIGS. 47A-47D show the concentration of small molecules in specific condensates can influence target engagement. FIG. 47A shows HCT116 cells were treated with DMSO or 50 μM cisplatin for 6 hours followed by cisplatin immunofluorescence. The antibody only recognizes platinated DNA in cells treated with cisplatin, supporting antibody specificity. FIG. 47B shows (Left) mEGFP-MED1 tagged HCT116 cells treated with JQ1 for 24 hours result in diminution of MED1 condensates. (Right) Metaplot of MED1 ChIP-Seq in DMSO vs JQ1 treated HCT116 cells. FIG. 47C shows cells were treated with JQ1 and then cisplatin to determine whether diminution of MED1 condensates leads to reduced DNA platination at MYC locus. MYC DNA FISH and MED1 immunofluorescence showed a loss of signal for platinated DNA after JQ1 treatment, indicating that the presence of a MED1 condensate contributes to DNA platination at this locus. FIG. 47D shows (Left) MED1 ChIP-Seq track at MYC in DMSO or JQ1 treated HCT116 cell showing loss of MED1 loading after JQ1 treatment. (Right) Quantification of cisplatin IF signal at MYC DNA FISH foci in HCT116 cells with DMSO or JQ1 treatment, error bars represent SEM.

FIGS. 48A-48G show genotyping of endogenously tagged cell lines. Schematic image and genotyping agarose gel showing mEGFP tagged (FIG. 48A) MED1, (FIG. 48B) HP1α, (FIG. 48C) FIB1 (FIG. 48D) NPM1, (FIG. 48F) BRD4, and (FIG. 48G) SRSF2 in HCT116 colon cancer cells. FIG. 48E is an agarose gel of FIB1 and NPM1 expression.

FIGS. 49A-49B show nuclear condensates in cells are highly dynamic. FRAP of mEGFP-tagged (FIG. 49A) MED1 and (FIG. 49B) HP1α in HCT116 cell lines (error bars represent SEM) (n=7).

FIGS. 50A-50B show Dissolution of MED1 condensates in cells upon prolonged cisplatin treatment. FIG. 50A shows HCT116 cells endogenously GFP-tagged MED1 treated with DMF or 50 μM cisplatin for 3, 6, or 12 hours. Quantification shown to the right, error bars are SD. FIG. 50B shows cell viability assay of HCT116 cells expressing GFP-MED1 treated for 12 hours with DMF or 50 μM Cisplatin.

FIG. 51 illustrates the effect of cisplatin on various nuclear condensates. FIG. 24 shows HCT116 cells bearing either endogenously GFP-tagged MED1, BRD4, HP1α, FIB1, NPM1, or SRSF2 treated with 50 μM cisplatin for 12 hours. Cisplatin specifically disrupts MED1 and BRD4 condensates, consistent with cisplatin and BRD4 being selectively concentrated in MED1 condensates.

FIG. 52 shows decreased MED1 genomic occupancy upon cisplatin treatment. The graph shows MED1 ChIP-seq after 6 hours of DMSO or 50 μM cisplatin treatment, MED1 genomic levels are reduced after cisplatin treatment.

FIGS. 53A-53D show the characterization of MED1 condensates in MCF7 cells. FIG. 53A shows a Western blot of MED1 in MCF7 cells and MCF cells infected with MED1-mEGFP lentiviral vector. FIG. 53B shows FRAP of MED1-mEGFP in MCF7 cells expressing this fusion protein by virtue of a lentiviral vector. Quantification shown to the right, black bars represent 95% confidence interval of the best fit line. FIG. 53C shows MCF7 cells expressing MED1-mEGFP were grown in estrogen-free conditions then stimulated with 100 nM estrogen for 15 minutes and imaged for 4 minutes on a confocal fluorescent microscope. FIG. 53D shows the quantification of size and intensity of fusing MED1 condensates shown in (FIG. 53C).

FIGS. 54A-54B show estrogen and tamoxifen dependent MED1 condensate formation at the MYC oncogene. FIG. 54A shows DNA FISH and immunofluorescence in estrogen-starved MCF7 cells treated with 100 nM estrogen or 100 nM estrogen and 5 μM tamoxifen for 24 hours. Average image analysis and random image analysis shown to the right. FIG. 54B shows RT-qPCR showing relative MYC RNA expression in estrogen-starved, estrogen stimulated, or estrogen and tamoxifen treated MCF7 cells, error bars represent SEM.

FIG. 55 shows FLTX1 concentrates in MED1 condensates in cells. (Left) Schematic of MED1 or HP1α tethered to the LAC array in U2OS cells generating a MED1 or HP1α condensate. (Middle) Representative images of isolated U2OS cell nuclei with either MED1 or HP1α tethered to the LAC array exposed to FLTX1. Zoomed image of the Lac array shown inset, merged images shown on the right. (Right) Quantification of FLTX1 enrichment at the LAC array with either MED1 or HP1α tethered, error bars represent SEM. ESR1 is not expressed in this osteosarcoma cell line.

FIG. 56 shows patient derived hormonal therapy resistant mutations of ESR1. Plot of ER mutation frequency derived from a 220 patient set from the cBioPortal database showing locations of ER point mutations with hotspots at 537 and 538.

FIGS. 57A-57B show enrichment ratios of ER and ER mutants in MED1 droplets. FIG. 57A shows the quantification of ER or ER mutant enrichment ratios in MED1 droplets in the presence of either estrogen or estrogen and tamoxifen. FIG. 57B shows (Left) representative images of ER mutants partitioning in MED1 droplets, enrichment ratios shown to the right. Experiments for both (FIG. 57A) and (FIG. 57B) are performed in 125 mM NaCl, 10% PEG, 10 μM of each protein, 100 μM estrogen with or without 100 μM of the indicated ligand. All error bars represent SD.

FIGS. 58A-58C show MED1 overexpression in tamoxifen resistant breast cancer cells. FIG. 58A shows a schematic demonstrating drug concentration in a condensate upon increase in condensate volume by scaffold protein overexpression. Assuming limited drug in a system, the concentration of drug in a MED1 droplet is expected to decrease upon condensate volume expansion (FIG. 58B) Western blot of MED1 and Actin in MCF7 cells (tamoxifen sensitive) and TAMR7 cells (tamoxifen resistant derivative of MCF7) showing that MED1 levels are higher TAMR7 cells. Quantification from the western blot is shown below, which is an average of 3 experiments. FIG. 58C shows the quantification of MED1 condensates in tamoxifen sensitive and resistant cell lines showing the volume of the MED1 condensates and the number of condensates per nucleus.

FIGS. 59A-59B show MED1 condensates increase in size with increasing MED1 concentration. FIG. 59A shows droplet size in pixels from in vitro droplet assays performed with either 5 μM (Low) or 20 μM (High) MED1-GFP in 125 mM NaCl and 10% PEG. Quantification shown to the right, error bars represent SD. FIG. 59B shows a schematic phase diagram of MED1, demonstrating that when the total concentration of MED1 increases, the size of droplet increases while maintaining the concentration of protein within the droplet phase.

FIG. 60 shows MED1 Condensation at the Lac Array. FIG. 60 shows (Left) a schematic of the Lac array assay. U2OS cells bearing 50,000 copies of the Lac binding site are transfected with a construct expressing the Lac DNA binding domain (DBD) to the estrogen receptor ligand binding domain (LBD). When the transcriptional apparatus is recruited to that site a mediator condensate is detectable by immunofluorescence (Middle) U20S-Lac cells were transfected with a construct expressing the Lac DBD fused to the ER LBD and GFP+/− a construct overexpressing MED1. Cells were grown in estrogen deprived media, and treated with 10 nM estrogen+/−10 nM tamoxifen then fixed and subjected to MED1 IF. Top panel shows the location of ER-LBD at the Lac array, bottom panel shows MED1 IF. Inset image shows zoom. (Right) Quantification of MED1 enrichment relative at the Lac array, error bars represent SD.

FIGS. 61A-61D show an in silico model of small molecule partitioning in condensates. To demonstrate the behavior of a small molecule drug engaging a target contained within a condensate, a simple model was developed in which a drug and target are both contained within a condensate with percent target engagement as the readout. In this model, target partitioning is not affected by drug binding. FIG. 61A shows a table of the values used to build a model of drug engagement within condensates, derived from known values of ER and tamoxifen. Condensate volume fraction value derived from analysis of MED1 IF on human ER+ breast carcinoma biopsies. FIG. 61B shows target binding as a function of drug concentration in simulations. The dashed line represents a system in which target and drug are freely diffusing through the cells. Red and blue lines represent a system in which target and drug are concentrated into a condensate. The blue line represents target engagement in the condensate where the drug and target are concentrated, the red line represents target engagement in the dilute phase of the nucleoplasm. Overall, these data show that drug engages a higher percent of target molecules inside a condensate that outside, at a given concentration. FIG. 61C shows a fraction of bound target at a given concentration of drug at various partitioning coefficients of drug. Dotted line represents the target engagement in a diffuse regime. Overall, this simulation shows that as the partitioning coefficient of drug in a condensate increases the percent of target bound at a given concentration. FIG. 61D shows target engagement by drug in the setting of larger condensates. Simulation of target binding as a function of drug concentration in the setting of normal condensate volume (2% of the volume of the nucleus) versus larger condensate volume (4% of the volume of the nucleus). Diffuse control shown by the dashed line. Overall, these data show that a drug may be less effective in binding its target in larger condensates.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

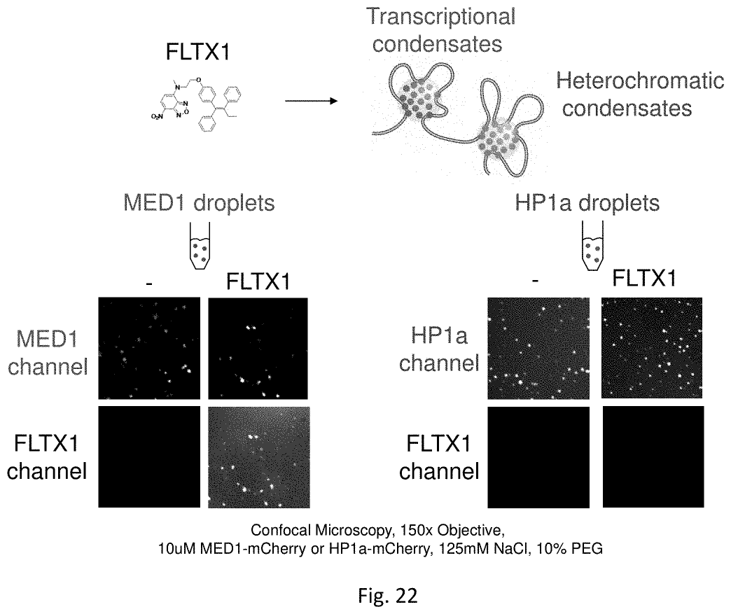

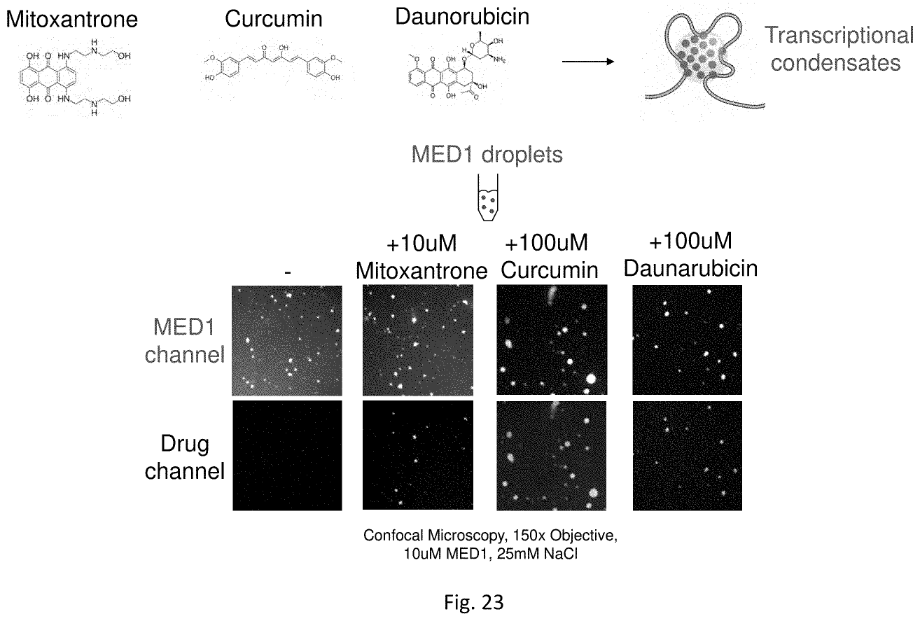

The inventors have surprisingly found that some agents are incorporated into condensates that do not have a target for the agent. See, e.g., FIG. 22. This has important ramifications for drug efficacy. For example, the effectiveness of a drug may be less if the drug is sequestered into a condensate, preventing interactions with the target. Alternatively, the effectiveness of a drug may be less if a condensate suppresses access of the drug to the target. This phenomenon may help explain why some candidate agents show high activity on a therapeutic target in vitro but do not show the same activity in a cell or organism. This may also explain the surprising observation that inhibition of global gene regulators such as BRD4 or CDK7 can have selective effects on oncogenes that have acquired large super-enhancers; selective partitioning of inhibitors like JQ1 and THZ1 into super-enhancer condensates will preferentially disrupt transcription at those loci. The inventors have further surprisingly found that condensates concentrate some clinically important small molecule cancer therapeutics, such that their pharmacodynamic properties are altered. Thus, condensates can concentrate small molecules, thereby directing their biological activity.

Thus, some aspects of the invention are directed to a method of characterizing an agent, comprising contacting the agent with a composition (e.g., a solution) comprising a condensate having at least one component, and measuring incorporation of the agent in the condensate. In some embodiments, the method further comprises determining if the agent is a possible therapeutic based on whether, in the appropriate cell, the target and the agent are both at effective concentrations either in or outside the condensate. In some embodiments, the method further comprises characterizing a plurality of agents (e.g., drug candidates) and selecting one or more lead agents having a desirable or optimal condensate partitioning profile (e.g., concentrating in a condensate of an appropriate cell when the target of the agent is present in the condensate, or concentrating outside the condensate when the target of the agent is present outside the condensate). The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. In some embodiments, the agent is sufficiently small to diffuse into a condensate. In some embodiments, the agent is less than about 4.4 kDa. In some embodiments, the agent has a partition coefficient for a condensate described herein of at least 100, 150, 200, 300, 350, 400, 450, 500, 550, 600, 650, 700 or more. In some embodiments, the agent has a partition coefficient for a condensate described herein of less than about 10, 20, 50, 100, 150, 200, 300, 350, 400, 450, 500, 550, or 600.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 Daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, the small molecule comprises at least one, at least two, at least three, or more aromatic side chains.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc. In some embodiments, the agent is a protein or polypeptide comprising at least 5, at least 10, at least 15, at least 20, at least 25, at least 30, or more aromatic amino acids.

In some embodiments, the agent consists of or comprises DNA or RNA.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

The agent may be a known drug. The type of drug is not limited any may be any suitable drug. In some embodiments, the agent may be an anti-cancer drug. In some embodiments, the known drug is to treat a human disease or condition.

In some embodiments, the agent is a chemotherapeutic or a derivative thereof. In some embodiments, the chemotherapeutic agent is selected from actinomycin D, aldesleukin, alitretinoin, all-trans retinoic acid/ATRA, altretamine, amascrine, asparaginase, azacitidine, azathioprine, bacillus calmette-guerin/BCG, bendamustine hydrochloride, bexarotene, bicalutamide, bleomycin, bortezomib, busulfan, capecitabine, carboplatin, carfilzomib, carmustine, chlorambucil, cisplatin/cisplatinum, cladribine, cyclophosphamide/cytophosphane, cytabarine, dacarbazine, daunorubicin/daunomycin, denileukin diftitox, dexrazoxane, docetaxel, doxorubicin, epirubicin, etoposide, fludarabine, fluorouracil (5-FU), gemcitabine, goserelin, hydrocortisone, hydroxyurea, idarubicin, ifosfamide, interferon alfa, irinotecan CPT-11, lapatinib, lenalidomide, leuprolide, mechlorethamine/chlormethine/mustine/HN2, mercaptopurine, methotrexate, methylprednisolone, mitomycin, mitotane, mitoxantrone, octreotide, oprelvekin, oxaliplatin, paclitaxel, pamidronate, pegaspargase, pegfilgrastim, PEG interferon, pemetrexed, pentostatin, phenylalanine mustard, plicamycin/mithramycin, prednisone, prednisolone, procarbazine, raloxifene, romiplostim, sargramostim, streptozocin, tamoxifen, temozolomide, temsirolimus, teniposide, thalidomide, thioguanine, thiophosphoamide/thiotepa, thiotepa, topotecan hydrochloride, toremifene, tretinoin, valrubicin, vinblastine, vincristine, vindesine, vinorelbine, vorinostat, zoledronic acid, and combinations thereof. In some embodiments, the agent is or comprises cisplatin or a derivative thereof. In some embodiments, the agent is or comprises JQ1 ((S)-tert-butyl 2-(4-(4-chlorophenyl)-2,3,9-trimethyl-6H-thieno[3,2-ƒ][1,2,4]triazolo[4,3-a][1,4]diazepin-6-yl)acetate) or a derivative thereof. In some embodiments, the agent is or comprises tamoxifen or a derivative thereof.

In some embodiments, the agent comprises a protein transduction domain (PTD). A PTD or cell penetrating peptide (CPP) is a peptide or peptoid that can traverse the plasma membrane of many, if not all, mammalian cells. A PTD can enhance uptake of a moiety to which it is attached or in which it is present. Often such peptides are rich in arginine. For example, the PTD of the Tat protein of human immunodeficiency viruses types 1 and 2 (HIV-1 and HIV-2) has been widely studied and used to transport cargoes into mammalian cells. See, e.g., Fonseca S B, et al., Adv Drug Deliv Rev., 61(11):953-64, 2009; Heitz F, et al., Br J Pharmacol., 157(2):195-206, 2009, and references in either of the foregoing, which are incorporated herein by reference. In some embodiments, the cell penetrating peptide is HIV-TAT.

In some embodiments, the agent is capable of binding to a target. In some embodiments, the target is present in the composition comprising the condensate. In some embodiments, the target is predominantly present (e.g., at least 51%, at least 60%, at least 70%, at least 80%, at least 90%, at least 95%, at least 97%, at least 99%, at least 99.5%, at least 99.9%, at least 99.99%, or more) outside of the condensate. In some embodiments, the concentration of the target outside of the condensate is at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more than the concentration of the target inside the condensate. In some embodiments, the target is predominantly present (e.g., at least 51%, at least 60%, at least 70%, at least 80%, at least 90%, at least 95%, at least 97%, at least 99%, at least 99.5%, at least 99.9%, at least 99.99%, or more) in the condensate. In some embodiments, the concentration of the target in the condensate is at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more than the concentration of the target outside the condensate.

In some embodiments, the agent is a candidate agent as described herein. In some embodiments, the agent is resultant from an agent has been modified to modulate incorporation into a condensate of interest. In some embodiments, the agent is resultant from the coupling or linking of a first agent and second agent as described herein.

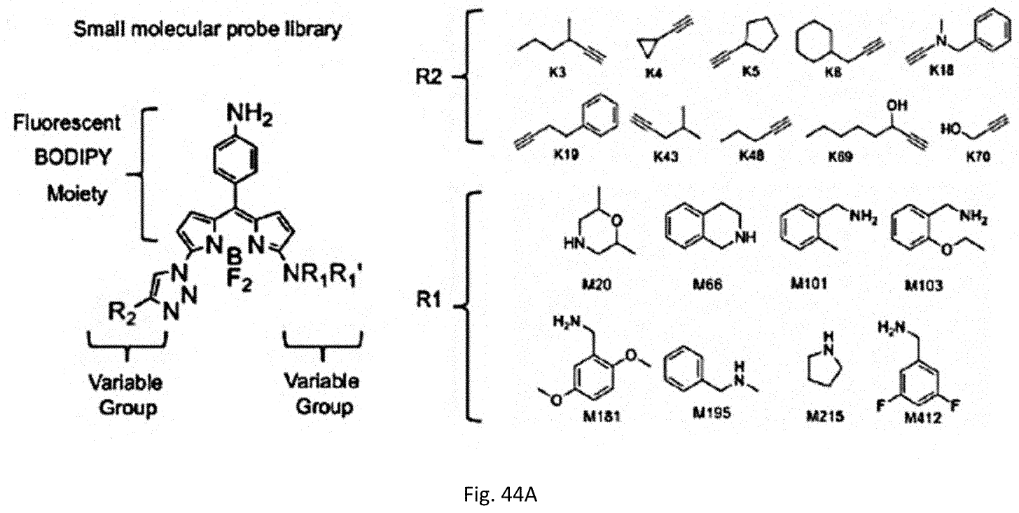

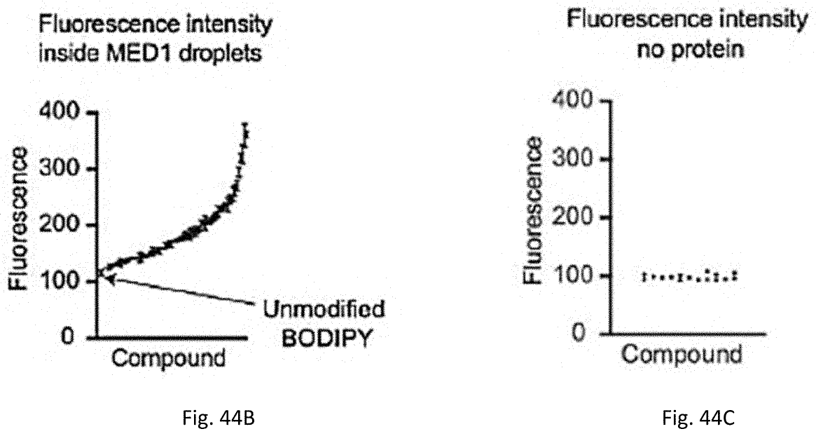

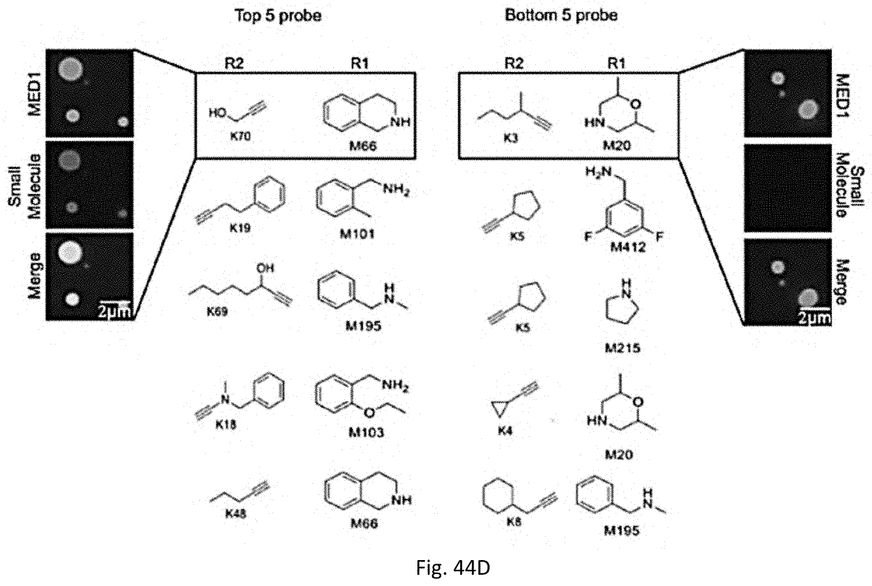

As shown in the examples below, molecules with aromatic rings where found to preferentially concentrate in MED1 condensates. Thus, in some embodiments, the agent is modified to increase or decrease the number of aromatic rings. In some embodiments, the agent is modified to increase the number of aromatic rings by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more. In some embodiments, the agent (e.g., consisting of or comprising a small molecule) is modified to comprise at least one or at least two aromatic rings or more as shown in groups R1 and R2 provided in FIG. 44A. In some embodiments, the agent (e.g., consisting of or comprising a small molecule) is modified to comprise at least one or at least two aromatic rings or more selected from constituents M66, K19, M101, M195, K18, M103, and M66 shown in FIG. 44A. In some embodiments, the agent (e.g., consisting of or comprising a small molecule) is modified to comprise at least two or three structures as shown in each row under “Top 5 probe” provided in FIG. 44D.

In some embodiments, the agent consists of or comprises a peptide, polypeptide or protein and the number of aromatic rings is increased by substituting one or more non-aromatic amino acid residues with an aromatic amino acid residue (e.g., phenylalanine, tryptophan, tyrosine, and/or histidine). In some embodiments, the agent consists of or comprises a peptide, polypeptide or protein and the number of aromatic rings is increased by adding one or more aromatic amino acids. In some embodiments, the aromatic amino acid residue is not histidine. In some embodiments, the aromatic amino acid residue is phenylalanine. In some embodiments, the aromatic amino acid residue is a non-naturally occurring amino acid residue or a nonstandard amino acid residue (e.g., L-DOPA (1-3,4-dihydroxyphenylalanine)).

In some embodiments, the agent consists of or comprises a peptide, polypeptide or protein and the number of aromatic rings is decreased by replacing one or more aromatic amino acids with non-aromatic amino acids (e.g., alanine). In some embodiments, the number of aromatic rings is decreased by deleting or modifying one or more aromatic amino acids.

In some embodiment, the number of aromatic rings is decreased by deleting, modifying, and/or replacing two or more aromatic amino acids.

In some embodiments, the modified agent has increased affinity for a condensate (e.g., a transcriptional condensate, a heterochromatin condensate, splicing speckle condensate, nucleolus, chromatin condensate, polycomb condensate, DNA damage repair condensate, or a condensate physically associated with mRNA initiation or elongation complexes). In some embodiments, the modified agent has increased affinity for a condensate comprising a specific condensate component (e.g., mediator, a mediator component, MED1, BRD4, POLII, SRSF2, FIB1, NPM1, or HP1α). In some embodiments, the modified agent has increased affinity for a condensate comprising a specific mediator component or mediator components (e.g., MED1). In some embodiments, the condensate comprises a condensate component having one or more aromatic side rings (e.g., MED1). In some embodiments, the modified agent has at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold greater affinity for a condensate than a corresponding unmodified agent.

In some embodiments, the modified agent has decreased affinity for a condensate (e.g., a transcriptional condensate, a heterochromatin condensate, splicing speckle condensate, nucleolus, chromatin condensate, polycomb condensate, DNA damage repair condensate, or a condensate physically associated with mRNA initiation or elongation complexes). In some embodiments, the modified agent has decreased affinity for a condensate comprising a specific condensate component (e.g., mediator, a mediator component, MED1, BRD4, POLII, SRSF2, FIB1, NPM1, or HP1α). In some embodiments, the modified agent has decreased affinity for a condensate comprising a specific mediator component or mediator components (e.g., MED1). In some embodiments, the condensate comprises a condensate component having one or more aromatic side rings. In some embodiments, the modified agent has at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold less affinity for a condensate than a corresponding unmodified agent.

In some embodiments, the modified agent has affinity for a second agent. In some embodiment, the modified agent is capable of increasing the concentration or amount of the second agent in a condensate by least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more as compared to the concentration or amount of the second agent in the condensate not in the presence of the modified agent. In some embodiment, the modified agent is capable of decreasing the concentration or amount of the second agent in a condensate by least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more as compared to the concentration or amount of the second agent in the condensate not in the presence of the modified agent.

The target is not limited. In some embodiments, the target is an anti-cancer target. In some embodiments, the target is an enzyme (e.g., oxidoreductase, transferase, hydrolase, lyases, isomerase, ligase, kinase, cyclin dependent kinase, MAPKs, phosphatidylinositol kinase, sphingosine kinase, carbohydrate kinase, nucleoside-phosphate kinase, nucleoside-diphosphate kinase), receptor (e.g., nuclear receptor), oncogene, transcription factor, or signaling factor. In some embodiments, the target is genomic DNA. In some embodiments, the target is any component described herein.

As used herein, condensates refer to phase-separated multi-molecular assemblies. In some embodiments, condensates refer to in vitro condensates (sometimes referred to herein as “droplets”). In some embodiments, in vitro condensates are artificially created with one or more condensate components in a solution. In some embodiments, the in vitro condensate comprises components mimicking a condensate found in a cell. In some embodiments, an in vitro condensate is isolated from a cell.

Any suitable means of isolation of a condensate from a cell or composition is encompassed herein. In some embodiments, a condensate is chemically or immunologically precipitated. In some embodiments, a condensate is isolated by centrifugation (e.g., at about 5,000×g, 10,000×g, 15,000×g for about 5-15 minutes; about 10.000×g for about 10 min). A condensate may be isolated from a cell by lysis of the nucleus of a cell with a homogenizer (i.e., Dounce homogenizer) under suitable buffer conditions, followed by centrifugation and/or filtration to separate the condensate.

In some embodiments, the condensate is present in a cell. The condensate may be a naturally occurring condensate. In other embodiments, the condensate may occur in a transgenic cell or an otherwise manipulated cell. In some embodiments, the condensate may comprise a detectable tag. In some embodiments, the detectable tag is present on a condensate component. In some embodiments, the detectable tag is incorporated in the condensate. The detectable tag (herein also sometimes referred to as a detectable label) is not limited and may be any detectable tag described herein. In some embodiments wherein multiple detectable tags are present, the detectable tags can be differently detectable.

In some embodiments, the condensate may be a transcriptional condensate, a heterochromatin condensate, splicing speckle condensate, nucleolus, chromatin condensate, polycomb condensate, DNA damage repair condensate, or a condensate physically associated with mRNA initiation or elongation complexes. In some embodiments, the condensate may be an in vitro condensate having one or more components of a transcriptional condensate, a heterochromatin condensate, splicing speckle condensate, nucleolus, chromatin condensate, polycomb condensate, DNA damage repair condensate, or a condensate physically associated with mRNA initiation or elongation complexes. In some embodiments, the condensate is physically associated with DNA (e.g., genomic DNA, genomic DNA in a cell). In some embodiments, the condensate, components of the condensate, agents, or methods of assessing condensate properties are those described in PCT/US2019/023694, filed Mar. 22, 2019, incorporated herein by reference in its entirety. In some embodiments, the condensate (e.g., in vivo condensate, ex vivo condensate, in vitro condensate or droplet) comprises a condensate component that is over-expressed in a cancer cell resistant to an anti-cancer agent, wherein the over-expression is associated with resistance to the anti-cancer agent. In some embodiments, the amount of condensate component that is over-expressed in a cancer cell resistant to an anti-cancer agent is greater in the condensate than is present in a condensate from a cancer cell not over-expressing the condensate component. In some embodiments, the volume of the condensate comprising a condensate component that is over-expressed in a cancer cell resistant to an anti-cancer agent is greater than the volume of a condensate found in a cancer cell not over-expressing the condensate component.

In some embodiments, at least one component of the condensate is mediator, a mediator component, MEN, BRD4, POLII, SRSF2, FIB1, NPM1, or HP1α. In some embodiments, the at least one component is a component of a nuclear condensate. In some embodiments, the at least one component is a component of a super-enhancer condensate, splicing speckle condensate, heterochromatin condensate, nucleolus, splicing speckle condensate, nucleolus, chromatin condensate, polycomb condensate, DNA damage repair condensate, or a functional fragment of such component. In some embodiments, the at least one component is a component, or function fragment thereof, of a condensate located in the nucleus. In some embodiments, at least one component of the condensate comprises an intrinsically disordered region (IDR).

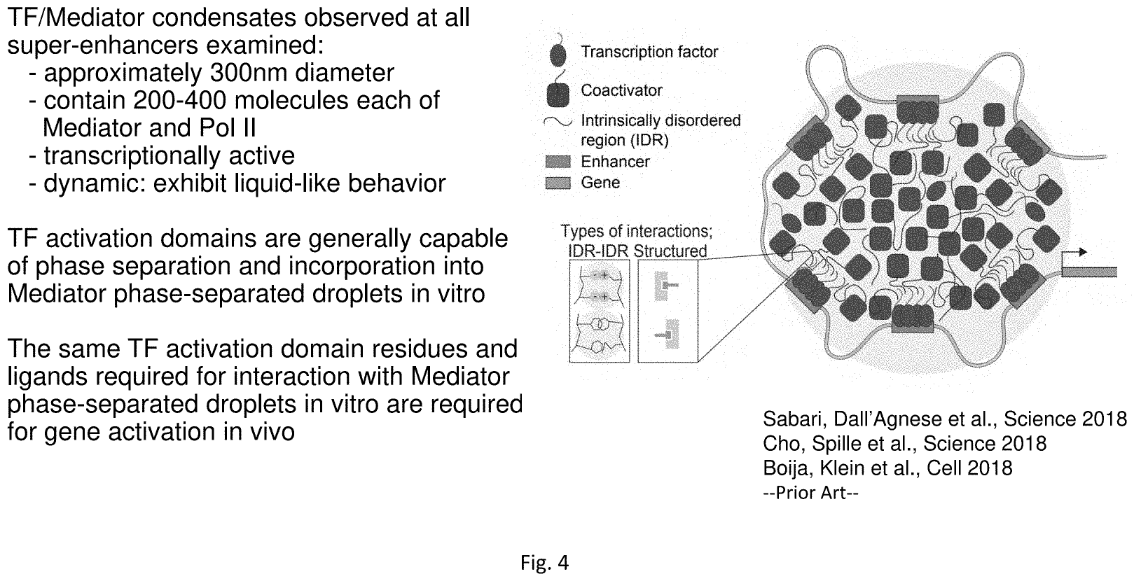

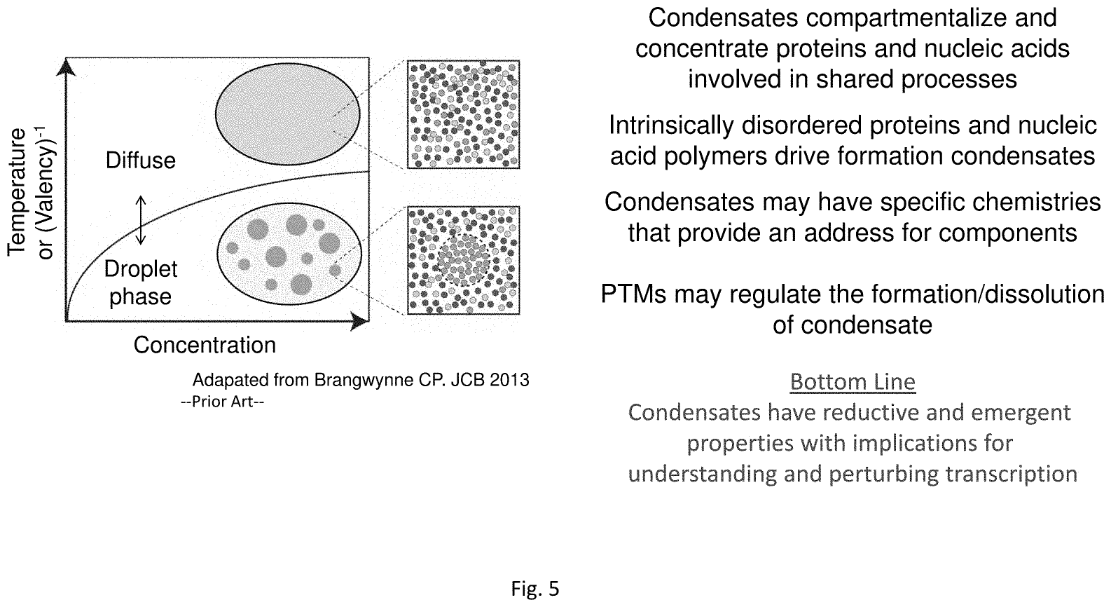

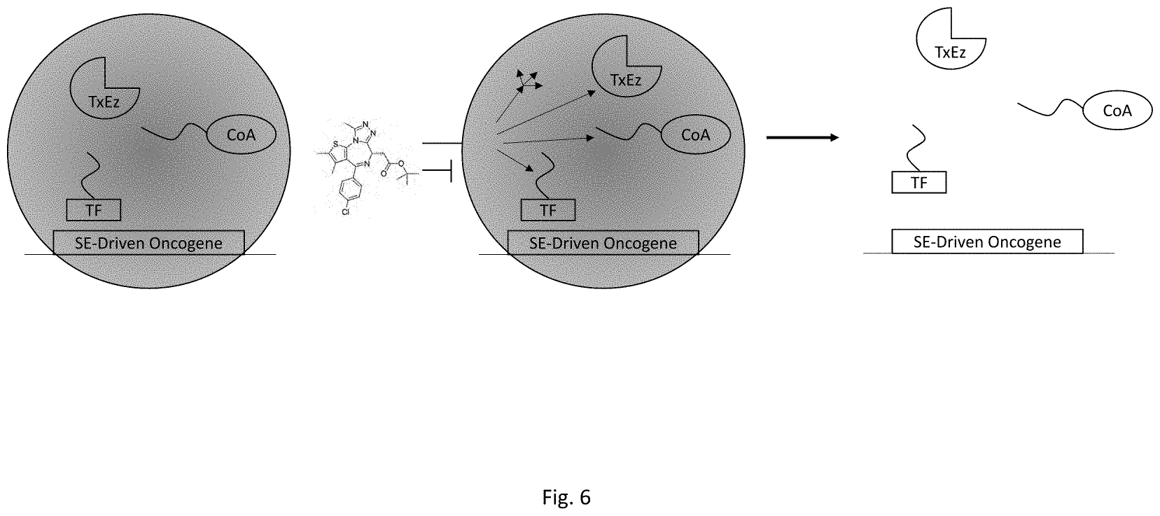

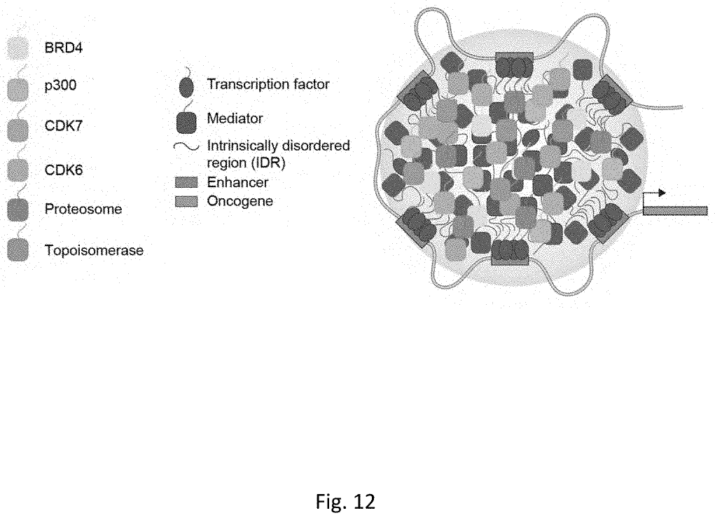

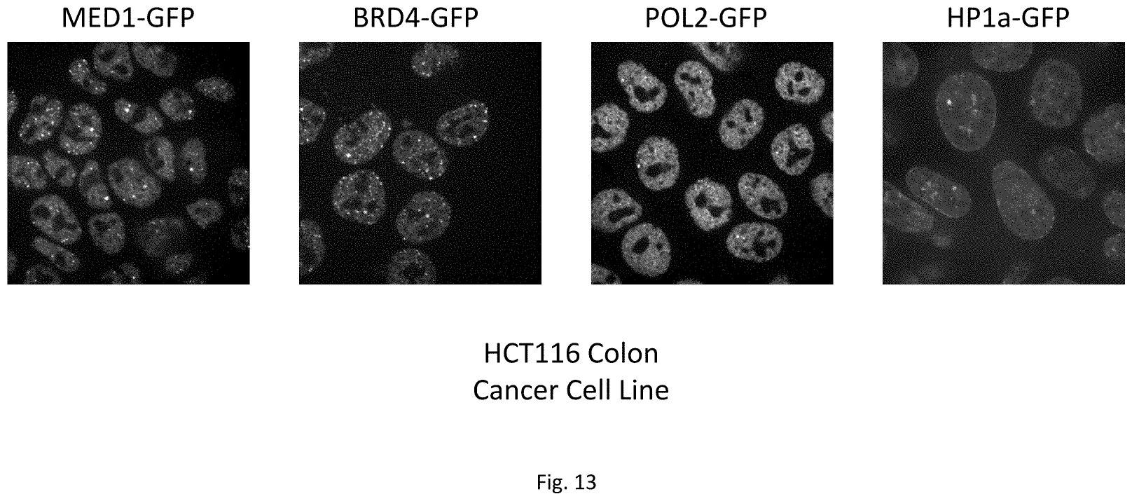

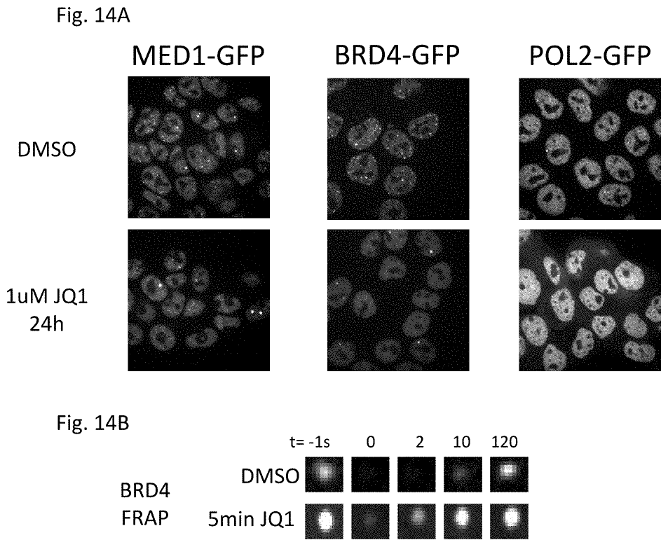

As used herein, “transcriptional condensates” are phase-separated multi-molecular assemblies that occur at the sites of transcription and are high density cooperative assemblies of multiple components that can include transcription factors, co-factors (e.g., co-activator), chromatin regulators, DNA, non-coding RNA, nascent RNA, RNA polymerase II, kinases, proteasomes, topoisomerase, and/or enhancers (see, e.g., FIGS. 4, 11, and 12). As used herein, a “super-enhancer condensate” is a transcriptional condensate occurring at a super-enhancer. Super-enhancers are known in the art. See, e.g., US patent application publication No. 20140287932 A1, incorporated herein by reference. As used herein, “heterochromatin condensates” are phase-separated multi-molecular assemblies that are physically associated with (e.g., occur on) heterochromatin. Heterochromatin condensates have been shown to be associated with repression of gene transcription. As used herein, condensates physically associated with an mRNA initiation or elongation complex are phase-separated multi-molecular assemblies occurring at the relevant complex. In some embodiments, a condensate physically associated with an elongation complex comprises splicing factors. In some embodiments, a condensate physically associated with an elongation complex is a splicing speckle. As used herein, a “splicing speckle” (also sometimes referred to as a nuclear speckle or interchromatin granule cluster) is a condensate enriched in splicing factors. See, e.g., Y. Chen, A. S. Belmont, Genome organization around nuclear speckles. Curr. Opin. Genet. Dev. 55, 91-99 (2019), incorporated herein by reference. As used herein, a “nucleolus” or “nucleoli” (plural form) is a condensate comprising RNA and protein occurring in the nucleus. See, e.g., M. Feric et al., Coexisting Liquid Phases Underlie Nucleolar Subcompartments. Cell. 165, 1686-1697 (2016), incorporated herein by reference. As used herein, “chromatin condensates” are phase-separated multi-molecular assemblies that are physically associated with chromatin. See, Gibson et al., Organization of Chromatin by Intrinsic and Regulated Phase Separation, Cell (2019), incorporated herein by reference. As used herein, “polycomb condensates” are phase-separated multi-molecular assemblies that physically associate with chromatin and can suppress gene transcription. See, Plys, et al., Phase separation of Polycomb-repressive complex 1 is governed by a charged disordered region of CBX2, Genes Dev. 2019 Jul. 1; 33(13-14):799-813, incorporated herein by reference. As used herein, “DNA damage repair condensates” are phase-separated multi-molecular assemblies that physically associate with double stranded DNA breaks. See, Pessina et al., Functional transcription promoters at DNA double-strand breaks mediate RNA-driven phase separation of damage-response factors, Nature Cell Biology volume 21, pages 1286-1299 (2019), incorporated herein by reference.

In some preferred embodiments of the methods disclosed herein, the condensate is a transcriptional condensate or an in vitro condensate comprising one or more components of a transcriptional condensate. In some preferred embodiments of the methods disclosed herein, the condensate is a super-enhancer condensate or an in vitro condensate comprising one or more components of a super-enhancer condensate. In some preferred embodiments of the methods disclosed herein, the condensate is a splicing speckle condensate or an in vitro condensate comprising one or more components of a splicing speckle condensate. In some preferred embodiments of the methods disclosed herein, the condensate is a heterochromatin condensate or an in vitro condensate comprising one or more components of a heterochromatin condensate. In some preferred embodiments of the methods disclosed herein, the condensate is a heterochromatin condensate or an in vitro condensate comprising one or more components of a heterochromatin condensate. In some preferred embodiments of the methods disclosed herein, the condensate is a nucleolus or an in vitro condensate comprising one or more components of a nucleolus. In some preferred embodiments of the methods disclosed herein, the condensate is a chromatin condensate or an in vitro condensate comprising one or more components of a chromatin condensate. In some preferred embodiments of the methods disclosed herein, the condensate is a polycomb condensate or an in vitro condensate comprising one or more components of a polycomb condensate. In some preferred embodiments of the methods disclosed herein, the condensate is a DNA damage repair condensate or an in vitro condensate comprising one or more components of a DNA damage repair condensate.

As used herein, the phrase “a condensate component” or the like refers to a peptide, protein, nucleic acid, signaling molecule, lipid, or the like that is part of a condensate or has the capability of being part of a condensate (e.g., transcriptional condensate, super-enhancer condensate, splicing speckle condensate, heterochromatin condensate, nucleolus, chromatin condensate, polycomb condensate, or DNA damage repair condensate). In some embodiments, the component is within the condensate. In some embodiments, the component is necessary for condensate formation or stability. In some embodiments, the component is not necessary for condensate formation or stability. In some embodiments, the component is a protein or peptide and comprises one or more intrinsically ordered domains (e.g., an IDR of an activation domain of a transcription factor, an IDR that interacts with an IDR of an activation domain of a transcription factor, an IDR of a signaling factor, an IDR of a methyl-DNA binding protein, an IDR of a gene silencing factor, an IDR of a polymerase, an IDR of a splicing factor, an IDR of a nucleolar small nuclear ribonucleoprotein, an IDR of nucleophosmin, an IDR of a histone, an IDR of CBX2, an IDR of 53BP1). In some embodiments, the component is a non-structural member of a condensate (e.g., not necessary for condensate integrity). In some embodiments, a condensate comprises, consists of, or consists essentially of 1, 2, 3, 4, 5, 6, 7, 8, 9, 10 or more components. In some embodiments, a condensate (e.g., an in vitro condensate) does not comprise a nucleic acid. In some embodiments, a condensate (e.g., an in vitro condensate) does not comprise RNA. In some embodiments, the component is a fragment of a protein or nucleic acid.

As shown in the examples below, replacement of basic amino acids in MED1 with alanine impaired the ability of the mutant MED1 to form droplets (i.e., in vitro condensates) in solution. Thus, in some embodiments, the condensate component is a naturally occurring protein or polypeptide that has been modified to increase or decrease the number of basic amino acid residues and thereby modulate the ability of the condensate component to form a condensate (e.g., droplet). In some embodiments, at least about 10%, at least about 20%, at least about 30%, at least about 40%, at least about 50%, at least about 60%, at least about 70%, at least about 80%, at least about 90%, or all basic amino acid residues have been replaced with non-basic amino acid residues (e.g., alanine or other neutral amino acid, e.g., asparagine, cysteine, glutamine, glycine, isoleucine, leucine, methionine, phenylalanine, proline, serine, threonine, tryptophan, tyrosine, valine, etc.) In some embodiments, at least 1, 2, 3, 4, 5, 6, 7, 8, 9, 10, 11, 12, 13, 14, 15, 16, 17, 18, 19, 20, 25, 30, or more basic amino acid residues are replaced with non-basic amino acid residues. In some embodiments, at least 1, 2, 3, 4, 5, 6, 7, 8, 9, 10, 11, 12, 13, 14, 15, 16, 17, 18, 19, 20, 25, 30, 35, 40, 45, 50 or more basic amino acid residues are added to the condensate component.

In some embodiments, the ability of the modified condensate component to form a condensate (e.g., droplet) is decreased by about at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to an unmodified condensate component. In some embodiments, the ability of the modified condensate component to form a condensate (e.g., droplet) is increased by about at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to an unmodified condensate component.

As shown in the Examples below, replacement of aromatic amino acids in MED1 with alanine impaired the ability of agents comprising aromatic substituents to incorporate into droplets of the modified MED1. Thus, in some embodiments, the condensate component is a naturally occurring protein or polypeptide that has been modified to increase or decrease the number of aromatic amino acid residues and thereby modulate the ability of condensates comprising the condensate component to incorporate agents comprising aromatic substituents. In some embodiments, at least about 10%, at least about 20%, at least about 30%, at least about 40%, at least about 50%, at least about 60%, at least about 70%, at least about 80%, at least about 90%, or all aromatic amino acid residues have been replaced with non-aromatic amino acid residues (e.g., alanine or other neutral amino acid, e.g., asparagine, cysteine, glutamine, glycine, isoleucine, leucine, methionine, phenylalanine, proline, serine, threonine, tryptophan, tyrosine, valine, etc.) In some embodiments, at least 1, 2, 3, 4, 5, 6, 7, 8, 9, 10, 11, 12, 13, 14, 15, 16, 17, 18, 19, 20, 25, 30, or more aromatic amino acid residues are replaced with non-aromatic amino acid residues. In some embodiments, at least 1, 2, 3, 4, 5, 6, 7, 8, 9, 10, 11, 12, 13, 14, 15, 16, 17, 18, 19, 20, 25, 30, 35, 40, 45, 50 or more aromatic amino acid residues are added to the condensate component.

In some embodiments, the ability of condensates comprising the modified condensate component to incorporate agents comprising aromatic substituents is decreased by about at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to a corresponding condensate comprising an unmodified condensate component. In some embodiments, the ability of condensates comprising the modified condensate component to incorporate agents comprising aromatic substituents is increased by about at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to a corresponding condensate comprising an unmodified condensate component.

In some specific embodiments, provided herein is a method for obtaining an agent with a desired partition coefficient comprising (a) providing a first agent having a partition coefficient and at least a second agent that is the same as the first agent except that one or more non-aromatic amino acids are replaced with aromatic amino acids and/or one or more aromatic amino acids are added, and (b) measuring the partition coefficient of the second agent to thereby obtain an agent with a desired partition coefficient. In some specific embodiments, provided herein is a method for obtaining an agent with a desired partition coefficient comprising (a) providing a first agent having a partition coefficient and at least a second agent that is the same as the first agent except that one or more aromatic amino acids are replaced with non-aromatic amino acids and/or one or more aromatic amino acids are removed, and (b) measuring the partition coefficient of the second agent to thereby obtain an agent with a desired partition coefficient.

Regions of intrinsic disorder, also termed intrinsic (or intrinsically) disordered regions (IDR) or intrinsic (or intrinsically) disordered domains can be found in many protein condensate components. Each of these terms is used interchangeably throughout the disclosure. IDR lack stable secondary and tertiary structure. In some embodiments, an IDR may be identified by the methods disclosed in Ali, M., & Ivarsson, Y. (2018). High-throughput discovery of functional disordered regions. Molecular Systems Biology, 14(5), e8377. IDRs are known in the art and any suitable method may be used to identify an IDR.

In some embodiments, the component is a signaling factor, a methyl-DNA binding protein, BRD4, Mediator, a mediator component, MED1, MED15, a transcription factor, an RNA polymerase, a DNA sequence (e.g., an enhancer DNA sequence, a methylated DNA sequence, a super-enhancer DNA sequence, 3′ end of a transcribed gene, a signal response element, a hormone response element, an oncogene or portion thereof), a gene silencing factor, a splicing factor, an elongation factor, an initiation factor, a histone (e.g., a modified histone), a co-factor, an RNA (e.g., ncRNA), mediator, an RNA polymerase (e.g., RNA polymerase II), a kinase (e.g., cyclin dependent kinase, CDK7, CDK8), proteasome, or topoisomerase. In some embodiments, the component is MED1, BRD4, POLII, SRSF2, FIB1, NPM1, histone, CBX2, 53BP1, or HP1α, or a functional fragment (e.g., a fragment comprising an IDR) thereof. In some embodiments, the co-factor comprises an LXXLL motif. In some embodiments, the co-factor comprises an LXXLL motif and has increased valency for a TF (e.g., a nuclear receptor, a master transcription factor) when bound to a ligand (e.g., a cognate ligand, a naturally occurring ligand, a synthetic ligand). Co-factors having LXXLL motifs are known in the art. In some embodiments, the component is a fragment of a co-factor comprising an IDR and LXXLL motif. In some embodiments, the component is a protein or nucleic acid. The component is not limited and may be any condensate component identified in the art.

As used herein, “a mediator component” comprises or consists of a polypeptide whose amino acid sequence is identical to the amino acid sequence of a naturally occurring Mediator complex polypeptide. The naturally occurring Mediator complex polypeptide can be, e.g., any of the approximately 30 polypeptides found in a Mediator complex that occurs in a cell or is purified from a cell (see, e.g., Conaway et al., 2005; Kornberg, 2005; Malik and Roeder, 2005). In some embodiments a naturally occurring Mediator component is any of Med1-Med 31 or any naturally occurring Mediator polypeptide known in the art. For example, a naturally occurring Mediator complex polypeptide can be Med6, Med7, Med10, Med12, Med14, Med15, Med17, Med21, Med24, Med27, Med28 or Med30. In some embodiments a Mediator polypeptide is a subunit found in a Med11, Med17, Med20, Med22, Med 8, Med 18, Med 19, Med 6, Med 30, Med 21, Med 4, Med 7, Med 31, Med 10, Med 1, Med 27, Med 26, Med14, Med15 complex. In some embodiments a Mediator polypeptide is a subunit found in a Med12/Med13/CDK8/cyclin complex. Mediator is described in further detail in PCT International Application No. WO 2011/100374, the teachings of which are incorporated herein by reference in their entirety.

In some embodiments, a component of the condensate is a signaling factor selected from the group consisting of TCF7L2, TCF7, TCF7L1, LEF1, Beta-Catenin, SMAD2, SMAD3, SMAD4, STAT1, STAT2, STAT3, STAT4, STAT5A, STAT5B, STAT6, and NF-κB. In some embodiments, the signaling factor comprises one or more intrinsic disorder domains. In some embodiments, the condensate comprises a master transcription factor.

In some embodiments, the component of the condensate is a methyl-DNA binding protein that preferentially binds to methylated DNA. In some embodiments, the methyl-DNA binding protein is MECP2, MBD1, MBD2, MBD3, or MBD4. In some embodiments, the methyl-DNA binding protein is associated with gene silencing. In some embodiments, the component is a suppressor associated with heterochromatin. In some embodiments, the methyl-DNA binding protein is HP1α, TBL1R (transducin beta-like protein), HDAC3 (histone deacetylase 3) or SMRT (silencing mediator of retinoic and thyroid receptor).

In some embodiments, the component of the condensate is an RNA polymerase associated with mRNA initiation and elongation. In some embodiments, the RNA polymerase is RNA polymerase II or an RNA polymerase II C-terminal region. In some embodiments, the RNA polymerase II C-terminal region comprises an intrinsically disordered region (IDR). In some embodiments, the IDR comprises a phosphorylation site. In some embodiments, the component is a splicing factor selected from SRSF2, SRRM1, or SRSF1.

In some embodiments, the component of the condensate is a transcription factor. In some embodiments, the transcription factor is OCT4, p53, MYC or GCN4, NANOG, MyoD, KLF4, a SOX family transcription factor, a GATA family transcription factor, or a nuclear receptor (e.g., a nuclear hormone receptor, Estrogen Receptor, Retinoic Acid Receptor-Alpha).

In some embodiments, the nuclear receptor (NR) is a nuclear receptor subfamily 0 member, nuclear receptor subfamily 1 member, nuclear receptor subfamily 2 member, nuclear receptor subfamily 3 member, nuclear receptor subfamily 4 member, nuclear receptor subfamily 5 member, or nuclear receptor subfamily 6 member. In some embodiments, the nuclear receptor is NR1D1 (nuclear receptor subfamily 1, group D, member 1), NR1D2 (nuclear receptor subfamily 1, group D, member 2), NR1H2 (nuclear receptor subfamily 1, group H, member 2; synonym: liver X receptor beta), NR1H3 (nuclear receptor subfamily 1, group H, member 3; synonym: liver X receptor alpha), NR1H4 (nuclear receptor subfamily 1, group H, member 4), NR1I2 (nuclear receptor subfamily 1, group I, member 2; synonym: pregnane X receptor), NR1I3 (nuclear receptor subfamily 1, group I, member 3; synonym: constitutive androstane receptor), NR1I4 (nuclear receptor subfamily 1, group I, member 4), NR2C1 (nuclear receptor subfamily 2, group C, member 1), NR2C2 (nuclear receptor subfamily 2, group C, member 2), NR2E1 (nuclear receptor subfamily 2, group E, member 1), NR2E3 (nuclear receptor subfamily 2, group E, member 3), NR2F1 (nuclear receptor subfamily 2, group F, member 1), NR2F2 (nuclear receptor subfamily 2, group F, member 2), NR2F6 (nuclear receptor subfamily 2, group F, member 6), NR3C1 (nuclear receptor subfamily 3, group C, member 1; synonym: glucocorticoid receptor), NR3C2 (nuclear receptor subfamily 3, group C, member 2; synonym: aldosterone receptor, mineralocorticoid receptor), NR4A1 (nuclear receptor subfamily 4, group A, member 1), NR4A2 (nuclear receptor subfamily 4, group A, member 2), NR4A3 (nuclear receptor subfamily 4, group A, member 3), NR5A1 (nuclear receptor subfamily 5, group A, member 1), NR5A2 (nuclear receptor subfamily 5, group A, member 2), NR6A1 (nuclear receptor subfamily 6, group A, member 1), NROB1 (nuclear receptor subfamily 0, group B, member 1), NROB2 (nuclear receptor subfamily 0, group B, member 2), RARA (retinoic acid receptor, alpha), RARB (retinoic acid receptor, beta), RARG (retinoic acid receptor, gamma), RXRA (retinoid X receptor, alpha; synonym: nuclear receptor subfamily 2 group B member 1), RXRB (retinoid X receptor, beta; synonym: nuclear receptor subfamily 2 group B member 2), RXRG (retinoid X receptor, gamma; synonym: nuclear receptor subfamily 2 group B member 3), THRA (thyroid hormone receptor, alpha), THRB (thyroid hormone receptor, beta), AR (androgen receptor), ESR1 (estrogen receptor 1), ESR2 (estrogen receptor 2; synonym: ER beta), ESRRA (estrogen-related receptor alpha), ESRRB (estrogen-related receptor beta), ESRRG (estrogen-related receptor gamma), PGR (progesterone receptor), PPARA (peroxisome proliferator-activated receptor alpha), PPARD (peroxisome proliferator-activated receptor delta), PPARG (peroxisome proliferator-activated receptor gamma), or VDR (vitamin D (1,25-dihydroxyvitamin D3) receptor).

In some embodiments, the nuclear receptor is a naturally occurring truncated form of a nuclear receptor generated by proteolytic cleavage, such as truncated RXR alpha, or truncated estrogen receptor. In some embodiments, the nuclear receptor is an HSP70 client. For example, androgen receptor (AR) and glucocorticoid receptor (GR) are HSP70 clients. Extensive information regarding NRs may be found in Germain, P., et al., Pharmacological Reviews, 58:685-704, 2006, which provides a review of nuclear receptor nomenclature and structure, and other articles in the same issue of Pharmacological Reviews for reviews on NR subfamilies). In some embodiments, an HSP90A client is a steroid hormone receptor (e.g., an estrogen, progesterone, glucocorticoid, mineralocorticoid, or androgen receptor), PPAR alpha, or PXR. In some embodiments, the nuclear receptor (NR) is a ligand-dependent NR. A ligand-dependent NR is characterized in that binding of a ligand to the NR modulates activity of the NR. In some embodiments binding of a ligand to ligand-dependent NF causes a conformational change in the NR that results in, e.g., nuclear translocation of the NR, dissociation of one or more proteins from the NR, activation of the NR, or repression of the NR. In some embodiments, the NR is a mutant that lacks one or more activities of the wild-type NR upon ligand binding (e.g., nuclear translocation of the NR, dissociation of one or more proteins from the NR, activation of the NR, or repression of the NR). In some embodiments, the NR is a mutant having a ligand-binding independent activity (e.g., nuclear translocation of the NR, dissociation of one or more proteins from the NR, activation of the NR, or repression of the NR) that is ligand dependent in the wild-type NR. In some embodiments, the nuclear receptor activates transcription when bound to a cognate ligand. In some embodiments, the nuclear receptor is a mutant nuclear receptor that activates transcription in the absence of the cognate ligand.

In some embodiments of the methods disclosed herein, the transcription factor is a human transcription factor identified in Lambert, et al., Cell. 2018 Feb. 8; 172(4):650-665. In some embodiments, the nuclear receptor activates transcription when bound to a cognate ligand. In some embodiments, the nuclear receptor is a mutant nuclear receptor that activates transcription in the absence of a cognate ligand, or has a higher level of transcription activity (e.g., at least 1.5-fold, at least 2-fold, at least 3-fold, or more) in the absence of a cognate ligand than the wild-type nuclear receptor in the presence of the natural ligand (e.g., cognate ligand). In some embodiments, the nuclear receptor is a mutant nuclear transcription factor that modulates transcription in the presence of a cognate ligand to a different degree than the wild-type nuclear receptor. In some embodiments, the transcription factor is a fusion oncogenic transcription factor. In some embodiments, the fusion oncogenic transcription factor is selected from MLL-rearrangements, EWS-FLI, ETS fusions, BRD4-NUT, and NUP98 fusions. The oncogenic transcription factor may be any oncogenic transcription factor identified in the art.

In some embodiments, the components of the condensate are components found in transcriptional condensates. In some embodiments, the transcriptional condensate components comprise transcription factors, co-factors, chromatin regulators, DNA, non-coding RNA, nascent RNA, RNA polymerase II, kinases, proteasomes, topoisomerase, and/or enhancers. In some embodiments, the transcription factor is, e.g., OCT4, p53, MYC, GCN4, NANOG, MyoD, KLF4, a SOX family transcription factor, a GATA family transcription factor, a nuclear receptor, or a fusion oncogenic transcription factor.

In some embodiments, the components of the condensates are components found in nucleoli. In some embodiments, the nucleoli components are rRNA processing factors, POL1, FIB1, nucleophosmin, ribosomal DNA gene clusters, and/or POLR1E.

In some embodiments, incorporation of the agent into the condensate is detected without using a detectable tag. In some embodiments, the agent naturally fluoresces. In some embodiments, the agent has a color that differentiates it from condensate and/or from the background or area outside the condensate. In some embodiments, incorporation of the agent is detected by Raman spectroscopy (see, e.g., Smith et al., Analyst, 2016, 141, pp. 3590-3600). In some embodiments, incorporation of the agent is detected by nuclear magnetic resonance (NMR). In some embodiments, incorporation of the agent is detected by mass spectrometry. In some embodiments, incorporation of the agent is detected by spectrophotometry and quantitative phase microscopy. In some embodiments, incorporation of the agent is detected by coherence-controlled holographic microscopy. In some embodiments, incorporation of the agent is detected by spin down assay. It will also be appreciated that incorporation of an agent into a condensate may be detected by detecting the amount or proportion of agent that is not incorporated into the condensate.

In some embodiments, incorporation of the agent into the condensate is detected by isolating the condensate from agent not incorporated in the condensate and then measuring agent remaining in the condensate. Any suitable method of isolating a condensate may be used and is not limited. In some embodiments, the condensate is isolated by removal of the condensate from a cell having the condensate. In some embodiments, the condensate is isolated by removal of the condensate from an in vitro composition (e.g., solution) comprising the condensate. In some embodiments, the condensate is crosslinked to assist in isolation of the condensate. In some embodiments, the isolated condensate is disrupted and the amount or proportion of agent measured. Any suitable method of disruption may be used including physical and/or chemical means. In some embodiments, condensates may be disrupted by increasing or decreasing the concentration of salt or crowding agent in the solution. In some embodiments, condensates may be disrupted via sonication, centrifugation, or by varying the temperature. In some embodiments, the agent from the disrupted condensate is measured by chromatography (e.g., HPLC).

In some embodiments, incorporation of the agent in the condensate is measured relative to a control. The control may a compound that is known to incorporate into the condensate under appropriate physiological conditions. The control may also be a compound having similar physical or chemical properties as the agent and having a known incorporation profile into a condensate. In some embodiments, the enrichment ratio or partition coefficient of the agent is determined (i.e., the relative concentrations of the agent in and outside the condensate). In some embodiments, the enrichment ratio is determined by measuring the fluorescence of a fluorescent tag on the agent both in and outside the condensate. In some embodiments, the enrichment ratio is detected by a method described in the Examples section. Methods of determining enrichment ratios and partition coefficients are known in the art and are not limited. In some embodiments, the amount of agent that is partitioned into a condensate is determined. In some embodiments, the agent comprises a detectable tag. In some embodiments, incorporation of the agent in the condensate is measured using the detectable tag. The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label. In some embodiments, the detectable tag is a fluorescent tag. In some embodiments, both a condensate component and the agent comprise a detectable tag. In some embodiments, the component comprises a different detectable tag than the agent.

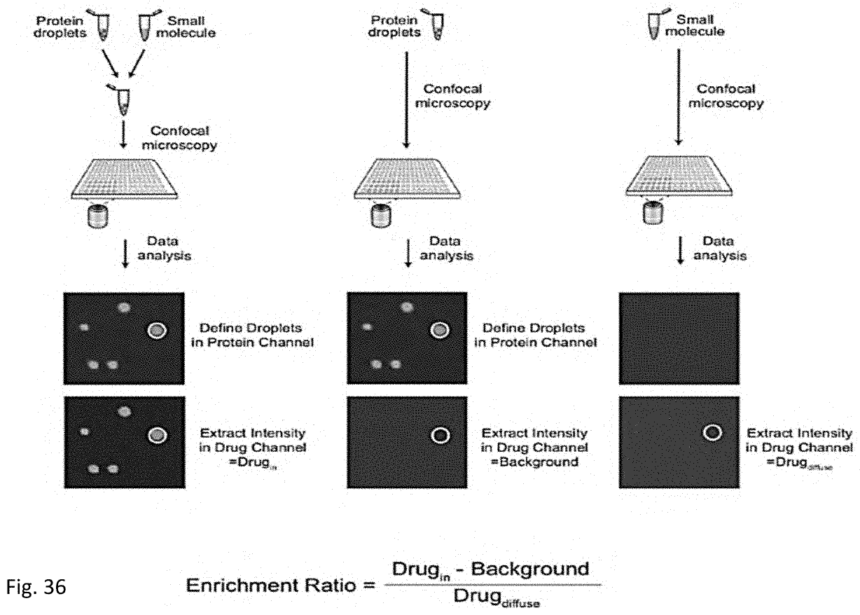

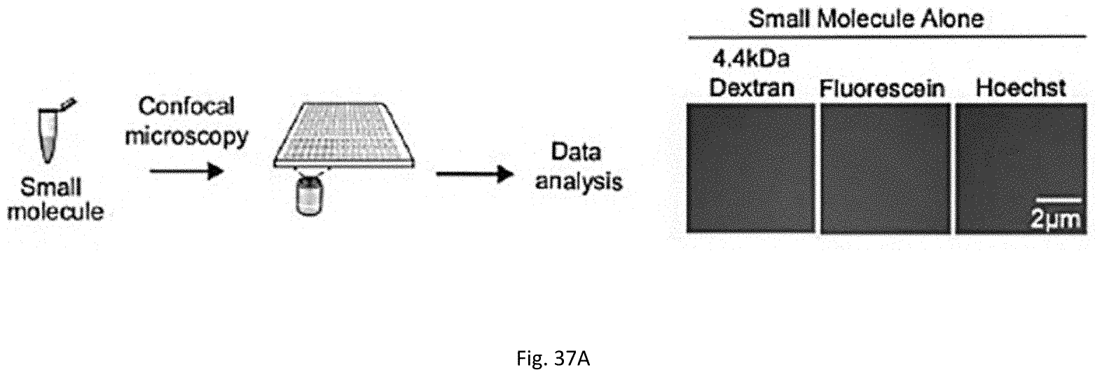

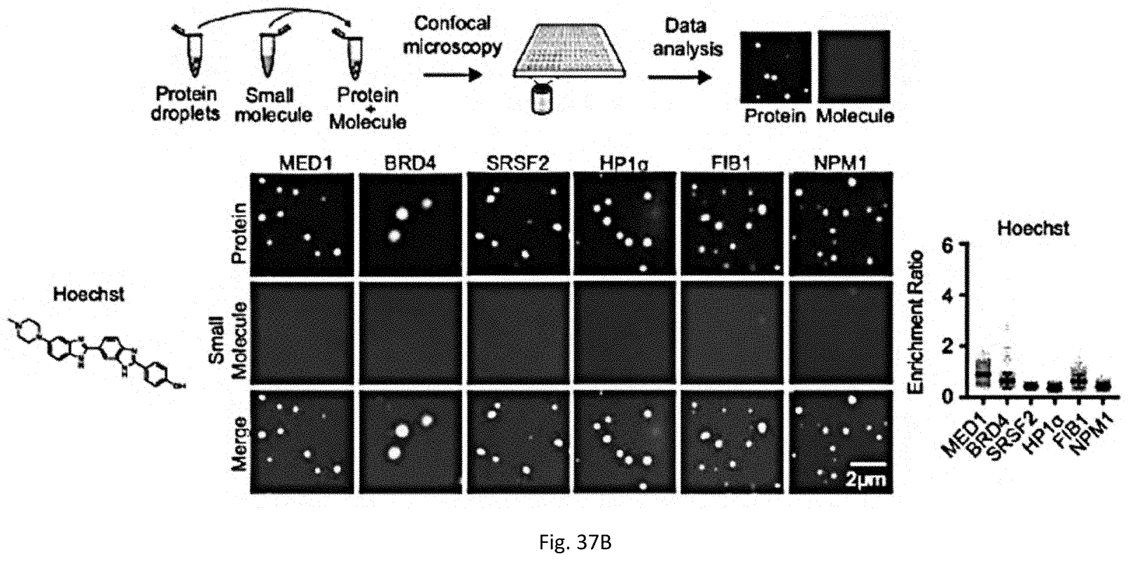

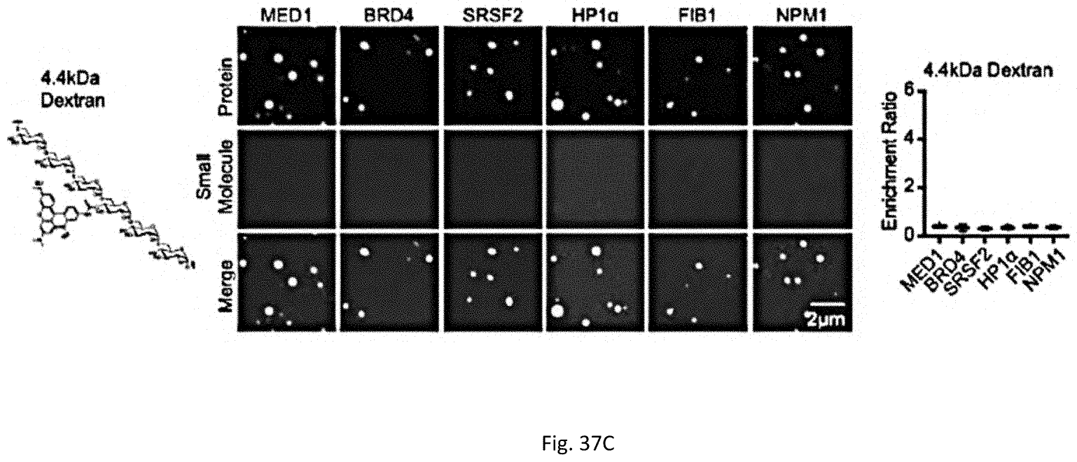

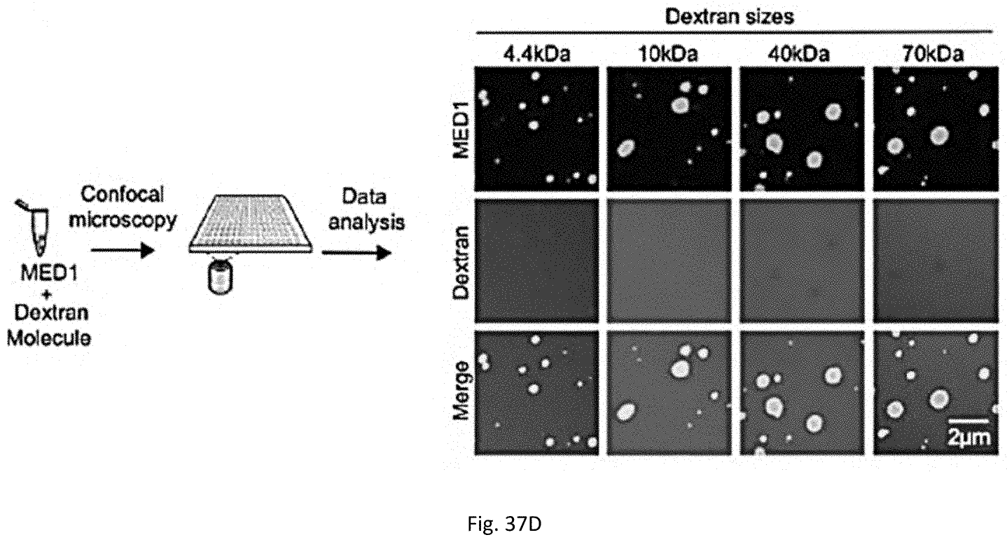

Methods of calculating the incorporation of the agent into a condensate are not limited and may be any method known in the art. In some embodiments, the enrichment ratio for the agent is determined by the method shown in FIG. 36. In some embodiments, the enrichment ratio of an agent (e.g., an agent having a detectable tag or an agent having a detectable property) for a particular condensate is determined by providing the condensate in solution with the agent and detecting the intensity of the agent in the condensate by confocal microscopy to obtain a Drugᵢₙ value; providing the condensate in solution without the agent and detecting the intensity of the background within the condensates to obtain a Background value; and providing the agent in solution without the condensate and detecting the intensity of the agent to obtain a Drug_{diffuse} value; wherein the enrichment ratio is equal to (Drugᵢₙ−Background)/(Drug_{diffuse}). In some embodiments, agent partitioning can be determined experimentally by spectrophotometry and quantitative phase microscopy. In some embodiments, a sample composed of two coexisting phases is considered, named dilute and condensed, with volume fractions ϕdilute and ϕcond=1. If an agent is also present in the sample at an average concentration of ctotal, then mass conservation requires that

cₜₒₜₐₗ=c_{dilute}ϕ_{dilute}+c_{cond}ϕ_{cond}, (1)

where c_{dilute} and c_{cond} are the concentrations of the agent in the dilute and condensed phases, respectively. The partition coefficient is defined for the agent into the condensed phase as P=c_{cond}/c_{dilute}. With this definition and the requirement that the phase volume fractions sum to 1, Eq 1 can be written as

cₜₒₜₐₗ=c_{dilute}(1−ϕ_{cond})+c_{dilute}Pϕ_{cond}, (2)

which can be simplified and rearranged to yield

$$P = 1 + ( \frac{{c}_{total}}{{c}_{dilute}} - 1 ) {( {ϕ}_{cond} )}^{- 1} . \qquad ( 3 )$$

the ratio cₜₒₜₐₗ/c_{dilute} is estimated from fluorescence spectroscopy measurements, as described below, while ϕ_{cond}, it is inferred from the lever rule (M. Rubinstein, R. H. Colby, Polymer Phyics (Oxford University Press, 2003)) as follows: denoting the concentration of condensate protein (e.g. MED1) by s, mass conservation gives stotal=s_{dilute}ϕ_{dilute}+s_{cond}ϕ_{cond}, in analogy with Eq. 1. Again, using the requirement that the volume fractions of coexisting phases sum to 1, this can be rearranged to yield

$${ϕ}_{cond} = \frac{{s}_{total} - {s}_{dilute}}{{s}_{cond} - {s}_{dilute}} . \qquad ( 4 )$$

where sₜₒₜₐₗ and s_{dilute} are measured spectrophotometrically from optical absorbance, e.g. at 280 nm, and s_{cond} is measured from quantitative phase microscopy, using e.g., a coherence-controlled holographic microscope.

Uv-vis spectroscopy can be used to estimate the absolute concentration of agent in solution using Beer-Lambert law with Eq 5,

A=Log 10(I0/I)=εcL (5)

wherein A is the measured absorbance (in Absorbance Units (AU)), I0 is the intensity of the incident light at a given wavelength, I is the transmitted intensity, L the path length through the sample, and c the concentration of the absorbing species. For each species and wavelength, c is a constant known as the molar absorptivity or extinction coefficient. This constant is a fundamental molecular property in a given solvent, at a particular temperature and pressure, and has units of 1/M*cm.

In some embodiments, the amount of partitioned agent can be measured by using a spin down assay. Specifically, a known concentration of agent is added with condensate components and droplets are allowed to form. Then the mixture is centrifuged to pellet the droplets, the supernatant collected, and the concentration of agent in the supernatant measured. Amount of agent partitioned can then be determined by subtracting the concentration of agent in the supernatant from the total known concentration of drug added.

In some embodiments, quantitative phase measurements can be performed using a coherence-controlled holographic microscope, e.g., as detailed below in the examples. Software can be used to construct compensated phase images from acquired holograms. In some embodiments, each phase image is spatially segmented based on intensity, and a window containing each segmented object is fit to a spatial function of the form

$$φ ( x , y ) = \frac{2 π}{λ} Δ nH ( x , y \vert R ) . \qquad ( 6 )$$

where φ(x, y) is the phase intensity at pixel location (x, y), λ is the illumination wavelength, Δn is the refractive index difference between condensates and the surrounding dilute phase, and H(x, y|R) is the projected height of a sphere of radius R. The fitting parameters in Eq. 6 are Δn and R. It is assumed that no PEG partitions into the condensates and calculate the average scaffold concentration in each filtered condensate as

$${s}_{cond} = \frac{Δ n + ( {n}_{dilute} - {n}_{0} )}{dn / ds} x \qquad ( 7 )$$

Here n₀ is the refractive index of buffer in the absence of scaffold and PEG, n_{dilute} is the refractive index of the dilute phase, and both are measured at using digital refractometer. The refractive index increment of the condensate protein, dn/ds, can be estimated from amino acid composition.

In some embodiments, agent-target interactions in the presence of a condensate may be modeled. Such modeling may be useful, e.g., for determining an effective partitioning coefficient and/or concentration of an agent to be therapeutically effective against a target. In some embodiments, the modeling may be a simplified model as shown in the examples herein. This simplified model was developed of drug-target interactions in the presence of a condensate. The relevant species are the drug (D) (i.e., agent), target (T), and the drug-target complex (D-T). It is assumed that there are only 2-types of phases, the bulk/dilute nuclear phase (n) and the condensate phase (c), which is present with volume fraction ƒ=V_{condensate}/V_{nucleus}. At equilibrium, the following partitioning conditions are obeyed:

$$\frac{{[ D ]}_{c}}{{[ D ]}_{n}} = {p}_{D} ; \frac{{[ T ]}_{c}}{{[ T ]}_{n}} = {p}_{T} ;$$

where p_{D}, p_{T} are the partition coefficients of the drug and target. [D]_{c} represents the concentration of species D in condensate phase (and similarly for other components/phases). In this model, the drug and target complex with phase-independent disassociation constant of K_{D}.

$$[ D ] + [ T ] {↔}^{{K}_{D}} [ D - T ] {K}_{D} = \frac{[ D ] [ T ]}{[ D - T ]}$$

To solve for equilibrium concentrations of various species, which are present at overall levels [D]₀, [T]₀, the species balance is written down as:

ƒ([D]_{c}+[D−T]_{c})+(1−ƒ)([D]ₙ+[D−T]ₙ)=[D]₀

ƒ([T]_{c}+[D−T]_{c})+(1−ƒ)([T]ₙ+[D−T]ₙ)=[DT]₀

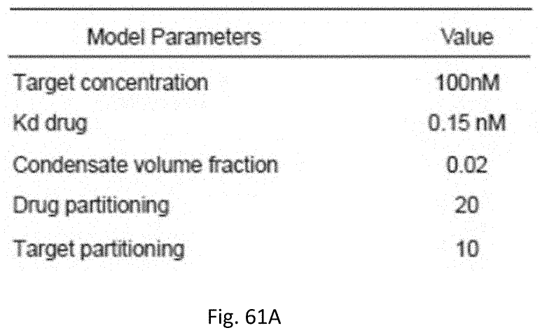

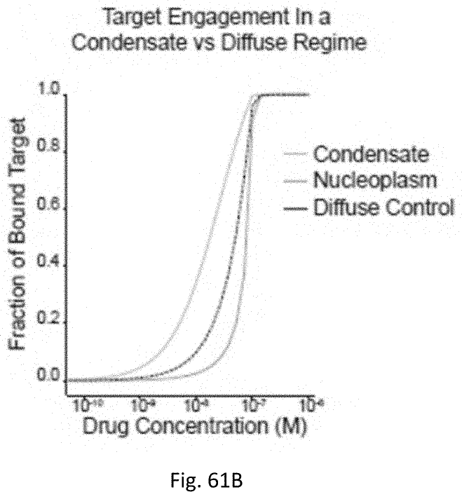

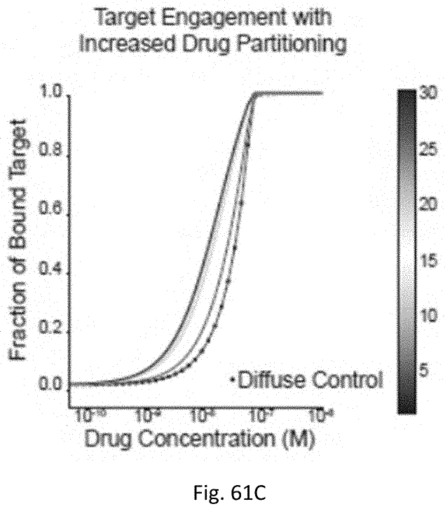

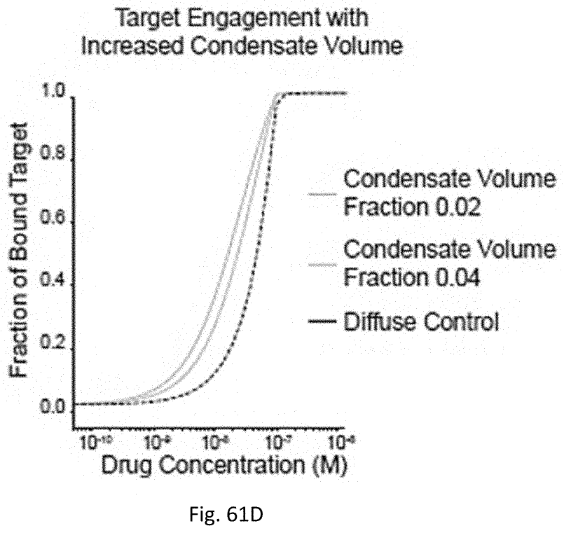

These 6 concentrations are solved with 2-equations and 4 constraints (2 from partitioning and 2 from reaction equilibria). In FIG. 61A-61D, the fraction of bound target is defined as:

$${Fraction}_{bound} , c = \frac{{[ D - T ]}_{c}}{{[ D ]}_{c} + {[ T ]}_{c}}$$

A similar expression is used for the fraction of bound target in the nuclear (bulk or dilute) phase. In case of controls plotted, plot fraction is plotted when there is only 1 phase (f=0).

The presence of a detectable tag on an agent may, in some cases, alter the incorporation activity of the agent into a condensate. However, if the labeled agent incorporated into a condensate that can be flushed out with an excess of unlabeled agent, then the incorporation of the labeled agent into the condensate is not mediated by the label. Thus, in some embodiments, the methods disclosed herein comprise contacting an agent having a detectable tag with the composition comprising the condensate, measuring incorporation of the agent having a detectable tag into the condensate, contacting the composition (e.g., solution) comprising the condensate and the agent having a detectable tag with a control agent not having a detectable tag (i.e., an identical agent not having the detectable tag), and again measuring incorporation of the agent having a detectable tag into the condensate. In some embodiments, at least an equal concentration of control agent is contacted. In some embodiments, an excess of control agent is contacted (e.g., at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold, or more of control agent). In some embodiments, a condensate incorporating a tagged agent is contacted with an increasing gradient of control agent and loss of tagged agent is measured continuously or at discrete intervals. In some embodiments, the method can further comprise contacting a condensate (e.g., droplet) with a tagged agent and an isomer of the agent with a lower partition coefficient for the condensate. In some embodiments, the isomer of the agent does not detectably partition into the condensate. In some embodiments, the tagged agent upon contact with a target in the condensate causes the ejection of the target from the condensate. In some embodiments, contact of the condensate with the tagged agent and an isomer of the tagged agent that does not appreciably partition into the condensate does not reduce the amount of target ejected after binding with the tagged target as compared to the tagged target not in the presence of the isomer. In some embodiments, the isomer is transplatin, which is an isomer of cisplatin, the tagged agent is tagged cisplatin and the target is estrogen receptor.

In some embodiments, a component of the condensate comprises a detectable tag. In some embodiments, both the agent and a component of the condensate comprise a detectable tag. The detectable tag is not limited and may be any detectable tag disclosed herein. In some embodiments, DNA or RNA incorporated into or associated with the condensate comprises a detectable tag.

Some Specific Embodiments of Characterizing an Agent as Disclosed Herein are as Follows:

Chase/Competition Droplet Assay:

In some embodiments, provided herein is a method for determining whether a first agent modulates the incorporation of a second agent in a condensate, the method comprising: (a) measuring the incorporation of the second agent in the condensate in the presence of the first agent; and (b) comparing the incorporation of the second agent in the condensate in the presence of the first agent to a reference, thereby determining whether the first agent modulates the incorporation of the second agent in the condensate. In some embodiments, the reference is based on the incorporation of the second agent in the condensate without the presence of the first agent. The first and second agent may be any agent described herein and are not limited. In some embodiments, at least the first or second agent is a small molecule as described herein.

In some embodiments, provided herein is a method for determining whether a first agent modulates the incorporation of a second agent in a condensate, the method comprising: (a) measuring the incorporation of the second agent in the condensate without the presence of the first agent; (b) measuring the incorporation of the second agent in the condensate in the presence of the first agent; and (c) comparing the incorporation of the second agent in the condensate without the presence of the first agent to the incorporation of the second agent in the condensate in the presence of the first agent, thereby determining whether the first agent modulates the incorporation of the second agent in the condensate.

In some embodiments, provided herein is a method for determining whether a first agent modulates the incorporation of a second agent in a condensate, the method comprising: (a) admixing a condensate and a second agent to form a reaction composition, wherein the condensate component comprises a first detectable tag, wherein the second agent comprises a second detectable tag, and wherein the signals of the first and second detectable tags are distinguishable; (b) measuring the incorporation of the second agent in the condensate without the presence of the first agent; (c) admixing the first agent in the reaction composition; (d) measuring the incorporation of the second agent in the condensate in the presence of the first agent; and (e) comparing the incorporation of the second agent in the condensate without the presence of the first agent to the incorporation of the second agent in the condensate in the presence of the first agent, thereby determining whether the first agent modulates the incorporation of the second agent in the condensate.

In some embodiments, provided herein is a method for determining whether a first agent modulates the incorporation of a second agent in a condensate, the method comprising: (a) admixing a composition comprising a component of the condensate and a second agent to form a reaction composition and cause the formation of the condensate in the reaction composition, wherein the condensate component comprises a first detectable tag, wherein the second agent comprises a second detectable tag, and wherein the signals of the first and second detectable tags are distinguishable; (b) measuring the incorporation of the second agent in the condensate without the presence of the first agent; (c) admixing the first agent in the reaction composition; (d) measuring the incorporation of the second agent in the condensate in the presence of the first agent; and (e) comparing the incorporation of the second agent in the condensate without the presence of the first agent to the incorporation of the second agent in the condensate in the presence of the first agent, thereby determining whether the first agent modulates the incorporation of the second agent in the condensate. In some embodiments, measuring the incorporation of an agent in a condensate comprises use of a technique comprising Raman spectroscopy, spectrophotometry, quantitative phase microscopy, fluorescent microscopy, including quantitative fluorescent microscopy, and/or a spin down assay. In some embodiments, the first agent and/or second agent comprises a detectable tag, e.g., a fluorescent tag or label. In some embodiments, the condensate comprises a component comprising a detectable tag, e.g., a fluorescent tag or label. In some embodiments, the first agent is unlabeled, and the second agent comprises a detectable tag, such as a fluorescent label. In some embodiments, the second agent comprises the first agent and a detectable tag, such as a fluorescent label. In some embodiments, measuring the incorporation of an agent in a condensate comprises quantifying the signal intensity of the agent in the bounds of one or more condensates, wherein the bounds of the one or more condensate is based on a labeled component of the condensate, such as described in measuring techniques disclosed herein and/or shown in the FIGS attached herewith.

In some embodiments, provided herein is a method for determining whether an agent modulates the incorporation of a condensate component in a condensate, the method comprising: (a) measuring the incorporation of the condensate component in the condensate in the presence of the agent; and (b) comparing the incorporation of the condensate component in the condensate in the presence of the agent to a reference, thereby determining whether the agent modulates the incorporation of the condensate component in the condensate. In some embodiments, the reference is based on the incorporation of the condensate component in the condensate without the presence of the agent. In some embodiments, the condensate comprises more than one condensate component, e.g., a first component and a second component. In some embodiments, provided herein is a method for determining whether an agent modulates the incorporation of a first condensate component in a condensate, wherein, when not in the presence of the agent, the condensate comprises the first condensate component and a second condensate component, the method comprising: (a) measuring the incorporation of the first condensate component in the condensate without the presence of the agent; (b) measuring the incorporation of the first condensate component in the condensate in the presence of the agent; and (c) comparing the incorporation of the first condensate component in the condensate without the presence of the agent to the incorporation of the first condensate component in the condensate in the presence of the agent, thereby determining whether the agent modulates the incorporation of the first condensate component in the condensate. In some embodiments, measuring the incorporation of an agent and/or a condensate component in a condensate comprises use of a technique comprising Raman spectroscopy, spectrophotometry, quantitative phase microscopy, fluorescent microscopy, including quantitative fluorescent microscopy, and/or a spin down assay. In some embodiments, the agent comprises a detectable tag, e.g., a fluorescent tag or label. In some embodiments, one or more condensate components comprise a detectable tag, e.g., a fluorescent tag or label. In some embodiments, the first condensate component comprises a first detectable tag and the second condensate component comprises a second detectable tag, wherein the first detectable tag and the second detectable tag are distinguishable, e.g., fluoresce at different wavelengths. In some embodiments, measuring the incorporation of a condensate component in a condensate comprises quantifying the signal intensity of the condensate component in the bounds of one or more condensates, wherein the bounds of the one or more condensate is based on a labeled component of the condensate, such as described in measuring techniques disclosed herein and/or shown in the FIGS.

Identifying Agents with a Desired Condensate Coefficient

In some embodiments, provided herein is a method for identifying an agent having a desired condensate partition coefficient. In some embodiments, provided herein is a method for identifying an agent having a desired condensate partition coefficient, the method comprising: (a) measuring the condensate partition coefficient of the agent; and (b) comparing the condensate partition coefficient of the agent to a reference, thereby identifying the agent having a desired condensate partition coefficient. The agent may be any agent described herein and is not limited. In some embodiments, the agent is a small molecule as described herein.

In some embodiments, the method for identifying an agent having a desired condensate partition coefficient is used to screen a plurality of agents and/or select certain agents having a desired condensate partition coefficient. In some embodiments, the condensate partition coefficient of a first agent is measured without the presence of a second agent. For example, in some embodiments, provided herein is a method for identifying one or more agents having a desired condensate partition coefficient from a plurality of agents, wherein the plurality of agents comprises a first agent and a second agent, the method comprising: (a) measuring the condensate partition coefficient of the first agent; (b) measuring the condensate partition coefficient of the second agent; and (c) comparing the condensate partition coefficient of the first agent to the condensate partition coefficient of the second agent, thereby identifying one or more agents having a desired condensate partition coefficient from the plurality of agents. The first and second agent may be any agent described herein and are not limited. In some embodiments, at least the first or second agent is a small molecule as described herein.

In some embodiments, the condensate partition coefficient of a first agent in a condensate is measured in the present of a second agent, e.g., a competition assay. For example, in some embodiments, provided herein is a method for identifying one or more agents having a desired condensate partition coefficient from a plurality of agents, wherein the plurality of agents comprises a first agent and a second agent, the method comprising: (a) measuring the condensate partition coefficient of the first agent without the presence of the second agent; (b) measuring the condensate partition coefficient of the first agent in the presence of the second agent; and (c) comparing the condensate partition coefficient of the first agent without the presence of the second agent to the condensate partition coefficient of the first agent in the presence of the second agent, thereby identifying one or more agents having a desired condensate partition coefficient from the plurality of agents. In some embodiments, measuring the condensate partition coefficient of an agent in a condensate comprises use of a technique comprising Raman spectroscopy, spectrophotometry, quantitative phase microscopy, fluorescent microscopy, including quantitative fluorescent microscopy, and/or a spin down assay. In some embodiments, the first agent and/or second agent comprises a detectable tag, e.g., a fluorescent tag or label. In some embodiments, the condensate comprises a component comprising a detectable tag, e.g., a fluorescent tag or label. In some embodiments, measuring the condensate partition coefficient of an agent in a condensate comprises quantifying the signal intensity of the agent in the bounds of one or more condensates, wherein the bounds of the one or more condensate is based on a labeled component of the condensate, such as described in measuring techniques disclosed herein and/or shown in the FIGS.

Isomers

In some embodiments, a first agent and a second agent are isomers of each other (e.g., cisplatin and transplatin), such as any of constitutional isomers, stereoisomers, enantiomers, diastereomers, cis/trans isomers, conformers, or rotamers, and the methods described herein can be used to identify one or more isomers having a desired condensate partition coefficient by screening a plurality of isomers.

For example, in some embodiments, provided herein is a method for identifying one or more isomers having a desired condensate partition coefficient, the method comprising: (a) measuring the condensate partition coefficient of a first isomer agent; (b) measuring the condensate partition coefficient of a second isomer agent; and (c) comparing the condensate partition coefficient of the first isomer agent to the condensate partition coefficient of the second isomer agent, thereby identifying one or more isomers having a desired condensate partition coefficient. In some embodiments, the first isomer agent and the second isomer agent are isomers of each other. In some embodiments, the first isomer agent and second isomer agent are small molecules. The isomer agents are not limited and may be any agent described herein.

In some embodiments, provided herein is a method for identifying one or more isomers having a desired condensate partition coefficient, the method comprising: (a) measuring the condensate partition coefficient of a first isomer agent without the presence of a second isomer agent; (b) measuring the condensate partition coefficient of the first isomer agent in the presence of the second isomer agent; and (c) comparing the condensate partition coefficient of the first isomer agent without the presence of the second isomer agent to the condensate partition coefficient of the first isomer agent in the presence of the second isomer agent, thereby identifying one or more isomers having a desired condensate partition coefficient.

In some embodiments, the methods disclosed for identifying one or more isomers having a desired condensate partition coefficient may comprise reference to a composition comprising a mixture of different isomers, such a racemic mixture of isomers. For example, in some embodiments, there is provided a method for identifying an isomer having a desired condensate partition coefficient, the method comprising: (a) measuring the condensate partition coefficient of a first isomer agent; and (b) comparing the condensate partition coefficient of a racemic mixture comprising the first isomer agent, thereby identifying the isomer having a desired condensate partition coefficient. In some embodiments, the racemic mixture is a known therapeutic agent (e.g., anti-cancer agent). In some embodiments, a specific isomer of an agent will have a desired condensate partition coefficient, as compared to other isomeric forms of the agent. Thus, in some aspects, provided herein is a pure isomeric composition having a desired condensate partition coefficient, and methods of preparing said pure isomeric composition, the methods comprising identifying an isomeric agent having a desired condensate partition coefficient according to the methods disclosed herein.

Labeled Nucleic Acid

In some embodiments, provided herein are methods of contacting a condensate (e.g., a droplet) having a nucleic acid condensate component and/or containing a nucleic acid with an agent capable of adding a moiety to the nucleic acid and detecting addition of the moiety. In some embodiments, the amount of moiety added is compared to a control or reference level. In some embodiments, the agent is an agent modified by a method disclosed herein and the control or reference level is the amount of moiety added by an unmodified agent. In some embodiments, the moiety is or comprises a detectable tag that is used to detect addition of the moiety. In some embodiments, addition of the moiety modulates expression of a gene product associated with the nucleic acid and expression of the gene product is used to detect addition of the moiety. In some embodiments, the moiety is a plantination moiety. In some embodiments, after contact with the agent, addition of the moiety is measured by HPLC.

In some embodiments, provided herein are methods of contacting a condensate (e.g., a droplet) having a nucleic acid condensate component and/or containing a nucleic acid with an agent capable of removing a moiety to the nucleic acid and detecting removal of the moiety. In some embodiments, the amount of moiety removed is compared to a control or reference level. In some embodiments, the agent is an agent modified by a method disclosed herein and the control or reference level is the amount of moiety removed by an unmodified agent. In some embodiments, the moiety is or comprises a detectable tag that is used to detect removal of the moiety or moiety remaining on the nucleic acid. In some embodiments, removal of the moiety modulates expression of a gene product associated with the nucleic acid and expression of the gene product is used to detect removal of the moiety. In some embodiments, the moiety is methylation. In some embodiments, after contact with the agent, removal of the moiety is measured by HPLC.

Tethered Condensate Component Assays

In some embodiments, provided herein are methods of characterizing an agent comprising providing a fusion construct comprising a condensate component or functional fragment thereof and a nucleic acid binding domain contacted with a nucleic acid capable of binding with the nucleic acid binding domain, and contacting the fusion construct with the agent, thereby characterizing the agent. In some embodiments, the fusion construct anchors a condensate comprising the condensate component or functional fragment to the nucleic acid and the agent is contacted with the condensate. In some embodiments, the agent is contacted with the fusion construct along with one or more condensate components capable of forming a condensate with the fusion construct.

In some embodiments, the fusion construct comprises MED1 or an IDR of MED1. In some embodiments, the fusion construct comprises HP1α or an IDR of HP1α. In some embodiments, the fusion construct comprises ESR1 or the activation domain of ESR1. In some embodiments, the one or more condensate components capable of forming a condensate with the fusion construct comprise the same condensate component as the condensate component of the fusion construct. In some embodiments, the fusion construct comprises an IDR of MED1 and the one or more condensate components comprise MED1. In some embodiments, the fusion construct comprises an IDR of HP1α and the one or more condensate components comprise HP1α. In some embodiments, the fusion construct comprises an activation domain of HP1αESR1 and the one or more condensate components comprise MED1.

In some embodiments, the nucleic acid binding domain is Lad and the nucleic acid comprises lac operator sequences (e.g., a lac array).

In some embodiments, the fusion construct further comprises a detectable tag. The detectable tag is not limited and may be any detectable tag disclosed herein. In some embodiments, the detectable tag is a a fluorescent tag. In some embodiments, a component of the condensate other than the fusion construct condensate component or functional fragment comprises a detectable tag. The detectable tag is not limited and may be any detectable tag disclosed herein. In some embodiments, the detectable tag is a fluorescent tag. In some embodiments, both the fusion construct and the component of the condensate other than the fusion construct condensate component or functional fragment thereof each comprise a detectable tag. In some embodiments, the fusion construct and the component of the condensate other than the fusion construct condensate component or functional fragment thereof each comprise a detectable tag and the ability of the agent to modulate the amount of condensate component associated with the fusion construct is measured by detecting co-localization of each detectable tag.

In some embodiments, the fusion construct further comprises a linker between the nucleic acid binding domain and the condensate component or functional fragment. The linker is not limited and may be any linker described herein. In some embodiments, the linker is GAPGSAGSAAGGSG (SEQ ID NO: 16).

Agent-Resistant Condensates

Some aspects of the disclosure are directed to methods of assessing whether differential expression of one or more condensate components by a cell resistant to an agent causes or contributes to the resistance.

In some embodiments, the method comprises providing an agent-resistant cell, contacting the agent-resistant cell with the agent, and assessing localization, concentration, and/or therapeutic activity of the agent as compared to a control. In some embodiments, the control comprises a corresponding non-resistant cell. In some embodiments, the cell is a cancer cell. The cancer is not limited and may be any cancer disclosed herein. In some embodiments, the cell is a breast cancer cell. The methods of assessing localization, concentration, and/or therapeutic activity of the agent are not limited and may include any method disclosed herein. In some embodiments, the cell comprises a condensate having a detectable label. In some embodiments, the agent contacted with the cell comprises a detectable label. In some embodiments, both the condensate in the cell and the agent comprise a detectable label. The agent is not limited and may be any agent disclosed herein. In some embodiments, the agent is a small molecule.

In some embodiments, the method comprises providing a condensate isolated from an agent-resistance cell, contacting the condensate with the agent, and assessing localization, concentration, and/or therapeutic activity of the agent as compared to a control. In some embodiments, the control comprises a corresponding condensate from a non-resistant cell. In some embodiments, the cell is a cancer cell. The cancer is not limited and may be any cancer disclosed herein. In some embodiments, the cell is a breast cancer cell. The methods of assessing localization, concentration, and/or therapeutic activity of the agent are not limited and may include any method disclosed herein. In some embodiments, the condensate comprises a detectable label. In some embodiments, the agent comprises a detectable label. In some embodiments, both the condensate and the agent comprise a detectable label. The agent is not limited and may be any agent disclosed herein. In some embodiments, the agent is a small molecule.

In some embodiments, the method comprises providing an in vitro condensate (e.g., droplet) comprising a differential amount of a condensate component, or fragment thereof, that is differentially expressed in an agent-resistant cell, contacting the condensate with the agent, and assessing localization, concentration, and/or therapeutic activity of the agent as compared to a control. In some embodiments, the control comprises a corresponding condensate not comprising a differential amount of the condensate component, or fragment thereof. In some embodiments, the agent-resistant cell is a cancer cell. The cancer is not limited and may be any cancer disclosed herein. In some embodiments, the agent-resistant cell is a breast cancer cell. The methods of assessing localization, concentration, and/or therapeutic activity of the agent are not limited and may include any method disclosed herein. In some embodiments, the condensate comprises a detectable label. In some embodiments, the agent comprises a detectable label. In some embodiments, both the condensate and the agent comprise a detectable label. The agent is not limited and may be any agent disclosed herein. In some embodiments, the agent is a small molecule. In some embodiments, the condensate component is mediator, MED1, BRD4, SRSF2, HP1α, FIB1, NPM1, or a functional fragment thereof comprising an IDR. In some embodiments, the differential amount of condensate component is at least 1.5-fold, 2-fold, 3-fold, 4-fold, 5-fold, 6-fold, 7-fold, 8-fold, 9-fold, 10-fold, 15-fold, 20-fold, 50-fold, or more of the condensate component than found in a condensate in a non-resistant cell. In some embodiments, the differential amount of condensate component is less than about 1.5-fold, 2-fold, 3-fold, 4-fold, 5-fold, 6-fold, 7-fold, 8-fold, 9-fold, 10-fold, 15-fold, 20-fold, 50-fold, or more than the amount of condensate component found in a condensate in a non-resistant cell.

In some embodiments, the method comprises providing an in vitro condensate (e.g., droplet) comprising a mutant condensate component, or fragment thereof, corresponding to a mutant condensate component in an agent-resistant cell, contacting the condensate with the agent, and assessing localization, concentration, and/or therapeutic activity of the agent as compared to a control. In some embodiments, the control comprises a corresponding condensate comprising a non-mutant form of the condensate component, or fragment thereof. In some embodiments, the agent-resistant cell is a cancer cell. The cancer is not limited and may be any cancer disclosed herein. In some embodiments, the agent-resistant cell is a breast cancer cell. The methods of assessing localization, concentration, and/or therapeutic activity of the agent are not limited and may include any method disclosed herein. In some embodiments, the condensate comprises a detectable label. In some embodiments, the agent comprises a detectable label. In some embodiments, both the condensate and the agent comprise a detectable label. The agent is not limited and may be any agent disclosed herein. In some embodiments, the agent is a small molecule. In some embodiments, the mutant condensate component is mediator, MED1, BRD4, SRSF2, HP1α, FIB1, NPM1, or a functional fragment thereof comprising an IDR and having the mutation.

Some aspects of the present disclosure are related to characterizing an agent-resistance condensate comprising contacting the condensate with one or more second agents and assessing at least one of agent localization, concentration, or therapeutic activity and/or condensate morphology, stability, or dissolution. In some embodiments, the method comprises determining whether the second agent counters the effects of the resistance to the agent (e.g., drug resistance) caused by the first agent (e.g., it is determined whether contact with the second agent reduces the size of or eliminates the condensate).

In some embodiments, the method comprises providing an agent-resistant cell, contacting the agent-resistant cell with the second agent, and assessing at least one of second agent localization, concentration, or therapeutic activity and/or condensate morphology, stability, or dissolution. In some embodiments, the cell is a cancer cell. The cancer is not limited and may be any cancer disclosed herein. In some embodiments, the cell is a breast cancer cell. In some embodiments, the cell comprises a condensate having a detectable label. In some embodiments, the agent contacted with the cell comprises a detectable label. In some embodiments, both the condensate in the cell and the second agent comprise a detectable label. The second agent is not limited and may be any agent disclosed herein. In some embodiments, the second agent is a small molecule. In some embodiments, the cell is contacted with both the second agent and the agent the cell has resistance to. In some embodiments, the agent the cell has resistance to has a detectable label. In some embodiments, size or dissolution of the condensate is assessed as compared to a control. In some embodiments, the method comprises determining whether the second agent counters the effects of the resistance to the agent (e.g., drug resistance) caused by the first agent (e.g., it is determined whether contact with the second agent reduces the size of or eliminates the condensate).

In some embodiments, the method comprises providing a condensate isolated from an agent-resistance cell, contacting the condensate with the second agent, and assessing at least one of second agent localization, concentration, or therapeutic activity and/or condensate morphology, stability, or dissolution. The cancer is not limited and may be any cancer disclosed herein. In some embodiments, the cell is a breast cancer cell. In some embodiments, the condensate comprises a detectable label. In some embodiments, the second agent comprises a detectable label. In some embodiments, both the condensate and the second agent comprise a detectable label. The second agent is not limited and may be any agent disclosed herein. In some embodiments, the second agent is a small molecule. In some embodiments, the condensate is contacted with both the second agent and the agent the cell has resistance to. In some embodiments, the agent the cell has resistance to has a detectable label. In some embodiments, size or dissolution of the condensate is assessed as compared to a control. In some embodiments, the method comprises determining whether the second agent counters the effects of the resistance to the agent (e.g., drug resistance) caused by the first agent (e.g., it is determined whether contact with the second agent reduces the size of or eliminates the condensate).

In some embodiments, the method comprises providing an in vitro condensate (e.g., droplet) comprising a differential amount of a condensate component, or fragment thereof, that is differentially expressed in an agent-resistant cell, contacting the condensate with a second agent, and assessing at least one of second agent localization, concentration, or therapeutic activity and/or condensate morphology, stability, or dissolution. In some embodiments, the agent-resistant cell is a cancer cell. The cancer is not limited and may be any cancer disclosed herein. In some embodiments, the agent-resistant cell is a breast cancer cell. In some embodiments, the condensate comprises a detectable label. In some embodiments, the second agent comprises a detectable label. In some embodiments, both the condensate and the second agent comprise a detectable label. The second agent is not limited and may be any agent disclosed herein. In some embodiments, the second agent is a small molecule. In some embodiments, the condensate is contacted with both the second agent and the agent the cell has resistance to. In some embodiments, the agent the cell has resistance to has a detectable label. In some embodiments, the condensate component is mediator, MED1, BRD4, SRSF2, HP1α, FIB1, NPM1, or a functional fragment thereof comprising an IDR. In some embodiments, the differential amount of condensate component is at least 1.5-fold, 2-fold, 3-fold, 4-fold, 5-fold, 6-fold, 7-fold, 8-fold, 9-fold, 10-fold, 15-fold, 20-fold, 50-fold, or more of the condensate component than found in a condensate in a non-resistant cell. In some embodiments, the differential amount of condensate component is less than about 1.5-fold, 2-fold, 3-fold, 4-fold, 5-fold, 6-fold, 7-fold, 8-fold, 9-fold, 10-fold, 15-fold, 20-fold, 50-fold, or more than the amount of condensate component found in a condensate in a non-resistant cell. In some embodiments, size or dissolution of the condensate is assessed as compared to a control. In some embodiments, the method comprises determining whether the second agent counters the effects of the resistance to the agent (e.g., drug resistance) caused by the first agent (e.g., it is determined whether contact with the second agent reduces the size of or eliminates the condensate).

In some embodiments, the method comprises providing an in vitro condensate (e.g., droplet) comprising a mutant condensate component, or fragment thereof, corresponding to a mutant condensate component in an agent-resistant cell, contacting the condensate with a second agent, and assessing at least one of second agent localization, concentration, or therapeutic activity and/or condensate morphology, stability, or dissolution. In some embodiments, the control comprises a corresponding condensate comprising a non-mutant form of the condensate component, or fragment thereof. In some embodiments, the agent-resistant cell is a cancer cell. The cancer is not limited and may be any cancer disclosed herein. In some embodiments, the agent-resistant cell is a breast cancer cell. In some embodiments, the condensate comprises a detectable label. In some embodiments, the second agent comprises a detectable label. In some embodiments, both the condensate and the second agent comprise a detectable label. The second agent is not limited and may be any agent disclosed herein. In some embodiments, the second agent is a small molecule. In some embodiments, the condensate is contacted with both the second agent and the agent the cell has resistance to. In some embodiments, the agent the cell has resistance to has a detectable label. In some embodiments, the mutant condensate component is mediator, MED1, BRD4, SRSF2, HP1α, FIB1, NPM1, or a functional fragment thereof comprising an IDR and having the mutation. In some embodiments, size or dissolution of the condensate is assessed as compared to a control. In some embodiments, it is assessed whether the second agent counters the effect of drug resistance to the agent (e.g., contact with the second agent reduces the size of or eliminates the condensate).

High Throughput Screen

In some embodiments, a high throughput screen (HTS) is performed to characterize a plurality of agents and/or a plurality of different condensates (e.g., two or more of a super-enhancer condensate, splicing speckle condensate, heterochromatin condensate, nucleolus, chromatin condensate, polycomb condensate, or DNA damage repair condensate; or two or more in vitro condensate types comprising a super-enhancer condensate component, splicing speckle condensate component, heterochromatin condensate component, nucleolus component, chromatin condensate, polycomb condensate, or DNA damage repair condensate). A high throughput screen can utilize cell-free or cell-based assays (e.g., a condensate containing cell as described herein, an in vitro condensate). High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Wiser.

In some embodiments of the methods disclosed herein, a plurality of agents (e.g., 10, 50, 100, 1000, 10,000, 100,000, or more) are each contacted with a condensate and incorporation of the agents in the condensates are measured or determined. In some embodiments, the condensates contacted with the plurality of agents comprise identical components. In some embodiments, at least some of the condensates comprise different components.

In some embodiments of the methods disclosed herein, an agent is contacted (sequentially or, more preferably, in parallel) with a plurality of compositions each having a condensate having at least one different component. In some embodiments, each of the plurality of compositions is contained in a separate vessel (e.g., a separate well of a multiwall plate).

In some embodiments, a plurality of different agents are contacted with condensates each having the same components. In some embodiments, the incorporation of the plurality of different agents are compared. In some embodiments, the different agents each comprise incremental differences, thus enabling the identification of important properties of the agents that modulate condensate incorporation.

In some embodiments of the methods disclosed herein, an agent is contacted with a composition (e.g., solution) comprising a plurality of condensates having different components. In some embodiments, the condensates with different components are identified with different detectable tags. In some embodiments, the condensates comprise nucleic acid. In some embodiments, the nucleic acid is DNA or RNA. In some embodiments, the nucleic acid comprises a detectable tag (e.g., a fluorescent tag).

In some embodiments, the agent is contacted with the condensate for 1 minute to 48 hours. In some embodiments, the agent is contacted with the condensate for about 1 minute, about 5 minutes, about 10 minutes, about 15 minutes, about 30 minutes, about 45 minutes, about 1 hour, about 2 hours, about 3 hours, about 5 hours, about 8 hours, about 10 hours, about 12 hours, about 18 hours, about 24 hours, about 36 hours, about 48 hours, or more. In some embodiments, the incorporation of the agent into the condensate is evaluated over multiple time points as described herein or is monitored continuously (e.g., for up to 48 hours or longer, for the first 5 minutes, first 10 minutes, or the first 1 hour after contact). As is apparent to a person of skill in the art, the incorporation and effect of an agent on a condensate may comprise both rapid and long term phases.

Some aspects of the invention are directed to a method of modulating the partitioning of a first agent into a condensate comprising coupling the first agent to a second agent, thereby modulating the partitioning of the first agent into the condensate. In some embodiments, the condensate is a transcriptional condensate. In some embodiments, the condensate is selected from a super-enhancer condensate, splicing speckle condensate, heterochromatin condensate, nucleolus, chromatin condensate, polycomb condensate, or DNA damage repair condensate. The method of coupling the agent to the second agent is not limited and may be any suitable method disclosed in the art. In some embodiments, the first and second agent are coupled with a covalent bond. In some embodiments, the first and second agent are coupled with a non-covalent or ionic bond. In some embodiments, the first and second agent are coupled via a linker. In some embodiments, the first and second agent are conjugated together. In some embodiments, the first agent has a therapeutic activity.

The term “linker” as used herein, refers to a chemical group or molecule covalently linking the first and second agent. In some embodiments, the linker is positioned between, or flanked by, two groups, molecules, or moieties and connected to each one via a covalent bond, thus connecting the two agents. In some embodiments, the linker is an amino acid or a plurality of amino acids. In some embodiments, the linker is an organic molecule, group, or chemical moiety. In some embodiments a linker comprises or consists of a polypeptide. In some embodiments a linker may comprise or consist of one or more glycine residues and, in some embodiments, one or more serine, and/or threonine residues. In some embodiments, the linker comprises 1, 2, 3, 4, 5, 6, 7, 8, 9, 10, 11, 12, 13, 14, 15, 16, 17, 18, 19, 20, or more than 20 amino acids. In some embodiments, the linker comprises an oligoglycine sequence. Any suitable linker known in the art may be used and is not limited. For example, in some embodiments if the first and second agent are proteins, the linker may be a polypeptide (e.g., a polypeptide connecting the C-terminus of one agent to the N-terminus of the other agent).

In some embodiments, partitioning of the first agent into the condensate (e.g., the partition coefficient) is increased by coupling to the second agent. In some embodiments, the partition coefficient is increased by about at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to the uncoupled agent. As used herein, the partition coefficient or enrichment ratio is the ratio of concentrations of a compound (e.g., agent) in the condensate of interest and outside the condensate of interest (e.g., the surrounding solution). In some embodiments, the partition coefficient of the uncoupled agent is less than about 5, less than about 2, about 1, less than about 1, less than about 0.5, or less than about 0.1. In some embodiments, the partition coefficient of the coupled first agent is greater than 1, greater than about 1.5, greater than about 2, greater than about 3, greater than about 4, greater than about 5, greater than about 10, greater than about 20, greater than about 50, or greater than about 100. In some embodiments, the partition coefficient of the coupled agents is increased by about 5%, 10%, 15%, 20%, 25%, 30%, 35%, 40%, 45%, 50%, 55%, 60%, 65%, 70%, 75%, 80%, 85%, 90%, 95%, 99% or more as compared to the uncoupled agent. In some embodiments, the condensate comprises a therapeutic target for the first agent.

In some embodiments, the partitioning of the first agent into the condensate (e.g., the partition coefficient) is decreased. In some embodiments, the partition coefficient is decreased by about at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to the uncoupled agent. In some embodiments, the partition coefficient is decreased by about 5%, 10%, 15%, 20%, 25%, 30%, 35%, 40%, 45%, 50%, 55%, 60%, 65%, 70%, 75%, 80%, 85%, 90%, 95%, 99% or more as compared to the uncoupled agent. In some embodiments, the partition coefficient of the uncoupled agent is about 10 or more, about 5 or more, about 2 or more, about 1 or more, or about 0.5 or more. In some embodiments, the partition coefficient of the coupled first agent is less than about 10, less than about 5, less than about 2, less than about 1, less than about 0.5, less than about 0.1, or less than about 0.01. In some embodiments, the condensate does not comprise a therapeutic target for the first agent.

In some embodiments, the uncoupled second agent preferentially partitions into the condensate of interest. In some embodiments, the uncoupled second agent has a partition coefficient of greater than 1, greater than about 1.5, greater than about 2, greater than about 3, greater than about 4, greater than about 5, greater than about 10, greater than about 20, greater than about 50, or greater than about 100. In some embodiments, the second agent has a partition coefficient, with respect to a condensate of interest, that is at least 2-fold, at least 5-fold, at least 10-fold, at least 15-fold, at least 20-fold, at least 50-fold, at least 100-fold, or at least 400-fold more than the first agent. In some embodiments, the second agent has a partition coefficient, with respect to a condensate of interest, that is at least 2-fold, at least 5-fold, at least 10-fold, at least 15-fold, at least 20-fold, at least 50-fold, at least 100-fold, or at least 400-fold less than the first agent. In some embodiments, the second agent is a small molecule having a high partition coefficient for the condensate of interest. In some embodiments, the second agent is a small molecule having a partition coefficient greater than 10, greater than 20, greater than 30, greater than 50, or greater than 100 for the condensate of interest.

In some embodiments, the uncoupled second agent is preferentially excluded from the condensate of interest. In some embodiments, the uncoupled second agent has a partition coefficient of less than 0.9, 0.8, 0.5, 0.1, 0.05, or 0.01. In some embodiments, the second agent is a small molecule having a low partition coefficient for the condensate of interest. In some embodiments, the second agent is a small molecule having a partition coefficient of less than 0.5, less than 0.1, less than 0.05, or less than 0.01 for the condensate of interest. A second agent for causing a first agent attached to it to be concentrated in or excluded from a condensate of interest may be a small molecule that is non-toxic to a subject to whom it is administered and in some embodiments does not by itself have significant biological activity. The second agent, e.g., small molecule, may comprise one or more functional groups that are suitable for reacting with a second functional group in order to attach an agent of interest in order to modify the partitioning behavior of the agent of interest with respect to one or more condensates.

In some embodiments, the therapeutic efficacy of the coupled first agent is increased as compared to uncoupled first agent. In some embodiments, the therapeutically effective dose of the coupled first agent is decreased by about at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to the uncoupled first agent. In some embodiments, the therapeutically effective dose of the coupled first agent is decreased by about 5%, 10%, 15%, 20%, 25%, 30%, 35%, 40%, 45%, 50%, 55%, 60%, 65%, 70%, 75%, 80%, 85%, 90%, 95%, 99% or more as compared to the uncoupled first agent.

In some embodiments, one or more side effects of the coupled first agent are decreased (e.g., decreased in severity or duration, or eliminated) as compared to uncoupled first agent. In some embodiments, the coupled first agent has increased therapeutic efficacy and reduced side effects as compared to the uncoupled agent.

Some aspects of the present disclosure are directed to a method of screening for a candidate agent with modulated condensate partitioning comprising modifying an agent with a condensate partition coefficient and measuring the condensate partition coefficient of the modified agent, wherein if the modified agent has a different partition coefficient than the agent, then the modified agent is identified as a candidate agent with modulated condensate partitioning. Modifications may be by well-known medicinal chemistry manipulations and modifications. In some embodiments, the modification increases or decreases the solubility of the agent. In some embodiments, the modification modulates an electrostatic property of the agent. In some embodiments, the modification is the coupling of a moiety or second agent that preferentially partitions in a desired condensate. In some embodiments, the modification is the coupling of a moiety or second agent that preferentially does not partition in a one or more condensate types (e.g., super-enhancer condensate, nucleolus, etc.).

In some embodiments, the condensate partition coefficient of the modified agent is measured in an in vitro condensate. In some embodiments, the condensate partition coefficient of the modified agent is measured in a condensate in a cell.

In some embodiments, the candidate agent is identified as an improved candidate agent if the candidate agent has increased partitioning into a condensate having a therapeutic target for the candidate agent. In some embodiments, the candidate agent is identified as an improved candidate agent if partitioning is increased by about at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to the unmodified agent. In some embodiments, the candidate agent is identified as an improved candidate agent if partitioning is increased by about 5%, 10%, 15%, 20%, 25%, 30%, 35%, 40%, 45%, 50%, 55%, 60%, 65%, 70%, 75%, 80%, 85%, 90%, 95%, 99% or more as compared to the unmodified agent.

In some embodiments, the candidate agent is identified as an improved candidate agent if the candidate agent has decreased partitioning into a condensate not having a therapeutic target for the candidate agent. In some embodiments, the candidate agent is identified as an improved candidate agent if partitioning is decreased by about at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 100-fold as compared to the unmodified agent. In some embodiments, the candidate agent is identified as an improved candidate agent if partitioning is decreased by about 5%, 10%, 15%, 20%, 25%, 30%, 35%, 40%, 45%, 50%, 55%, 60%, 65%, 70%, 75%, 80%, 85%, 90%, 95%, 99% or more as compared to the unmodified agent.

In some embodiments, the candidate agent is identified as an improved candidate agent if the amount of candidate agent (e.g., total number of molecules of candidate agent, concentration of candidate agent) is modulated in a condensate of interest as compared to the unmodified agent. In some embodiments, the amount of candidate agent is increased in the condensate of interest. In some embodiments, this increase corresponds to an increase of the partition coefficient into the condensate of interest. However, this increase may also be due to increasing the availability of the candidate agent for incorporation into the condensate. For example, the candidate agent may have reduced partitioning in a condensate that is not of interest making it available for incorporation into the condensate of interest. In some embodiments, the amount of candidate agent is decreased in the condensate of interest.

In some embodiments, modulating the partitioning of a first agent into a condensate (e.g., by modifying the first agent, e.g., coupling the first agent to a second agent-thereby creating a candidate agent) results in an increased concentration of the modified or coupled first agent in the condensate relative to the concentration at which the unmodified/uncoupled first agent would be present in the condensate. In some embodiments, modifying or coupling the first agent increases the partition coefficient of the first agent into the condensate. In some embodiments, modifying or coupling the first agent causes the first agent to have reduced partitioning into a different condensate (e.g., condensate not of interest) in which it would otherwise become concentrated. In some embodiments, modifying or coupling the first agent decreases the partition coefficient of the first agent into the condensate. In some embodiments, modifying or coupling the first agent causes the first agent to have increased partitioning into a different condensate (e.g., condensate not of interest) in which it would otherwise become concentrated.

In some embodiments, the candidate agent with modulated condensate partitioning is a chemotherapeutic agent.

Some aspects of the invention are directed to a composition comprising a cell having a first condensate comprising a first detectable label and a second condensate having a different second detectable label. In some embodiments, the first and second condensate are different condensate types selected from a super-enhancer condensate, splicing speckle condensate, heterochromatin condensate, nucleolus, chromatin condensate, polycomb condensate, or DNA damage repair condensate. In some embodiments, at least one of the condensates is a transcriptional condensate. In some embodiments, the composition further comprises an agent contacted with the cell. In some embodiments, the agent is a known therapeutic agent. In some embodiments, the agent is a candidate therapeutic agent.

Some aspects of the invention are directed to a composition comprising a first in vitro condensate, a second in vitro condensate and an agent contacted with the first and second in vitro condensate. In some embodiments, the first and second in vitro condensates are isolated from each other. In some embodiments, at least one of the first in vitro condensate, second in vitro condensate, and the agent comprises a detectable label. In some embodiments, the composition further comprises a third in vitro condensate, and optionally a fourth in vitro condensate, each contacted with the agent. In some embodiments, at least one of the in vitro condensates comprises a component of a super-enhancer condensate, splicing speckle condensate, heterochromatin condensate, nucleolus, chromatin condensate, polycomb condensate, or DNA damage repair condensate. In some embodiments, disclosed herein is a multi-well plate or the like (e.g., a 96-well plate), having a first in vitro condensate contacted with an agent and a second in vitro condensate contacted with the same agent, wherein the first and second in vitro condensates each comprise a component of a different component and wherein the first and second in vitro condensates are in different wells of the multi-well plate.

Some embodiments are directed towards an article comprising a first in vitro condensate contacted with an agent, a second in vitro condensate contacted with the same agent, and a multi-well plate separating the first and second in vitro condensates into separate wells. In some embodiments, the article further comprises at least a third in vitro condensate contacted with the agent. In some embodiments, the article further comprises at least a fourth in vitro condensate contacted with the agent. The first, second, third and fourth in vitro condensates can each comprise a component of a different condensate (e.g., a super-enhancer condensate, splicing speckle condensate, heterochromatin condensate, nucleolus, chromatin condensate, polycomb condensate, or DNA damage repair condensate). The first, second, third and fourth in vitro condensates can each comprise a different detectable label.

In some embodiments, the agent disclosed herein is contacted with a condensate at an overall concentration of between about 1 nM and 500 μM. For example, the agent can be added to a solution comprising a condensate to provide an overall concentration in the solution of between about 1 nM and 500 μM. In some embodiments, the agent is contacted with a condensate at an overall concentration of between 10 nM and 100 nM, between 10 nM and 1 μM, between 1 μM and 10 μM, between 10 μM and 100 μM, or between 100 μM and 500 μM. In some embodiments, the agent is added to a composition (e.g., solution) comprising condensates to provide an overall concentration of between about 1 nM and 500 μM. In some embodiments, the agent is added to a composition comprising condensates to provide an overall concentration of between 10 nM and 100 nM, between 10 nM and 1 μM, between 1 μM and 10 μM, between 10 μM and 100 μM, or between 100 μM and 500 μM.

In some embodiments, the condensate is in a cell. The type of cell is not limited. In some embodiments the cell is a mammalian cell, e.g., a human or mouse cell. In some embodiments the cell is a somatic cell. In some embodiments the cell is a pluripotent stem cell. In some embodiments the cell is a germ cell, stem cell, or zygote. In some embodiments the cell is a primary cell. In some embodiments the cell is a diseased cell. In some embodiments the cell is a cancer cell. In some embodiments, the cell is a white blood cell or fibroblast. In some embodiments the cell is a cell that has been isolated from an embryo.

In some embodiments, the cell is a cell isolated from a patient having a disease, disorder, or condition. In some embodiments, the cell is derived from a cell of a patient having a disease, disorder, or condition. In some embodiments, the cell is a differentiated cell of an induced pluripotent stem cell derived from a cell of a patient having a disease, disorder, or condition. In some embodiments, the cell is an induced pluripotent stem cell derived from a cell of a patient having a disease, disorder, or condition. In some embodiments, the cell is a genetically modified cell expressing one or more condensate components having a detectable label. In some embodiments, the genetically modified cell expresses at least two different condensate components having different detectable labels and/or labels that are detectably distinguishable from each other. In some embodiments, the genetically modified cell expresses at least three different condensate components having different detectable labels and/or labels that are detectably distinguishable from each other. In some embodiments, the genetically modified cell expresses at least four different condensate components having different detectable labels and/or labels that are detectably distinguishable from each other. In some embodiments, each type of labeled condensate component is a component of a different condensate (e.g., a super-enhancer condensate, splicing speckle condensate, heterochromatin condensate, nucleolus, chromatin condensate, polycomb condensate, or DNA damage repair condensate). In some embodiments, the genetically modified cell expresses a labeled super-enhancer component and a labeled nucleolus component. In some embodiments, the labels of the different condensate components are detectably distinguishable from each other.

The terms “disease”, “disorder” or “condition” are used interchangeably and may refer to any alteration from a state of health and/or normal functioning of an organism, e.g., an abnormality of the body or mind that causes pain, discomfort, dysfunction, distress, degeneration, or death to the individual afflicted. Diseases include any disease known to those of ordinary skill in the art. In some embodiments a disease is a chronic disease, e.g., it typically lasts or has lasted for at least 3-6 months, or more, e.g., 1, 2, 3, 5, 10 or more years, or indefinitely. Disease may have a characteristic set of symptoms and/or signs that occur commonly in individuals suffering from the disease. Diseases and methods of diagnosis and treatment thereof are described in standard medical textbooks such as Longo, D., et al. (eds.), Harrison's Principles of Internal Medicine, 18th Edition; McGraw-Hill Professional, 2011 and/or Goldman's Cecil Medicine, Saunders; 24 edition (Aug. 5, 2011). In certain embodiments a disease is a multigenic disorder (also referred to as complex, multifactorial, or polygenic disorder). Such diseases may be associated with the effects of multiple genes, sometimes in combination with environmental factors (e.g., exposure to particular physical or chemical agents or biological agents such as viruses, lifestyle factors such as diet, smoking, etc.). A multigenic disorder may be any disease for which it is known or suspected that multiple genes (e.g., particular alleles of such genes, particular polymorphisms in such genes) may contribute to risk of developing the disease and/or may contribute to the way the disease manifests (e.g., its severity, age of onset, rate of progression, etc.) In some embodiments a multigenic disease is a disease that has a genetic component as shown by familial aggregation (occurs more commonly in certain families than in the general population) but does not follow Mendelian laws of inheritance, e.g., the disease does not clearly follow a dominant, recessive, X-linked, or Y-linked inheritance pattern. In some embodiments a multigenic disease is one that is not typically controlled by variants of large effect in a single gene (as is the case with Mendelian disorders). In some embodiments a multigenic disease may occur in familial form and sporadically. Examples include, e.g., Parkinson's disease, Alzheimer's disease, and various types of cancer. Examples of multigenic diseases include many common diseases such as hypertension, diabetes mellitus (e.g., type II diabetes mellitus), cardiovascular disease, cancer, and stroke (ischemic, hemorrhagic). In some embodiments a disease, e.g., a multigenic disease is a psychiatric, neurological, neurodevelopmental disease, neurodegenerative disease, cardiovascular disease, autoimmune disease, cancer, metabolic disease, or respiratory disease. In some embodiments at least one gene is implicated in a familial form of a multigenic disease.

In some embodiments a disease is cancer, which term is generally used interchangeably to refer to a disease characterized by one or more tumors, e.g., one or more malignant or potentially malignant tumors. The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclear cytoplasmic ratio, atypical mitoses, etc.); invasion of adjacent tissues (e.g., breaching a basement membrane); and/or metastasis. Malignant tumors have a tendency for sustained growth and an ability to spread, e.g., to invade locally and/or metastasize regionally and/or to distant locations, whereas benign tumors often remain localized at the site of origin and are often self-limiting in terms of growth. The term “tumor” includes malignant solid tumors, e.g., carcinomas (cancers arising from epithelial cells), sarcomas (cancers arising from cells of mesenchymal origin), and malignant growths in which there may be no detectable solid tumor mass (e.g., certain hematologic malignancies). Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., the WHO Classification of Tumours series, 4^{th} ed, or 3^{nd} ed (Pathology and Genetics of Tumours series), by the International Agency for Research on Cancer (IARC), WHO Press, Geneva, Switzerland, all volumes of which are incorporated herein by reference. In some embodiments a cancer is one for which mutation or overexpression of particular genes is known or suspected to play a role in development, progression, recurrence, etc., of a cancer. In some embodiments such genes are targets for genetic modification according to methods described herein. In some embodiments a gene is an oncogene, proto-oncogene, or tumor suppressor gene. The term “oncogene” encompasses nucleic acids that, when expressed, can increase the likelihood of or contribute to cancer initiation or progression. Normal cellular sequences (“proto-oncogenes”) can be activated to become oncogenes (sometimes termed “activated oncogenes”) by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in R A or protein level. Aberrant expression may occur, e.g., due to chromosomal rearrangement resulting in juxtaposition to regulatory elements such as enhancers, epigenetic mechanisms, or due to amplification, and may result in an increased amount of proto-oncogene product or production in an inappropriate cell type. Proto-oncogenes often encode proteins that control or participate in cell proliferation, differentiation, and/or apoptosis. These proteins include, e.g., various transcription factors, chromatin remodelers, growth factors, growth factor receptors, signal transducers, and apoptosis regulators. A TSG may be any gene wherein a loss or reduction in function of an expression product of the gene can increase the likelihood of or contribute to cancer initiation or progression. Loss or reduction in function can occur, e.g., due to mutation or epigenetic mechanisms. Many TSGs encode proteins that normally function to restrain or negatively regulate cell proliferation and/or to promote apoptosis. Exemplary oncogenes of the methods disclosed herein include, e.g., MYC, SRC, FOS, JUN, MYB, RAS, RAF, ABL, ALK, AKT, TRK, BCL2, WNT, HER2/NEU, EGFR, MAPK, ERK, MDM2, CDK4, GLI1, GLI2, IGF2, TP53, etc. Exemplary TSGs include, e.g., RB, TP53, APC, NF1, BRCA1, BRCA2, PTEN, CDK inhibitory proteins (e.g., p16, p21), PTCH, WT1, etc. It will be understood that a number of these oncogene and TSG names encompass multiple family members and that many other TSGs are known. In some embodiments, the cancer is breast cancer. In some embodiments, the breast cancer is ER+ breast cancer. In some embodiments, the breast cancer is resistant to tamoxifen and comprises a ER mutation. In some embodiments, the breast cancer is resistant to tamoxifen and over-expresses a condensate component.

In some embodiments a disease is a cardiovascular disease, e.g., atherosclerotic heart disease or vessel disease, congestive heart failure, myocardial infarction, cerebrovascular disease, peripheral artery disease, cardiomyopathy.

In some embodiments a disease is a psychiatric, neurological, or neurodevelopmental disease, e.g., schizophrenia, depression, bipolar disorder, epilepsy, autism, addiction. Neurodegenerative diseases include, e.g., Alzheimer's disease, Parkinson's disease, amyotrophic lateral sclerosis, frontotemporal dementia.

In some embodiments a disease is an autoimmune diseases e.g., acute disseminated encephalomyelitis, alopecia areata, antiphospholipid syndrome, autoimmune hepatitis, autoimmune myocarditis, autoimmune pancreatitis, autoimmune polyendocrine syndromes autoimmune uveitis, inflammatory bowel disease (Crohn's disease, ulcerative colitis), type I diabetes mellitus (e.g., juvenile onset diabetes), multiple sclerosis, scleroderma, ankylosing spondylitis, sarcoid, pemphigus vulgaris, pemphigoid, psoriasis, myasthenia gravis, systemic lupus erythemotasus, rheumatoid arthritis, juvenile arthritis, psoriatic arthritis, Behcet's syndrome, Reiter's disease, Berger's disease, dermatomyositis, polymyositis, antineutrophil cytoplasmic antibody-associated vasculitides (e.g., granulomatosis with polyangiitis (also known as Wegener's granulomatosis), microscopic polyangiitis, and Churg-Strauss syndrome), scleroderma, Sjogren's syndrome, anti-glomerular basement membrane disease (including Goodpasture's syndrome), dilated cardiomyopathy, primary biliary cirrhosis, thyroiditis (e.g., Hashimoto's thyroiditis, Graves' disease), transverse myelitis, and Guillane-Barre syndrome.

In some embodiments a disease is a respiratory disease, e.g., allergy affecting the respiratory system, asthma, chronic obstructive pulmonary disease, pulmonary hypertension, pulmonary fibrosis, and sarcoidosis.

In some embodiments a disease is a renal disease, e.g., polycystic kidney disease, lupus, nephropathy (nephrosis or nephritis) or glomerulonephritis (of any kind).

In some embodiments a disease is vision loss or hearing loss, e.g., associated with advanced age.

In some embodiments a disease is an infectious disease, e.g., any disease caused by a virus, bacteria, fungus, or parasite.

In some embodiments, a disease exhibits hypermethylation (e.g., aberrant hypermethylation) or unmethylation (e.g., aberrant unmethylation) in a genomic sequence. For example, Fragile X Syndrome exhibits hypermethylation of FMR-1. In some embodiments, methods described herein may be used to treat or prevent diseases or disorders exhibiting aberrant methylation (e.g., hypermethylation or unmethylation). In some embodiments, agents disclosed herein preferentially incorporate with condensates associated with aberrant methylation. For example, condensates (e.g., transcriptional condensates) may form in regions associated with aberrantly unmethylated or hypomethylated sites, causing aberrant gene transcription. In some embodiments, the agents described herein preferentially incorporate into such condensates and modulate (e.g., reduce) aberrant gene transcription. In some embodiments, the unmethylated or hypomethylated site is associated with an oncogene. In other embodiments, condensates (e.g., splicing speckle condensates, heterochromatin condensates) may form in regions associated with aberrant hypermethylation, causing aberrant gene transcription. In some embodiments, the agents described herein preferentially incorporate into such condensates and modulate aberrant gene transcription.

It will be understood that classification of diseases herein is not intended to be limiting. One of ordinary skill in the art will appreciate that various diseases may be appropriately classified in multiple different groups.

In some embodiments, the method further comprises characterizing the condensate incorporation (e.g., enrichment ratio) of a plurality of agents (e.g., possible drug candidates, possible drug candidates from families having distinct structural features) for, e.g., lead optimization, in vivo toxicology or efficacy studies, or Phase I clinical studies. In some embodiments, a step of profiling drug candidates against a condensate or a panel of condensates and (1) selecting a candidate that is not undesirably sequestered in condensate(s) that are not a site where the target is expected to be present or active, or (2) selecting a candidate that is concentrated or at least not excluded from condensate(s) that are a site where the target is expected to be present or active, is included. If one is optimizing a lead compound and has a number of different optimized candidates to choose from, this method can help avoid selecting a candidate that has a higher propensity than other candidates to become concentrated in condensates that do not contain the target (or to select a candidate that has a higher propensity than other candidates to become concentrated in condensates that do contain the target).

Some aspects of the invention are related to a method of characterizing an first agent, comprising contacting the first agent with a composition comprising a condensate having at least one component, wherein the condensate contains at least a second agent, and measuring the ability of the first agent to cause eviction of the second agent from the condensate. Such method can be useful, e.g., to identify agents (first agents) that release the second agent from condensates. Release of agents from condensates may enhance the therapeutic activity of the agent, for example, if the therapeutic target of the second agent is not in a condensate. Furthermore, such method could be useful to identify first agents having higher affinity for a target in a condensate than the second agent.

In some embodiments, measuring the ability of the first agent to cause eviction of the second agent from the condensate comprises measuring loss of the second agent from the condensate (e.g., by measuring a change in the amount, concentration, or proportion of the second agent in or outside the condensate). The measurement may be performed by any method described herein (e.g., via natural fluorescence or color of the second agent, Raman spectroscopy, NMR, mass spectroscopy, chromatography, etc.). In some embodiments, the second agent has a detectable tag. In some embodiments, the second agent is measured via the detectable tag.

The first and second agents are not limited and may be any agent described herein. The condensate component is also not limited and may be any condensate component described herein. In some embodiments, the condensate component is a transcriptional condensate component. In some embodiments, the condensate component is located in a cell. The cell is not limited and may be any cell described herein. In some embodiments, the condensate is an in vitro condensate.

In some embodiments, the condensate component is a target of the second agent (e.g., the second agent specifically binds to the condensate component). In some embodiments, the first agent displaces the second agent from the target (e.g., displaces the second agent from the condensate).

Some aspects of the disclosure are related to a composition comprising a condensate and an agent having a therapeutic target, wherein the condensate does not comprise, or does not preferentially comprise, the therapeutic target. In some embodiments, the condensate comprises a detectable tag (e.g., the condensate comprises a component having a detectable tag). In some embodiments, the agent has a detectable tag. In some embodiments, both the agent and condensate have a detectable tag (e.g., different detectable tags).

Disrupting Oncogenes

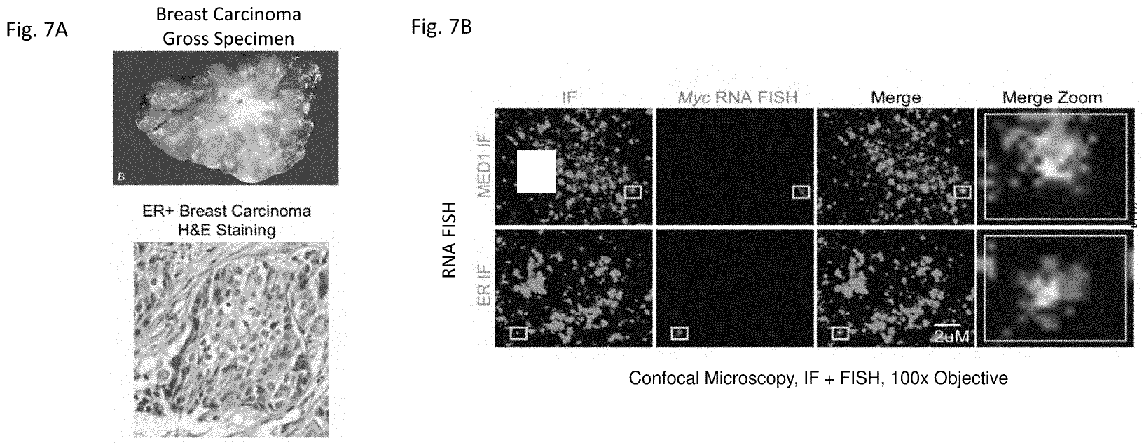

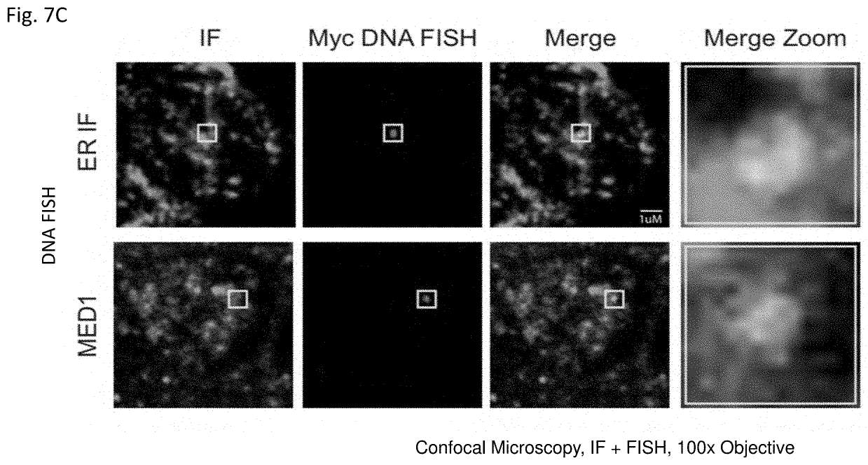

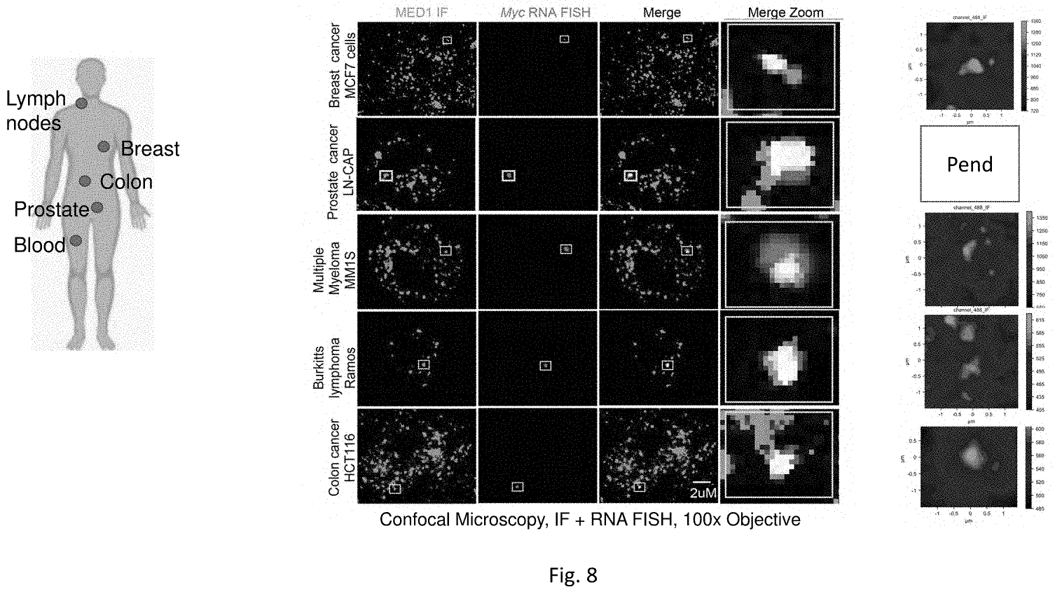

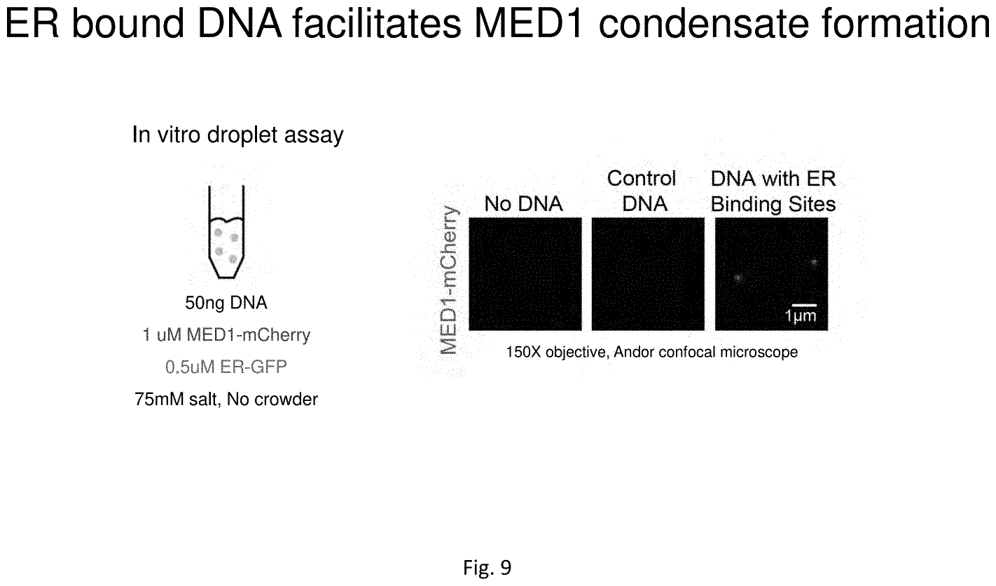

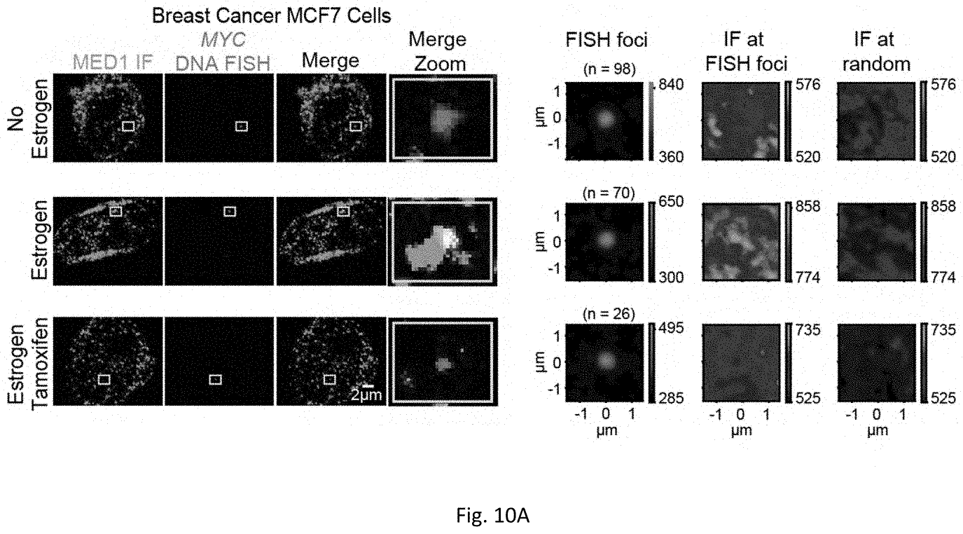

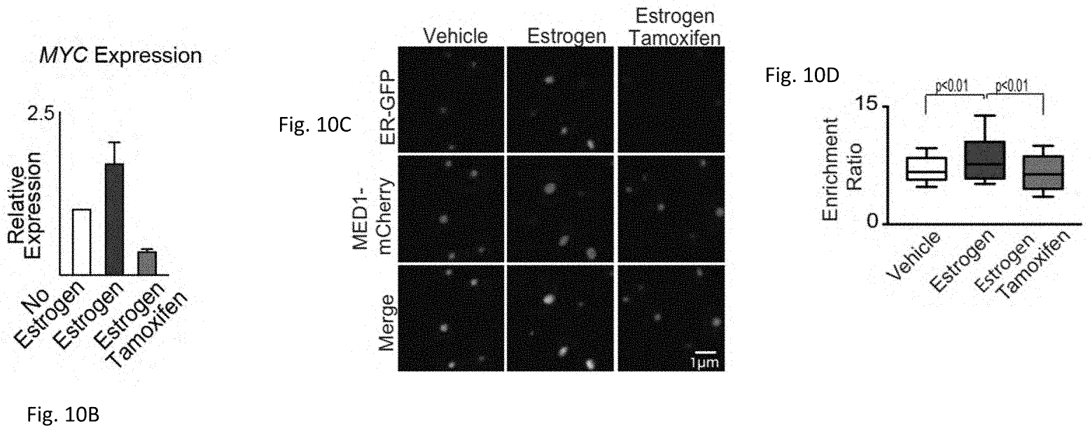

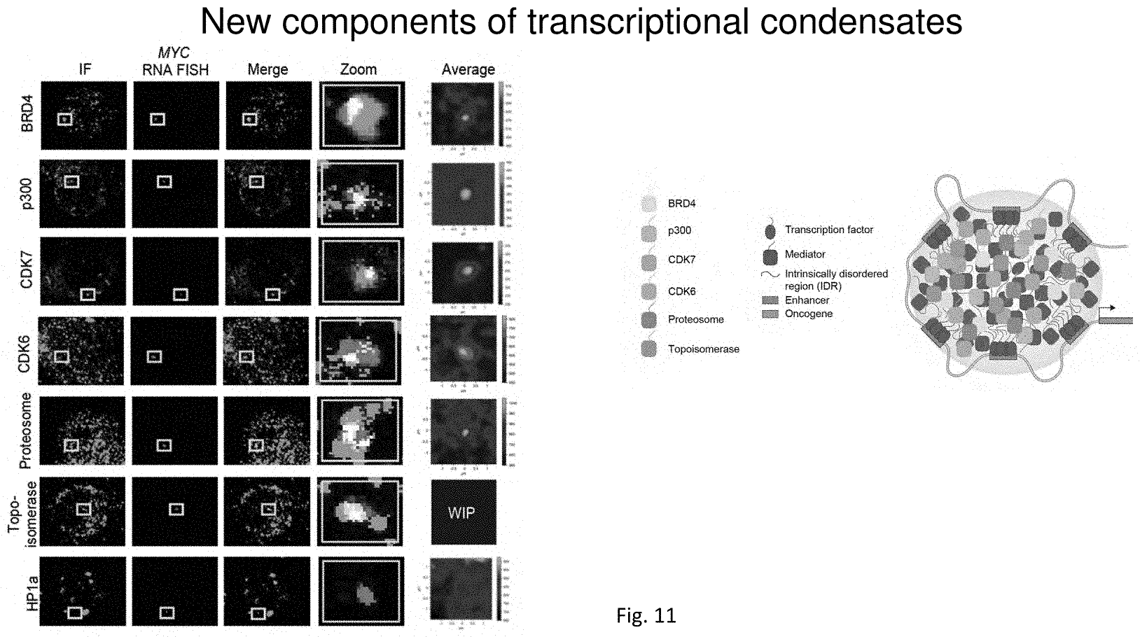

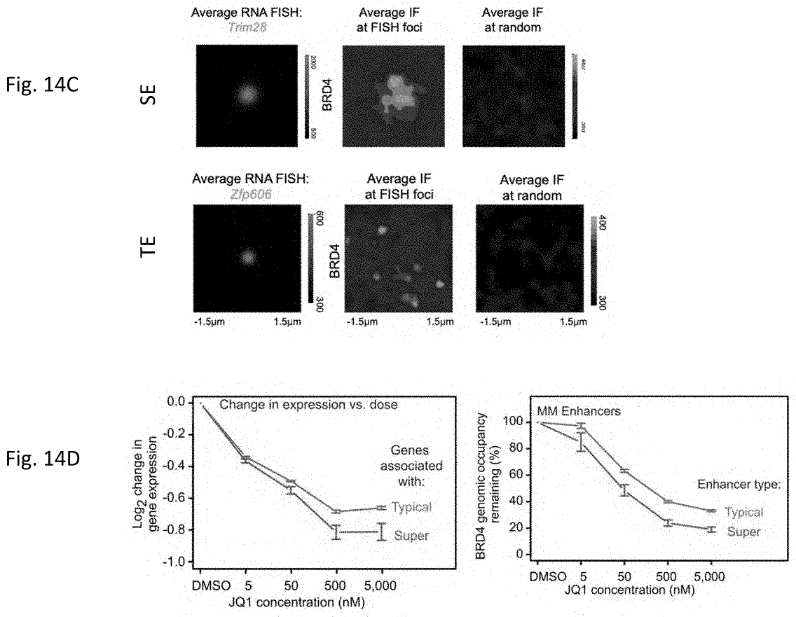

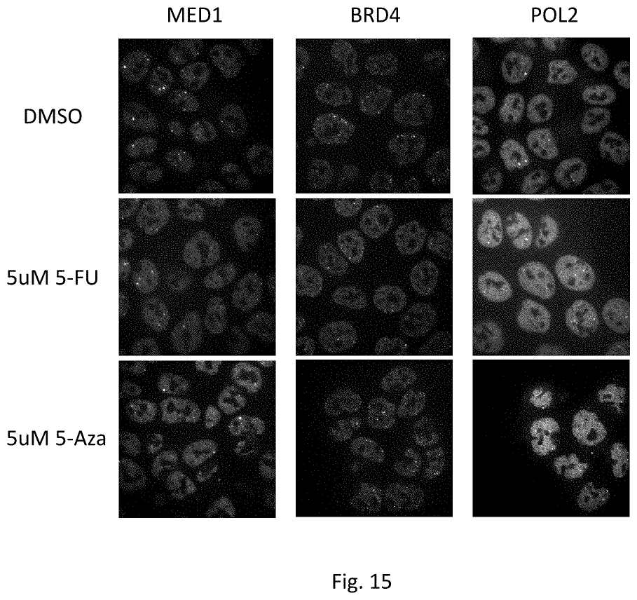

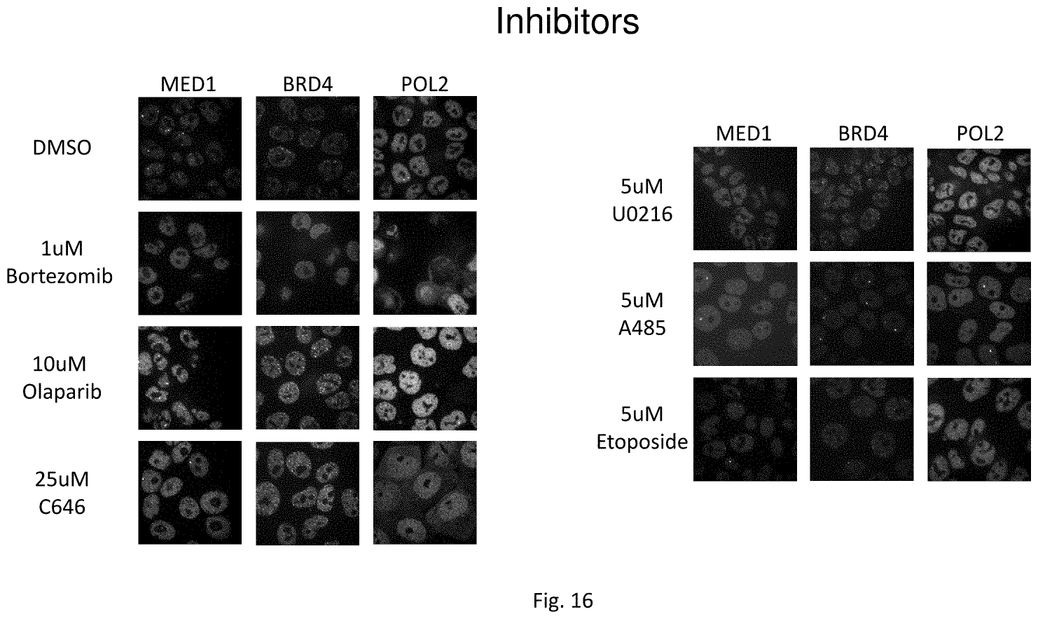

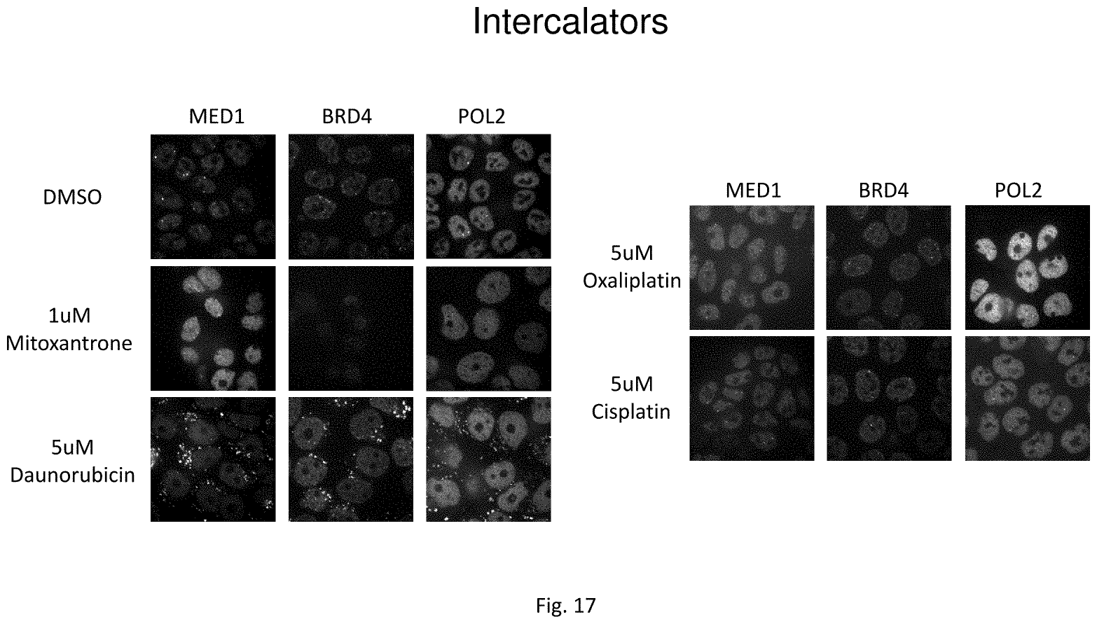

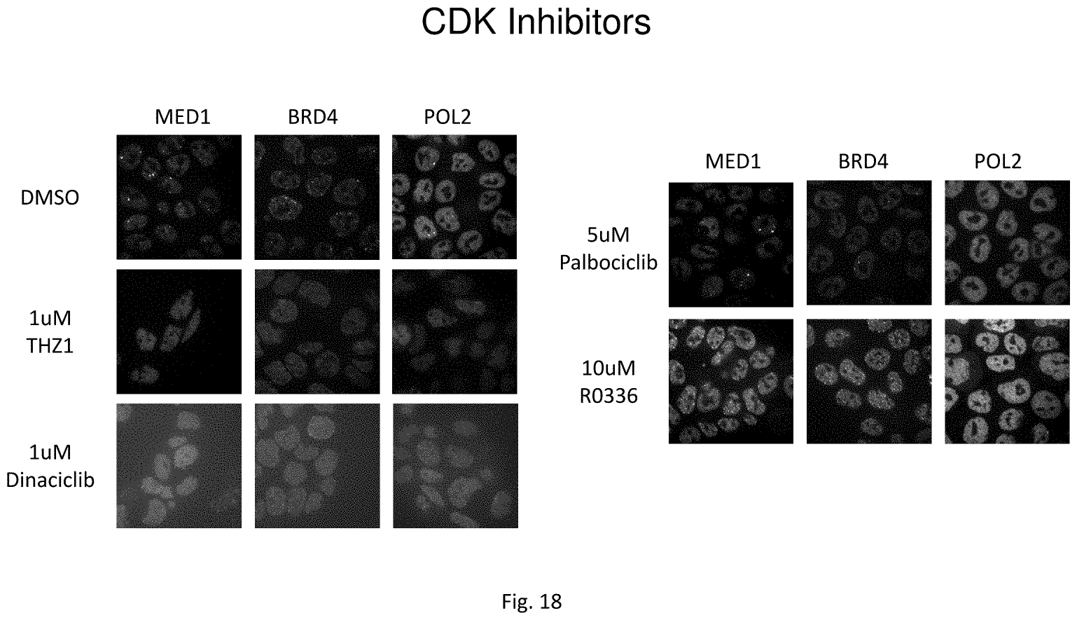

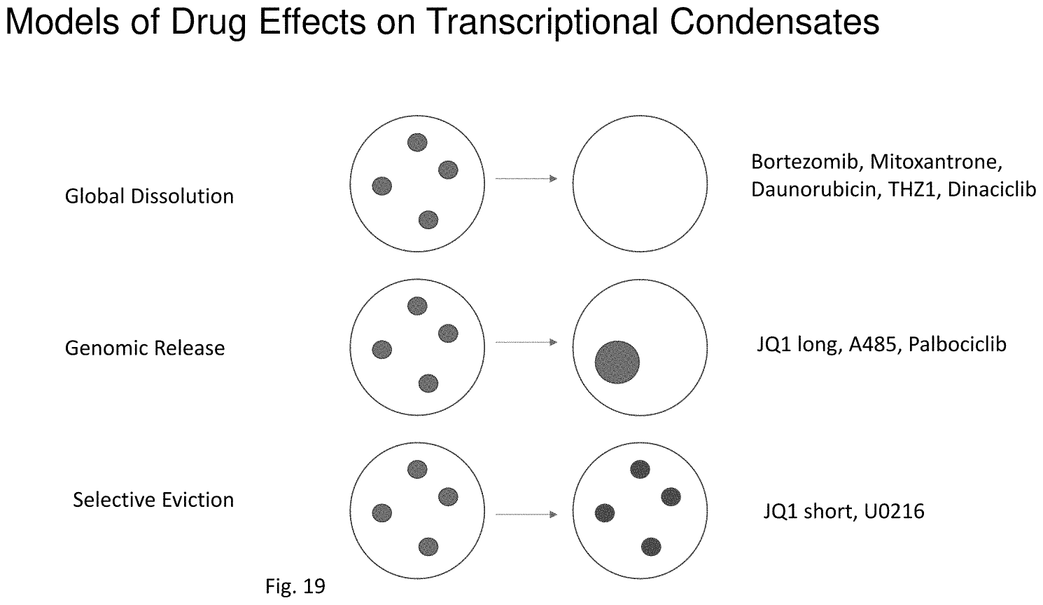

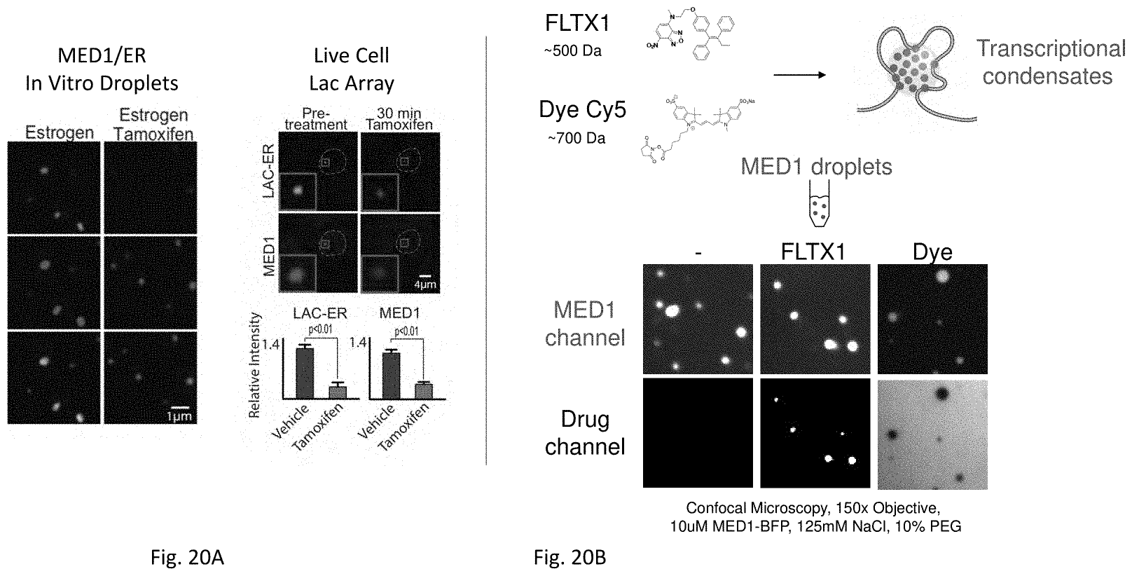

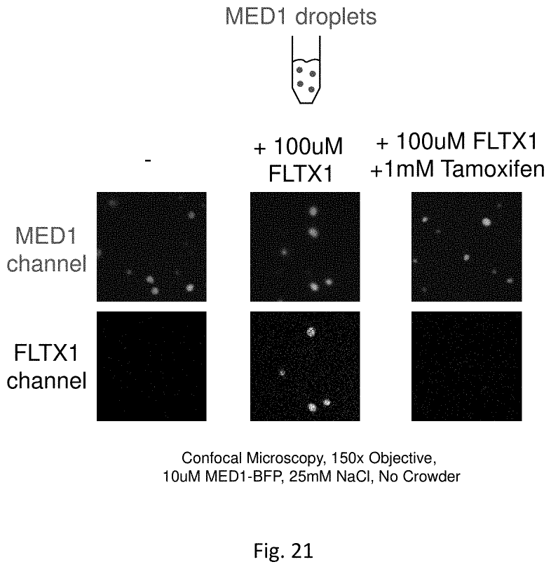

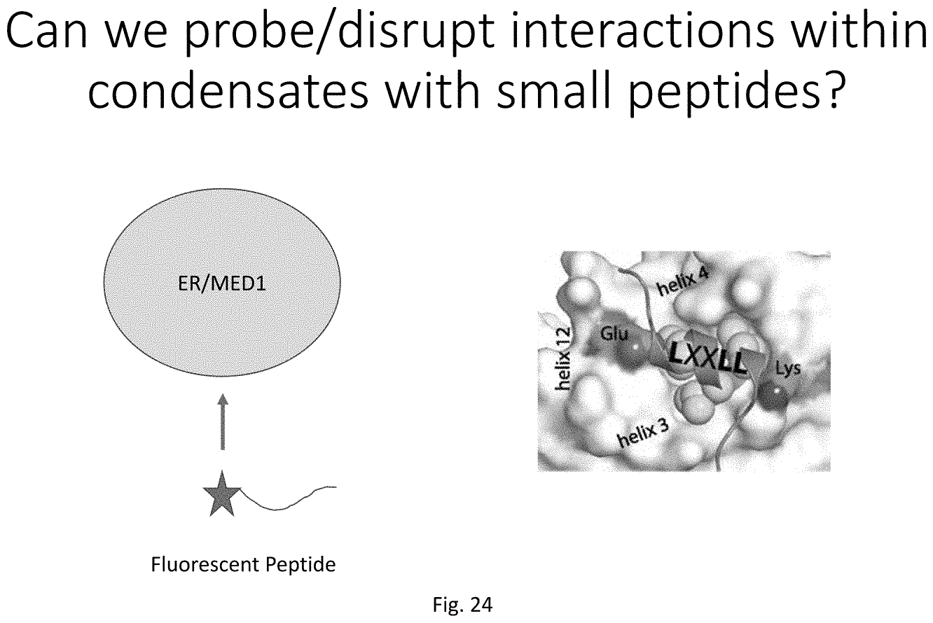

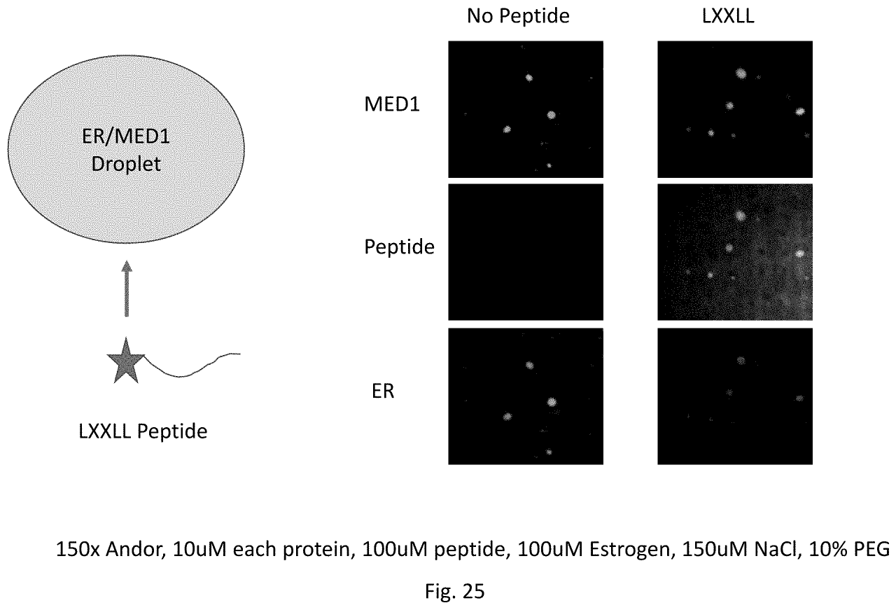

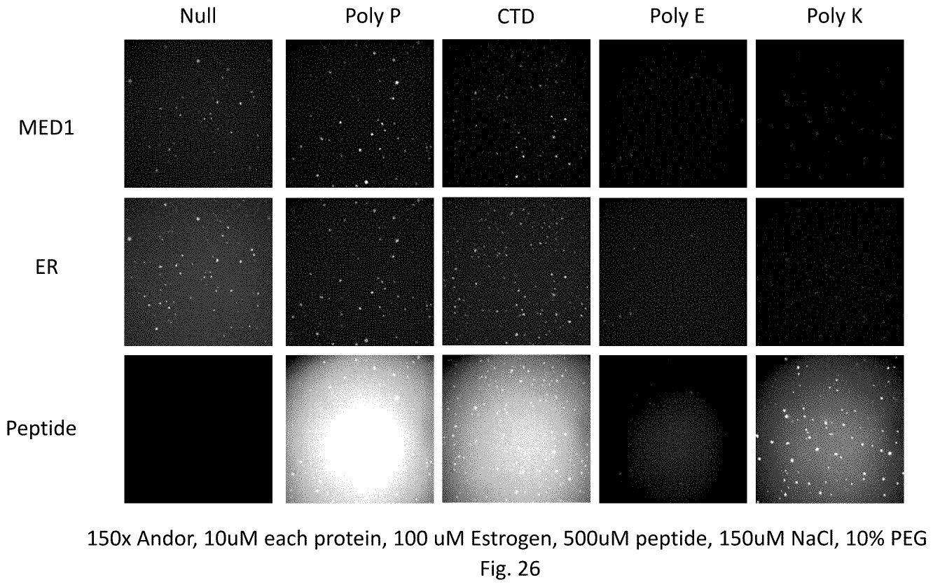

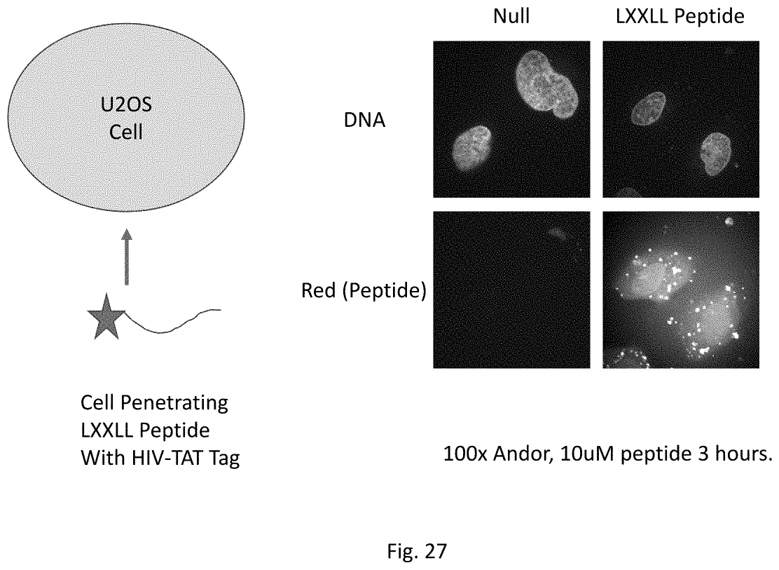

The inventors have shown for the first time herein the presence of MED1 and ER containing condensates at Myc RNA transcription sites of primary breast cancer carcinoma. See, e.g., FIG. 7. The presence of MED1 in condensates at the site of Myc RNA transcription is also confirmed in colon cancer, Burkitts lymphoma, multiple myeloma, prostate cancer, and breast cancer cell lines. See, e.g., FIGS. 8-9. Other condensate components including Topoisomerase, Proteosome, CDK6, CDK7, p300, and BRD4 have also been found in condensates at Myc RNA transcription sites. See, FIG. 11. Using a colon cancer cell line and GFP labeled MED1, BRD4, or POL2, various inhibitors, intercalators, and cyclin dependent kinase inhibitors are shown to dissolve, cause genomic release, or selectively evict components from condensates at Myc RNA transcription sites. See, FIGS. 14 and 16-19. Finally, it is shown herein that ER is not incorporated in condensates in the presence of tamoxifen and condensate dissolving drugs enrich in condensates prior to dissolving condensates. See, FIGS. 20, 22, and 24.

Thus, some aspects of the invention are related to a method of reducing transcription of an oncogene, comprising modulating the composition, dissolving, or disassociating a transcriptional condensate associated with the oncogene. In some embodiments, the transcriptional condensate is modulated by contacting the transcriptional condensate with an agent. In some embodiments, the agent dissolves the transcriptional condensate, causes the transcriptional condensate to uncouple from genomic DNA comprising the oncogene, or evict one or more components of the transcriptional condensate.

The agent is not limited and may be any agent described herein. In some embodiments, the agent is an inhibitor, intercalators, or cyclin dependent kinase inhibitor. In some embodiments, the agent binds to a component of the transcriptional condensate. In some embodiments, the component is BRD4, p300, CDK7, CDK6, Proteosome, Topoisomerase, a transcription factor (e.g., a nuclear receptor, estrogen receptor), mediator, a mediator component, or enhancer. In some embodiments, the agent binds to a component of the transcriptional condensate and dissolves the transcriptional condensate, causes the transcriptional condensate to uncouple from genomic DNA comprising the oncogene, or evicts one or more components of the transcriptional condensate (e.g., evicts the component the agent has bound to or a component that is a binding partner of the component that the agent has bound to).

In some embodiments, the agent preferentially dissolves the transcriptional condensate, causes the transcriptional condensate to uncouple from genomic DNA comprising the oncogene, or evicts one or more components of the transcriptional condensate when the condensate comprises one or more specific condensate components. The component may be any component described herein and is not limited. In some embodiments, the component is BRD4, p300, CDK7, CDK6, Proteosome, Topoisomerase, a transcription factor (e.g., a nuclear receptor, estrogen receptor), mediator, a mediator component, or enhancer.

In some embodiments, the condensate is an in vitro condensate as described herein. In some embodiments, the condensate may be in a cell. The cell is not limited and may be any cell described herein. In some embodiments, the cell is a cancer cell. In some embodiments, the cell is a colon cancer cell, lymphoma cell, multiple myeloma cell, prostate cancer cell, or breast cancer cell.

In some embodiments, the cell is in a subject. In some embodiments, the subject is a mammal (e.g., human, non-human primate, rodent, canine, feline, bovine). In some embodiments, the subject is a human having cancer. The cancer is not limited and may be any cancer described herein. In some embodiments, the cancer has dysregulated Myc gene expression. In some embodiments, the agent reduces transcription of the MYC oncogene in a cancer cell of the subject by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the cancer has dysregulated oncogene selected from SRC, FOS, JUN, MYB, RAS, ABL, HOXI1, HOXI1 1L2, TAL1/SCL, LMO1, LMO2, EGFR, MYCN, MDM2, CDK4, GLI1, IGF2, activated EGFR, mutated genes, such as FLT3-ITD, mutated of TP53, PAX3, PAX7, BCR/ABL, HER2/NEU, FLT3R, FLT6-ITD, SRC, ABL, TAN1, PTC, B-RAF, PML-RAR-alpha, E2A-PRX1, and NPM-ALK, as well as fusion of members of the PAX and FKHR gene families In some embodiments, the agent reduces transcription of the oncogene in a cancer cell of the subject by at least about 5%, 10%, 15%, 20%, 25%, 30%, 35%, 40%, 45%, 50%, 55%, 60%, 65%, 70%, 75%, 80%, 85%, 90%, 95%, 99% or more.

In some embodiments, the agent is administered to a subject having cancer, thereby treating the cancer. “Treatment” as used herein covers any treatment of a disease or condition of a mammal (e.g., cancer), particularly a human, and includes: (a) preventing symptoms of the disease or condition (e.g., cancer) from occurring in a subject which may be predisposed to the disease or condition but has not yet begun experiencing symptoms; (b) inhibiting the disease or condition (e.g., arresting its development); or (c) relieving the disease or condition (e.g., causing regression of the disease or condition, providing improvement in one or more symptoms). The method of administration is not limited and may be any suitable method of administration.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue. Direct tissue administration may be achieved by direct injection. The agents may be administered once, or alternatively they may be administered in a plurality of administrations. If administered multiple times, the peptides may be administered via different routes. For example, the first (or the first few) administrations may be made directly into the affected tissue while later administrations may be systemic.

For oral administration, compositions can be formulated readily by combining the agent with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like In the event that a response in a subject is insufficient at the initial doses applied, higher doses (or effectively higher doses by a different, more localized delivery route) may be employed to the extent that patient tolerance permits. Multiple doses per day are contemplated in some embodiments to achieve appropriate systemic levels of compounds. In some embodiments, the method further includes administering to the subject an effective amount of at least one chemotherapeutic agent. The chemotherapeutic agent is not limited and may be any suitable chemotherapeutic agent known in the art.

Some aspects of the invention are related to a method of treating a subject in need of treatment for cancer characterized by transcription of an oncogene, the method comprising administering to the subject an agent that modulates the composition of, dissolves, or disassociates a transcriptional condensate associated with the oncogene.

The agent is not limited and may be any agent described herein. In some embodiments, the agent is a small molecule, a polypeptide, or a nucleic acid. In some embodiments, the agent is an agent shown to preferentially sequester in a transcriptional condensate associated with an oncogene, or have components of a transcriptional condensate associated with an oncogene. In some embodiments, the agent is an inhibitor, intercalator, or cyclin dependent kinase inhibitor. In some embodiments, the agent binds to a component of the transcriptional condensate. The component is not limited and may be any transcriptional condensate component described herein (e.g., a mediator component, MED1). In some embodiments, the agent preferentially concentrates in the transcriptional condensate.

The cancer is not limited and may be any cancer described herein. In some embodiments, the cancer is colon cancer, lymphoma, multiple myeloma, prostate cancer, or breast cancer.

The subject is not limited and may be any subject described herein. In some embodiments, the subject is human.

The agent may be in a composition. The composition is not limited and may be any composition described herein. The method of administration of the agent is also not limited and may be any method of administration described herein. In some embodiments, the agent is administered orally, subcutaneously, topically, or intravenously to the subject.

Inhibiting Nuclear Receptor Mediated Transcription

Some aspects of the invention are directed to a method of inhibiting transcription associated with a transcriptional condensate, comprising inhibiting the binding of a nuclear receptor having an LXXLL binding domain associated with the transcriptional condensate to a cofactor having an LXXLL domain by contacting the condensate with a peptide that binds to the LXXLL binding domain of the nuclear receptor.

The nuclear receptor is not limited as long it is capable of binding, at least when bound to its ligand, with a cofactor having an LXXLL domain. In some embodiments, the nuclear receptor is a nuclear hormone receptor, an Estrogen Receptor, or a Retinoic Acid Receptor-Alpha. The cofactor is not limited as long as the cofactor has an LXXLL domain. Cofactors having LXXLL motifs are known in the art. In some embodiments, the cofactor is MED1.

In some embodiments, the binding of the nuclear receptor to the cofactor is inhibited by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, transcription associated with a transcriptional condensate is inhibited by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, transcription of an oncogene is inhibited. The oncogene is not limited and may be any oncogene described herein. In some embodiments, the oncogene is Myc.

In some embodiments, the transcriptional condensate is an in vitro transcriptional condensate. In some embodiments, the transcriptional condensate is in a cell. The cell is not limited and may be any cell described herein. In some embodiments, the cell is a cancer cell. The cancer is not limited and may be any cancer described herein. In some embodiments, the methods disclosed herein may be used to treat a disease or condition associated with aberrant nuclear receptor activity or expression. The disease or condition may be any describe herein. In some embodiments, the disease is cancer. In some embodiments, the disease is ER+ breast cancer.

The peptide is not limited as long as it binds to an LXXLL binding domain. In some embodiments, the peptide comprises, consists of, or consists essentially of the peptide sequence QNPILTSLLQITG (SEQ ID NO: 1). In some embodiments, the peptide comprises, consists of, or consists essentially of acidic (e.g., poly-glutamate) or basic residues (e.g., poly-lysine).

In some embodiments, the peptide comprises a protein transduction domain (PTD). The PTD is not limited and may be any PTD described herein. In some embodiments, the PTD is HIV-TAT.

In some embodiments, the peptide is administered to a subject to treat a disease or condition associated with aberrant nuclear receptor activity or expression. The disease or condition may be any describe herein. In some embodiments, the disease is cancer. In some embodiments, the disease is ER+ breast cancer. The method of administration is not limited and may be any method of administration of an agent as described herein. In some embodiments, the peptide is administered as a composition. The composition is not limited and may be any composition described herein for the administration of an agent.

Some aspects of the invention are directed to a method of inhibiting transcription associated with a transcriptional condensate, comprising inhibiting the binding of a nuclear receptor having an LXXLL binding domain and associated with the transcriptional condensate to a cofactor having an LXXLL domain, wherein the binding is inhibited by contacting the condensate with a peptide that binds to the LXXLL domain of the cofactor.

In some embodiments, the binding of the nuclear receptor to the cofactor is inhibited by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, transcription associated with a transcriptional condensate is inhibited by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, transcription of an oncogene is inhibited. The oncogene is not limited and may be any oncogene described herein. In some embodiments, the oncogene is Myc.

In some embodiments, the transcriptional condensate is an in vitro transcriptional condensate. In some embodiments, the transcriptional condensate is in a cell. The cell is not limited and may be any cell described herein. In some embodiments, the cell is a cancer cell. The cancer is not limited and may be any cancer described herein. In some embodiments, the methods disclosed herein may be used to treat a disease or condition associated with aberrant nuclear receptor activity or expression. The disease or condition may be any describe herein. In some embodiments, the disease is cancer. In some embodiments, the disease is ER+ breast cancer.

The peptide is not limited as long as it binds to an LXXLL domain. In some embodiments, the peptide comprises a protein transduction domain (PTD). The PTD is not limited and may be any PTD described herein. In some embodiments, the PTD is HIV-TAT.

In some embodiments, the peptide is administered to a subject to treat a disease or condition associated with aberrant nuclear receptor activity or expression. The disease or condition may be any describe herein. In some embodiments, the disease is cancer. In some embodiments, the disease is ER+ breast cancer. The method of administration is not limited and may be any method of administration of an agent as described herein. In some embodiments, the peptide is administered as a composition. The composition is not limited and may be any composition described herein for the administration of an agent.

Inhibiting Transcription Associated with Overexpression of Condensate Components

As shown below in the examples, a tamoxifen resistant ER+ breast cancer cell line which over-express MED1 comprises MED1 containing condensates having larger volumes than MED1 containing condensates in breast cancer cells that do not over-express MED1. Further, the examples show that when tamoxifen is contacted with MED1 in vitro condensates (e.g., droplets) having a 4-fold increase in MED1 levels, the condensates have much lower concentration of tamoxifen.

Thus, some aspects of the invention are directed to methods of inhibiting the growth or proliferation of a cancer cell that over-expresses a condensate component (e.g., MED1) and is resistant to an anticancer agent (e.g., tamoxifen). In some embodiments, the methods comprise inhibiting the expression or condensate forming activity of the condensate component. In some embodiments, the methods comprise contacting the condensate with a modified condensate component which increases partitioning of the anticancer agent into the condensate. For instance, in some embodiments, the condensate component may be modified to increase the content of aromatic side chains and thereby increase the affinity of a condensate comprising the modified component for an agent having an aromatic side chain. In some embodiments, the condensate having an increased level of a condensate component may be contacted with an agent having an affinity for the anticancer agent and the condensate, thereby increasing the concentration of the anti-cancer agent in the condensate. In some embodiments, the anti-cancer agent may be modified to increase its partitioning in the condensate. For example, in some embodiments, the anti-cancer agent (e.g. tamoxifen) may be modified to increase the number of aromatic side chains and thereby increasing its partitioning in condensates comprising condensate components having aromatic side chains.

Other aspects of the invention comprise determining whether a cancer that over-expresses a gene and is resistant to an anticancer agent comprises condensates that are larger than the corresponding condensates in cancer not over-expressing the gene. In some embodiments, the over-expressed gene is associated with resistance to the anticancer agent. In some embodiments, the concentration of the anti-cancer agent in enlarged condensates from the resistant cancer are compared to the concentration of anti-cancer agent in a non-resistant cancer not over-expressing the gene.

Some embodiments further comprise providing an enlarged condensate from the resistant cancer or an in vitro condensate (e.g. droplet) comprising the over-expressed gene product. In some embodiments the enlarged condensate or in vitro condensate is contacted with one or more modified anticancer agents and the concentration of the modified anti-cancer agent in the contacted condensate is determined. In some embodiments, a library of modified anti-cancer agents are contacted with the condensate and the concentrations of the modified anti-cancer agents are determined in order to screen for modified anti-cancer agents that are effective against the resistant cancer.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

Forming In Vitro Condensates:

A number of condensate components are known to form in vitro condensates. In general, one or more condensate components are added to solutions (e.g. aqueous solution) at varying concentrations in the presence of a salt (e.g., NaCl) and, optionally, a crowding agent (e.g., polyethylene glycol, Ficoll). See, e.g., Boija et al, Cell, vol. 175, no. 7, pp. 1842-1855 (2018); Sabari et al., Science, vol. 361, pp. 361-371 (2018); Bergeron-Sandoval et al., Cell, vol. 165, no. 5, pp. 1067-1079 (2016). May 19; 165(5):1067-1079, the relevant methods of which are specifically incorporated herein. In some embodiments, in vitro MED1 containing condensates are formed by adding about 10 μM of MED1 to a solution containing 150 μM of NaCl and 10% PEG (e.g., PEG-8000). In some embodiments, in vitro MED1 and Estrogen Receptor (ER) containing condensates are formed by adding about 10 μM of each of MED1 and ER to a solution containing 150 μM of NaCl and 10% PEG (e.g., PEG-8000) or 16% Ficoll-400.

Imaging Condensates

Methods of imaging condensates in vitro and in cells are taught in the art and are not limited. In some embodiments, deconvolution microscopy, structured illumination microscopy, or interference microscopy is used to image condensates. See, e.g., Boija et al, Cell, vol. 175, no. 7, pp. 1842-1855 (2018) and Sabari et al., Science, vol. 361, pp. 361-371 (2018), the relevant methods of which are specifically incorporated herein.

In some specific embodiments, cells containing relevant condensates are grown on 35 mm glass plates and imaged in 2i/LIF media using an LSM880 confocal microscope with Airyscan detector. Cells are imaged on a 37° C. heated stage supplemented with 37° C. humidified air. Additionally, the microscope is enclosed in an incubation chamber heated to 37° C. ZEN black edition version 2.3 (Zeiss, Thornwood N.Y.) can be used for acquisition. Images can be acquired with the Airyscan detector in super-resolution (SR) mode with a Plan-Apochromat 63×/1.4 oil objective. Raw Airyscan images can be processed using ZEN 2.3 (Zeiss, Thornwood N.Y.).

In some embodiments, DNA-FISH or RNA-FISH can be used to locate the position of a relevant condensate in a cell by tagging the location of relevant RNA transcription or genomic DNA (e.g., Myc). See, e.g., Boija et al, Cell, vol. 175, no. 7, pp. 1842-1855 (2018). This technique can be used in combination with other methods disclosed herein to determine if the agent co-localizes to the relevant condensate (e.g., via fluorescence microscopy of tagged agents).

To analyze in-vitro phase separation imaging experiments, MATLAB scripts can be written to identify droplets and characterize their size, aspect ratio, condensed fraction and partition factor. For any particular experimental condition, intensity thresholds based on the peak of the histogram and size thresholds (2-pixel radius) can be employed to segment the image, at which point regions of interest can be defined and signal intensity can be quantified in and out of droplets.

Calculation of Partition Coefficients

As used herein, the partition coefficient or enrichment ratio is the ratio of concentrations of a compound (e.g., agent) in the condensate and outside the condensate (e.g., the surrounding solution). The partition coefficient of an agent as described herein may be obtained using any suitable techniques for ascertaining the concentration of the agent, such as microscopy techniques described herein. In some embodiments, Partition coefficients in live-cell imaging can be calculated using Fiji. Using a single focal plane per cell, average signal intensity within a condensate can be quantified and compared to the average signal intensity from 8-12 non-heterochromatic regions within the nuclear boundary of the cell. Limitations of heterochromatic regions and nuclear boundaries can be defined in the Hoechst channel. For quality control, cells that have >3 heterochromatin foci in the selected plane can have a partition coefficient calculated.

### Example 2

The nucleus contains diverse phase-separated condensates that compartmentalize and concentrate biomolecules with distinct physicochemical properties. Here it was considered whether condensates concentrate small molecule cancer therapeutics such that their pharmacodynamic properties are altered. It was found that antineoplastic drugs become concentrated in specific protein condensates in vitro and that this occurs through physicochemical properties independent of the drug target. This behavior was also observed in tumor cells, where drug partitioning influenced drug activity. Altering the properties of the condensate was found to impact the concentration and activity of drugs. These results suggest that selective partitioning and concentration of small molecules within condensates contributes to drug pharmacodynamics and that further understanding of this phenomenon may facilitate advances in disease therapy.

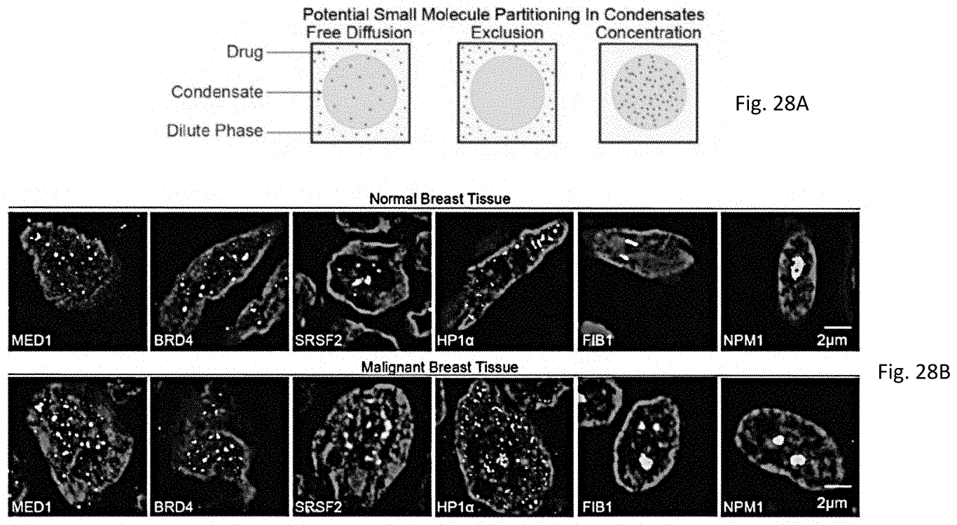

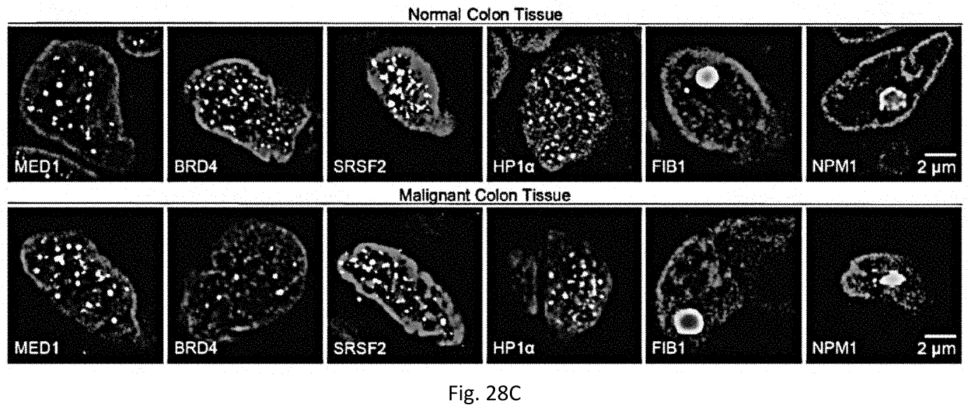

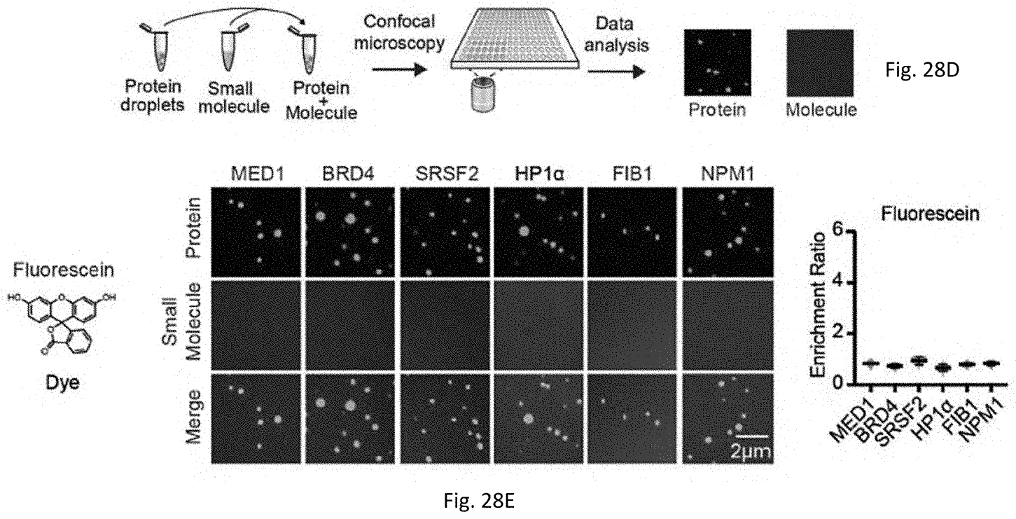

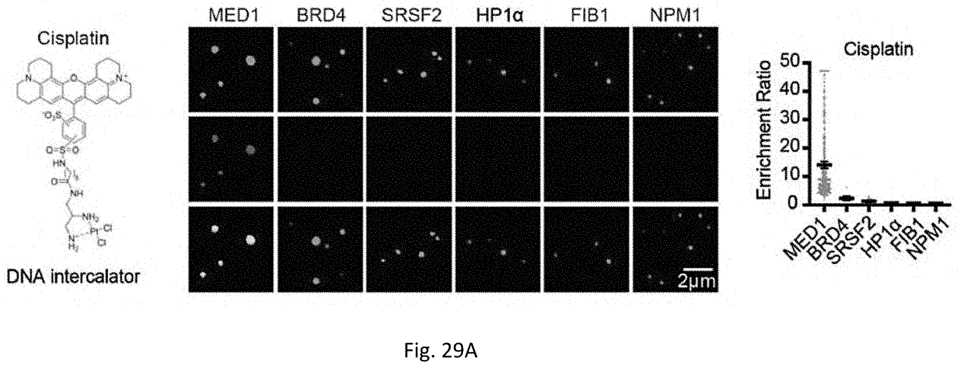

The 5-10 billion protein molecules of cells are compartmentalized into both membrane- and non-membrane-bound organelles (1-3). Many non-membrane-bound organelles are phase-separated biomolecular condensates with distinct physicochemical properties that can absorb and concentrate specific proteins and nucleic acids (4-17). It was reasoned that selective condensate partitioning might also occur with small molecule drugs whose targets occur within condensates (FIG. 28A), and that the therapeutic index and efficacy of such compounds might therefore relate to their ability to partition into condensates that harbor their target. To test this idea, this study focused was on a collection of nuclear condensates previously reported in various cell lines, demonstrated that they all occur in normal human cells and in tumor cells, and then developed in vitro condensate droplet assays with key components of each of the nuclear condensates to enable testing of small molecules.

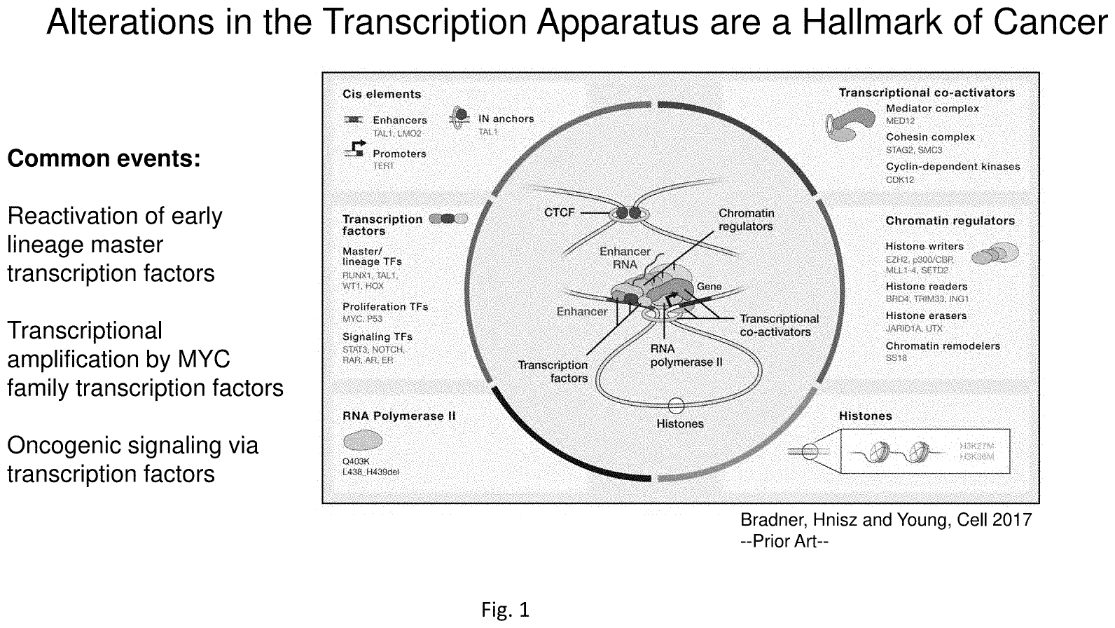

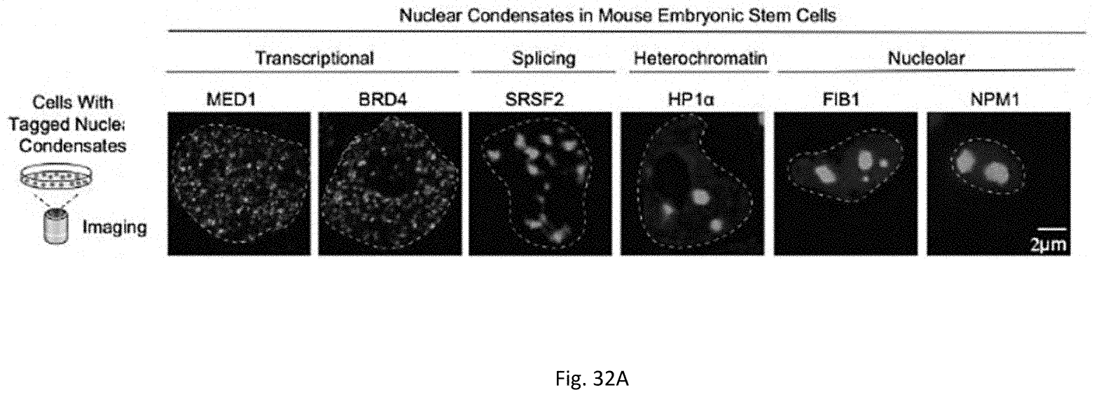

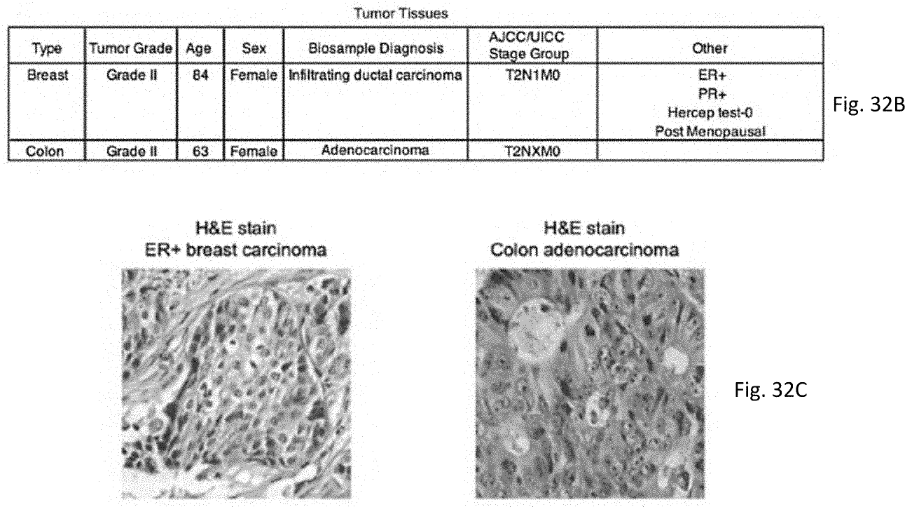

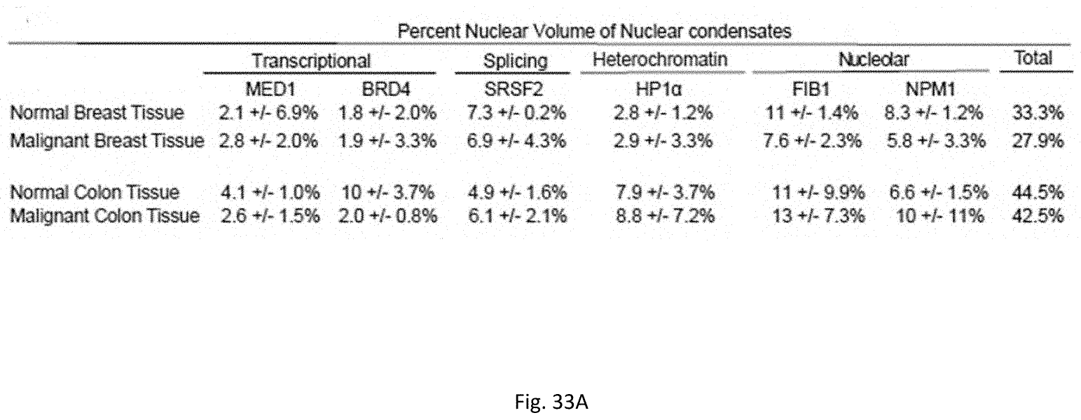

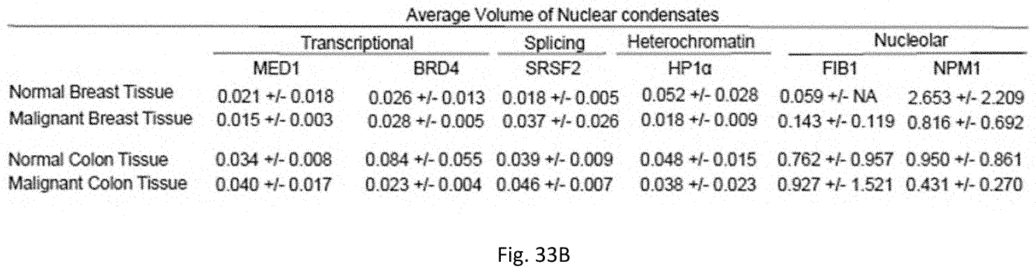

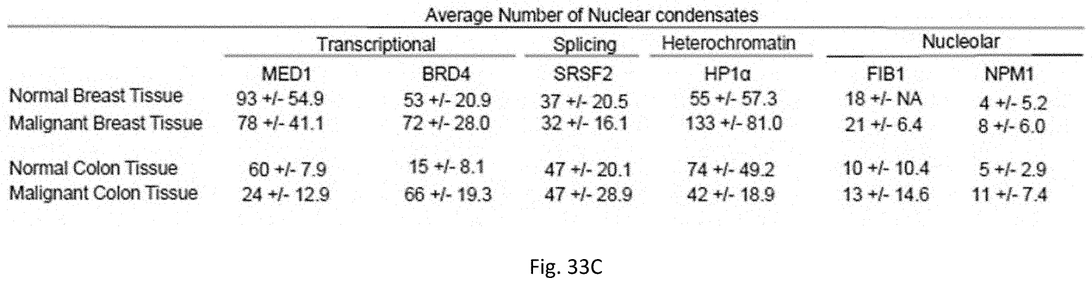

Nuclear condensates have been described in diverse cultured cell lines, but it has not yet been demonstrated that each of the transcriptional, splicing, heterochromatin, and nucleolar condensates occur in the cells of normal and malignant primary human tissue. Each of these condensates contain one or more proteins that can serve both as markers of the condensate and as a scaffold for condensate formation in droplet assays in vitro (10-12, 18-32). Specifically, transcriptional condensates are marked by the condensate forming proteins MED1 and BRD4 (10, 12, 19), splicing speckles by SRSF2 (11, 20), heterochromatin by HP1α (21, 22) and nucleoli by FIB1 and NPM1 (23-25) (FIG. 32A). To determine whether such condensates can also be observed in the cells of healthy and malignant human tissue, we obtained biopsies of breast ductal epithelium, invasive ductal carcinoma, normal colon, and colon cancer (FIGS. 32B, 32C). Immunofluorescence revealed nuclear bodies containing these marker proteins in both normal and transformed tissue (FIGS. 1B, 1C). There was a broad distribution of nuclear body sizes and numbers, as expected for dynamic biomolecular condensates, and no significant differences were observed between benign and malignant tissue (FIGS. 33A-33C). However, tumor cells acquire large super-enhancers at driver oncogenes (33) and, as described below, these can form tumor-specific transcriptional condensates.

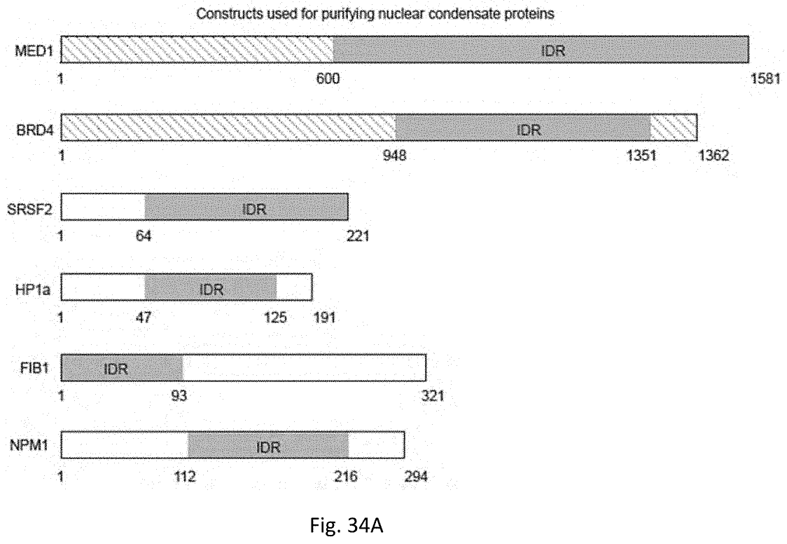

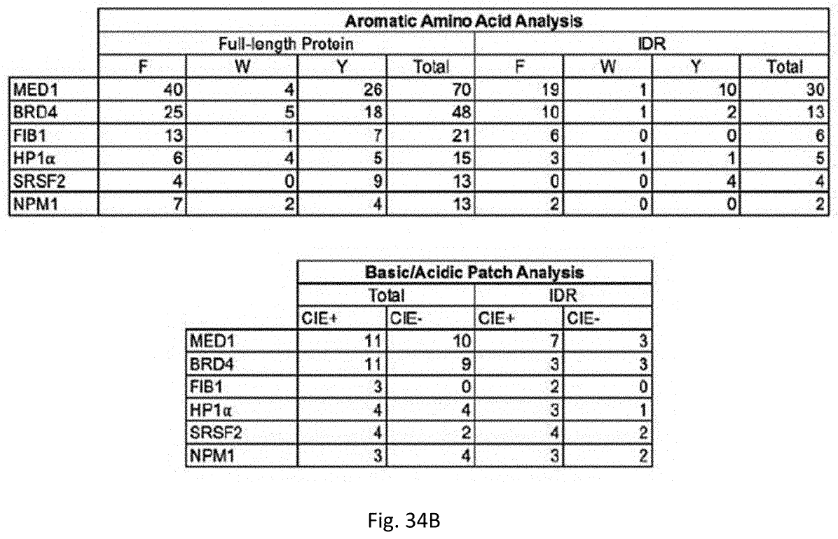

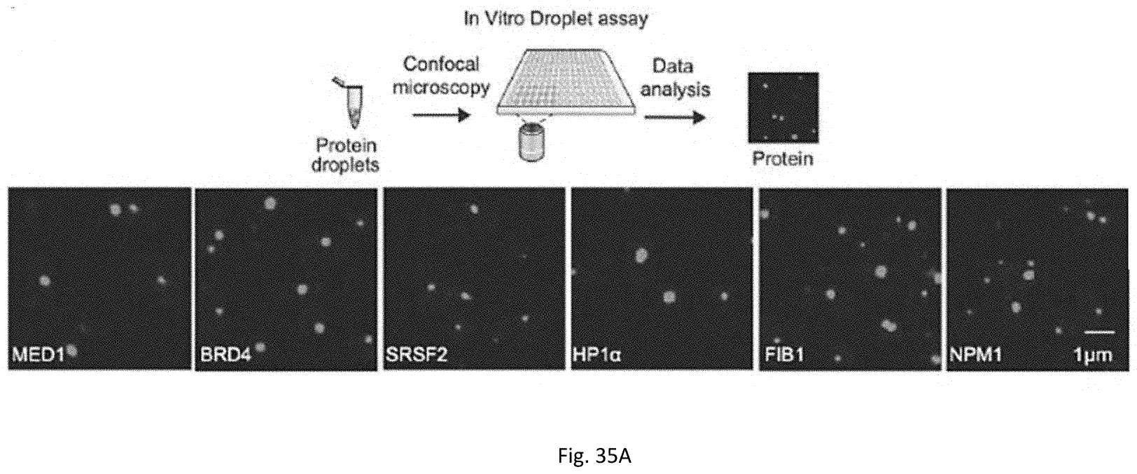

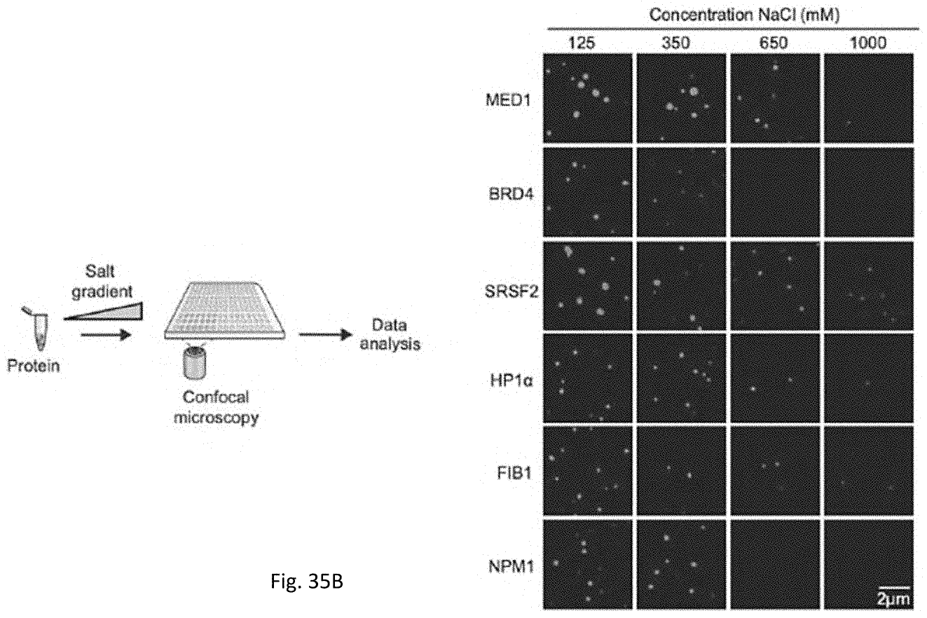

An assay was developed to model these nuclear condensates and study the behavior of small molecules within these droplets (FIG. 28D). The proteins that mark each nuclear condensate have been shown previously to form condensates alone in vitro (10, 11, 21, 23). Recombinant fluorescently-labeled versions of MED1, BRD4, SRSF2, HP1α, FIB1, and NPM1 (FIG. 34) were produced and purified, and the ability of these proteins to form droplets in an in vitro assay was confirmed (FIGS. 35A, 35B). To investigate the partitioning behavior of small molecules, the dyes Fluorescein (332 Da) and Hoechst (452 Da) were added first, as well as fluorescently-labeled dextrans averaging 4.4 kilodaltons (kDa), to solutions containing each of the six protein condensates. The dyes and dextrans appeared to diffuse through all the condensates without substantial partitioning (FIG. 28E, FIG. 36, 37A-37D). Small molecule drugs are generally smaller than 1 kDa, so these results suggested that small molecule drugs can freely diffuse through these nuclear condensates unless there are factors other than size that influence partitioning.

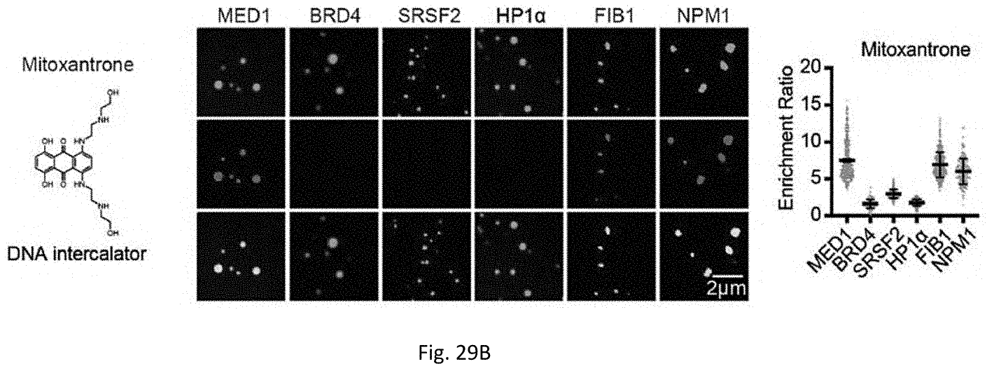

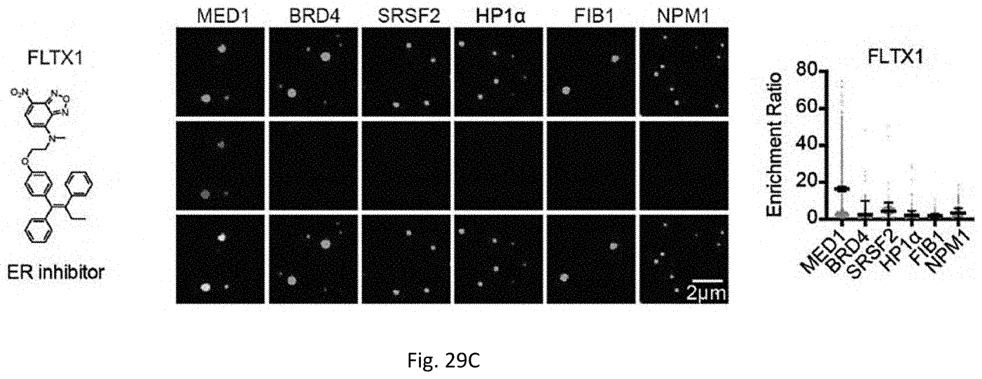

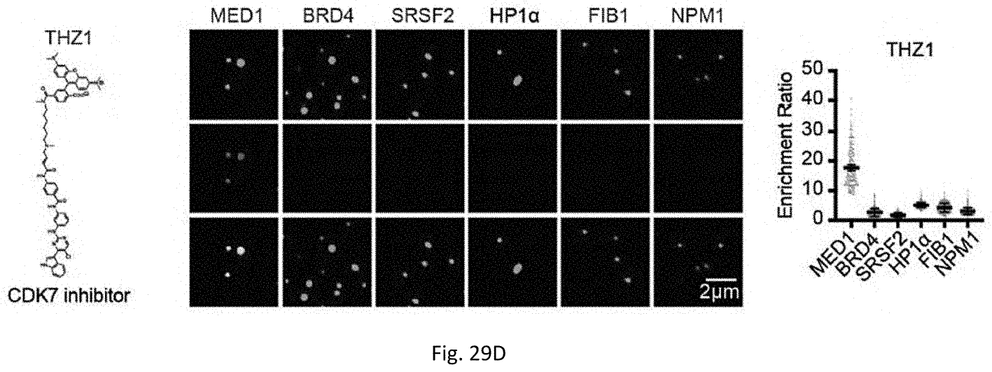

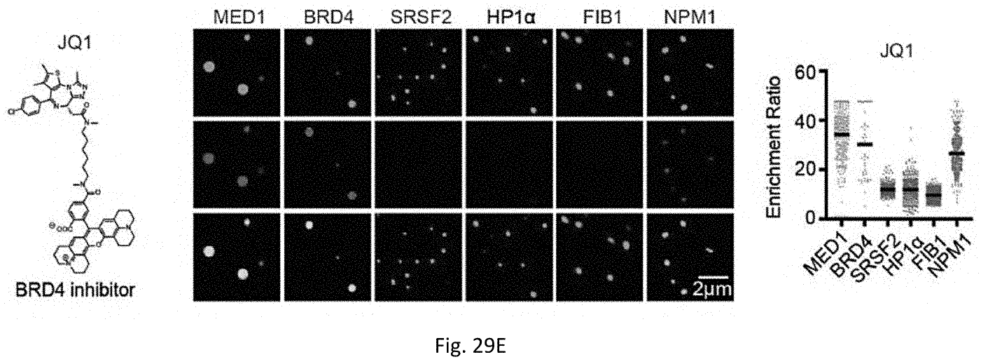

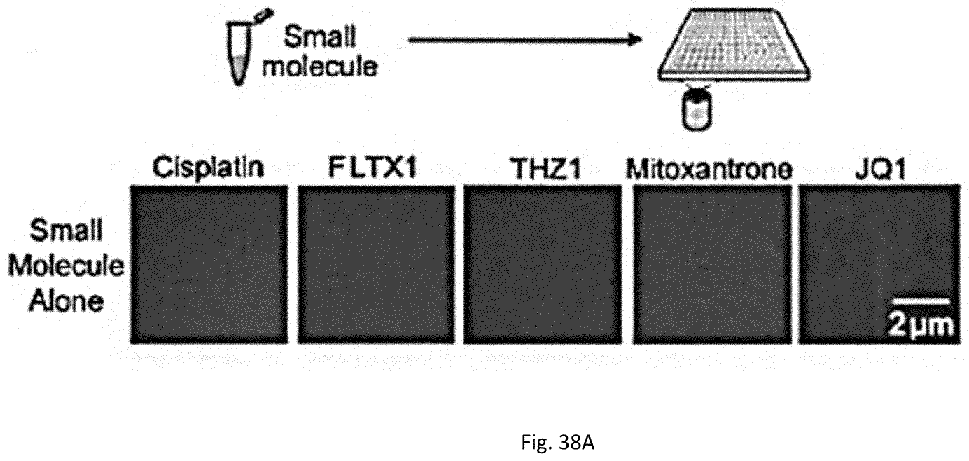

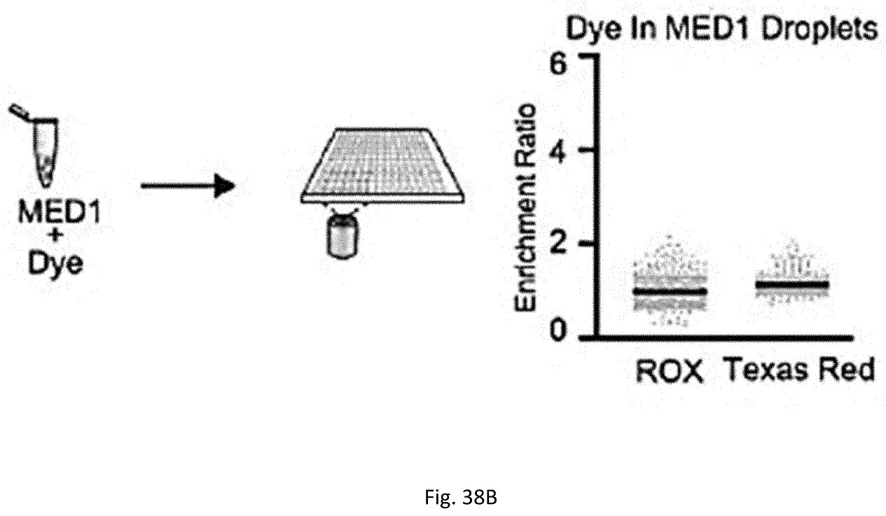

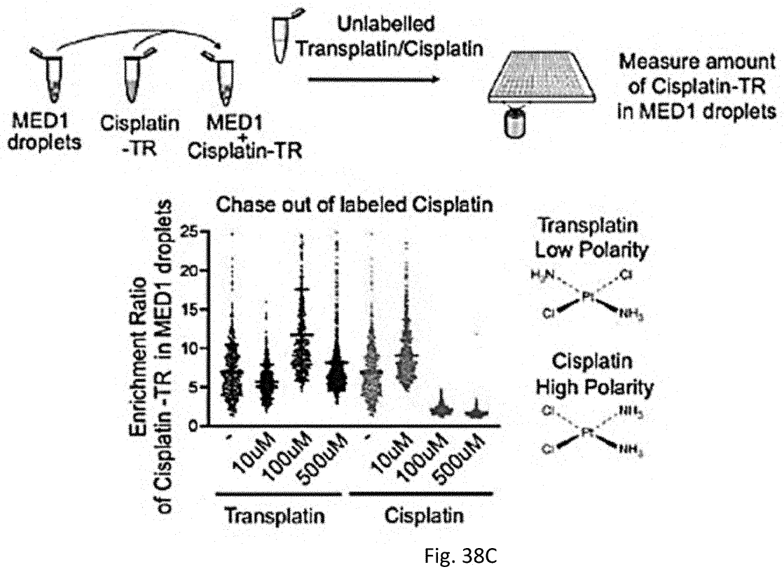

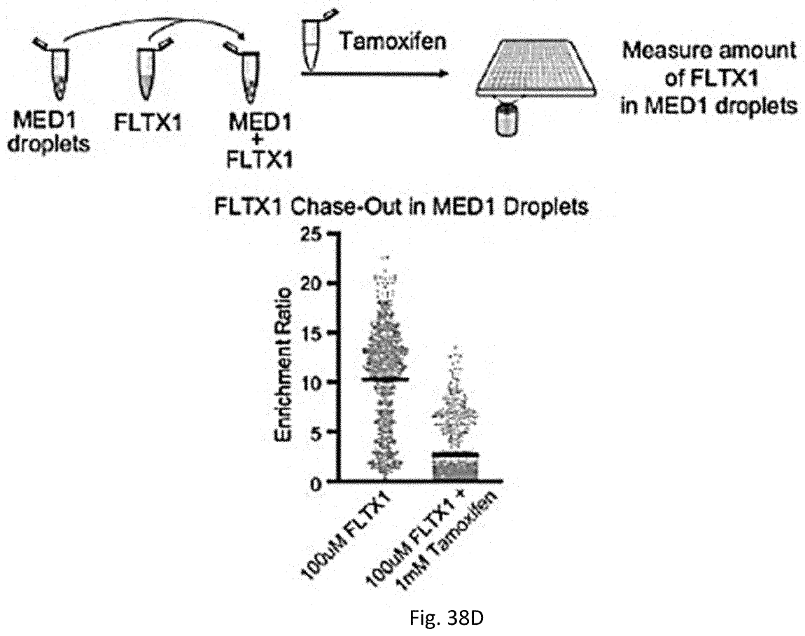

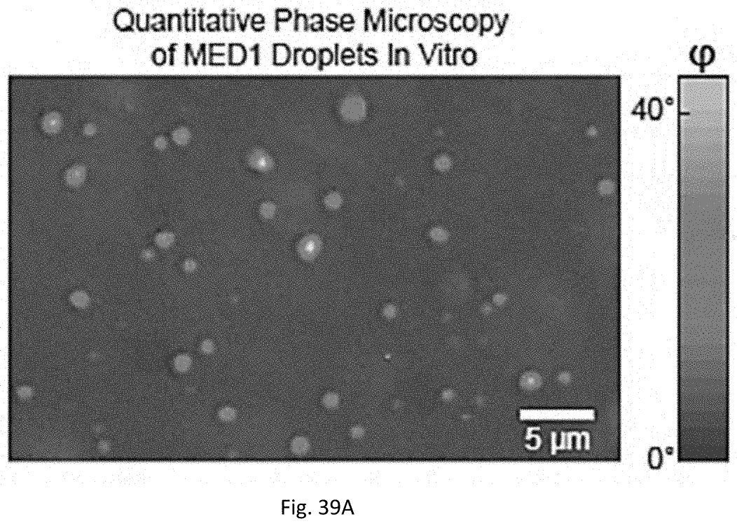

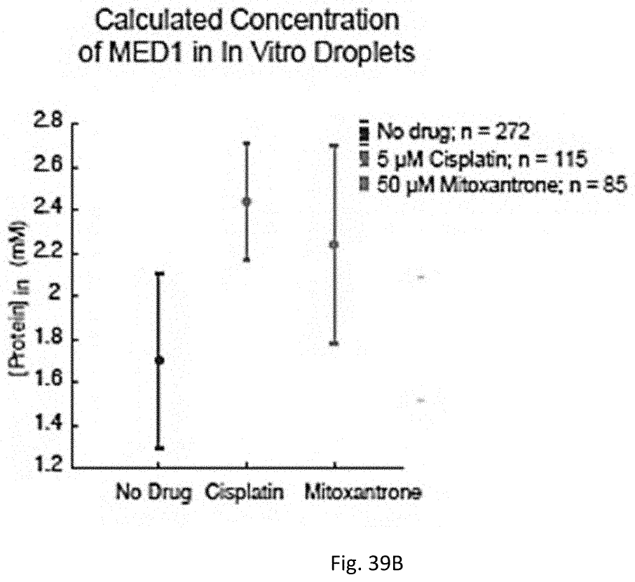

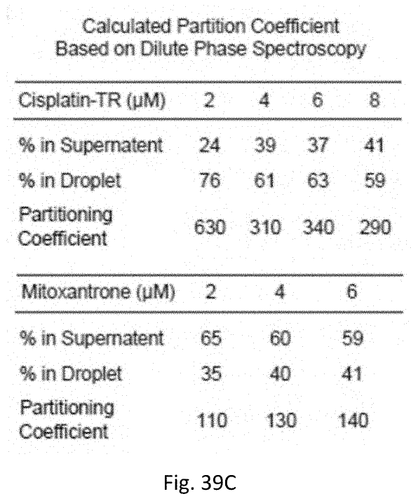

It was next sought to determine whether diverse clinically important drugs with targets that reside in nuclear condensates also exhibit free diffusion across these condensates. The initial focus was on cisplatin and mitoxantrone, members of a class of antineoplastic compounds that modify DNA through platination or intercalation, and that can be modified to have fluorescent properties (cisplatin) (34) or are inherently fluorescent (mitoxantrone). When added to droplet formation buffer with purified MED1, BRD4, SRSF2, HP1α, FIB1, or NPM1, cisplatin was found to be selectively concentrated in MED1 droplets (FIG. 29A, FIG. 38A), with a partition coefficient of up to 600 (FIGS. 39A-39C). Fluorescent modification of cisplatin did not appear to contribute to this behavior in vitro, as the modified drug could be chased out of the condensate with unmodified cisplatin, and an isomer of cisplatin did not exhibit the same behavior (FIGS. 38B-38D). Mitoxantrone was also concentrated in MED1 condensates, as well as in FIB1 and NPM1 condensates (FIG. 29B, FIG. 38A, FIG. 39A-39C). Consistent with these results, mitoxantrone is known to concentrate in the nucleolus where FIB1 and NPM1 reside (35, 36). These results indicate that condensates formed in vitro have physicochemical properties that can selectively concentrate specific small molecule drugs, even in the absence of the drug target.

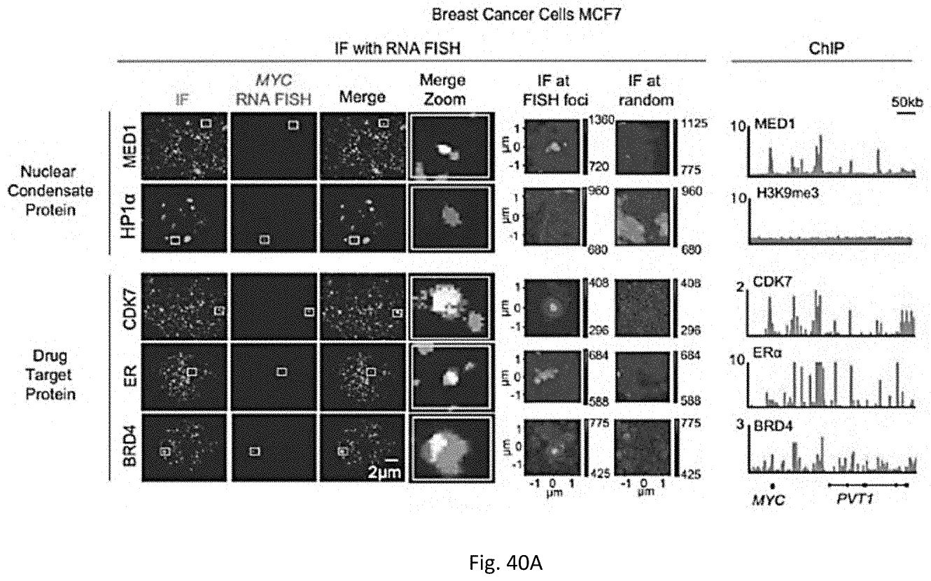

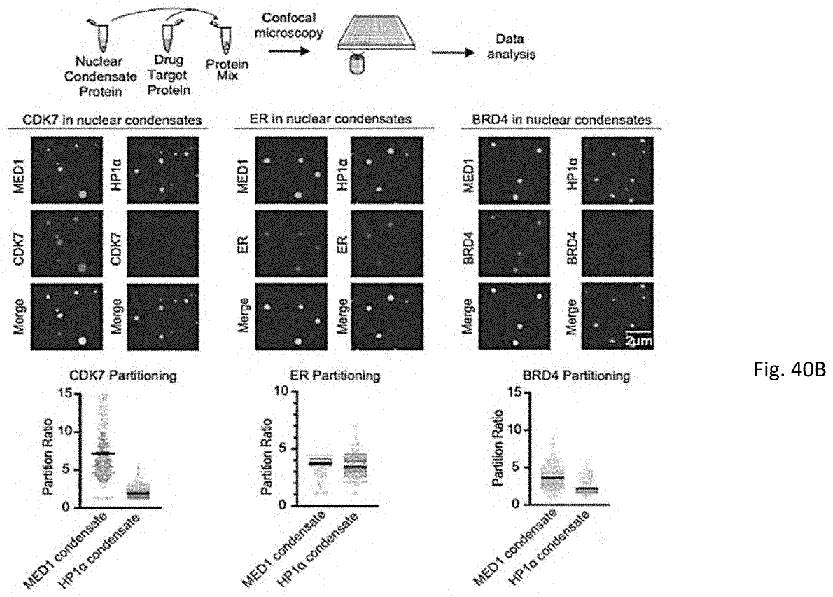

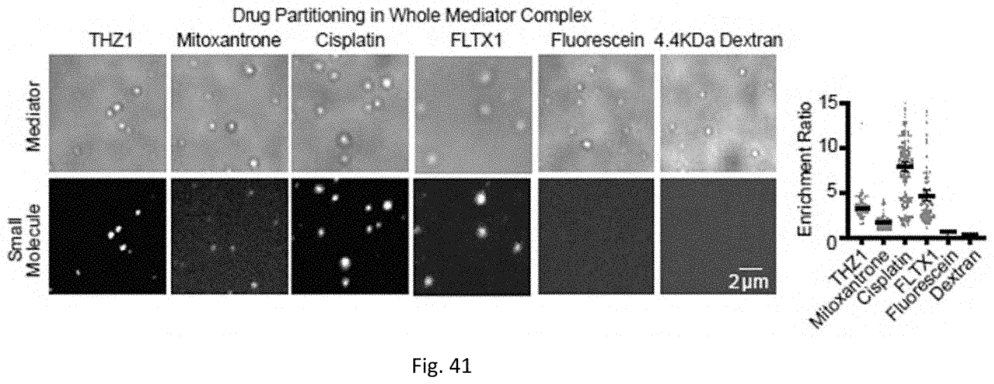

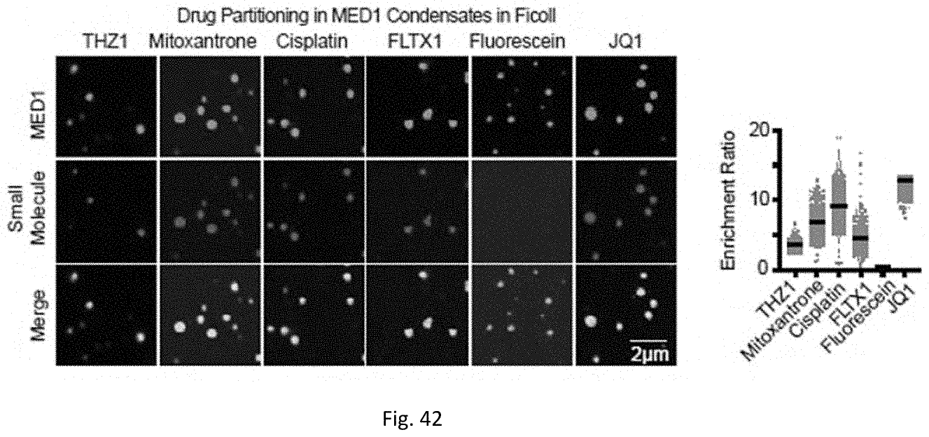

Antineoplastic drugs that target transcriptional regulators expected to be contained within transcriptional condensates in cells were selected for further study. These targets include: a) the estrogen receptor (ER), a transcription factor and nuclear hormone receptor, b) CDK7, a cyclin-dependent kinase that functions in transcription initiation and cell cycle control, and c) BRD4, a bromodomain protein and coactivator involved in oncogene regulation (FIG. 40). To monitor drug behavior with a confocal fluorescent microscope, a fluorescent tamoxifen analog (FLTX1) was used, which targets ER, and modified fluorescent THZ1 and JQ1, which target CDK7 and BRD4, respectively (37, 38). These compounds were added to parallel droplet formation assays with MED1, BRD4, SRSF2, HP1α, FIB1, and NPM1 proteins. FLTX1 and THZ1 concentrated preferentially in MED1 droplets (FIGS. 29C-29D, FIG. 38A), and this behavior was not attributable to the fluorescent moiety (FIG. 38B, FIG. 38D). JQ1 concentration presented a different pattern, being concentrated in MED1, BRD4, and NPM1 droplets (FIG. 29E, FIG. 38A, FIG. 38B). Reinforcing these results, it was found that the small molecules that concentrate in MED1 condensates were also concentrated in condensates formed from purified whole Mediator complexes (FIG. 41) and in MED1 condensates formed in an alternative crowding agent (FIG. 42). The targets of these three compounds (ERα, CDK7, and the bromodomains of BRD4) are not present in these in vitro condensates, but are present in the super-enhancers that form condensates with transcription factors and Mediator in vivo (10, 12, 39) (FIGS. 40A, 40B), suggesting that the ability of some small molecule to concentrate preferentially in the same condensate as their protein target may contribute to the pharmacological properties of these drugs.

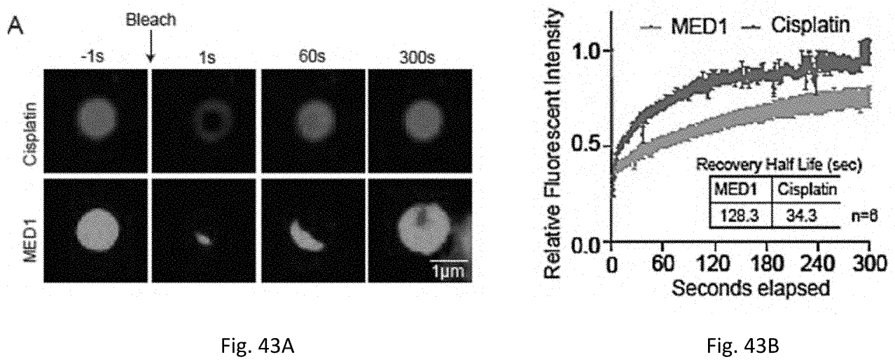

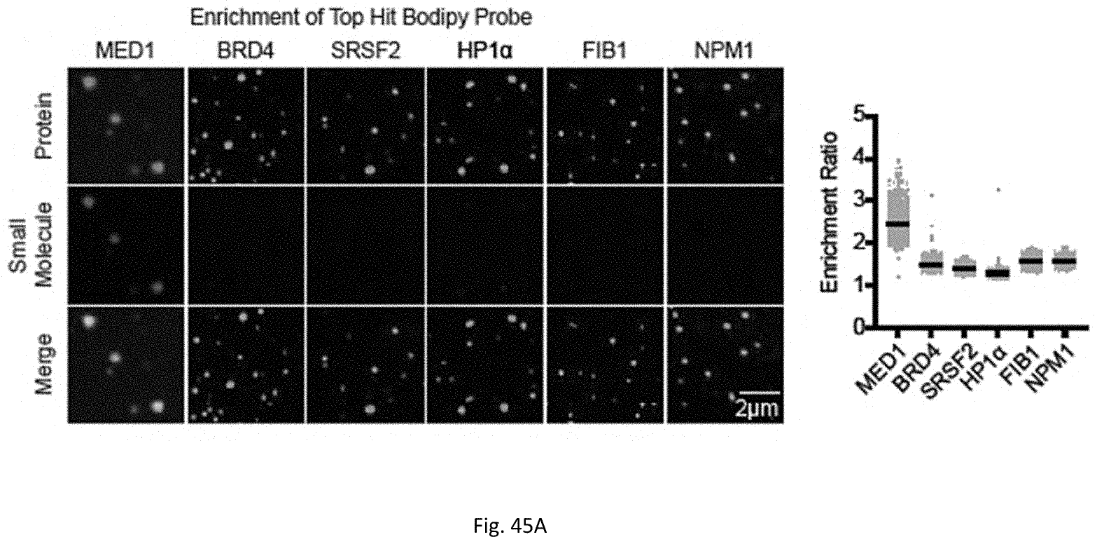

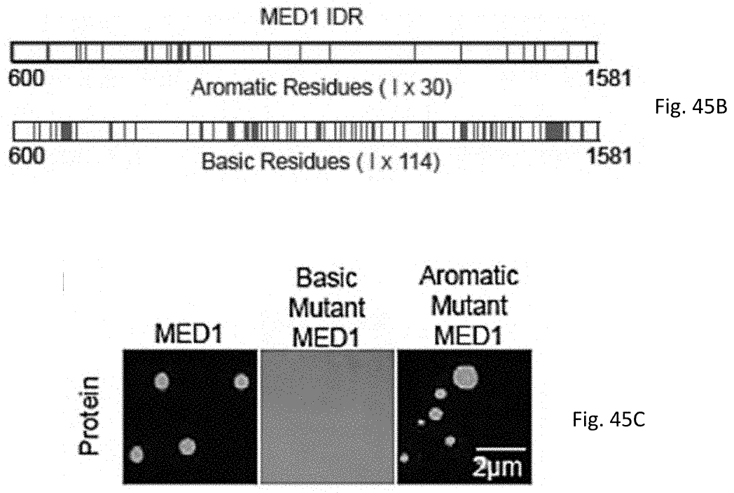

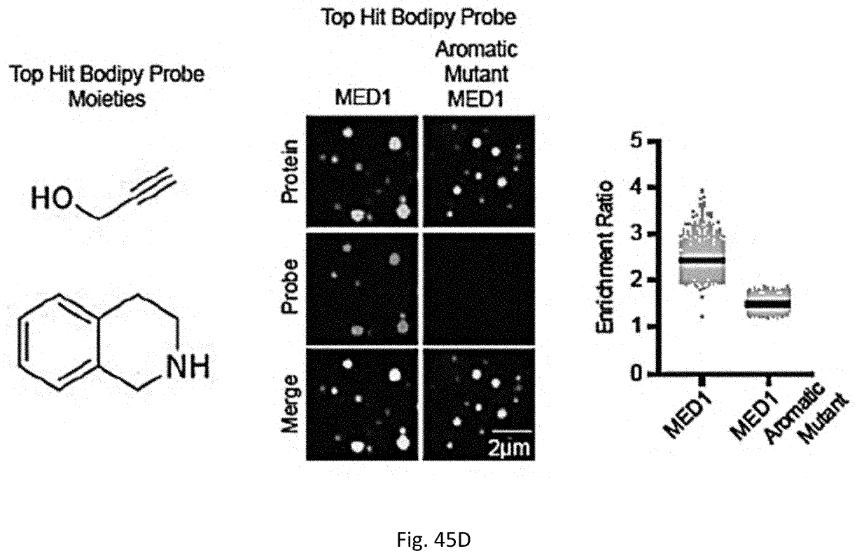

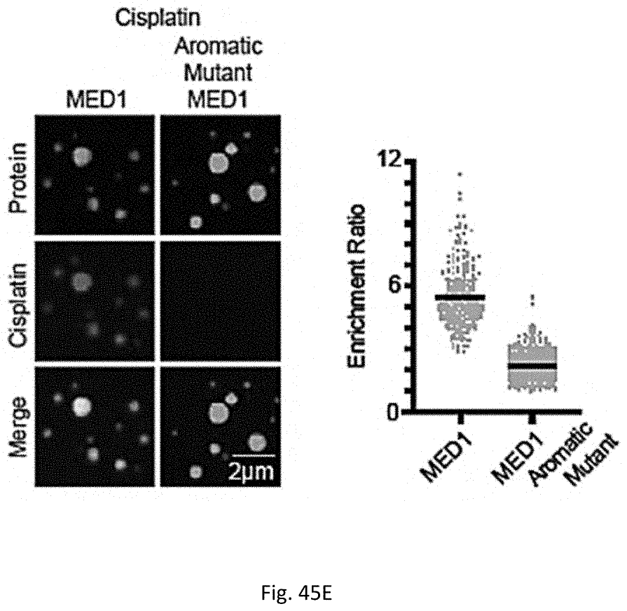

To gain additional insight into the nature of interactions governing small molecule enrichment in condensates, studies were focused on the MED1-IDR condensate. Fluorescence recovery after photobleaching (FRAP) experiments showed that cisplatin molecules are highly mobile in this condensate (FIGS. 43A, 43B), suggesting that the condensate produces a physiochemical environment that facilitates drug concentration in a state of high dynamic mobility. To gain insights into the chemical features of small molecules that may contribute to selective association with MED1 in condensates, a small molecule library of 81 compounds was used in which the fluorescent molecule boron-dipyrromethene (BODIPY) was modified with various combinations of chemical side groups (FIG. 44A), and measured the relative ability of these molecules to concentrate in MED1 condensates by confocal fluorescent microscopy. Molecules that contained aromatic rings were found to preferentially concentrate in MED1 condensates (FIGS. 44A-44D, FIG. 45A), suggesting that pi-pi or pi-cation interactions are among the physicochemical properties that favor small molecule partitioning into MED1 condensates. The aromatic amino acids in MED1, whose numbers exceed those in the other condensate forming proteins studied here (FIG. 34B) might contribute to such interactions, and to investigate this possibility a MED1 mutant protein was generated in which all 30 aromatic amino acids were mutated to alanine and tested its ability to form condensates and concentrate small molecules (FIG. 45B). The MED1 aromatic mutant protein retained the ability to form droplets in vitro, indicating that the aromatic amino acids are not required for droplet formation (FIG. 45C), but small molecule probes containing aromatic rings and the polar molecule cisplatin no longer partitioned into condensates formed by the MED1 aromatic mutant protein (FIGS. 45D, 45E). These results suggest that the aromatic residues of MED1 condensates contribute to the physicochemical properties that selectively concentrate these small molecules.

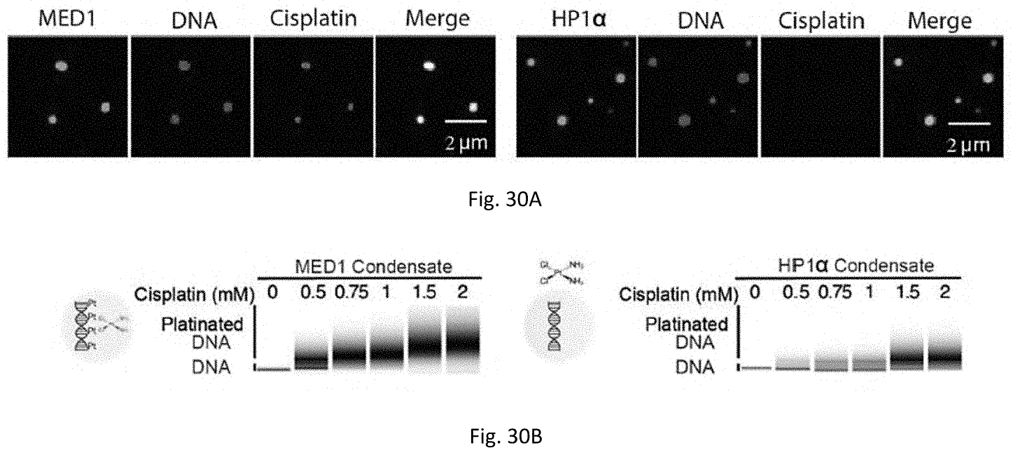

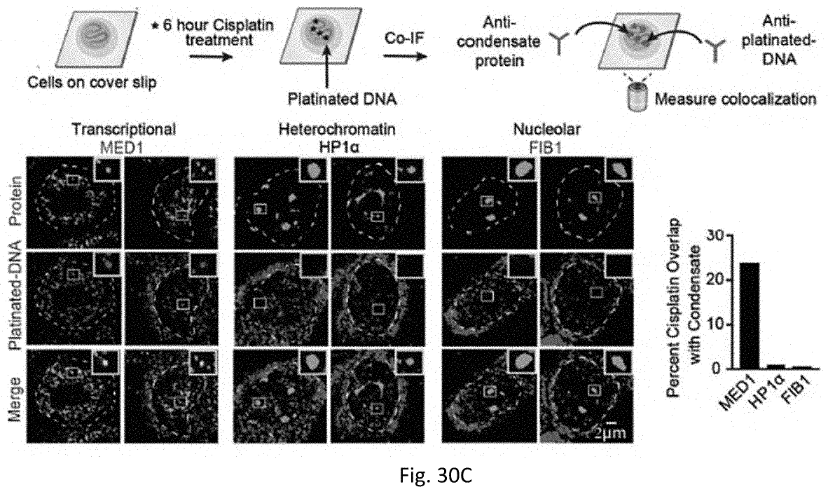

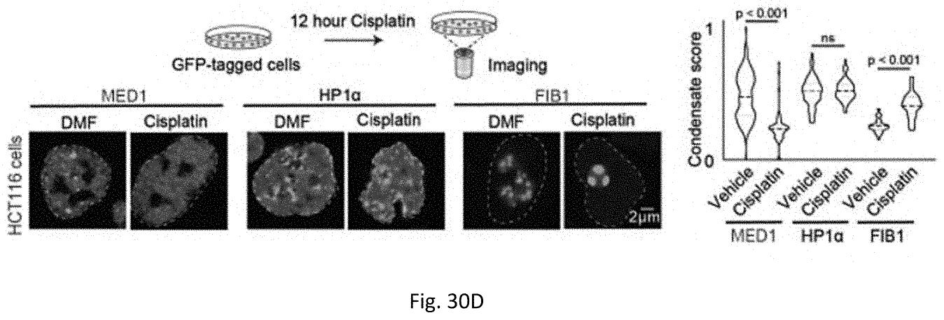

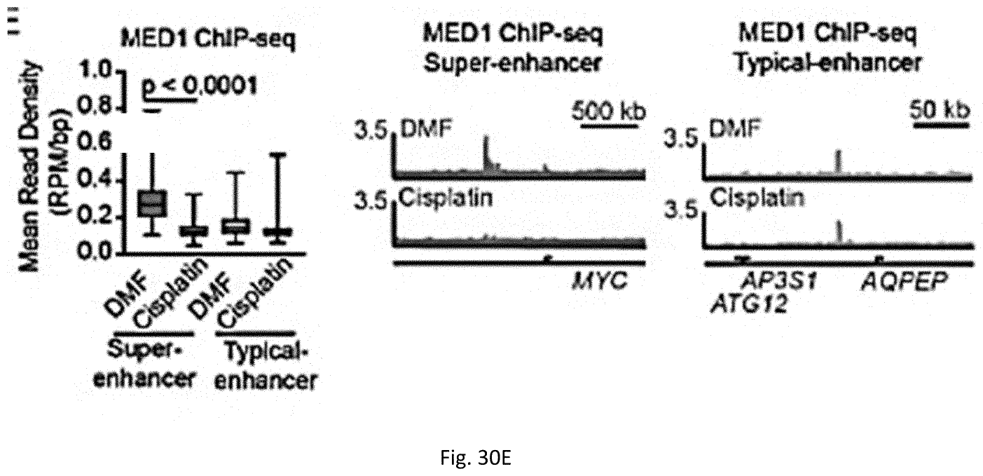

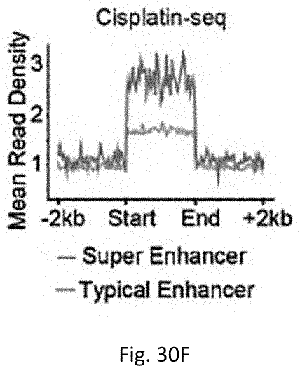

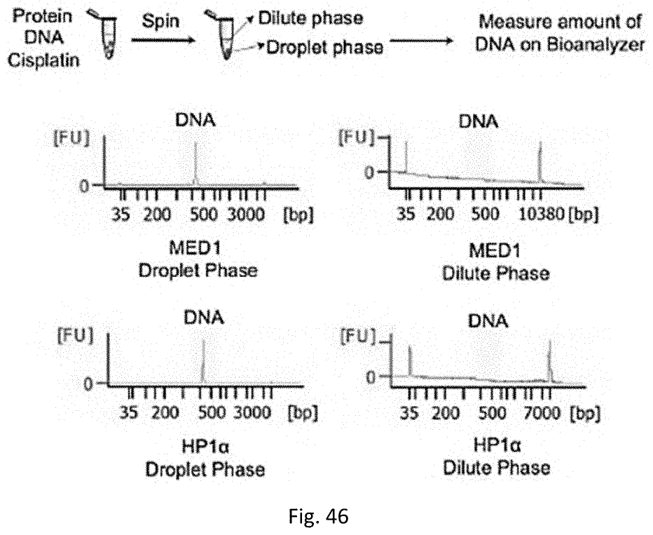

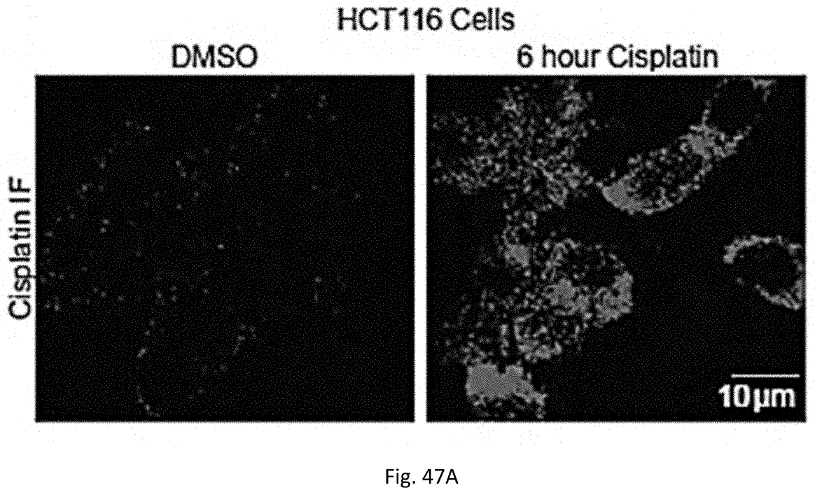

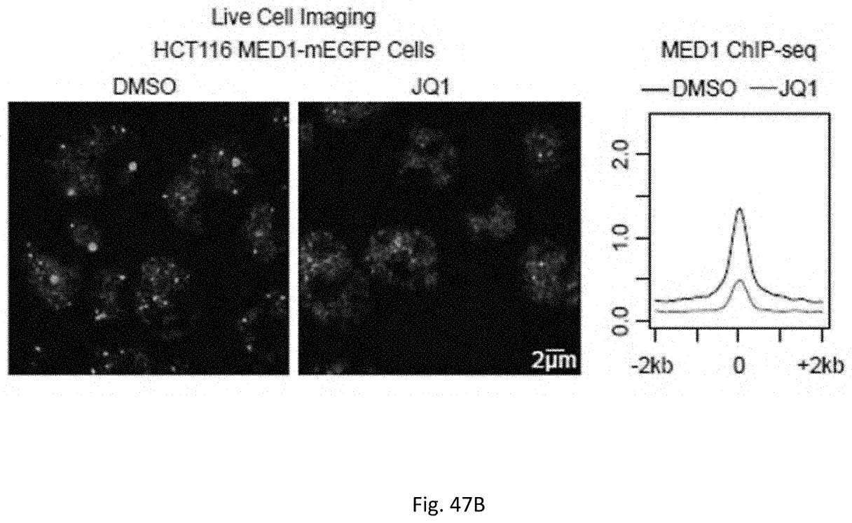

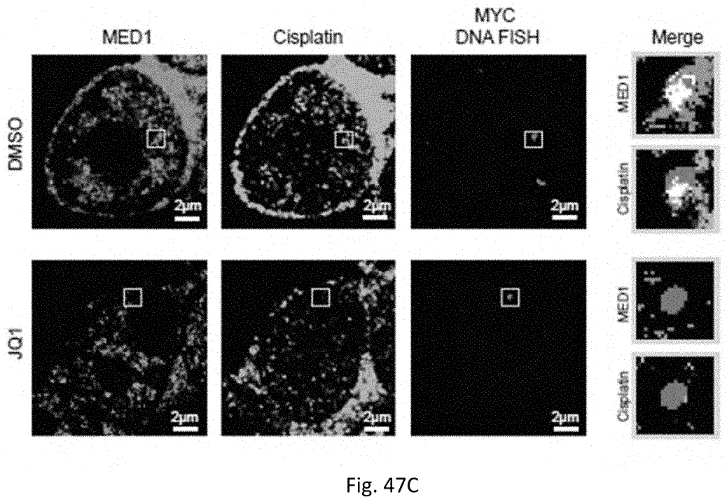

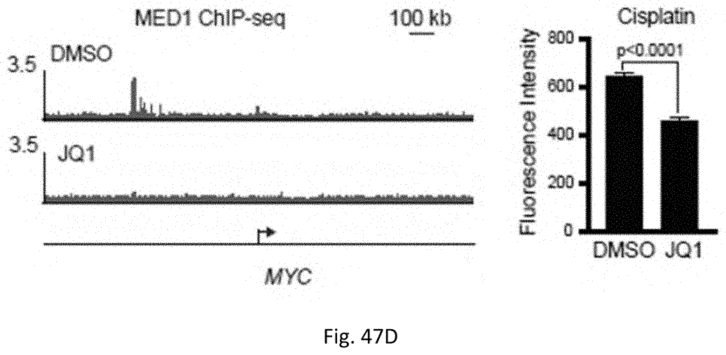

It was anticipated that the ability of small molecules to concentrate in specific condensates would influence target engagement and thus drug pharmacodynamics. To investigate this, the ability of MED1 and HP1α condensates to incorporate DNA (FIG. 30A) was taken advantage of, and the relative efficiency of DNA platination by cisplatin in MED1 condensates was measured, where cisplatin is concentrated, versus HP1α condensates, where cisplatin freely diffuses (FIG. 29A). DNA and protein were mixed under droplet-forming conditions, where DNA strongly partitioned into the droplet phase (FIG. 46), these condensates were treated with cisplatin, and DNA platination was visualized by size-shift on a bioanalyzer. The results show that DNA was more efficiently platinated in MED1 condensates than in HP1α condensates (FIG. 30B), consistent with the expectation that elevated concentrations of cisplatin in the MED1 condensates yield enhanced target engagement. If cisplatin becomes concentrated in Mediator condensates in cells, it would be expected that DNA colocalized with Mediator condensates would be preferentially platinated. To test this idea, co-immunofluorescence was performed in cisplatin-treated HCT116 colon cancer cells using an antibody that specifically recognizes platinated DNA (FIG. 47A) (40) together with antibodies specific for MED1, HP1α, or FIB1. Consistent with cisplatin's preference for MED1 condensates in vitro, it was found that platinated DNA was frequently colocalized with MED1 condensates, but not with HP1α or FIB1 condensates (FIG. 30C). To determine whether the ability of cisplatin to engage DNA is dependent on the presence of a MED1 condensate, cells were treated with JQ1, which caused a loss of MED1 condensates (FIG. 47B), and observed a concomitant reduction in platinated DNA at the MYC oncogene (FIGS. 47C, 47D). These results are consistent with the idea that concentration of small molecules in specific condensates can influence the efficiency of target engagement.

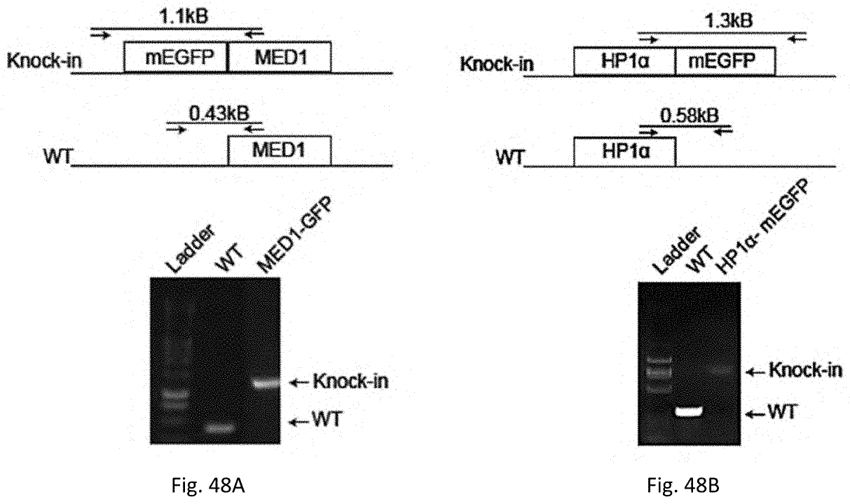

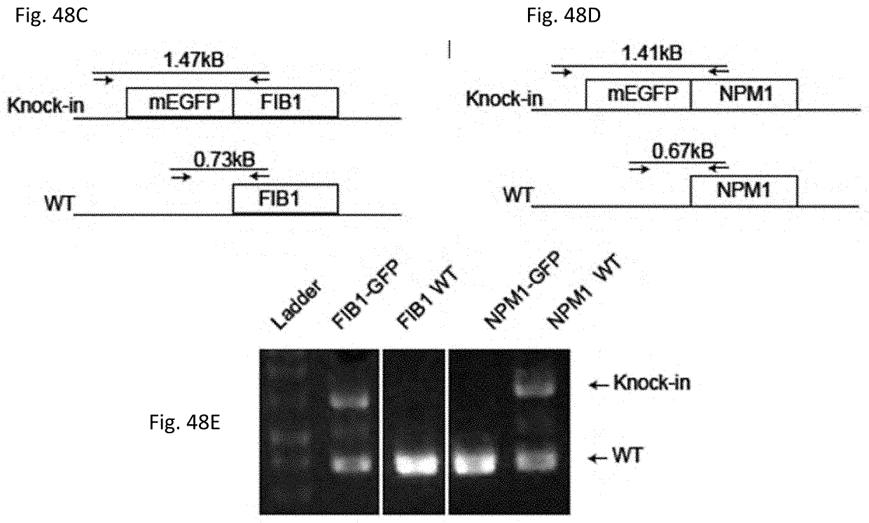

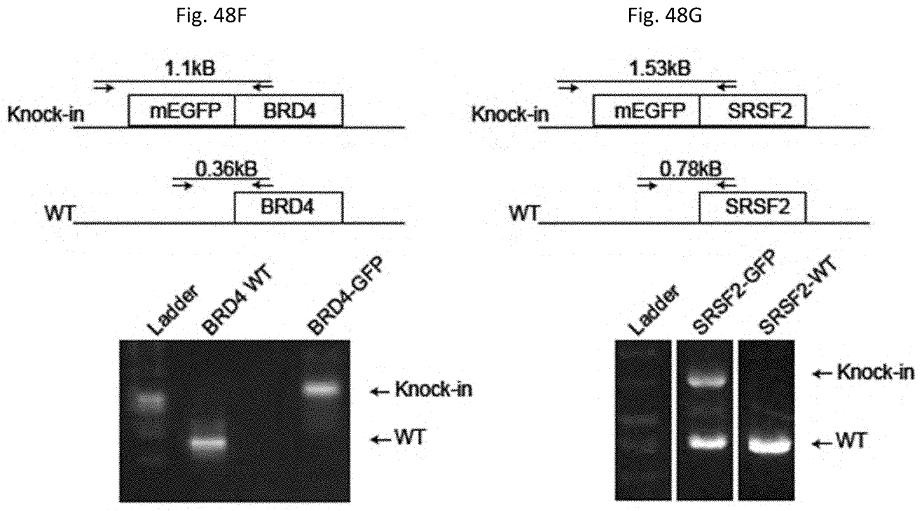

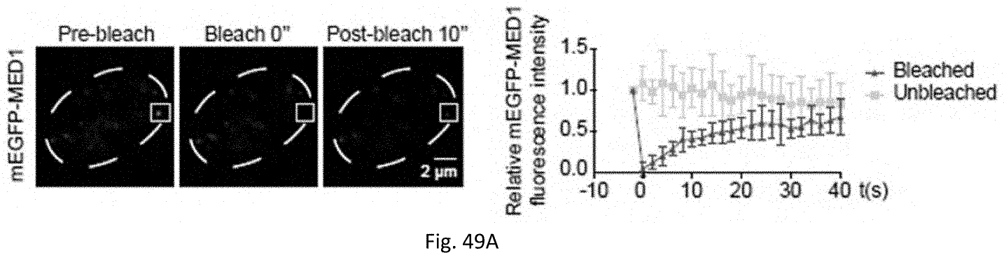

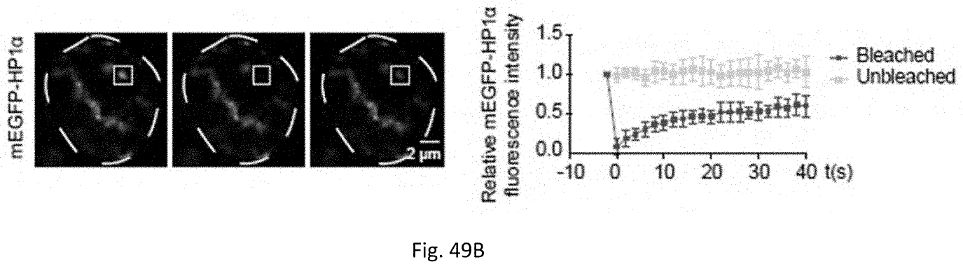

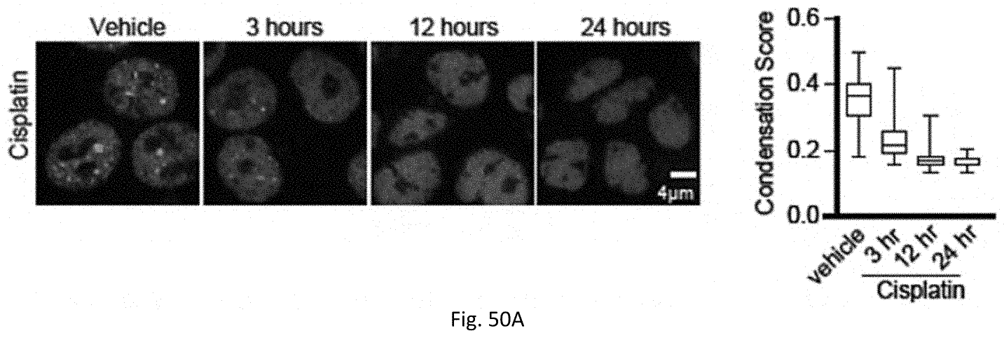

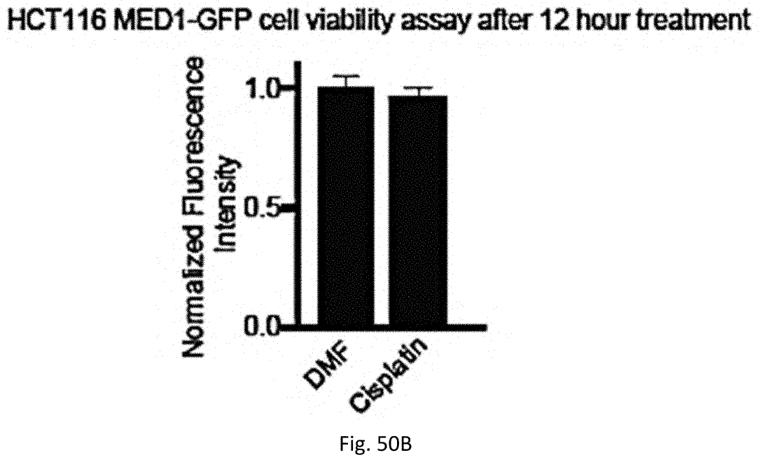

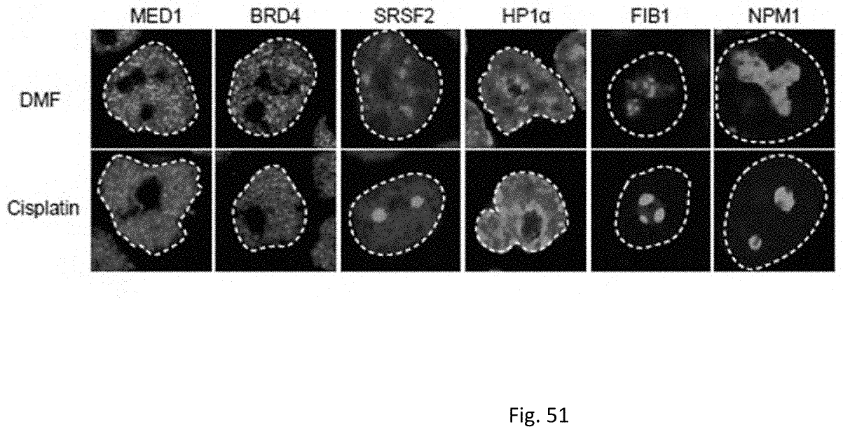

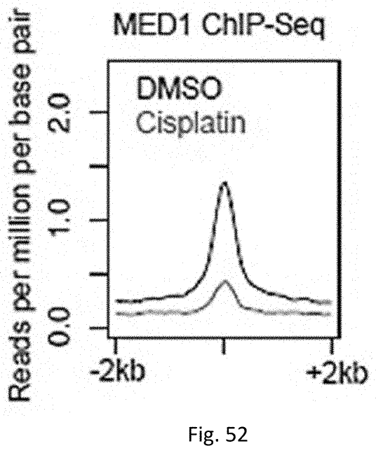

In cells, the preferential modification of DNA in MED1-containing condensates might be expected to selectively disrupt these condensates with prolonged treatment. To test this, HCT116 colon cancer cells were engineered to express GFP-tagged marker proteins for each of the 6 nuclear condensates (FIGS. 48A-48F, FIG. 49A, FIG. 49B). When exposed to cisplatin, a selective and progressive reduction in MED1 condensates was observed (FIG. 30D, FIG. 50A, FIG. 50B, FIG. 51). Consistent with this, cisplatin treatment led to a preferential loss of MED1 ChIP-seq signal at super-enhancers (FIG. 40E, FIG. 52). Furthermore, high throughput sequencing data from platinated-DNA pull-down (41) revealed that cisplatin-modified DNA preferentially occurs at super-enhancers (SEs), where MED1 is concentrated (42) (FIG. 30F). These results are consistent with reports that cisplatin preferentially modifies transcribed genes (41, 43), and argue that this effect is due to preferential condensate partitioning. Taken together, these results suggest a model where cisplatin preferentially modifies SE DNA, which in turn leads to dissolution of these condensates. Previous studies have shown that diverse tumor cells become highly dependent on super-enhancer driven oncogene expression (44-48), which might explain why platinum drugs, which are capable of general DNA modification, are effective therapeutics in diverse cancers (49).

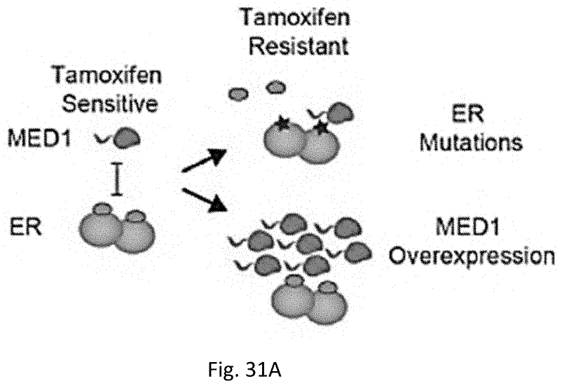

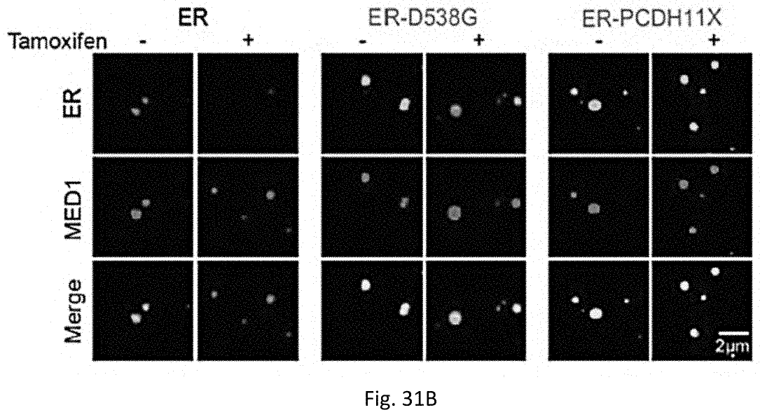

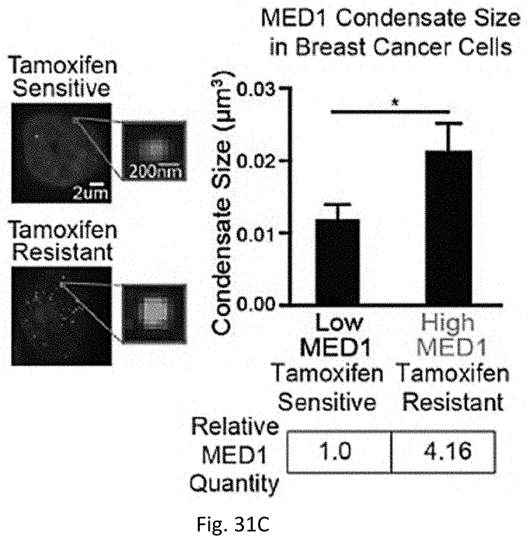

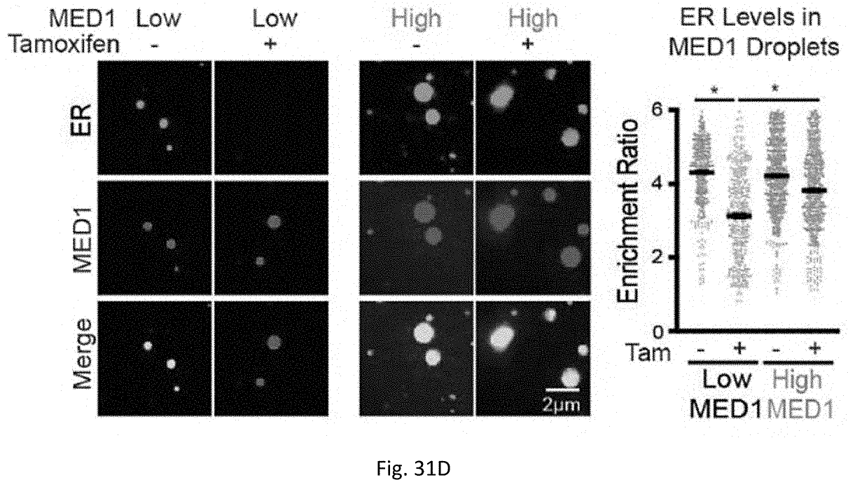

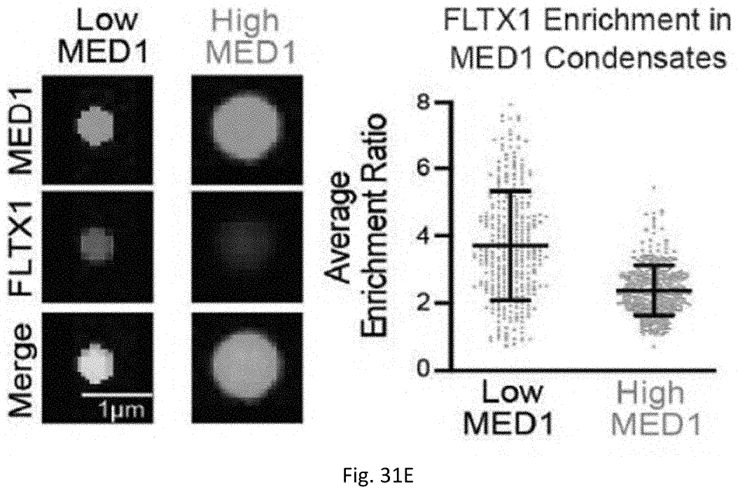

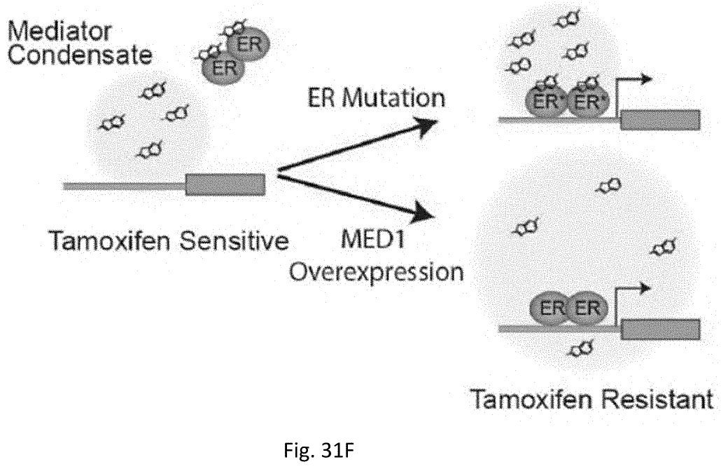

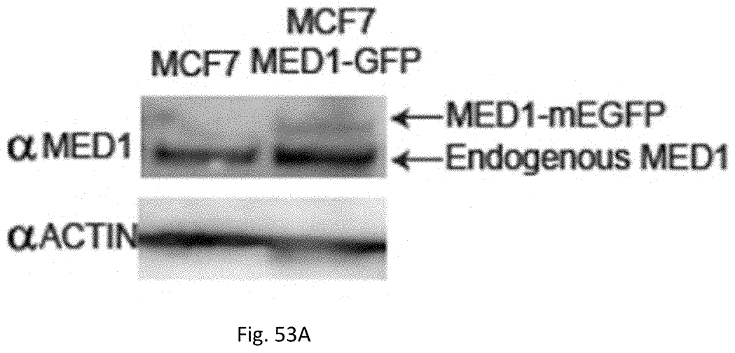

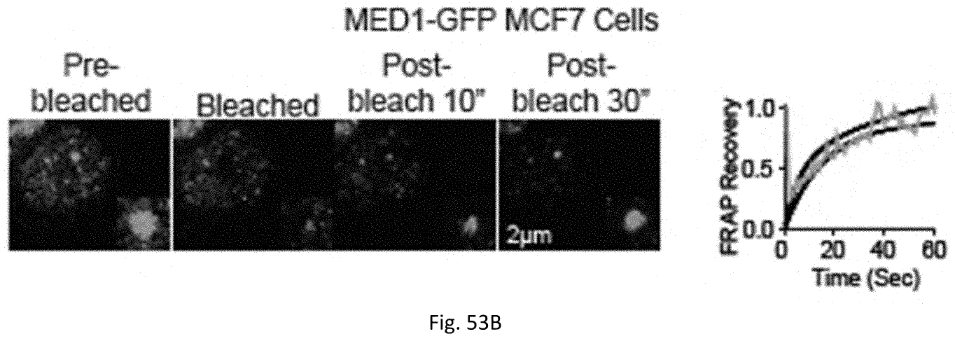

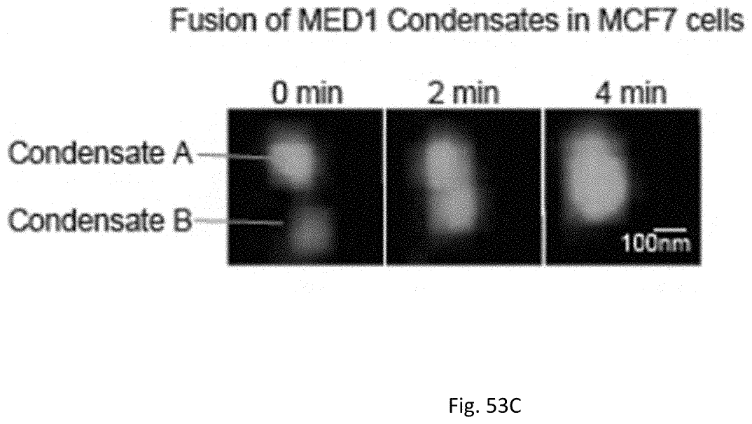

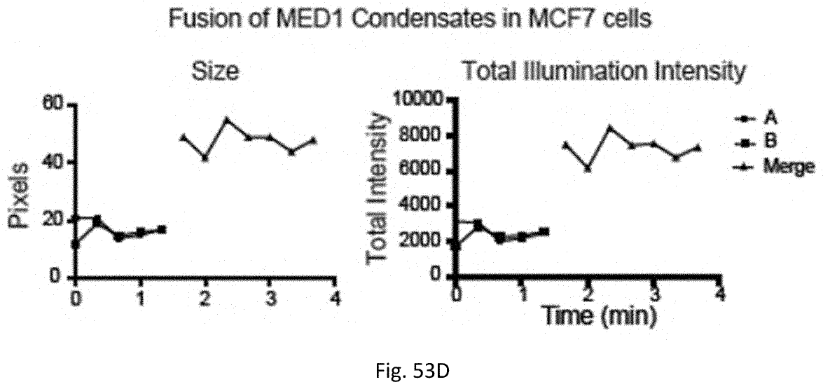

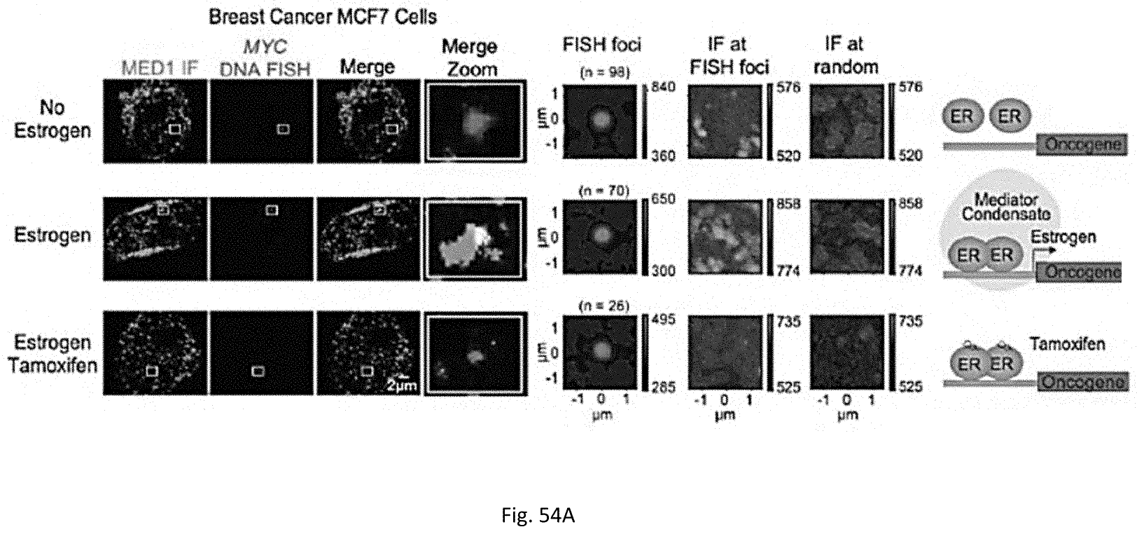

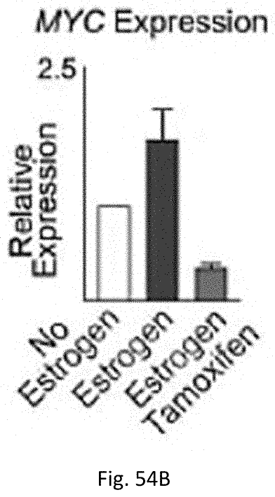

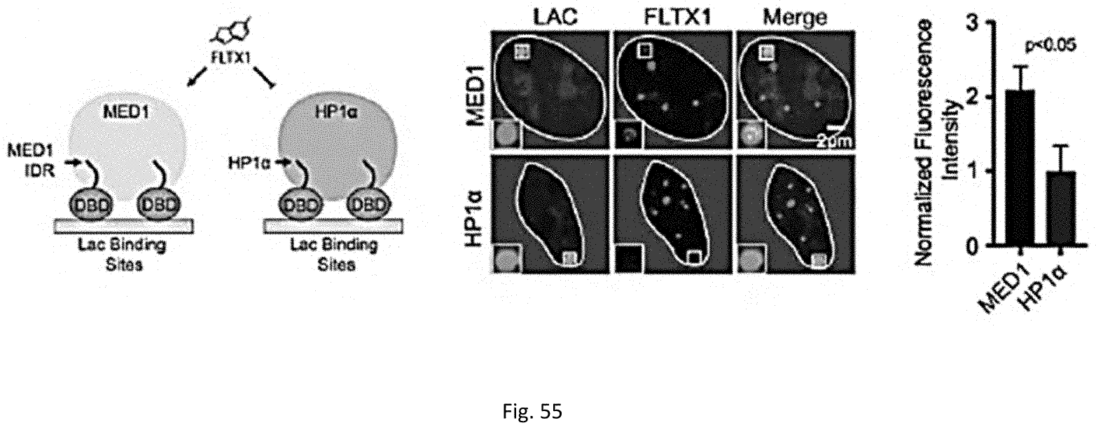

The behavior of another clinically important antineoplastic drug, tamoxifen, was explored to assess whether drug response and resistance are associated with partitioning in condensates (FIG. 31A). ERα incorporates into MED1 condensates in an estrogen-dependent manner in vitro (12); droplet assays confirmed this and revealed that the addition of tamoxifen leads to eviction of ERα from the MED1 condensates (FIG. 31B). The effects of estrogen and tamoxifen on MED1 condensates in breast cancer cells were further investigated, focusing on the MYC oncogene due to its prominent oncogenic role and responsiveness to estrogen (50). MED1 condensates were observed on the MYC oncogene in the ER+ breast cancer cell line MCF7 (FIG. 40A, FIG. 53A-53D). DNA FISH with MED1 IF revealed that estrogen enhances formation of MED1 condensates at the MYC oncogene and tamoxifen treatment reduces these (FIG. 54A, FIG. 54B). Artificial MED1 condensates without ER concentrated FLTX1 at the site of the condensate (FIG. 55), indicating that ER is not required for the partitioning of FLTX1 into MED1 condensates in cells. These results are consistent with the model that ERα interacts with MED1 condensates in an estrogen-dependent, tamoxifen-sensitive manner to drive oncogene expression in breast cancer cells.

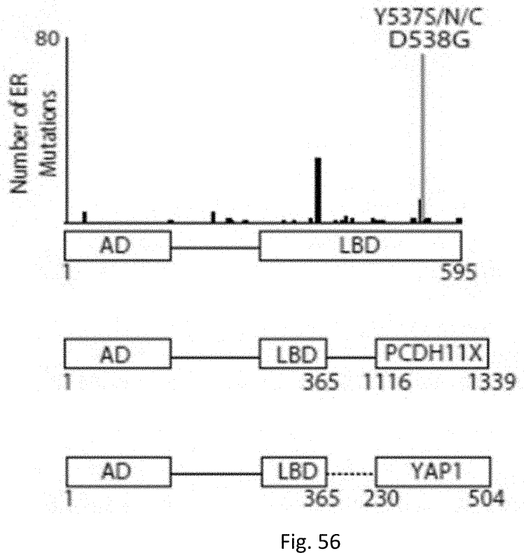

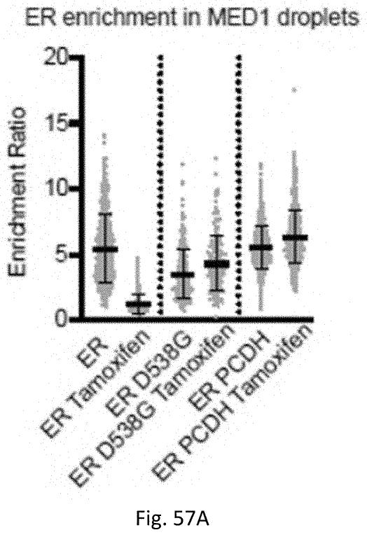

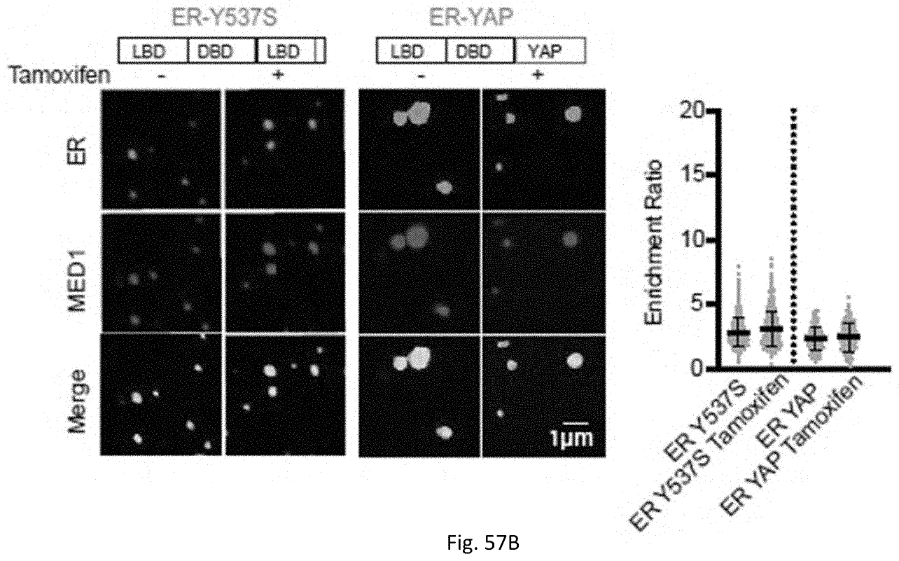

The mechanisms that produce drug resistance can provide clues to drug activity in the clinical setting. Tamoxifen resistance is an enduring clinical challenge and can be mediated by multiple mechanisms including ERα mutation and MED1 overexpression (FIG. 31A, FIG. 56) (51, 52). To investigate whether the ERα mutations alter ERα behavior in condensates, we produced 4 patient-derived ERα mutant proteins and tested their partitioning in the presence of tamoxifen. In contrast to WT ERα, condensates composed of patient-derived ERα mutants and MED1 were not disrupted upon tamoxifen treatment (FIG. 31B, FIG. 57A, FIG. 57B). The ERα point mutations reduce the affinity for tamoxifen approximately 10-fold (52), indicating that the drug concentration in the droplet is inadequate to evict these ER mutant proteins when this affinity is reduced.

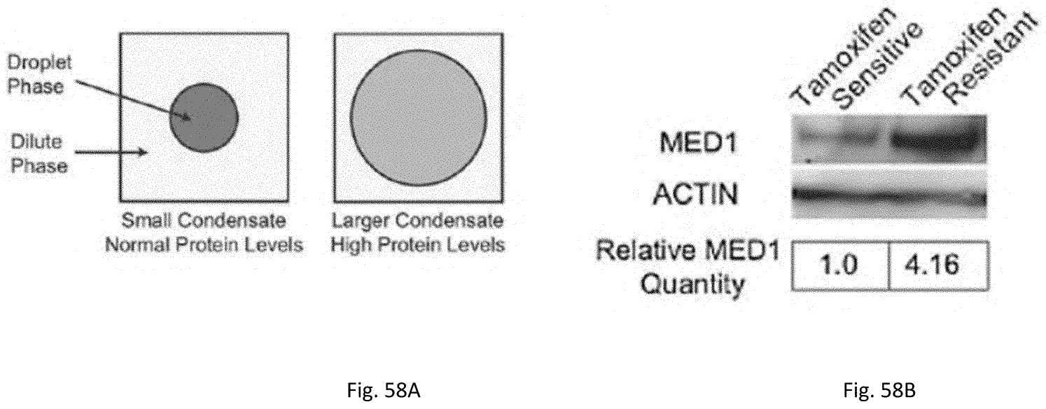

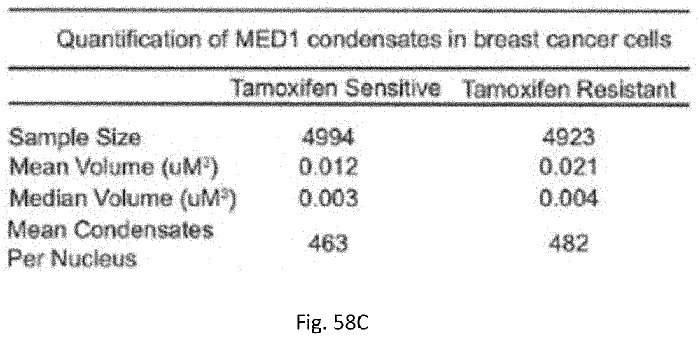

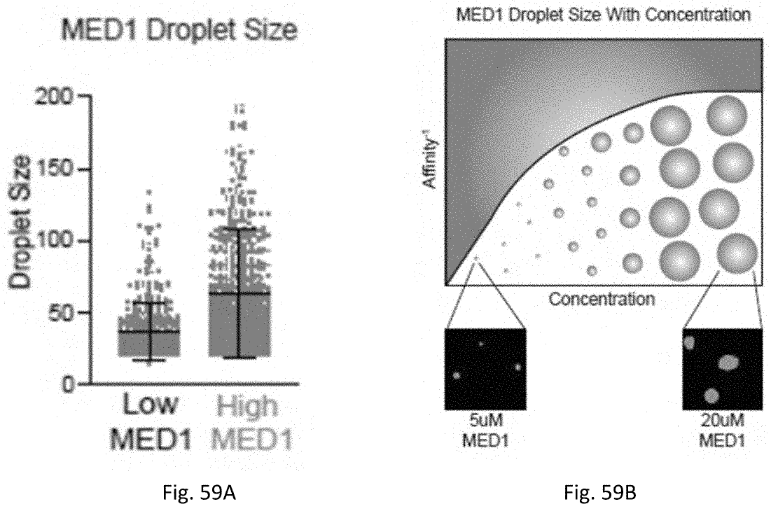

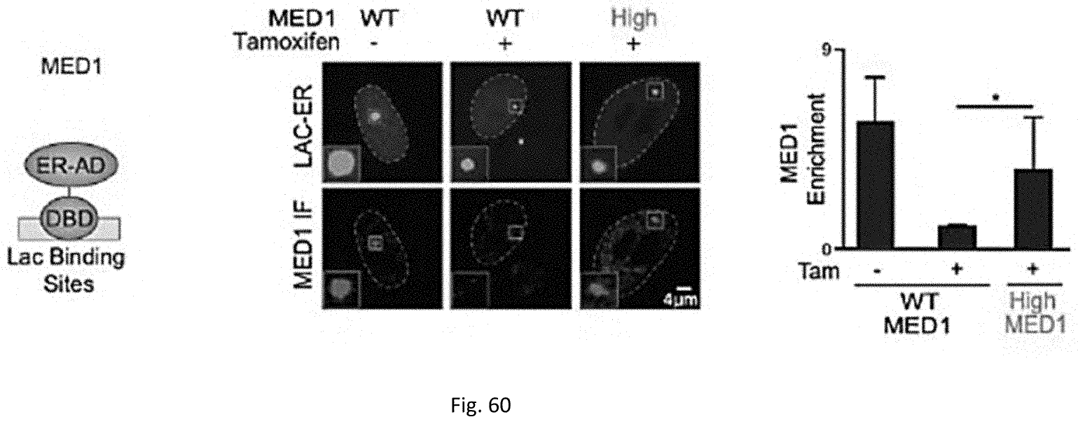

MED1 overexpression is associated with tamoxifen resistance and poor prognosis in breast cancer (51), but it is not clear why overexpression of one subunit of the Mediator complex produces resistance. The possibility that overexpressed MED1 is incorporated into transcriptional condensates was considered, which contain clusters of Mediator molecules (39), thereby expanding their volumes and diluting the available tamoxifen (FIG. 58A). It was found that the tamoxifen-resistant breast cancer cell line TAMR7 (53), which was derived from the tamoxifen-sensitive cell line MCF7, produces 4-fold elevated levels of MED1 protein (FIG. 58B). The volume of MED1-containing condensates is 2-fold larger in these cells (FIG. 31C, FIG. 58C). When modeled in an in vitro droplet assay, it was found that a 4-fold increase in MED1 levels led to a commensurate increase in droplet size (FIG. 59A, FIG. 59B). Furthermore, it was found that 100 μM tamoxifen prevented ERα incorporation into MED1 condensates (FIGS. 31B, 31D), but was much less effective in preventing ERα incorporation into the larger MED1 condensates produced with higher MED1 levels (FIG. 31D). To confirm that the levels of tamoxifen in the larger droplets are more dilute, the enrichment of the fluorescent tamoxifen analog FLTX1 in MED1 droplets was measured, and found that the larger condensates have lower concentrations of the drug (FIG. 31E). These results were mirrored in cells, where a collection of tethered ERα molecules form a MED1 condensate that is eliminated by tamoxifen, but when MED1 is overexpressed tamoxifen is unable to dissociate the ERα-MED1 condensate (FIG. 60). These results support a model of tamoxifen resistance where MED1 overexpression causes the formation of larger transcriptional condensates, in which tamoxifen is diluted and thereby less effective in dissociating ER from the condensate (FIG. 31F).

The results show that drugs partition selectively into condensates, that this can occur through physicochemical properties that exist independent of their molecular targets, and that cells can develop resistance to drugs through condensate altering mechanisms. This may explain the surprising observation that inhibition of global gene regulators such as BRD4 or CDK7 can have selective effects on oncogenes that have acquired large super-enhancers (46); selective partitioning of inhibitors like JQ1 and THZ1 into super-enhancer condensates will preferentially disrupt transcription at those loci. These results also have implications for future development of efficacious disease therapeutics; effective target-engagement will depend on measurable factors such as drug partitioning in condensates (FIGS. 61A-61D). Condensate assays of the type described here may thus help optimize condensate partitioning, target engagement, and the therapeutic index of small molecule drugs.

Materials and Methods

Cell Lines

Cell lines were obtained as indicated, TamR7 (ECACC 16022509). V6.5 murine embryonic stem cells were a gift from R. Jaenisch of the Whitehead Institute. V6.5 are male cells derived from a C57BL/6(F)×129/sv(M) cross. MCF7 cells were a gift from the R. Weinberg of the Whitehead Institute and HCT116 cells were from ATCC (CCL-247) were used. V6.5 murine embryonic stem endogenously tagged with MED1-mEGFP (10), BRD4-mEGFP (10), SRSF2-mEGFP (11), or HP1α-mEGFP were used. Cells were tested negative for mycoplasma. The CRISPR/Cas9 system was used to generate genetically modified endogenously tagged ESCs and HCT116 cells. Target specific sequences were cloned into a plasmid containing sgRNA backbone, a codon-optimized version of Cas9, and BFP or mCherry. A homology directed repair template was cloned into pUC19 using NEBuilder HiFi DNA Master Mix (NEB E2621S). The homology repair template consisted of mCherry or mEGFP cDNA sequence flanked on either side by 800 bp homology arms amplified from genomic DNA using PCR. To generate genetically modified cell lines, 750,000 cells were transfected with 833 ng Cas9 plasmid and 1,666 ng non-linearized homology repair PCR genotyping was performed using Phusion polymerase (Thermo Scientific F531S). Products were amplified according to kit recommendations and visualized on a 1% agarose gel. The following primers were used for PCR genotyping:

HP1α-mCherry_fwd (mES):
(SEQ ID NO: 2)
AACGTGAAGTGTCCACAGATTG
HP1α-mCherry_rev (mES):
(SEQ ID NO: 3)
TTATGGATGCGTTTAGGATGG
HP1α-GFP_fwd (HCT116):
(SEQ ID NO: 4)
CCAAGGTGAGGAGGAAATCA
HP1α-GFP_rev (HCT116):
(SEQ ID NO: 5)
CACAGGGAAGCAGAAGGAAG
MED1α-GFP_fwd (HCT116):
(SEQ ID NO: 6)
GAAGTTGAGAGTCCCCATCG
MED1-GFP_rev (HCT116):
(SEQ ID NO: 7)
CGAGCACCCTTCTCTTCTTG
BRD4-GFP_fwd (HCT116):
(SEQ ID NO: 8)
CTGCCTCTTGGGCTTGTTAG
BRD4-GFP_rev (HCT116):
(SEQ ID NO: 9)
TTTGGGGAGAGGAGACATTG
SRSF2-GFP_fwd (HCT116):
(SEQ ID NO: 10)
CAAGTCTCCTGAAGAGGAAGGA
SRSF2-GFP_rev (HCT116):
(SEQ ID NO: 11)
AAGGGCTGTATCCAAACAAAAAC
FIB1-GFP_fwd (HCT116):
(SEQ ID NO: 12)
CCTTTTAATCAGCAACCCACTC
FIB1-GFP_rev (HCT116):
(SEQ ID NO: 13)
GTGACCGAGTGAGAATTTACCC
NPM1-GFP_fwd (HCT116):
(SEQ ID NO: 14)
TCAAATTCCTGAGCTGAAGTGA
NPM1-GFP_rev (HCT116):
(SEQ ID NO: 15)
AACACGGTAGGGAAAGTTCTCA

Cell Culture

V6.5 murine embryonic stem (mES) cells were grown in 2i+LIF conditions. mES cells were grown on 0.2% gelatinized (Sigma, G1890) tissue culture plates. The media used for 2i+LIF media conditions is as follows: 967.5 mL DMEM/F12 (GIBCO 11320), 5 mL N2 supplement (GIBCO 17502048), 10 mL B27 supplement (GIBCO 17504044), 0.5 mML-glutamine (GIBCO 25030), 0.5× non-essential amino acids (GIBCO 11140), 100 U/mL Penicillin-Streptomycin (GIBCO 15140), 0.1 mM b-mercaptoethanol (Sigma), 1 uM PD0325901 (Stemgent 04-0006), 3 uM CHIR99021 (Stemgent 04-0004), and 1000 U/mL recombinant LIF (ESGRO ESG1107). TrypLE Express Enzyme (Life Technologies, 12604021) was used to detach cells from plates. TrypLE was quenched with FBS/LIF-media ((DMEM K/0 (GIBCO, 10829-018), 1× nonessential amino acids, 1% Penicillin Streptomycin, 2 mM L-Glutamine, 0.1 mM b-mercaptoethanol and 15% Fetal Bovine Serum, FBS, (Sigma Aldrich, F4135)). Cells were spun at 1000 rpm for 3 minutes at RT, resuspended in 2i media and 5×10⁶ cells were plated in a 15 cm dish.

MCF7 cells and HCT116 cells were grown in complete DMEM media (DMEM (Life Technologies 11995073), 10% Fetal Bovine Serum, FBS, (Sigma Aldrich, F4135), 1% L-glutamine (GIBCO, 25030-081), 1% Penicillin Streptomycin (Life Technologies, 15140163)). For growth in estrogen-free conditions MCF7 cells in regular media were washed 3× with PBS then the media was changed to estrogen free media containing phenol red-free DMEM (Life Technologies 21063029), 10% charcoal stripped FBS (Life Technologies A3382101), 1% L-glutamine (GIBCO, 25030-081) and 1% Penicillin Streptomycin (Life Technologies, 15140163) for 48 hours prior to use.

TamR7 cells were grown in TAMR7 media (Phenol red-free DMEM/F12 (Life Technologies 21041025, 1% L-glutamine (GIBCO, 25030-081) 1% Penicillin Streptomycin (Life Technologies, 15140163), 1% Fetal Bovine Serum, FBS, (Sigma Aldrich, F4135), 6 ng/mL insulin (Santa Cruz Biotechnology, sc-360248)). For passaging, cells were washed in PBS (Life Technologies, AM9625). TrypLE Express Enzyme (Life Technologies, 12604021) was used to detach cells from plates. TrypLE was quenched with indicated media.

Live Cell Imaging

Cells were grown on glass dishes (Maack P35G-1.5-20-C), Before imaging the cells, culture medium was replaced with phenol red-free 2i media, and imaged using the Andor Revolution Spinning Disk Confocal microscope, Raw Andor images were processed using FIJI. For imaging mESC, coated glass dishes were used (5 μg/ml of poly-L-ornithine (Sigma-Aldrich, P4957) for 30 minutes at 37° C., and with 5 μg/ml of laminin (Corning, 354232) for 2-16 hours at 37° C.), For imaging FIB1 and NPM1 in mES cells, vectors encoding GFP-tagged NPM1 or FIB1 were transfected as described above with Lipofectamine 3000 per package instructions.

Immunofluorescence of Tissue Samples

Fresh frozen breast, and colon tissues were purchased from BioIVT. Frozen breast tissue was fixed in 2% PFA in PBS for 30 minutes-1 hour. Fixed tissue was incubated in 30% sucrose in PBS at 4° C. for 4 days. Tissue was embedded in OCT and frozen. Fresh frozen colon tissue was embedded in OCT and frozen. Tissue was sections into 10 um sections using the cryostat with temperature set at −25° C. or −30° C. Sections were stored at −20° C. For IF, sections were let reach room temperature, they were fixed in 4% PFA in PBS for 10 minutes. Following three washes in PBS, tissues were permeabilized using 0.5% TX100 in PBS, washed three times in PBS and blocked with 4% BSA in PBS for 30 minutes. Primary antibodies were diluted into 4% BSA in PBS and added to the tissue sample for O/N incubation at RT. Following three washes in PBS, samples were incubated with secondary antibodies diluted 1:500 in 4% BSA in PBS. Samples was washed in PBS, DNA was stained using 20 μm/mL Hoechst 33258 (Life Technologies, H3569) for 5 minutes and mounted using Vectashield (VWR, 101098-042). Images were acquired using the Elyra Super-Resolution Microscope at Harvard Center for Biological Imaging. Images were post-processed using Fiji Is Just ImageJ (https://fiji.sc/).

Nuclear Volume Quantification of Condensates

For image acquisition: 10 z-slices were imaged. The outline of the nuclei were defined manually in Fiji Is Just ImageJ (https://fiji.sc/) and the volume of each nucleus was calculated as nuclear area (μm)*number of z-slices imaged (10)*voxel depth (0.1 μm).

The volume of condensates in the nucleus were measured using a custom Python script and the scikit-image package. Condensates were segmented from 3D images of the protein channel on two criteria: (1) an intensity threshold that was three s.d. above the mean of the image; (2) size thresholds (10 pixel minimum condensate size). The estimated volume of the segmented objects was then calculated by multiplying the width (μm)*height (μm)*voxel depth (0.1 μm). For each protein factor, the average and s.d. volume of condensates in the healthy and malignant tissue was reported. The number of condensates per nucleus was defined as the number of segmented objects contained within the perimeter of the defined nucleus. For each protein factor, the average and s.d. number of condensates per nucleus in the healthy and malignant tissue was reported. Percentage of nuclear volume occupied by the condensates was calculated as follows: (E volume of all detected condensates in the nucleus)/(estimated nuclear volume).

Antibodies

The following antibodies were used for Immunofluorescence: NPM1 (ab10530), BRD4 (ab128874), MED1 (ab64965), HP1a (ab109028), FIB1 (ab5821), SRSF2 (ab11826), ER (ab32063), CDK7 (sc-7344), Cisplatin modified DNA (ab103261), 568 goat anti rat A11077, Goat anti-Rabbit IgG Alexa Fluor 488, Life Technologies A11008.

Protein Purification

Human cDNA was cloned into a modified version of a T7 pET expression vector. The base vector was engineered to include a 5′ 6×HIS followed by either BFP, mEGFP or mCherry and a 14 amino acid linker sequence “GAPGSAGSAAGGSG.” (SEQ ID NO: 16). NEBuilder® HiFi DNA Assembly Master Mix (NEB E2621S) was used to insert these sequences (generated by PCR) in-frame with the linker amino acids. All expression constructs were sequenced to ensure sequence identity.

For protein expression plasmids were transformed into LOBSTR cells (gift of Chessman Lab) and grown as follows. A fresh bacterial colony containing the tagged MED1 constructs were inoculated into LB media containing kanamycin and chloramphenicol and grown overnight at 37° C. Cells were diluted 1:30 in 500 ml room temperature LB with freshly added kanamycin and chloramphenicol and grown 1.5 hours at 16° C. IPTG was added to 1 mM and growth continued for 20 hours. Cells were collected and stored frozen. Cells containing all other expression plasmids were treated in a similar manner except they were grown for 5 hours at 37° C. after IPTG induction.

Cell pellets of SRSF1 and SRSF2-IDR were resuspended in 15 ml of denaturing buffer (50 mM Tris 7.5, 300 mM NaCl, 10 mM imidazole, 8M Urea) with complete protease inhibitors (Roche, 11873580001) and sonicated (ten cycles of 15 seconds on, 60 sec off). The lysates were cleared by centrifugation at 12,000 g for 30 minutes and added to 1 ml of Ni-NTA agarose (Invitrogen, R901-15) that had been pre-equilibrated with 10 volumes of the same buffer. Tubes containing this agarose lysate slurry were rotated for 1.5 hours at room temperature, then centrifuged for 10 minutes at 3,000 rpm, washed with 2×5 ml of lysis buffer and eluted with 3×2 ml lysis buffer with 250 mM imidazole. Elutions were incubated for at least 10 minutes rotating at room temperature and centrifuged for 10 minutes at 3,000 rpm to collect protein. Fractions were run on a 12% acrylamide gel and proteins of the correct size were dialyzed first against buffer containing 50 mM Tris pH 7.5, 500 mM NaCl, 1 Mm DTT and 4M Urea, followed by the same buffer containing 2M Urea and lastly 2 changes of buffer with 10% Glycerol, no Urea. Any precipitate after dialysis was removed by centrifugation at 3.000 rpm for 10 minutes. All other proteins were purified in a similar manner by resuspending cell pellets in 15 ml of buffer containing 50 mM Tris pH7.5, 500 mM NaCl, complete protease inhibitors, sonicating, and centrifuging at 12,000×g for 30 minutes at 4° C. The lysate was added to 1 ml of pre-equilibrated Ni-NTA agarose, and rotated at 4° C. for 1.5 hours. The resin slurry was centrifuged at 3,000 rpm for 10 minutes, washed with 2×5 ml lysis buffer with 50 mM imidazole and eluted by incubation for 10 or more minutes rotating 3× with 2 ml lysis buffer containing 250 mM imidazole followed by centrifugation and gel analysis. Fractions containing protein of the correct size were dialyzed against two changes of buffer containing 50 mM Tris 7.5, 125 mM NaCl, 10% glycerol and 1 mM DTT at 4° C. or the same buffer with 500 mM NaCl for the HP1α construct.

The following human proteins or protein fragments were used for production:

NPM1—full length, amino acids 1-294.

SRSF2—full length, amino acids 1-221.

HP1α—full length, amino acids 1-191.

MED1—amino acids 600-1581.

MED1—aromatic mutant amino acids 600-1581, all aromatic residues changed to alanine.

MED1—basic mutant amino acids 600-1581, all basic residues changed to alanine.

BRD4—amino acids 674-1351.

FIB1—full length, amino acids 1-321.

ER and ER mutants—full length, amino acids 1-595 (WT).

Cbioportal Data Acquisition

For frequency of patient mutations, cbioportal (www.cbioportal.org/) was queried for mutations in ESR1 that are present in any breast cancer sequencing data set.

Drugs and Small Molecules

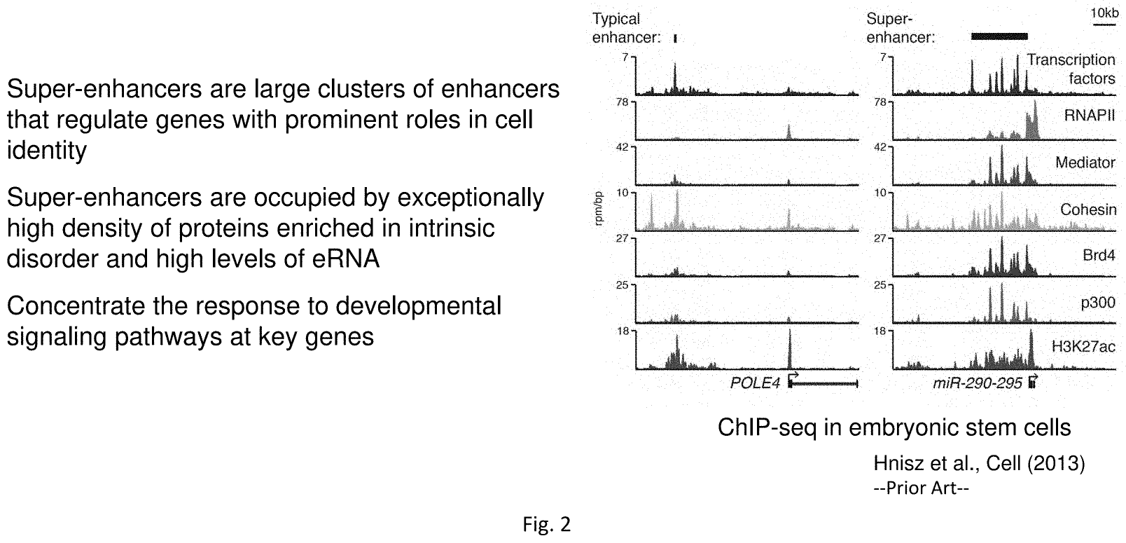

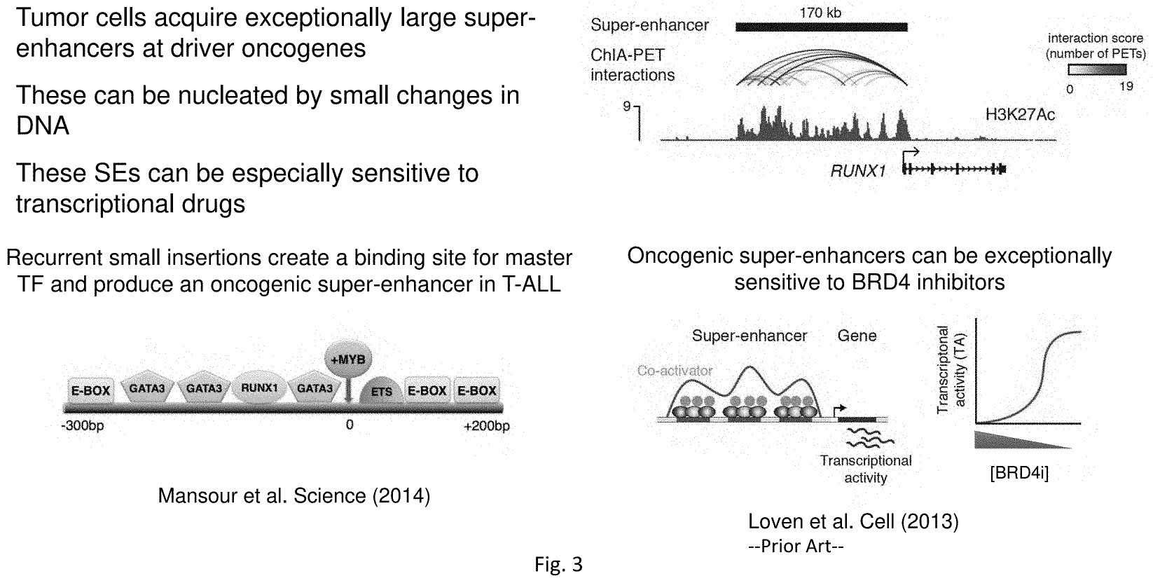

Drugs and small molecules were obtained and processed as follows. Hoescht 33258 (Life Technologies H3569) was obtained and utilized in liquid form, Fluorescein (Sigma F2456) was dissolved in DMSO at 10 mM then diluted further in droplet formation buffer for use. Dextrans measuring 4.4 kDa (Sigma T1037), 10 kDa (Invitrogen D1816), 40 kDa (Invitrogen D1842), or 70 kDa (Invitrogen D1864) conjugated to either TRITC or FITC, ROX (Life technologies 12223012), and Texas Red (Sigma Aldrich 60311-02-6), were diluted in droplet formation buffer. FLTX1 (AOBIO 4054) was dissolved in DMSO then diluted further in droplet formation buffer. THZ1-TMR and JQ1-ROX was synthesized in as below to achieve the molecular structure displayed in FIG. 2D-E. Cisplatin conjugated to texas red (Ursa Bioscience) was dissolved in DMSO to 2 mM and diluted for further use in droplet formation buffer. Mitoxantrone (Sigma F6545) was dissolved in DMSO and diluted for further use in droplet formation buffer. Chemical structures were made using ChemDraw software.

Non-labeled molecules used live cell chase out experiments as below: JQ1 (Cayman Chemical 11187), cisplatin (Selleck S1166), transplatin (Toku-E T108), tamoxifen (Sigma Aldrich T5648), 4-hydroxytamoxifen (Sigma H7904).

In Vitro Droplet Assay

Recombinant BFP, GFP, or mCherry fusion proteins were concentrated and desalted to an appropriate protein concentration and 125 mM NaCl using Amicon Ultra centrifugal filters (30K MWCO, Millipore). Recombinant protein was added too. Droplet Formation buffer (50 mM Tris-HCl pH 7.5, 10% glycerol, 1 mM DTT) with the indicated amount of salt and the indicated crowding agent (Ficol or PEG). The protein solution was immediately loaded onto glass bottom 384 well plate (Cellvis P384-1.5H-N) and imaged with an Andor confocal microscope with a 150× objective. Unless indicated, images presented are of droplets settled on the glass coverslip.

Drug and small molecule concentrations used in the droplet experiments are as follows:

Texas red-cisplatin—5 uM
FLTX1—100 μM
Mitoxantrone—50 μM
Fluorescein—5 μM
Hoescht—1 mg/mL
Labeled dextrans—0.05 mg/mL
THZ1-TMR—5 μM
JQ1-ROX—1 μM
ROX—1 μM
TR—5 μM

For chase-out experiments 5 μM labeled cisplatin-TR was added to a MED1 droplets reaction (10 μM MED1, 50 mM Tris-HCl pH 7.5, 10% glycerol, 1 mM DTT, 10% PEG) in order to form MED1 droplets concentrated with Cisplatin-TR. Unlabeled transplatin or unlabeled cisplatin (vehicle, 10 μM, 100 μM, or 500 μM) were added to the droplet mixture and the amount of labeled cisplatin-TR remaining in the droplet is measured after chase out. 100 μM fluorescent FLTX1 was added to a MED1 droplets reaction (10 μM MED1, 50 mM Tris-HCl pH 7.5, 10% glycerol, 1 mM DTT, 10% PEG) in order to form MED1 droplets concentrated with FLTX1. 1 mM of the non-fluorescent version of the drug, tamoxifen, was added to the droplet mixture and the amount of fluorescent FLTX1 remaining in the droplet is measured after chase out. For assaying eviction of ER from MED1 condensates, fluorescently labeled ER and MED1 were mixed in droplet formation buffer at the indicated concentrations with the indicated components in the presence of 100 μM estrogen (Sigma E8875). For conditions with tamoxifen treatment, 4-hydroxytamoxifen (Sigma H7904) was then added to a final concentration of 100 uM and imaged as above on a confocal fluorescent microscope.

For droplet assay with fluorescent DNA a 451 base pair DNA fragment was commercially synthesized in a vector with flanking M13F and M13R primer binding sites. Primers M13F and M13R were commercially synthesized covalently bound to a Cy5 fluorophore and this fragment was amplified using these primers. The DNA fragment was then purified from PCR reactions and diluted in droplet formation buffer for use in the droplet assay as described. For testing the ability of recombinant CDK7 to partition in MED1 or HP1α droplets recombinant CDK activating complex (Millipore 14-476) supplied at 0.4 mg/mL in 150 mM NaCl at pH 7.5. One vial of Cy5 monoreactive dye (Amersham Pa.23001) was resuspended in 30 uL of 0.2 M Sodium Bicarbonate at pH 9.3 in 150 mM NaCl. 5 uL of this reaction was added to 5 uL of protein and incubated at RT for 1 hour. Free dye was removed by passing through a Zeba Spin Desalting Columns, 40 MWCO (87764, Thermo Scientific) as described in the package insert into droplet formation buffer with 1 mM DTT in 125 mM NaCl at a final concentration of 1 uM. This protein was used in the droplet assay as needed.

For screening of a modified BODIPY library of 80 modified BODIPY molecules was selected from a larger library collection as previous described (54). These molecules were diluted to 1 mM in DMSO then to 10 uM in droplet formation buffer. Droplets of MED1-IDR-BFP were formed in Droplet formation buffer with 125 mM NaCl and 10% PEG with 5 uM protein, probe was added to this reaction to a final concentration of 1 uM, the mixture was added to one well of a 384-well plate and imaged on an Andor confocal fluorescent microscope at 150× in the 488 (BODIPY) and 405 (protein) channels. These images were quantified by the aforementioned pipelines to quantify the maximum 488 signal intensity in droplets defined by the 405 channel. These values were then ranked to quantify the top and bottom “hits”. To ensure that the fluorescent intensity of the probes were equivalent, 1 uM of 18 random probes in droplet formation buffer was imaged as above and the average fluorescent intensity in the field determined. The same approach was taken to measure the fluorescent intensity of BODIPY alone (Sigma 795526), both in MED1 droplet and in the diffuse state.

FRAP of In Vitro Droplets with Drug

For FRAP of in vitro droplets, 5 pulses of laser at a 50 us dwell time was applied to the MED1 channel and 20 pulses of laser at a 100 μs dwell time was applied to the Cisplatin channel. Recovery was imaged on an Andor microscope every 1 s for the indicated time periods. Fluorescence intensity was measured using FIJI. Post bleach FRAP recovery data was averaged over 6 replicates for each channel.

Calculating Drug Enrichment Ratios

To analyze in vitro droplet experiments, custom Python scripts using the scikit-image package were written to identify droplets and characterize their size, shape and intensity. Droplets were segmented from average images of captured channels on various criteria: (1) an intensity threshold that was three s.d. above the mean of the image; (2) size thresholds (20 pixel minimum droplet size); and (3) a minimum circularity (circularity=4π·areaperimeter2) (circularity=4π·areaperimeter2) of 0.8 (1 being a perfect circle). After segmentation, mean intensity for each droplet was calculated while excluding pixels near the phase interface, and background-corrected by subtracting intensity of dark images of droplet formation buffer only. Droplets identified in the channel of the fluorescent protein from ten independent fields of view were quantified for each experiment. The maximum intensity of signal within the droplets was calculated for each channel, the maximum intensity in the drug channel was termed “maximum drug intensity”. To obtain the intensity of drug or dye alone in the diffuse state (termed “diffuse drug intensity”), the compound was added to droplet formation buffer at same concentration used in the droplet assay. This was then imaged on a confocal fluorescent microscope. The resulting image was processed in FIJI to obtain the fluorescent intensity of the field. To obtain the fluorescent intensity of protein droplets that bleed through in the drug channel (termed “background intensity”) protein droplets were imaged in the fluorescent channel in which the drug fluoresces and processed as above to obtain the average maximum intensity within the droplet across 10 images. The enrichment ratio was obtained by the following formula [(maximum drug intensity)−(background intensity)]/(diffuse drug intensity). The box plots show the distributions of all droplets. Each dot represents an individual droplet.

Chromatin Immunoprecipitation (ChIP) and Sequencing

MCF7 cells were grown in complete DMEM media to 80% confluence. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1000 g for 3 minutes at 4° C., flash frozen in liquid nitrogen and stored at 80° C. All buffers contained freshly prepared cOmplete protease inhibitors (Roche, 11873580001). Frozen crosslinked cells were thawed on ice and then resuspended in lysis buffer I (50 mM HEPES-KOH, pH 7.5, 140 mM NaCl, 1 mM EDTA, 10% glycerol, 0.5% NP-40, 0.25% Triton X-100, protease inhibitors) and rotated for 10 minutes at 4° C., then spun at 1350 rcf., for 5 minutes at 4° C. The pellet was resuspended in lysis buffer II (10 mM Tris-HCl, pH 8.0, 200 mM NaCl, 1 mM EDTA, 0.5 mM EGTA, protease inhibitors) and rotated for 10 minutes at 4° C. and spun at 1350 rcf. for 5 minutes at 4° C. The pellet was resuspended in sonication buffer (20 mM Tris-HCl pH 8.0, 150 mM NaCl, 2 mM EDTA pH 8.0, 0.1% SDS, and 1% Triton X-100, protease inhibitors) and then sonicated on a Misonix 3000 sonicator for 10 cycles at 30 s each on ice (18-21 W) with 60 s on ice between cycles. Sonicated lysates were cleared once by centrifugation at 16,000 rcf. for 10 minutes at 4° C. Input material was reserved and the remainder was incubated overnight at 4° C. with magnetic beads bound with CDK7 Bethyl A300-405A antibody to enrich for DNA fragments bound by CDK7. Beads were washed twice with each of the following buffers: wash buffer A (50 mM HEPES-KOH pH 7.5, 140 mM NaCl, 1 mM EDTA pH 8.0, 0.1% Na-Deoxycholate, 1% Triton X-100, 0.1% SDS), wash buffer B (50 mM HEPES-KOH pH 7.9, 500 mM NaCl, 1 mM EDTA pH 8.0, 0.1% Na-Deoxycholate, 1% Triton X-100, 0.1% SDS), wash buffer C (20 mM Tris-HCl pH8.0, 250 mM LiCl, 1 mM EDTA pH 8.0, 0.5% Na-Deoxycholate, 0.5% IGEPAL C-630, 0.1% SDS), wash buffer D (TE with 0.2% Triton X-100), and TE buffer. DNA was eluted off the beads by incubation at 65° C. for 1 hour with intermittent vortexing in elution buffer (50 mM Tris-HCl pH 8.0, 10 mM EDTA, 1% SDS). Cross-links were reversed overnight at 65° C. To purify eluted DNA, 200 mL TE was added and then RNA was degraded by the addition of 2.5 mL of 33 mg/mL RNase A (Sigma, R4642) and incubation at 37 C for 2 hours. Protein was degraded by the addition of 10 mL of 20 mg/mL proteinase K (Invitrogen, 25530049) and incubation at 55° C. for 2 hours. A phenol:chloroform:isoamyl alcohol extraction was performed followed by an ethanol precipitation. The DNA was then resuspended in 50 mL TE and used for sequencing. ChIP libraries were prepared with the Swift Biosciences Accel-NGS 2S Plus DNA Library Kit, according to the kit instructions. Following library preparation, ChIP libraries were run on a 2% gel on the PippinHT with a size-collection window of 200-600 bases. Final libraries were quantified by qPCR with the KAPA Library Quantification kit from Roche, and sequenced in single-read mode for 40 bases on an Illumina HiSeq 2500.

HCT116 cells were grown in complete DMEM media to 80% confluence followed by treatment with JQ1 or DMSO for 24 hours, followed by cell permeabilization (10 min at 37° C. with the solution of t×100 in PBS at 1:1000 in media) and subsequently treated with DMF or Cisplatin for 6 hours. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1000 g for 3 minutes at 4° C., flash frozen in liquid nitrogen and stored at 80° C. All buffers contained freshly prepared cOmplete protease inhibitors (Roche, 11873580001). Frozen crosslinked cells were thawed on ice and then resuspended in lysis buffer I (50 mM HEPES-KOH, pH 7.5, 140 mM NaCl, 1 mM EDTA, 10% glycerol, 0.5% NP-40, 0.25% Triton X-100, protease inhibitors) and rotated for 10 minutes at 4° C., then spun at 1350 rcf., for 5 minutes at 4° C. The pellet was resuspended in lysis buffer II (10 mM Tris-HCl, pH 8.0, 200 mM NaCl, 1 mM EDTA, 0.5 mM EGTA, protease inhibitors) and then sonicated on a Misonix 3000 sonicator for 10 cycles at 30 s each on ice (18-21 W) with 60 s on ice between cycles. Sonicated lysates were cleared once by centrifugation at 16,000 rcf. for 10 minutes at 4° C. Input material was reserved and the remainder was incubated overnight at 4° C. with magnetic beads bound with CDK7 Bethyl A300-405A antibody to enrich for DNA fragments bound by CDK7. Beads were washed twice with each of the following buffers: wash buffer A (50 mM HEPES-KOH pH 7.5, 140 mM NaCl, 1 mM EDTA pH 8.0, 0.1% Na Deoxycholate, 1% Triton X-100, 0.1% SDS), wash buffer B (50 mM HEPES-KOH pH 7.9, 500 mM NaCl, 1 mM EDTA pH 8.0, 0.1% Na-Deoxycholate, 1% Triton X-100, 0.1% SDS), wash buffer C (20 mM Tris-HCl pH8.0, 250 mM LiCl, 1 mM EDTA pH 8.0, 0.5% Na-Deoxycholate, 0.5% IGEPAL C-630, 0.1% SDS), wash buffer D (TE with 0.2% Triton X-100), and TE buffer. DNA was eluted off the beads by incubation at 65° C. for 1 hour with intermittent vortexing in elution buffer (50 mM Tris-HCl pH 8.0, 10 mM EDTA, 1% SDS). Cross-links were reversed overnight at 65° C. To purify eluted DNA, 200 mL TE was added and then RNA was degraded by the addition of 2.5 mL of 33 mg/mL RNase A (Sigma, R4642) and incubation at 37 C for 2 hours. Protein was degraded by the addition of 10 mL of 20 mg/mL proteinase K (Invitrogen, 25530049) and incubation at 55° C. for 2 hours. A phenol:chloroform:isoamyl alcohol extraction was performed followed by an ethanol precipitation. The DNA was then resuspended in 50 mL TE and used for sequencing. ChIP libraries were prepared with the Swift Biosciences Accel-NGS 2S Plus DNA Library Kit, according to the kit instructions. Following library preparation, ChIP libraries were run on a 2% gel on the PippinHT with a size-collection window of 200-600 bases. Final libraries were quantified by qPCR with the KAPA Library Quantification kit from Roche, and sequenced in single-read mode for 40 bases on an Illumina HiSeq 2500.

HCT116 cells were grown in complete DMEM media to 80% confluence followed by treatment with JQ1 or DMSO for 24 hours, followed by cell permeabilization (10 min at 37° C. with the solution of t×100 in PBS at 1:1000 in media) and subsequently treated with DMF or Cisplatin for 6 hours. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1000 g for 3 minutes at 4° C., flash frozen in liquid nitrogen and stored at 80° C. All buffers contained freshly prepared cOmplete protease inhibitors (Roche, 11873580001). Frozen crosslinked cells were thawed on ice and then resuspended in lysis buffer I (50 mM HEPES-KOH, pH 7.5, 140 mM NaCl, 1 mM EDTA, 10% glycerol, 0.5% NP-40, 0.25% Triton X-100, protease inhibitors) and rotated for 10 minutes at 4° C., then spun at 1350 rcf., for 5 minutes at 4° C. The pellet was resuspended in lysis buffer II (10 mM Tris-HCl, pH 8.0, 200 mM NaCl, 1 mM EDTA, 0.5 mM EGTA, protease inhibitors) and rotated for 10 minutes at 4° C. and spun at 1350 rcf. for 5 minutes at 4° C. The pellet was resuspended in sonication buffer (20 mM Hepes pH 7.5, 140 mM NaCl, 1 mM EDTA 1 mM EGTA, 1% Triton X-100, 0.1% Na-deoxycholate, 0.1% SDS, protease inhibitors) and then sonicated on a Misonix 3000 sonicator for 10 cycles at 30 s each on ice (18-21 W) with 60 s on ice between cycles. Sonicated lysates were cleared once by centrifugation at 16,000 rcf. for 10 minutes at 4° C. Input material was reserved and the remainder was incubated overnight at 4° C. with magnetic beads bound with MED1 antibody (Bethyl A300-793A) to enrich for DNA fragments bound by MED1. Beads were washed with each of the following buffers: washed twice with sonication buffer (20 mM Hepes pH 7.5, 140 mM NaCl, 1 mM EDTA 1 mM EGTA, 1% Triton X-100, 0.1% Na-deoxycholate, 0.1% SDS), once with sonication buffer with high salt (20 mM Hepes pH 7.5, 500 mM NaCl, 1 mM EDTA 1 mM EGTA, 1% Triton X-100, 0.1% Nadeoxycholate, 0.1% SDS), once with LiCl wash buffer (20 mM Tris pH 8.0, 1 mM EDTA, 250 mM LiCl, 0.5% NP-40, 0.5% Na-deoxycholate), and once with TE buffer. DNA was eluted off the beads by incubation with agitation at 65° C. for 15 minutes in elution buffer (50 mM Tris-HCl pH 8.0, 10 mM EDTA, 1% SDS). Cross-links were reversed for 12 hours at 65° C. To purify eluted DNA, 200 mL TE was added and then RNA was degraded by the addition of 2.5 mL of 33 mg/mL RNase A (Sigma, R4642) and incubation at 37° C. for 2 hours. Protein was degraded by the addition of 4 ul of 20 mg/mL proteinase K (Invitrogen, 25530049) and incubated at 55° C. for 30 minutes. DNA was purified using Qiagen PCR purification kit, eluted in 30 μl Buffer EB, and used for sequencing. ChIP libraries were prepared with the Swift Biosciences Accel-NGS 2S Plus DNA Library Kit, according to the kit instructions. Following library preparation, ChIP libraries were run on a 2% gel on the PippinHT with a size-collection window of 200-400 bases. Final libraries were quantified by qPCR with the KAPA Library Quantification kit from Roche, and sequenced in single-read mode for 50 bases on an Illumina HiSeq 2500.

ChIP-Seq data were aligned to the mm9 version of the mouse reference genome using bowtie with parameters -k l -m l -best and -l set to read length. Wiggle files for display of read coverage in bins were created using MACS with parameters -w -S -space=50-nomodel-shiftsize=200, and read counts per bin were normalized to the millions of mapped reads used to make the wiggle file. Reads-per-million-normalized wiggle files were displayed in the UCSC genome browser. For ER, MED1, BRD4, and H3K9me3 ChIP-Seq in MCF7 cells, published datasets were used (GEO GSE60270, GSM1348516, and GSM945857, respectively).

Purification of CDK8-Mediator

The CDK8-Mediator samples were purified as described (55) with modifications. Prior to affinity purification, the P0.5M/QFT fraction was concentrated, to 12 mg/mL, by ammonium sulfate precipitation (35%). The pellet was resuspended in pH 7.9 buffer containing 20 mM KCl, 20 mM HEPES, 0.1 mM EDTA, 2 mM MgCl₂, 20% glycerol and then dialyzed against pH 7.9 buffer containing 0.15M KCl, 20 mM HEPES, 0.1 mM EDTA, 20% glycerol and 0.02% NP-40 prior to the affinity purification step. Affinity purification was carried out as described, eluted material was loaded onto a 2.2 mL centrifuge tube containing 2 mL 0.15M KCl HEMG (20 mM HEPES, 0.1 mM EDTA, 2 mM MgCl₂, 10% glycerol) and centrifuged at 50K RPM for 4 h at 4° C. This served to remove excess free GST-SREBP and to concentrate the CDK8-Mediator in the final fraction. Prior to droplet assays, purified CDK8-Mediator was concentrated using Microcon-30 kDa Centrifugal Filter Unit with Ultracel-30 membrane (Millipore MRCF0R030) to reach 300 nM of Mediator complex. Concentrated CDK8-Mediator was added to the droplet assay to a final concentration of 200 nM. Droplet reactions contained 10% PEG-8000 and 125 mM salt.

Immunofluorescence with RNA FISH

Cells were plated on coverslips and grown for 24 hours followed by fixation using 4% paraformaldehyde, PFA, (VWR, BT140770) in PBS for 10 minutes. After washing cells three times in PBS, the coverslips were put into a humidifying chamber or stored at 4° C. in PBS. Permeabilization of cells were performed using 0.5% Triton X-100 (Sigma Aldrich, X100) in PBS for 10 minutes followed by three PBS washes. Cells were blocked with 4% IgG-free Bovine Serum Albumin, BSA, (VWR, 102643-516) for 30 minutes and the primary antibody was added at a concentration of 1:500 in PBS for 4-16 hours. Cells were washed with PBS three times followed by incubation with secondary antibody at a concentration of 1:5000 in PBS for 1 hour. After washing twice with PBS, cells were fixed using 4% paraformaldehyde, PFA, (VWR, BT140770) in PBS for 10 minutes. After two washes of PBS, Wash buffer A (20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) was added to cells and incubated for 5 minutes. 12.5 mM RNA probe (Stellaris) in Hybridization buffer (90% Stellaris RNA FISH Hybridization Buffer (Biosearch Technologies, SMF-HB1-10) and 10% Deionized Formamide) was added to cells and incubated overnight at 37° C. After washing with Wash buffer A for 30 minutes at 37° C., the nuclei were stained with 20 mm/mL Hoechst 33258 (Life Technologies, H3569) for 5 minutes, followed by a 5 minute wash in Wash buffer B (Biosearch Technologies, SMF-WB1-20). Cells were washed once in water followed by mounting the coverslip onto glass slides with Vectashield (VWR, 101098-042) and finally sealing the coverslip with nail polish (Electron Microscopy Science Nm, 72180). Images were acquired at an RPI Spinning Disk confocal microscope with a 100× objective using MetaMorph acquisition software and a Hammamatsu ORCA-ER CCD camera (W. M. Keck Microscopy Facility, MIT). Images were post-processed using Fiji Is Just ImageJ (FIJI).

RNA FISH Image Analysis

For analysis of RNA FISH with immunofluorescence, custom Python scripts were written to process and analyze 3D image data gathered in FISH and immunofluorescence channels. FISH foci were automatically called using the scipy ndimage package. The ndimage find_objects function was then used to call contiguous FISH foci in 3D. These FISH foci were then filtered by various criteria, including size, circularity of a maximum-zprojection (circularity=4π·areaperimeter2; 0.7)(circularity=4π·area perimeter 2; 0.7), and being present in a nucleus (determined by nuclear mask). The FISH foci were then centered in a 3D box (length size (l)=3.0 μm). The immunofluorescence signals centered at FISH foci for each FISH and immunofluorescence pair were then combined, and an average intensity projection was calculated, providing averaged data for immunofluorescence signal intensity within a l×l square centered at FISH foci. As a control, this same process was carried out for immunofluorescence signals centered at an equal number of randomly selected nuclear positions. These average-intensity projections were then used to generate 2D contour maps of the signal intensity. Contour plots were generated using the matplotlib Python package. For the contour plots, the intensity-color ranges presented were customized across a linear range of colors (n=15). For the FISH channel, black to magenta was used. For the immunofluorescence channel, chroma.js (an online color generator) was used to generate colors across 15 bins, with the key transition colors chosen as black, blue—violet, medium blue and lime. This was done to ensure that the reader's eye could more-readily detect the contrast in signal. The generated color map was used in 15 evenly spaced intensity bins for all immunofluorescence plots. The averaged immunofluorescence, centered at FISH or at randomly selected nuclear locations, is plotted using the same color scale, set to include the minimum and maximum signal from each plot.

Cisplatin Treatments Followed by Immunofluorescence

HCT116 cells were plated in 24-well plate at 50 k cells per well to yield 100 k cells after 21 hours (doubling time of HCTs). Cells were permeabilized using a solution of Tx100 in media at 0.55 pmol/cell for 12 minutes at 37° C. Cells were then washed with 500 ul media and treated with 500 ul of 50 uM cisplatin in media for 6 hours. After 6 hours, the cells were washed once with room temperature PBS and then fixed with 500 uL 4% formaldehyde in PBS for 12 min at room temperature. The cells were then washed 3 more times with PBS. Coverslips were put into a humidifying chamber or stored at 4° C. in PBS. Permeabilization of cells were performed using 0.5% Triton X-100 (Sigma Aldrich, X100) in PBS for 10 minutes followed by three PBS washes. Cells were blocked with 4% IgG-free Bovine Serum Albumin, BSA, (VWR, 102643-516) for 30 minutes and the primary antibody was added at a concentration of 1:500 in PBS for 4-16 hours. Cells were washed with PBS three times followed by incubation with secondary antibody at a concentration of 1:5000 in PBS for 1 hour. Samples was washed in PBS, DNA was stained using 20 μm/mL Hoechst 33258 (Life Technologies, H3569) for 5 minutes and mounted using Vectashield (VWR, 101098-042). Images were acquired at an RPI Spinning Disk confocal microscope with a 100× objective using MetaMorph acquisition software and a Hammamatsu ORCA-ER CCD camera (W.M. Keck Microscopy Facility, MIT). Images were post-processed using Fiji Is Just ImageJ (FIJI).

Cisplatin/Condensate Co-IF

For the analysis of co-immunofluorescence data, custom python scripts were written to both process and analyze the 3D image data from IF and DAPI channels. Nuclei were detected using the Triangle thresholding method and a nuclear mask was applied the IF channels. Manual minimal thresholds were applied to the 488 channel to determine nuclear puncta for protein of interest (MED1, HP1a, or FIB1). The triangle thresholding method was applied to the 561 channel to determine nuclear puncta for cisplatin. Percentage of cisplatin overlap was calculated by the number of defined nuclear cisplatin puncta that overlapped with the protein of interest puncta divided by the total number of nuclear cisplatin puncta.

Cisplatin-Seq Analysis

Cisplatin-seq fastq files for rep1 24-hour treated cells were downloaded from www.ncbi.nlm.nih.gov/sra/SRX1962532[accn] (sequencing run ID SRR3933212) (41). Reads were aligned to the human genome build hg19 (GRCh37) using Bowtie2 to get aligned .bam files (56). H3k27Ac chip-seq reads in HELA cells were used to call super-enhancers using the ROSE algorithm (47, 57). Super-enhancers were separated from typical enhancers using the super-enhancer table output by ROSE algorithm. The typical enhancers were broken down further by their H3k27Ac signal. The last decile of enhancers were extracted based on H3k27Ac signal to get the low H3k27Ac category of enhancers. Each category of enhancer (super-enhancers, typical enhancers, and low h3k27ac signal enhancers) was broken down into their constituents, and constituents that overlapped with blacklist regions were excluded. Black list regions were downloaded from ENCODE file www.encodeproject.org/files/ENCFF001TDO/. Each enhancer constituent was then extended by 2 kb at either end. The 24-hour treated cisplatin-seq reads were mapped to each of the three categories of 2 kb-extended enhancers using the bamToGFF.py script. For each category of enhancer, the constituent region and flanking regions were separately split into 50 equally-sized bins and the reads in each bin were counted. The average read count per bin across all enhancer constituents and flanking regions was used to create the meta-plot.

Cisplatin Treatments Followed by Live Cell Imaging

HCT116 cells with the indicated GFP knock-in were plated at 35 k per well of a glass bottom 8-well chamber slide. Following incubation at 37° C. overnight, cells were treated with 50 uM cisplatin in DMEM or a 1:1000 dilution of DMSO for 12 hours. Prior to imaging, cells were additionally treated with a 1:5000 dilution of Hoechst 33342 to stain DNA and 2 uM propidium iodide to stain dead cells. For the quantified dataset of GFP-tagged MED1, HP1 or FIB1 in HCT116 cells, cells were imaged using an Andor confocal microscope at 100× magnification. For representative images of each of the six tagged lines treated with vehicle or 50 μM cisplatin, cells were imaged on the Zeiss LSM 880 confocal microscope with Airyscan detector with 63× objective at 37° C.

Condensate Score Analysis

Nuclei were segmented from images of treated cells by custom Python scripts using the scikit-image, open-cv, and scipy-ndimage Python packages. Nuclei were segmented by median filter, thresholding, separated by the watershed algorithm, and labeled by the scikit-image label function. For each nuclei, the fluorescence signal in the GFP channel (corresponding to either MED1, HP1α, or FIB1) was maximally-projected if z-stacks were acquired. A grey-level co-occurrence matrix (GLCM) was then generated from the projected signal, and the ‘correlation’ texture property from the GLCM was calculated per nucleus. One-way ANOVA followed by Sidak's multiple comparisons test was performed on the correlation values across conditions using GraphPad Prism version 8.2.0 for Mac (www.graphpad.com). Finally, to derive the condensation score, these values were subtracted from 1.

FRAP of HCT116 mEGFP Tagged Cell Lines

FRAP was performed on Andor confocal microscope with 488 nm laser. Bleaching was performed over a r_{bleach}≈1 um using 100% laser power and images were collected every two seconds. Fluorescence intensity was measured using FIJI. Background intensity was subtracted, and values are reported relative to pre-bleaching time points. Post bleach FRAP recovery data was averaged over 7 replicates for each cell-line and condition.

Determination of Partitioning by Spectrophotometry and Quantitative Phase Microscopy

Derivation of Expression for Drug Partition Coefficient in Condensates

Here, an expression is derived briefly for the partition coefficient of a client molecule into a condensed phase in terms of quantities that are readily measurable experimentally. A sample composed of two coexisting phases is considered, named dilute and condensed, with volume fractions ϕdilute and ϕcond=1. If a client molecule (e.g. a drug) is also present in the sample at an average concentration of ctotal, then mass conservation requires that

cₜₒₜₐₗ=c_{dilute}ϕ_{dilute}+c_{cond}ϕ_{cond}, (1)

where c_{dilute} and c_{cond} are the concentrations of the client in the dilute and condensed phases, respectively. Finally, the partition coefficient is defined of the client into the condensed phase as P=c_{cond}/c_{dilute}. With this definition and the requirement that the phase volume fractions sum to 1, Eq 1 can be written as

cₜₒₜₐₗ=c_{dilute}(1−ϕ_{cond})+c_{dilute}Pϕ_{cond}, (2)

which can be simplified and rearranged to yield

$$P = 1 + ( \frac{{c}_{total}}{{c}_{dilute}} - 1 ) {( {ϕ}_{cond} )}^{- 1} . \qquad ( 3 )$$

the ratio is estimated cₜₒₜₐₗ/c_{dilute} from fluorescence spectroscopy measurements, as described in a subsequent section, while ϕ_{cond}, it is inferred from the lever rule (M. Rubinstein, R. H. Colby, Polymer Phyics (Oxford University Press, 2003) as follows: denoting the concentration of scaffold protein (e.g. MED1) by s, mass conservation gives stotal=s_{dilute}ϕ_{dilute}+s_{cond}ϕ_{cond}, in analogy with Eq. 1. Again using the requirement that the volume fractions of coexisting phases sum to 1, this can be rearranged to yield

$${ϕ}_{cond} = \frac{{s}_{total} - {s}_{dilute}}{{s}_{cond} - {s}_{dilute}} . \qquad ( 4 )$$

where sₜₒₜₐₗ and s_{dilute} are measured spectrophotometrically from optical absorbance at 280 nm, and s_{cond} is measured from quantitative phase microscopy, using a coherence-controlled holographic microscope (Q-Phase, Telight (formerly TESCAN), Bmo, CZ) equipped with 40× dry objectives (NA=0.90).

UV-Vis Fluorescence Spectroscopy Measurements and Analysis

Uv-vis spectroscopy (TECAN Spark20M) was used to estimate the absolute concentration of drug in solution using Beer-Lambert law with Eq 5,

A=Log 10(I0/I)=εcL (5)

where A is the measured absorbance (in Absorbance Units (AU)), I0 is the intensity of the incident light at a given wavelength, I is the transmitted intensity, L the path length through the sample, and c the concentration of the absorbing species. For each species and wavelength, c is a constant known as the molar absorptivity or extinction coefficient. This constant is a fundamental molecular property in a given solvent, at a particular temperature and pressure, and has units of 1/M*cm. The partitioned drug was measured by using spin down assay. Known concentration of drug was added with the protein and kept for the droplet formation. After 30 minutes, the mixture was centrifuged at 15,000 rpm for 10 minutes. The supernatant was collected and measured the concentrations of the drug. The partitioned drug was calculated by subtracting from the total known concentration of drug added.

Quantitative Phase Microscopy Measurements and Analysis

Quantitative phase measurements were performed using a coherence-controlled holographic microscope (Q-Phase, Telight (formerly TESCAN), Brno, CZ) equipped with 40× dry objectives (NA=0.90) as follows. Immediately following phase separation, samples were loaded into a custom temperature-controlled flowcell, sealed and allowed to settle under gravity prior to imaging. Flowcells were constructed with a PEGylated coverslip and a sapphire slide as bottom and top surfaces, respectively, using parafilm strips as spacers. Peltier elements affixed to the sapphire slide enabled regulation of flowcell temperature, as previously described (59). Temperature was maintained at 21.00±0.02° C. during measurements.

Q-PHASE software was used to construct compensated phase images from acquired holograms, which were subsequently analyzed in MATLAB using custom code. As details regarding the calculation of protein concentration from quantitative phase images will be discussed extensively elsewhere (McCall et al, forthcoming), only a conceptual overview will be given here. Briefly, each phase image is spatially segmented based on intensity, and a window containing each segmented object is fit to a spatial function of the form

$$φ ( x , y ) = \frac{2 π}{λ} Δ nH ( x , y \vert R ) . \qquad ( 6 )$$

where φ(x, y) is the phase intensity at pixel location (x, y), λ is the illumination wavelength, Δn is the refractive index difference between MED1 condensates and the surrounding dilute phase, and H(x, y|R) is the projected height of a sphere of radius R. The fitting parameters in Eq. 6 are Δn and R. It is assumed that no PEG partitions into the condensates and calculate the average scaffold concentration in each filtered condensate as

$${s}_{cond} = \frac{Δ n + ( {n}_{dilute} - {n}_{0} )}{dn / ds} x \qquad ( 7 )$$

Here no is the refractive index of buffer in the absence of scaffold and PEG, n_{dilute} is the refractive index of the dilute phase, and both are measured at 21.00±0.01° C. using a J457 digital refractometer (Rudolph Research Analytic, Hackettstown, N.J.). The refractive index increment of the scaffold protein, dn/ds, is estimated from amino acid composition (60).

Cisplatin-DNA Engagement Assay

MED1-IDR-BFP and HP1a-BFP droplets were formed by mixing 10 μM protein with the droplet formation buffer containing 50 mM Tris-HCl pH 7.5, 100 mM NaCl, 10% PEG 8000, 10% glycerol, 1 mM DTT and 5 ng/ul DNA in a 10 μl reaction volume. The droplet reactions were incubated for 30 min at RT. Next, increasing concentrations of activated Cisplatin (0, 0.5, 0.75, 1, 1.5, and 2 mM) were added to the droplet reactions and incubated for another 30 min at RT. The reactions are then treated with 1 μl of Proteinase K (Invitrogen, 20 mg/ml) for 4 hr at 55° C. Platination of DNA was visualized by size-shift on a bioanalyzer.

Amino Acid and Basic/Acidic Patch Analysis

Basic and acidic patches were determined by identifying charged interaction elements (CIEs) as previously described by (61). For each protein, the net charge per residue (NCPR) along the protein sequence was calculated using a sliding window of 5 amino acids with a step size of 1 amino acid using the localCIDER software (62). Stretches of 4 or more amino acids with NCPR<−0.35 were identified as acidic patches (CIE−), while stretches of 4 or more amino acids with NCPR>+0.35 were identified as basic patches (CIE+). The number of acidic and basic patches within the total protein and the IDR specifically was counted. Separately, the number of aromatic residues within the whole protein and the IDR was also counted.

Cell Survival Assay

HCT116 cells were plated in 24-well plate at 50 k cells per well to yield 100 k cells after 21 hours (doubling time of HCTs). Cells were then treated with either 50 μM cisplatin or DMF in DMEM media for 12 hours. At 12 hours, CellTiter-Glo Reagent was added to each well, following the CellTiter-Glo Luminescent Viability Assay. Luminescence was then measured, averaging 5 wells for each condition.

In Silico Modeling

A simplified model was developed of drug-target interactions in the presence of a condensate. The relevant species are the drug (D), target (T), and the drug-target complex (D-T). It is assumed that there are only 2-types of phases, the bulk/dilute nuclear phase (n) and the condensate phase (c), which is present with volume fraction ƒ=V_{condensate}/V_{nucleus}. At equilibrium, the following partitioning conditions are obeyed:

$$\frac{{[ D ]}_{c}}{{[ D ]}_{n}} = {p}_{D} ; \frac{{[ T ]}_{c}}{{[ T ]}_{n}} = {p}_{T} ;$$

where p_{D}, p_{T} are the partition coefficients of the drug and target. [D]_{c} represents the concentration of species D in condensate phase (and similarly for other components/phases). In this model, the drug and target complex with phase-independent disassociation constant of K_{D}.

$$[ D ] + [ T ] {↔}^{{K}_{D}} [ D - T ] {K}_{D} = \frac{[ D ] [ T ]}{[ D - T ]}$$

To solve for equilibrium concentrations of various species, which are present at overall levels [D]₀, [T]₀, the species balance is written down as:

ƒ([D]_{c}+[D−T]_{c})+(1−ƒ)([D]ₙ+[D−T]ₙ)=[D]₀

ƒ([T]_{c}+[D−T]_{c})+(1−ƒ)([T]ₙ+[D−T]ₙ)=[DT]₀

These 6 concentrations are solved with 2-equations and 4 constraints (2 from partitioning and 2 from reaction equilibria). In FIG. 61B-D, the fraction of bound target is defined as:

$${Fraction}_{bound} , c = \frac{{[ D - T ]}_{c}}{{[ D ]}_{c} + {[ T ]}_{c}}$$

A similar expression is used for the fraction of bound target in the nuclear (bulk or dilute) phase. In case of controls plotted, plot fraction is plotted when there is only 1 phase (f=0).

Generation and Analysis of MCF7 mEGFP-MED1 Cells

To generate MCF7 mEGFP-MED1 cells, a lentiviral construct containing the full length MED1 with a N-terminal mEGFP fusion connected by a 10 amino acid GS linker was cloned, containing a puromycin selection marker. Lentiviral particles were generated in HEK293T cells. 250,000 MCF7 cells were plated in one well of a 6 well plate and viral supernatant was added. 48 hours later puromycin was added at 1 ug/mL for 5 days for selection.

For live-cell FRAP experiments, the endogenously tagged MED1-mEGFP MCF7 cells were plated on Poly-L-Ornithine coated glass-bottom tissue culture plate. 20 pulses of laser at a 50 us dwell time were applied to the array, and recovery was imaged on an Andor microscope every 1 s for the indicated time periods. Quantification was performed in FIJI. The instrument background was subtracted from the average signal intensity in the bleached puncta then divided by the instrument background subtracted from a control puncta. These values were plotted every second, and a best fit line with 95% confidence intervals was calculated. For observing fusions of MED1-GFP foci, MED1-mEGFP MCF7 cells were grown for 3 days in estrogen-free conditions then plated on glass-bottomed plates. 15 minutes prior to imaging, cells were treated with 100 nM estrogen and placed on the Andor confocal microscope and imaged at 150× for 4 minutes. Images were postprocessed in FIJI. Fluorescent intensity calculations were made in FIJI.

Chemistry

Unless otherwise noted, reagents and solvents were obtained from commercial suppliers and were used without further purification. Mass spectra were obtained on a Waters Micromass ZQ instrument. Preparative HPLC was performed on a Waters Sunfire C18 column (19 mm×50 mm, 5 μM) using a gradient of 15-95% methanol in water containing 0.05% trifluoroacetic acid (TFA) over 22 min (28 min run time) at a flow rate of 20 mL/min.

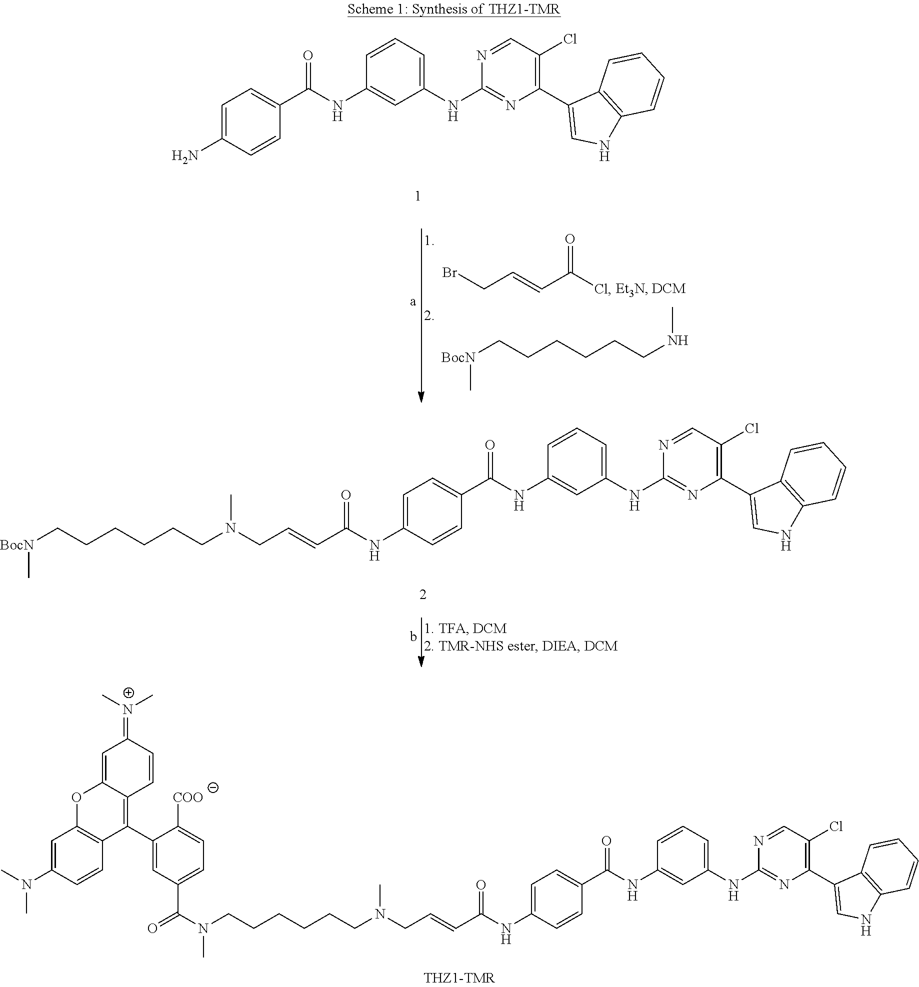

Reagents and conditions: (a) (E)-4-bromobut-2-enoyl chloride, triethyl amine, DCM, 0° C.˜r.t., then tert-butyl methyl(6-(methylamino)hexyl)carbamate, r.t.˜50° C.; (b) trifluoroacetic acid, DCM, r.t., then TMR-NHS ester, diisopropylethyl amine, DCM, r.t.˜40° C. Cert-butyl E)-(6-((4-((4-((3-((5-chloro-4-(1H-indol-3-yl)pyrimidin-2-yl)amino)phenyl) carbamoyl)phenyl)amino)-4-oxobut-2-en-yl)(methyl)amino)hexyl)(methyl)carbamate (2). To a solution of 1 (20 mg, 0.044 mmol, prepared according to patent WO2014/63068) and triethyl amine (29 mg, 0.27 mmol) in 0.8 mL DCM was added (E)-4-bromobut-2-enoyl chloride (0.24 mL, 0.2 M in DCM). The solution was stirred for 6 hours. Then tert-butyl methyl(6-(methylamino)hexyl)carbamate (13 mg, 0.052 mmol) in 0.4 mL DCM was added. The mixture was warmed to 50° C. and kept overnight. The mixture was concentrated in vacuo, then purified by preparative HPLC to provide intermediate 2 (6 mg, 19%). LC/MS (ESI) m/z=765 (M+H)+. (E)-4-((6-((4-((4-((3-((5-chloro-4-(1H-indol-3-yl)pyrimidin-2-yl)amino)phenyl)carbamoyl)phenyl)amino)-4-oxobut-2-en-1-yl)(methyl)amino)hexyl)(methyl)carbamoyl)-2-(6-(dimethylamino)-3-(dimethyliminio)-3Hxanthen-9-yl)benzoate (THZ1-TMR). To a solution of 2 (6 mg, 0.0078 mmol) in 0.5 mL DCM was added 0.1 mL TFA. The resultant solution was stirred at room temperature for 1 h, and then concentrated in vacuo to obtain free amine as TFA salt, which was dissolved in 0.5 mL DCM again. To this solution DIEA (5 mg, 0.039 mmol) and TMR-NHS ester (5 mg, 0.0094 mmol) were added in sequence. The mixture was warmed to 40° C. and kept overnight. The mixture was concentrated in vacuo, then purified by preparative HPLC to provide THZ1-TMR (2 mg, 23%). LC/MS (ESI) m/z=1077 (M+H)+.

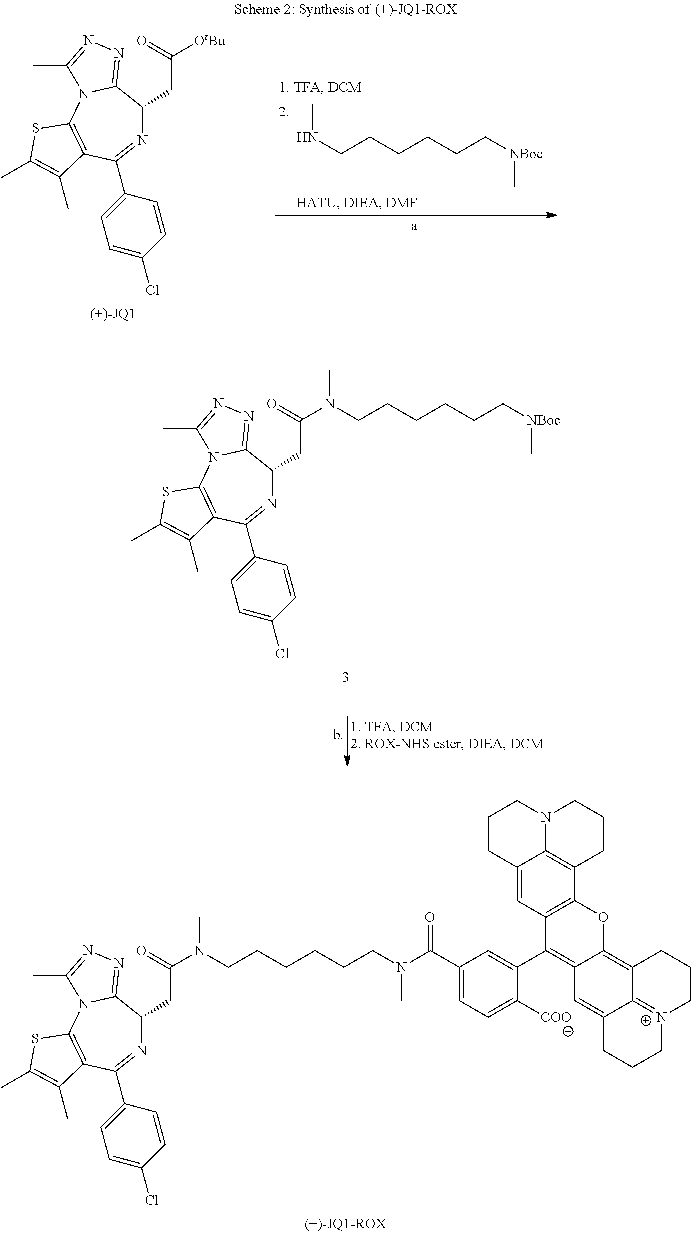

Reagents and conditions: (a) trifluoroacetic acid, DCM, r.t., then tert-butyl methyl(6-(methylamino)hexyl)carbamate, 1-[bis(dimethylamino)methylene]-1H-1,2,3-triazolo[4,5-b]pyridinium 3-oxid hexafluorophosphate, diisopropylethyl amine, DMF, r.t.; (b) trifluoroacetic acid, DCM, r.t., then ROX-NHS eater, diisopropylethyl amine, DCM, r.t.˜40° C. Cert-butyl (S)-(6-(2-(4-(4-chlorophenyl)-2,3,9-trimethyl-6H-thieno[3,2-f][1,2,4]triazolo[4,3-a][1,4]diazepin-6-yl)-N methylacetamido)hexyl)(methyl)carbamate (3) To a solution of (+)-JQ1 (25 mg, 0.055 mmol) in 2 mL DCM was added 0.4 mL TFA. The resultant solution was stirred at room temperature for 1 h, and then concentrated in vacuo to obtain free amine as TFA salt, which was dissolved in 0.8 mL DMF. To this solution was added tert-butyl methyl(6-(methylamino)hexyl)carbamate (16 mg, 0.065 mmol) in 0.5 mL DMF, DIEA (35 mg, 0.28 mmol) and HATU (24 mg, 0.064 mmol) in sequence. The mixture was stirred at r.t. for 6 hours. Then purified by preparative HPLC to provide intermediate 3 (15 mg, 43%). LC/MS (ESI) m/z=627 (M+H)+.

(+) JQ1-ROX. To a solution of 3 (15 mg, 0.024 mmol) in 2 mL DCM was added 0.4 mL TFA. The resultant solution was stirred at room temperature for 1 h, and then concentrated in vacuo to obtain free amine as TFA salt, which was dissolved in 1 mL DCM again. To this solution DIEA (16 mg, 0.12 mmol) and ROX-NHS ester (13 mg, 0.021 mmol) were added in sequence. The mixture was warmed to 40° C. and kept overnight. The mixture was concentrated in vacuo, then purified by preparative HPLC to provide (+)JQ1-ROX (6 mg, 28), LC/MS (ESI) m/z=1043 (M+H)+.

Immunofluorescence with DNA FISH

MCF7 cells were grown in estrogen-free DMEM for 3 days on Poly-L-ornithine coated coverslips in 24 well plates at an initial seeding density of 50,000 cells per well. Cells were then treated with vehicle, 10 uM estradiol, or 10 uM estradiol and 5 uM 4-hydroxytamoxifen for 45 minutes. HCT116 cells were treated with 1 μM JQ1 for 24 hours, followed by cell permeabilization (10 min at 37° C. with the solution of t×100 in PBS at 1:1000 in media) and subsequently DMF or 50 μM Cisplatin for 6 hours.

Cells on cover slips were then fixed in 4% paraformaldehyde. Immunofluorescence was performed as described above. After incubating the cells with the secondary antibodies, cells were washed three times in PBS for 5 min at RT, fixed with 4% PFA in PBS for 10 min and washed three times in PBS. Cells were incubated in 70% ethanol, 85% ethanol and then 100% ethanol for 1 minute at RT. Probe hybridization mixture was made mixing 74, of FISH Hybridization Buffer (Agilent G9400A), 1 μl of FISH probes (SureFISH 8q24.21 MYC 294 kb G101211R-8) and 2 μL of water.

5 μL of mixture was added on a slide and coverslip was placed on top (cell-side toward the hybridization mixture). Coverslip was sealed using rubber cement. Once rubber cement solidified, genomic DNA and probes were denatured at 78° C. for 5 minutes and slides were incubated at 16° C. in the dark O/N. The coverslip was removed from slide and incubated in pre-warmed Wash Buffer 1 (Agilent, G9401A) at 73° C. for 2 minutes and in Wash Buffer 2 (Agilent, G9402A) for 1 minute at RT. Slides were air dried and nuclei were stained in 20 μm/mL Hoechst 33258 (Life Technologies, H3569) in PBS for 5 minutes at RT. Coverslips were washed three times in PBS, followed by mounting the coverslip onto glass slides, sealing, imaging, and post-processing as described above.

RT-qPCR

MCF7 cells were estrogen deprived for 3 days then stimulated with either 10 nM estrogen or 10 nM estrogen and 5 uM 4-hydroxytamoxifen for 24 hours. RNA was isolated by AllPrep Kit (Qiagen 80204) followed by cDNA synthesis using High-Capacity cDNA Reverse Transcription Kit (Applies Biosystems 4368814). qPCR was performed in biological and technical triplicate using Power SYBR Green mix (Life Technologies #4367659) on a QuantStudio 6 System (Life Technologies). The following oligos was used in the qPCR; Myc fwd AACCTCACAACCTTGGCTGA, MYC rev TTCTTTTATGCCCAAAGTCCAA, GAPDH fwd TGCACCACCAACTGCTTAGC, GAPDH rev GGCATGGACTGTGGTCATGAG. Fold change was calculated and MYC expression values were normalized to GAPDH expression.

LAC Binding Assay

Constructs were assembled by NEB HIFI cloning in pSV2 mammalian expression vector containing an SV40 promoter driving expression of a mCherry-LacI fusion protein. The intrinsically disordered region of MED1, HP1α, or the activation domain of ESR1 was fused by the c-terminus to this recombinant protein, joined by the linker sequence GAPGSAGSAAGGSG (SEQ ID NO: 16). For experiments comparing FLTX1 enrichment at the array, U20S-Lac cells were plated onto chambered coverglass (1.5 Borosilicate Glass, Nunc Lab-Tek, 155409) and transfected with either MED1 IDR or HP1α constructs with lipofectamine 3000 (Thermofisher L3000015). After 24 hours, cells were treated with either 1 uM FLTX1 or vehicle (DMF). After 30 minutes, cells were imaged on the Zeiss LSM 880 confocal microscope with Airyscan detector with 63× objective at 37° C. For experiments with high MED1, cells grown in DMEM were plated on glass coverslips and transfected using lipofectamine 3000 (Thermofisher L3000015). A construct with a mammalian expression vector containing a PGK promoter driving the expression of MED1 fused to GFP was co-transfected in high MED1 conditions. 24 hours after transfection, cells were treated for 45 minutes with 4-Hydroxytamoxifen (Sigma-Aldrich H7904) reconstituted in DMSO. Following treatment, cells were fixed and immunofluorescence was performed with a MED1 antibody as described above. Cells were then imaged using the RPI Spinning Disk confocal microscope with a 100× objective.

For analysis of Lac array data comparing MED1 or HP1α tethered, a region of interest was called using the signal in the Lac array (561 channel). The average fluorescent signal for FLTX1 (488 channel) was then measured in the region of interest and divided by the average fluorescence in the region of interest at the Lac array. This value was then divided in the drug treated condition by the vehicle treated condition and all values were normalized to the HP1α condition. For analysis of Lac array data for MED1 overexpression, enrichment was calculated by dividing the average fluorescent signal for MED1 immunofluorescence at the region of interest, defined by the ER tethered at the lac array, by MED1 immunofluorescence signal at a random nuclear region. Enrichment of MED1 was plotted over each concentration of tamoxifen in wildtype or high MED1 conditions.

Western Blot

Cells were lysed in Cell Lytic M (Sigma-Aldrich C2978) with protease inhibitors (Roche, 11697498001). Lysate was run on a 3%-8% Tris-acetate gel or 10% Bis-Tris gel or 3-8% Bis-Tris gels at 80 V for ˜2 hrs, followed by 120 V until dye front reached the end of the gel. Protein was then wet transferred to a 0.45 μm PVDF membrane (Millipore, IPVH00010) in ice-cold transfer buffer (25 mM Tris, 192 mM glycine, 10% methanol) at 300 mA for 2 hours at 4° C. After transfer the membrane was blocked with 5% non-fat milk in TBS for 1 hour at room temperature, shaking. Membrane was then incubated with 1:1,000 of the indicated antibody (ER ab32063, MED1 ab64965) diluted in 5% non-fat milk in TBST and incubated overnight at 4° C., with shaking. In the morning, the membrane was washed three times with TBST for 5 minutes at room temperature shaking for each wash. Membrane was incubated with 1:5,000 secondary antibodies for 1 hr at RT and washed three times in TBST for 5 minutes. Membranes were developed with ECL substrate (Thermo Scientific, 34080) and imaged using a CCD camera or exposed using film or with high sensitivity ECL. Quantification of western blot was performed using BioRad image lab.

### REFERENCES

1. Y. Shin, C. P. Brangwynne, Liquid phase condensation in cell physiology and disease. Science. 357, eaaf4382 (2017).
2. S. F. Banani, H. O. Lee, A. A. Hyman, M. K. Rosen, Biomolecular condensates: organizers of cellular biochemistry. Nat. Rev. Mol. Cell Biol. 18, 285-298 (2017).
3. A. A. Hyman, C. A. Weber, F. Jülicher, Liquid-Liquid Phase Separation in Biology. Annu. Rev. Cell Dev. Biol. 30, 39-58 (2014).
4. R. J. Ries, S. Zaccara, P. Klein, A. Olarerin-George, S. Namkoong, B. F. Pickering, D. P. Patil, H. Kwak, J. H. Lee, S. R. Jaffrey, m6A enhances the phase separation potential of mRNA. Nature. 571, 424-428 (2019).
5. E. M. Langdon, A. S. Gladfelter, A New Lens for RNA Localization: Liquid-Liquid Phase Separation. Annu. Rev. Microbiol. 72, 255-271 (2018).
6. E. M. Langdon, Y. Qiu, A. Ghanbari Niaki, G. A. McLaughlin, C. A. Weidmann, T. M. Gerbich, J. A. Smith, J. M. Crutchley, C. M. Termini, K. M. Weeks, S. Myong, A. S. Gladfelter, mRNA structure determines specificity of a polyQ-driven phase separation. Science (80-.). 360, 922-927 (2018).
7. M.-T. Wei, Y.-C. Chang, S. F. Shimobayashi, Y. Shin, C. P. Brangwynne, Nucleated transcriptional condensates amplify gene expression. bioRxiv, 737387 (2019).
8. T. J. J. Nott, E. Petsalaki, P. Farber, D. Jervis, E. Fussner, A. Plochowietz, T. D. Craggs, D. P. P. Bazett-Jones, T. Pawson, J. D. D. Forman-Kay, A. J. J. Baldwin, Phase Transition of a Disordered Nuage Protein Generates Environmentally Responsive Membraneless Organelles. Mol. Cell. 57, 936-947 (2015).
9. T. J. Nott, T. D. Craggs, A. J. Baldwin, Membraneless organelles can melt nucleic acid duplexes and act as biomolecular filters. Nat. Chem. 8, 569-575 (2016).
10. B. R. Sabari, A. Dall'Agnese, A. Boija, I. A. Klein, E. L. Coffey, K. Shrinivas, B. J. Abraham, N. M. Hannett, A. V. Zamudio, J. C. Manteiga, C. H. Li, Y. E. Guo, D. S. Day, J. Schuijers, E. Vasile, S. Malik, D. Hnisz, I. L. Tong, I. I. Cisse, R. G. Roeder, P. A. Sharp, A. K. Chakraborty, R. A. Young, A. Dall'Agnese, A. Boija, I. A. Klein, E. L. Coffey, K. Shrinivas, B. J. Abraham, N. M. Hannett, A. V. Zamudio, J. C. Manteiga, C. H. Li, Y. E. Guo, D. S. Day, J. Schuijers, E. Vasile, S. Malik, D. Hnisz, T. I. Lee, I. I. Cisse, R. G. Roeder, P. A. Sharp, A. K. Chakraborty, R. A. Young, Coactivator condensation at super-enhancers links phase separation and gene control. Science (80-.). 361, eaar3958 (2018).
11. Y. E. Guo, J. C. Manteiga, J. E. Henninger, B. R. Sabari, A. Dall'Agnese, N. M. Hannett, J.-H. Spille, L. K. Afeyan, A. V. Zamudio, K. Shrinivas, B. J. Abraham, A. Boija, T.-M. Decker, J. K. Rimel, C. B. Fant, T. I. Lee, I. I. Cisse, P. A. Sharp, D. J. Taatjes, R. A. Young, Pol II phosphorylation regulates a switch between transcriptional and splicing condensates. Nature. 572, 543-548 (2019).
12. A. Boija, I. A. Klein, B. R. Sabari, A. Dall'Agnese, E. L. Coffey, A. V. Zamudio, C. H. Li, K. Shrinivas, J. C. Manteiga, N. M. Hannett, B. J. Abraham, L. K. Afeyan, Y. E. Guo, J. K. Rimel, C. B. Fant, J. Schuijers, T. I. Lee, D. J. Taatjes, R. A. Young, Transcription Factors Activate Genes through the Phase-Separation Capacity of Their Activation Domains. Cell. 175, 1842-1855.e16 (2018).
13. J. J. Bouchard, J. H. Otero, D. C. Scott, E. Szulc, E. W. Martin, N. Sabri, D. Granata, M. R. Marzahn, K. Lindorff-Larsen, X. Salvatella, B. A. Schulman, T. Mittag, Cancer Mutations of the Tumor Suppressor SPOP Disrupt the Formation of Active, Phase-Separated Compartments. Mol. Cell. 72, 19-36.e8 (2018).
14. K. Shrinivas, B. R. Sabari, E. L. Coffey, I. A. Klein, A. Boija, A. V Zamudio, J. Schuijers, N. M. Hannett, P. A. Sharp, R. A. Young, A. K. Chakraborty, Enhancer Features that Drive Formation of Transcriptional Condensates. Mol. Cell. 75, 549-561.e7 (2019).
15. E. P. Bentley, B. B. Frey, A. A. Deniz, Physical Chemistry of Cellular Liquid-Phase Separation. Chemistry. 25, 5600-5610 (2019).
16. M. Boehning, C. Dugast-Darzacq, M. Rankovic, A. S. Hansen, T. Yu, H. Marie-Nelly, D. T. McSwiggen, G. Kokic, G. M. Dailey, P. Cramer, X. Darzacq, M. Zweckstetter, RNA polymerase II clustering through carboxy-terminal domain phase separation. Nat. Struct. Mol. Biol. 25, 833-840 (2018).
17. P. Cramer, Organization and regulation of gene transcription. Nature. 573, 45-54 (2019).
18. S. Alberti, S. Saha, J. B. Woodruff, T. M. Franzmann, J. Wang, A. A. Hyman, A User's Guide for Phase Separation Assays with Purified Proteins. J. Mol. Biol. 430, 4806-4820 (2018).
19. D. Hnisz, K. Shrinivas, R. A. Young, A. K. Chakraborty, P. A. Sharp, Perspective A Phase Separation Model for Transcriptional Control. Cell. 169, 13-23 (2017).
20. Y. Chen, A. S. Belmont, Genome organization around nuclear speckles. Curr. Opin. Genet. Dev. 55, 91-99 (2019).
21. A. G. Larson, D. Elnatan, M. M. Keenen, M. J. Trnka, J. B. Johnston, A. L. Burlingame, D. A. Agard, S. Redding, G. J. Narlikar, Liquid droplet formation by HP1α suggests a role for phase separation in heterochromatin. Nature. 547, 236-240 (2017).
22. A. R. Strom, A. V. Emelyanov, M. Mir, D. V. Fyodorov, X. Darzacq, G. H. Karpen, Phase separation drives heterochromatin domain formation. Nature. 547, 241-245 (2017).
23. M. Feric, N. Vaidya, T. S. Harmon, D. M. Mitrea, L. Zhu, T. M. Richardson, R. W. Kriwacki, R. V. Pappu, C. P. Brangwynne, Coexisting Liquid Phases Underlie Nucleolar Subcompartments. Cell. 165, 1686-1697 (2016).
24. D. M. Mitrea, J. A. Cika, C. S. Guy, D. Ban, P. R. Banerjee, C. B. Stanley, A. Nourse, A. A. Deniz, R. W. Kriwacki, Nucleophosmin integrates within the nucleolus via multi-modal interactions with proteins displaying R-rich linear motifs and rRNA. Elife. 5 (2016), doi:10.7554/eLife.13571.
25. D. M. Mitrea, J. A. Cika, C. B. Stanley, A. Nourse, P. L. Onuchic, P. R. Banerjee, A. H. Phillips, C.-G. Park, A. A. Deniz, R. W. Kriwacki, Self-interaction of NPM1 modulates multiple mechanisms of liquid-liquid phase separation. Nat. Commun. 9, 842 (2018).
26. S. F. Banani, A. M. Rice, W. B. Peeples, Y. Lin, S. Jain, R. Parker, M. K. Rosen, Compositional Control of Phase-Separated Cellular Bodies. Cell. 166, 651-663 (2016).
27. A. Patel, H. O. Lee, L. Jawerth, S. Maharana, M. Jahnel, M. Y. Hein, S. Stoynov, J. Mahamid, S. Saha, T. M. Franzmann, A. Pozniakovski, I. Poser, N. Maghelli, L. A. Royer, M. Weigert, E. W. Myers, S. Grill, D. Drechsel, A. A. Hyman, S. Alberti, A Liquid-to-Solid Phase Transition of the ALS Protein FUS Accelerated by Disease Mutation. Cell. 162, 1066-1077 (2015).
28. S. Alberti, The wisdom of crowds: regulating cell function through condensed states of living matter. J. Cell Sci. 130, 2789-2796 (2017).
29. A. E. Posey, A. S. Holehouse, R. V. Pappu, in Methods in enzymology (2018; http://www.ncbi.nlm.nih.gov/pubmed/30471685), vol. 611, pp. 1-30.
30. Y. Shin, C. P. Brangwynne, Liquid phase condensation in cell physiology and disease. Science (80-.). 357, eaaf4382 (2017).
31. V. N. Uversky, Intrinsically disordered proteins in overcrowded milieu Membrane-less organelles, phase separation, and intrinsic disorder. Curr. Opin. Struct. Biol. 44, 18-30 (2017).
32. Y. S. Mao, B. Zhang, D. L. Spector, Biogenesis and function of nuclear bodies. Trends Genet. 27, 295-306 (2011).
33. D. Hnisz, B. J. Abraham, T. I. Lee, A. Lau, V. Saint-André, A. A. Sigova, H. A. Hoke, R. A. Young, XSuper-enhancers in the control of cell identity and disease. Cell. 155, 934-47 (2013).
34. Y. H. Chu, M. Sibrian-Vazquez, J. O. Escobedo, A. R. Phillips, D. T. Dickey, Q. Wang, M. Ralle, P. S. Steyger, R. M. Strongin, Systemic Delivery and Biodistribution of Cisplatin in Vivo. Mol. Pharm. 13, 2677-2682 (2016).
35. S. Vibet, K. Mahéo, J. Gore, P. Dubois, P. Bougnoux, I. Chourpa, Differential subcellular distribution of mitoxantrone in relation to chemosensitization in two human breast cancer cell lines. Drug Metab. Dispos. 35, 822-8 (2007).
36. P. J. Smith, H. R. Sykes, M. E. Fox, I. J. Furlong, Subcellular distribution of the anticancer drug mitoxantrone in human and drug-resistant murine cells analyzed by flow cytometry and confocal microscopy and its relationship to the induction of DNA damage. Cancer Res. 52, 4000-8 (1992).
37. N. Kwiatkowski, T. Zhang, P. B. Rahl, B. J. Abraham, J. Reddy, S. B. Ficarro, A. Dastur, A. Amzallag, S. Ramaswamy, B. Tesar, C. E. Jenkins, N. M. Hannett, D. McMillin, T. Sanda, T. Sim, N. D. Kim, T. Look, C. S. Mitsiades, A. P. Weng, J. R. Brown, C. H. Benes, J. A. Marto, R. A. Young, N. S. Gray, Targeting transcription regulation in cancer with a covalent CDK7 inhibitor. Nature. 511, 616-20 (2014).
38. J. Marrero-Alonso, A. Morales, B. Garcia Marrero, A. Boto, R. Marin, D. Cury, T. Gómez, L. Fernández-Pérez, F. Lahoz, M. Díaz, Unique SERM-like properties of the novel fluorescent tamoxifen derivative FLTX1. Eur. J. Pharm. Biopharm. 85, 898-910 (2013).
39. W.-K. Cho, J.-H. Spille, M. Hecht, C. Lee, C. Li, V. Grube, I. I. Cisse, Mediator and RNA polymerase II clusters associate in transcription-dependent condensates. Science.
40. M. J. Tilby, C. Johnson, R. J. Knox, J. Cordell, J. J. Roberts, C. J. Dean, Sensitive detection of DNA modifications induced by cisplatin and carboplatin in vitro and in vivo using a monoclonal antibody. Cancer Res. 51, 123-9 (1991).
41. X. Shu, X. Xiong, J. Song, C. He, C. Yi, Base-Resolution Analysis of Cisplatin-DNA Adducts at the Genome Scale. Angew. Chem. Int. Ed. Engl. 55, 14246-14249 (2016).
42. W. A. Whyte, D. A. Orlando, D. Hnisz, B. J. Abraham, C. Y. Lin, M. H. Kagey, P. B. Rahl, T. I. Lee, R. A. Young, Master transcription factors and mediator establish super-enhancers at key cell identity genes. Cell. 153, 307-319 (2013).
43. X. Rovira-Clave, S. Jiang, Y. Bai, G. L. Barlow, S. Bhate, A. Coskun, G. Han, B. Zhu, C.-M. Ho, C. Hitzman, S.-Y. Chen, F.-A. Bava, G. Nolan, Subcellular localization of drug distribution by super-resolution ion beam imaging. bioRxiv, 557603 (2019).
44. Y. Wang, T. Zhang, N. Kwiatkowski, B. J. Abraham, T. I. Lee, S. Xie, H. Yuzugullu, T. Von, H. Li, Z. Lin, D. G. Stover, E. Lim, Z. C. Wang, J. D. Iglehart, R. A. Young, N. S. Gray, J. J. Zhao, CDK7-dependent transcriptional addiction in triple-negative breast cancer. Cell. 163, 174-86 (2015).
45. M. R. Mansour, J. M. Abraham, L. Anders, A. Berezovskaya, A. Gutierrez, A. D. Durbin, J. Etchin, L. Lawton, S. E. Sallan, L. B. Silverman, M. L. Loh, S. P. Hunger, T. Sanda, R. A. Young, A. T. Look, B. J. Abraham, L. Anders, A. Berezovskaya, A. Gutierrez, A. D. Durbin, J. Etchin, L. Lawton, S. E. Sallan, L. B. Silverman, M. L. Loh, S. P. Hunger, T. Sanda, R. A. Young, A. T. Look, An Oncogenic Super-Enhancer Formed Through Somatic Mutation of a Noncoding Intergenic Element. Science (80-.). 346, 1373-1377 (2014).
46. J. E. Bradner, D. Hnisz, R. A. Young, Transcriptional Addiction in Cancer. Cell. 168, 629-643 (2017).361, 412-415 (2018).
47. J. Lovén, H. A. Hoke, C. Y. Lin, A. Lau, D. A. Orlando, C. R. Vakoc, J. E. Bradner, T. I. Lee, R. A. Young, Selective inhibition of tumor oncogenes by disruption of super-enhancers. Cell. 153, 320-334 (2013).
48. Z. Nie, G. Hu, G. Wei, K. Cui, A. Yamane, W. Resch, R. Wang, D. R. Green, L. Tessarollo, R. Casellas, K. Zhao, D. Levens, c-Myc Is a Universal Amplifier of Expressed Genes in Lymphocytes and Embryonic Stem Cells. Cell. 151, 68-79 (2012).
49. S. Dasari, P. Bernard Tchounwou, Cisplatin in cancer therapy: Molecular mechanisms of action. Eur. J. Pharmacol. 740 (2014), pp. 364-378.
50. D. Dubik, T. C. Dembinski, R. P. C. Shiu4, “Stimulation of c-myc Oncogene Expression Associated with Estrogen-induced Proliferation of Human Breast Cancer Cells1” (1987).
51. A. Nagalingam, M. Tighiouart, L. Ryden, L. Joseph, G. Landberg, N. K. Saxena, D. Sharma, Med1 plays a critical role in the development of tamoxifen resistance. Carcinogenesis. 33, 918-30 (2012).
52. S. W. Fanning, C. G. Mayne, V. Dharmarajan, K. E. Carlson, T. A. Martin, S. J. Novick, W. Toy, B. Green, S. Panchamukhi, B. S. Katzenellenbogen, E. Tajkhorshid, P. R. Griffin, Y. Shen, S. Chandarlapaty, J. A. Katzenellenbogen, G. L. Greene, Estrogen receptor alpha somatic mutations Y537S and D538G confer breast cancer endocrine resistance by stabilizing the activating function-2 binding conformation. Elife. 5 (2016), doi:10.7554/eLife.12792.
53. S. Hole, A. M. Pedersen, S. K. Hansen, J. Lundqvist, C. W. Yde, A. E. Lykkesfeldt, New cell culture model for aromatase inhibitor-resistant breast cancer shows sensitivity to fulvestrant treatment and cross-resistance between letrozole and exemestane. Int. J. Oncol. 46, 1481-1490 (2015).
54. L. Zhang, J. C. Er, H. Jiang, X. Li, Z. Luo, T. Ramezani, Y. Feng, M. K. Tang, Y. T. Chang, M. Vendrell, A highly selective fluorogenic probe for the detection and: In vivo imaging of Cu/Zn superoxide dismutase. Chem. Commun. 52, 9093-9096 (2016).
55. K. D. Meyer, A. J. Donner, M. T. Knuesel, A. G. York, J. M. Espinosa, and D. J. Taatjes, Cooperative activity of cdk8 and GCN5L within Mediator directs tandem phosphoacetylation of histone H3. EMBO J. 27, 1447-57 (2008).
56. B. Langmead, S. L. Salzberg, Fast gapped-read alignment with Bowtie 2. Nat. Methods. 9, 357-359 (2012).
57. W. A. Whyte, S. Bilodeau, D. A. Orlando, H. A. Hoke, G. M. Frampton, C. T. Foster, S. M. Cowley, R. A. Young, Enhancer decommissioning by LSD1 during embryonic stem cell differentiation. Nature. 482, 221-225 (2012).
58. M. Rubinstein, R. H. Colby, Polymer Phyics (Oxford University Press, 2003).
59. M. Mittasch, P. Gross, M. Nestler, A. W. Fritsch, C. Iserman, M. Kar, M. Munder, A. Voigt, S. Alberti, S. W. Grill, M. Kreysing, Non-invasive perturbations of intracellular flow reveal physical principles of cell organization. Nat. Cell Biol. 20, 344-351 (2018).
60. H. Zhao, P. H. Brown, P. Schuck, On the distribution of protein refractive index increments. Biophys. J. 100, 2309-2317 (2011).
61. C. W. Pak, M. Kosno, A. S. Holehouse, S. B. Padrick, A. Mittal, R. Ali, A. A. Yunus, D. R. Liu, R. V Pappu, M. K. Rosen, Sequence Determinants of Intracellular Phase Separation by Complex Coacervation of a Disordered Protein. Mol. Cell. 63, 72-85 (2016).
62. A. S. Holehouse, R. K. Das, J. N. Ahad, M. O. G. Richardson, R. V Pappu, CIDER: Resources to Analyze Sequence-Ensemble Relationships of Intrinsically Disordered Proteins. Biophys. J. 112, 16-21 (2017).
63. M.-T. Wei, S. Elbaum-Garfinkle, A. S. Holehouse, C. Chih-Hsiung Chen, M. Feric, C. B. Arnold, R. D. Priestley, R. V Pappu, C. P. Brangwynne, Phase behaviour of disordered proteins underlying low density and high permeability of liquid organelles. nature.com (2017), doi:10.1038/NCHEM.2803.
64. R. Delage-Mourroux, P. G. V. Martini, I. Choi, D. M. Kraichely, J. Hoeksema, B. S. Katzenellenbogen, Analysis of estrogen receptor interaction with a repressor of estrogen receptor activity (REA) and the regulation of estrogen receptor transcriptional activity by REA. J. Biol. Chem. 275, 35848-35856 (2000).
65. Broad Institute Cancer Cell Line Encyclopedia (CCLE), (available at https://portals.broadinstitute.org/ccle).
66. cBioPortal for Cancer Genomics, (available at https://www.cbioportal.org/).

1-105. (canceled)

106. A method of characterizing a first agent, comprising
i) contacting the first agent with a composition comprising a condensate, wherein the condensate comprises a second agent before contacting with the first agent, and
ii) measuring the ability of the first agent to cause eviction of the second agent from the condensate;
wherein the eviction of the second agent from the condensate characterizes the first agent as having one or more of following properties:
a) the first agent has higher affinity for a component of the condensate than the second agent,
b) the first agent can release the second agent from the condensate, wherein the second agent does not have a target component in the condensate,
c) the first agent has similar or higher condensate partition property than the second agent, or
d) the first agent and the second agent have similar structure;
wherein the first agent and the second agent are both small molecules.

107. The method of claim 106, wherein the first agent and/or the second agent comprises a detectable tag.

108. The method of claim 107, wherein the detectable tag is a fluorescent tag.

109. The method of claim 107, wherein the detectable tag does not affect the condensate partition property of the first agent and/or the second agent.

110. The method of claim 106, wherein the condensate comprises a target component for the second agent.

111. The method of claim 106, wherein the condensate does not comprise a target component for the second agent.

112. The method of claim 106, wherein the first agent is an isomer of the second agent.

113. The method of claim 106, wherein the composition comprising the condensate is a cellular composition comprising the condensate within a cell.

114. The method of claim 106, wherein the composition comprising the condensate does not comprise a cell.

115. The method of claim 106, wherein the ability of the first agent to cause eviction of the second agent from the condensate is measured by the amount of the second agent remaining in the condensate after contacting the composition with the first agent.

116. The method of claim 106, wherein the ability of the first agent to cause eviction of the second agent from the condensate is measured by the amount of the second agent outside of the condensate after contacting the composition with the first agent.

117. The method of claim 106, wherein the ability of the first agent to cause eviction of the second agent from the condensate is measured by the ratio of the second agent inside and outside of the condensate after contacting the composition with the first agent.

118. The method of claim 106, wherein measuring the ability of the first agent to cause eviction of the second agent from the condensate comprises use of a technique selected from one or more of Raman spectroscopy, spectrophotometry, mass spectrometry, nuclear magnetic resonance, chromatography, quantitative phase microscopy, fluorescent microscopy, and a spin down assay.

119. The method of claim 106, wherein the composition is contacted with an increasing amount of the first agent, and the eviction of the second agent from the condensate is measured continuously or at discrete intervals.

120. A method of reducing transcription of an oncogene, comprising modulating the composition of, dissolving, or disassociating a transcriptional condensate associated with the oncogene by contacting the transcriptional condensate with an agent.

121. A method of inhibiting transcription associated with a transcriptional condensate, comprising inhibiting the binding of a nuclear receptor having an LXXLL binding domain and associated with the transcriptional condensate to a cofactor having an LXXLL domain, wherein the binding is inhibited by contacting the transcriptional condensate with a peptide that binds to the LXXLL binding domain of the nuclear receptor, or the LXXLL domain of the cofactor.

122. A composition comprising a cell, wherein the cell comprises a first condensate comprising a first detectable label, and a second condensate comprising a second detectable label, wherein the first condensate and the second condensate are different condensate types selected from the group consisting of a transcriptional condensate, a super-enhancer condensate, a splicing speckle condensate, a heterochromatin condensate, and nucleolus, and wherein the first detectable label and the second detectable label are different.

123. A method of assessing whether differential expression of one or more condensate components by a cell resistant to an agent causes or contributes to the resistance, comprising:
i) providing an agent-resistant cell, or a condensate comprising a differential amount of a condensate component or fragment thereof that is differentially expressed in an agent-resistant cell, or a condensate comprising a mutant condensate component or fragment thereof corresponding to a mutant condensate component in an agent-resistant cell,
ii) contacting the agent-resistant cell or the condensate with the agent, and
iii) assessing localization, concentration, and/or therapeutic activity of the agent, and/or morphology, stability, and/or dissolution of the condensate, as compared to a control.

124. The method of claim 123, wherein the condensate is isolated from the agent-resistance cell.

125. The method of claim 123, wherein the control is any of below:
i) a corresponding cell not resistant to the agent,
ii) a corresponding condensate not comprising a differential amount of the condensate component or fragment thereof,
iii) a corresponding condensate not comprising the mutant condensate component or fragment thereof,
iv) the morphology, stability, and/or dissolution of a corresponding condensate not contacted with the agent,
v) the agent-resistant cell or the condensate contacted with a second agent.
